(19) United States
(12) Patent Application Publication
(10) Pub. No.: US 2022/0265719 A1
(43) Pub. Date: Aug. 25, 2022

(54) IMMUNOTHERAPIES

(71) Applicant: Kite Pharma, Inc., Santa Monica, CA (US)

(72) Inventors: Adrian Bot, Beverly Hills, CA (US); Szu-Ting Chou, Los Angeles, CA (US); Vicki Plaks, Santa Monica, CA (US); Soumya Poddar, Los Angeles, CA (US); John Rossi, Newbury Park, CA (US)

(21) Appl. No.: 17/675,294

(22) Filed: Feb. 18, 2022

Related U.S. Application Data
(60) Provisional application No. 63/274,342, filed on Nov. 1, 2021.
(60) Provisional application No. 63/250,634, filed on Sep. 30, 2021.
(60) Provisional application No. 63/227,733, filed on Jul. 30, 2021.
(60) Provisional application No. 63/215,838, filed on Jun. 28, 2021.
(60) Provisional application No. 63/210,962, filed on Jun. 15, 2021.
(60) Provisional application No. 63/196,620, filed on Jun. 3, 2021.
(60) Provisional application No. 63/151,710, filed on Feb. 20, 2021.

(51) Int. Cl. A61K 35/17; A61P 35/00; A61K 45/06; C07K 16/28

(52) U.S. Cl. CPC A61K 35/17; A61P 35/00; A61K 45/06; C07K 16/2845; C07K 16/2896; C07K 16/2815; C07K 16/283; C07K 2317/622; C07K 2319/03; C07K 2317/24; C07K 2319/33

(57) ABSTRACT

The disclosure relates to methods of diagnosis and prognosis, compositions for immunotherapies, methods of improving said compositions, and immunotherapies using the same (e.g., T cells, non-T cells, TCR-based therapies, CAR-based therapies, bispecific T-cell engagers (BiTEs), and/or immune checkpoint blockade).

### CROSS REFERENCE TO RELATED APPLICATIONS

This application claims priority to U.S. Provisional Patent Application No. 63/151,710 filed on Feb. 20, 2021, U.S. Provisional Patent Application No. 63/196,620 filed on Jun. 3, 2021, U.S. Provisional Patent Application No. 63/210,962 filed on Jun. 15, 2021, U.S. Provisional Patent Application No. 63/215,838 filed on Jun. 28, 2021, U.S. Provisional Patent Application No. 63/227,733 filed on Jul. 30, 2021, U.S. Provisional Patent Application No. 63/250,634 filed on Sep. 30, 2021, and U.S. Provisional Patent Application No. 63/274,342 filed on Nov. 1, 2021, each of which is hereby incorporated by reference in its entirety.

### FIELD

The disclosure relates to methods of diagnosis and prognosis, compositions for immunotherapies, methods of improving said compositions, and immunotherapies using the same.

### BACKGROUND

Human cancers are by their nature comprised of normal cells that have undergone a genetic or epigenetic conversion to become abnormal cancer cells. In doing so, cancer cells begin to express proteins (including, but not limited to, antigens) that are distinct from those expressed by normal cells. These aberrant tumor antigens may be used by the body's innate immune system to specifically target and kill cancer cells. However, cancer cells employ various mechanisms to prevent immune cells, such as T and B lymphocytes, from successfully targeting cancer cells.

Human T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. For example, chimeric antigen receptors (CARs) and T Cell Receptors (TCRs), which comprise binding domains capable of interacting with a particular tumor antigen, allow T cells to target and kill cancer cells that express the particular tumor antigen. However, a major obstacle for adequate activity of CAR-T cells is the hostile tumor microenvironment that is comprised of immunosuppressive modulators.

There is a need to understand how attributes of CAR-positive T cells, TCR-positive T cells and other cell-based immunotherapies, patients' immunological status, and the tumor microenvironment correlate with clinical outcomes.

### SUMMARY

It is to be understood that the disclosure is not limited in its application to the details set forth in the following embodiments, claims, description and figures. The disclosure is capable of other embodiments and of being practiced or carried out in numerous other ways.

Provided herein are immunotherapies (e.g., T cells, non-T cells, TCR-based therapies, CAR-based therapies, bispecific T-cell engagers (BiTEs), and/or immune checkpoint blockade), including methods and uses of cells (e.g., engineered T cells) and/or compositions thereof, for the treatment of subjects having a disease or condition, which generally is or includes a cancer or a tumor, such as a leukemia or a lymphoma. In some aspects, the methods and uses provide for or achieve improved response and/or more durable responses or efficacy and/or a reduced risk of toxicity or other side effects, in subjects treated with some methods, as compared to certain alternative methods. In some embodiments, the methods comprise the administration of specified numbers or relative numbers of the engineered cells, the administration of defined ratios of particular types of the cells, treatment of particular patient populations, such as those having a particular risk profile, staging, and/or prior treatment history, administration of additional therapeutic agents and/or combinations thereof.

Also provided are methods that involve assessing particular parameters, e.g., expression of specific biomarkers or analytes, that can be correlated with an outcome, such as a therapeutic outcome, including a response, such as a complete response (CR) or a partial response (PR); or a safety outcome, such as a development of a toxicity, for example, neurotoxicity or CRS, after administration of a cell therapy. Also provided are methods to assess the likelihood of response and/or likelihood of risk of toxicity, based on assessment of the parameters, such as expression of biomarkers or analytes in the patient and in the tumor microenvironment.

In one embodiment, the disclosure provides that myeloid associated gene signature is upregulated in relapsed and nonresponders compared with ongoing responders. In one embodiment, the disclosure provides that patients with higher ARG2 expression (determined by the median of 30 patients) in pretreatment tumors have worse overall and progression free survival than those with lower ARG2 expression. The boxplots show ongoing responders expressing lower level of ARG2 in pretreatment tumor than relapsed and/or non-responders. In one embodiment, the disclosure provides that patients with higher TREM2 expression (determined by the median of 30 patients) in pretreatment tumors have worse overall and progression free survival than those with lower TREM2 expression. The boxplots show ongoing responders expressing lower level of TREM2 in pretreatment tumor than relapsed and/or non-responders. In one embodiment, the disclosure provides that patients with higher IL8 expression (determined by the median of 30 patients) in pretreatment tumors have worse overall and progression free survival than those with lower IL8 expression. The boxplots show ongoing responders expressing lower level of IL8 pretreatment tumor than relapsed and/or non-responders. In one embodiment, the disclosure provides that patients with higher IL13 expression (determined by the median of 30 patients) in pretreatment tumors have worse overall and progression free survival than those with lower IL13 expression. The boxplots show ongoing responders expressing lower level of IL13 pretreatment tumor than relapsed and/or non-responders. In one embodiment, the disclosure provides that patients with higher CCL20 expression (determined by the median of 30 patients) in pretreatment tumors have worse overall and progression free survival than those with lower CCL20 expression. The boxplots show ongoing responders expressing lower level of CCL20 in pretreatment tumor than relapsed and/or non-responders. In one embodiment, the disclosure provides that patients in durable response show lower expression of ARG2 and TREM2 while relapsed and nonresponders show higher expression of ARG2 and TREM2, particularly in patients with higher baseline tumor burden. In one embodiment, the disclosure provides that CAR-T peak expansion is positively associated with ongoing response, particularly in patients with large baseline tumor burden. In one embodiment, the disclosure provides that the ratio of T/Myeloid Index is positively associated with ongoing response, particularly in patients with large baseline tumor burden. In one embodiment, the disclosure provides that CAR-T peak expansion is positively associated with T cell index and T/Myeloid ratio. In one embodiment, the disclosure provides that peak level of CAR-T cells relative to baseline tumor burden is positively associated with T cell index and T/Myeloid ratio.

The following are non-limiting embodiments of the disclosure.

An embodiment of the disclosure relates to a method for treating a malignancy in a patient including: assessing a level of myeloid inflammation in a tumor of the patient; determining whether the patient should be administered an effective dose of engineered lymphocytes, or an effective dose of engineered lymphocytes and a combination therapy at least in part from the level of myeloid inflammation; and administering the effective dose of engineered lymphocytes, or the effective dose of engineered lymphocytes and the combination therapy based on the determining step. In such an embodiment, the patient is administered the effective dose of engineered lymphocytes if the level of myeloid inflammation is below a reference value, and where the patient is administered the effective dose of engineered lymphocytes and the combination therapy if the level of myeloid inflammation is above the reference value.

An embodiment of the disclosure related to the method above, where assessing the level of myeloid inflammation in a tumor of the patient includes measuring a gene expression level of at least one gene selected from the group consisting of Arginase 2 (ARG2), triggering receptor expressed on myeloid cells 2 (TREM2), interleukin 8 (IL8), interleukin 13 (IL13), Complement C8 Gamma Chain (C8G), C-C Motif Chemokine Ligand 20 (CCL20), Interferon Lambda 2 (IFNL2), Oncostatin M (OSM), interleukin 11 receptor alpha (IL11RA), C-C Motif Chemokine Ligand 11 (CCL11), Melanoma Cell Adhesion Molecule (MCAM), Prostaglandin D2 Receptor 2 (PTGDR2), and C-C Motif Chemokine Ligand 16 (CCL16), and where the level of myeloid inflammation is related to the level of gene expression. An embodiment of the disclosure is related to a method for treating a malignancy in a patient including: assessing a level of myeloid inflammation in a tumor of the patient by measuring a gene expression level of at least one gene selected from the group consisting of ARG2, TREM2, IL8, IL13, C8G, CCL20, IFNL2, OSM, IL11RA, CCL11, MCAM, PTGDR2, and CCL16; determining whether the patient should be administered an effective dose of engineered lymphocytes, or an effective dose of engineered lymphocytes and a combination therapy at least in part from the measuring the gene expression level of at least one gene; and administering the effective dose of engineered lymphocytes, or the effective dose of engineered lymphocytes and the combination therapy based on the determining step. In such an embodiment, the patient is administered the effective dose of engineered lymphocytes if the gene expression level of the at least one gene is below a predetermined level, and the patient is administered the effective dose of engineered lymphocytes and the combination therapy if the gene expression level of the at least one gene is above the predetermined level.

An embodiment of the disclosure is related to the method above, where the predetermined level is a median expression level of the at least one gene in a representative tumor population.

An embodiment of the disclosure related to the method above, where the combination therapy includes at least one of an agent that enhances T-cell proliferation, and an agent that reduces a myeloid population in the tumor.

An embodiment of the disclosure related to the method above, where the at least one agent includes an anti-CD47 antagonist, a stimulator of interferon genes (STING) agonist, an ARG1/2 inhibitor, a CD73xTGFβ mAb, a CD40 agonist, a FLT3 agonist, a CSF/CSF1R inhibitor, an IDO1 inhibitor, a TLR agonist, a PD-1 inhibitor, an immunomodulatory imide drug, a CD20xCD3 bispecific antibody, an agent that targets an epigenetic landscape within the tumor or a T-cell costimulatory agonist, or combinations thereof.

An embodiment of the disclosure related to the method above, further including: determining a tumor burden in the patient; and administering the effective dose of engineered lymphocytes, or the effective dose of engineered lymphocytes and the combination therapy based on the determining the tumor burden in the patient. In such an embodiment, the patient is administered the effective dose of engineered lymphocytes if the tumor burden is below a reference tumor burden value, and where the patient is administered the effective dose of engineered lymphocytes and the combination therapy if the tumor burden is above the reference tumor burden value.

An embodiment of the disclosure related to the method above, where the reference tumor burden value includes a baseline tumor burden (SPD) of greater than 2500 mm² or a tumor metabolic volume above a median for a representative tumor population.

An embodiment of the disclosure related to the method above, where the combination therapy includes at least one of an agent that enhances T-cell proliferation, and an agent that reduces a myeloid population in the tumor.

An embodiment of the disclosure related to the method above, further including: quantifying a tumor myeloid cell density in the tumor; and administering the effective dose of engineered lymphocytes, or the effective dose of engineered lymphocytes and the combination therapy based on the quantifying a tumor myeloid cell density in the tumor. In such an embodiment, the patient is administered the effective dose of engineered lymphocytes if the tumor myeloid cell density in the tumor is below a predetermined myeloid cell density level, and the patient is administered the effective dose of engineered lymphocytes and the combination therapy if the tumor myeloid cell density in the tumor is above the predetermined myeloid cell density level.

An embodiment of the disclosure related to the method above, where the tumor myeloid cell density is quantified including measuring levels of CD14+ cells, CD68+ cells, CD68+CD163+ cells, CD68+CD206+ cells, CD11b+CD15+CD14− LOX-1+ cells, or CD11b+CD15− CD14+ S100A9+ CD68− cells.

An embodiment of the disclosure related to the method above, where the reference value is a median value for a representative tumor population.

An embodiment of the disclosure related to the method above, where the engineered lymphocytes are chimeric antigen receptor T-cells.

An embodiment of the disclosure related to the method above, where the effective dose of engineered lymphocytes or the effective dose of engineered lymphocytes and a combination therapy are administered as a first line therapy or as a second line therapy.

An embodiment of the disclosure related to the method above, where the malignancy is a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder, monoclonal gammapathy of undetermined significance (MGUS), plasmacytomas, systemic amyloid light chain amyloidosis, POEMS syndrome, head and neck cancers, cervical cancers, ovarian cancers, non-small cell lung carcinomas, hepatocellular carcinomas, prostate cancers, breast cancers, or a combination thereof.

An embodiment of the disclosure related to a method of predicting a clinical efficacy of an immunotherapy in a patient in need thereof including: assessing a level of myeloid inflammation in a tumor of the patient including measuring a gene expression level of at least one gene selected from the group consisting of ARG2, TREM2, IL8, IL13, CBG, CCL20, IFNL2, OSM, IL11RA, CCL11, MCAM, PTGDR2, and CCL16; and determining a likelihood of clinical efficacy of the immunotherapy in the patient at least in part from the gene expression level. In such an embodiment, the likelihood of clinical efficacy is inversely related to the gene expression level.

An embodiment of the disclosure related to the method above, further including measuring a ratio of activated T-cells to suppressive myeloid cells in the tumor. In such an embodiment, the likelihood of clinical efficacy is related to the ratio of activated T cells to suppressive myeloid cells in the tumor such that a higher ratio of an activated T cells index to a suppressive myeloid cells index in the tumor is indicative of an increased likelihood of clinical efficacy.

An embodiment of the disclosure related to the method above, where the activated T-cell index is determined including measuring a gene expression level of one or more of CD3D, CD8A, CTLA4, and TIGIT in the tumor.

An embodiment of the disclosure related to the method above, further including determining a tumor burden of the patient. In such an embodiment, the likelihood of clinical efficacy is related to the tumor burden of the patient such that a tumor burden above a reference tumor burden value is indicative of a reduced likelihood of clinical efficacy and a tumor burden below a reference tumor burden value is indicative of an increased likelihood of clinical efficacy, and where the reference tumor burden is 2500 mm².

An embodiment of the disclosure related to the method above, where the clinical efficacy is assessed including evaluating a complete response rate, an objective response rate, an ongoing response rate, a median durability of response, a median progression-free survival, a median overall survival, or any combination thereof.

An embodiment of the disclosure related to a method of predicting a suppressive tumor microenvironment (TME) in a patient including: assessing a level of myeloid inflammation in a tumor of the patient including measuring a gene expression level of at least one gene selected from the group consisting of ARG2, TREM2, IL8, IL13, C8G, CCL20, IFNL2, OSM, IL11RA, CCL11, MCAM, PTGDR2, and CCL16; and determining a level of the tumor suppressive microenvironment at least in part from the gene expression level. In such an embodiment, the level of the tumor suppressive microenvironment is related to the gene expression level such that a higher gene expression level is indicative of a higher suppressive tumor microenvironment.

An embodiment of the disclosure related to the method above, further including: quantifying a tumor myeloid cell density in the tumor. In such an embodiment, the level of the tumor suppressive microenvironment is related to the tumor myeloid cell density, such that a higher tumor myeloid cell density is indicative of a higher suppressive tumor microenvironment.

An embodiment of the disclosure is related to the method above, further including measuring a ratio of activated T-cells to suppressive myeloid cells in the tumor, where the level of the tumor suppressive microenvironment is related to the ratio of activated T-cells to suppressive myeloid cells in the tumor, such that a lower ratio of an activated T-cells index to a suppressive myeloid cells index in the tumor is indicative of a higher suppressive tumor microenvironment.

Additional non-limiting embodiments include:
1. A method of predicting a suppressive tumor microenvironment (TME) induced by myeloid cells in a tumor of a cancer patient and/or predicting the clinical efficacy of immunotherapy for treating the patient's cancer, the method comprising quantifying myeloid inflammation in the TME in the tumor; wherein:
   (i) the higher the tumor level of myeloid inflammation, the more suppressive the tumor microenvironment is; and
   (ii) the higher the level of tumor myeloid inflammation the lower the clinical efficacy of the immunotherapy.
2. The method of embodiment 1, wherein the tumor myeloid inflammation level is estimated by measuring the gene expression level of one or more ofARG2, TREM2, IL8, IL13, C8G, CCL20, IFNL2, OSM, ILIIRA, CCL11, MCAM, PTGDR2, and CCL16 in the tumor; wherein the higher expression of one or more of these genes, the higher the myeloid inflammation level.
3. A method of treating cancer with immunotherapy in a cancer patient in need thereof, wherein the patient is selected for treatment when the level of myeloid inflammation in a patient's tumor microenvironment, as measured by the gene expression level of one or more ofARG2, TREM2, IL8, IL13, C8G, CCL20, IFNL2, OSM, IL11RA, CCL11, MCAM, PTGDR2, and CCL16:
   (i) below the median for a representative tumor population; and/or
   (ii) within the following values for each of the respective genes: 0-27 (ARG2), 0-10 (TREM2), 0-42 (IL8), 0-9 (IL13), 0-11 (C8G), 0-1 (CCL20), 0-11 (IFNL2), 0-8 (OSM), 0-77 (IL11RA), 0-27 (CCL11), 59-132 (MCAM), 0-1 (PTGDR2), and 0-1 (CCL16), preferably as measured by Nanostring, plus or minus standard deviation or plus or minus 20%.
4. A method to stratify patients having a tumor with a TME for combination therapy including immunotherapy, the method comprising administering immunotherapy in combination with an agent that enhances the proliferation of T cells, wherein the combination therapy enhances the proliferation of the T cells and/or wherein the combination therapy reduces the suppressive myeloid population in the TME, wherein the patient is selected for combination therapy when the patient has high tumor burden, low T-cell to suppressive myeloid cell markers (T/M) ratio, and/or high level of TME myeloid inflammation, preferably wherein the TME myeloid inflammation level is estimated by measuring the gene expression level of one or more of ARG2, TREM2, IL8, IL13, C8G, CCL20, IFNL2, OSM, IL11RA, CCL11, MCAM, PTGDR2, and CCL16 in the tumor; optionally, wherein agent is administered to the patient prior to CAR-T infusion, at the peak of CAR-T expansion (e.g., Day 7-14 post infusion), and/or after peak CAR-T expansion (e.g., Day 14-28).
5. The method of embodiment 4, wherein the agent is selected from anti-CD47 antagonist (e.g., magrolimab), a STING agonist (e.g., GSK3745417), an ARG1/2 inhibitor (e.g., INCB001158), a CD73xTGFβ mAb (e.g., GS-1423), a CD40 agonist (e.g., Selicrelumab), a FLT3 agonist (e.g., GS3583), a CSF/CSF1R inhibitor (e.g., Pexidartinib), an IDO1 inhibitor (e.g., epacadostat), a TLR agonist (e.g., GS9620), a PD-1 inhibitor (e.g., pembrolizumab), Immunomodulatory imide drug, (e.g., lenalidomide), CD20xCD3 bispecific antibody (e.g., epcoritamab), and T Cell costimulatory agonists (e.g., utoliumab).
6. A method of treating a tumor in a subject with a high tumor burden, wherein the high tumor burden in the subject is reduced by administering one or more agents or treatments that result in a favorable immune TME (e.g., higher T/M ratio and/or lower TME myeloid inflammation) and/or by increasing CAR T cell expansion.
7. The method of embodiment 6, wherein the immune TME is favorable with respect to favorable for treatment with immunotherapy.
8. The method of any one of embodiments 6 and 7, wherein the subject has a high tumor burden (as assessed by SPD and/or tumor metabolic volume) when the baseline tumor burden (SPD) is greater than 2500, 3000, 3500, or 4000, preferably greater than 3000 mm² and/or the tumor metabolic volume is above the median for a representative tumor population (e.g., above 100, or above 150 ml).
9. The method of any one of embodiments 6 through 8, wherein the immune TME is favorable when the TME presents reduced suppressive myeloid cell activity (e.g., low ARG2 and TREM2 expression) and increased T cell/Myeloid cell ratio (e.g., 1-4), relative to those values prior to administration of the agent.
10. The method of embodiment 9, wherein the reduced suppressive myeloid activity is present when the TME shows low ARG2 and/or low TREM2 expression, preferably wherein low means below the median for a representative tumor population.
11. The method of embodiment 9, wherein ARG2 and/or TREM2 gene expression are low when the expression levels are between 0 and 27, as measured by NanoString, plus or minus standard deviation or plus or minus 20%.
12. The method of any one of embodiments 6 through 11, wherein the agent reduces tumor myeloid suppressive activity and/or reduces tumor myeloid cell density.
13. The method of embodiment 12, wherein tumor myeloid cell density is quantified by measuring CD14+ cells, CD68+ cells, CD68+CD163+ cells, CD68+CD206+ cells, CD11b+CD15+CD14− LOX-1+ cells, and/or CD11b+CD15− CD14+S100A9+CD68− cells by immunohistochemistry in a tumor biopsy.
14. The method of any one of embodiments 6 through 13, wherein the agent is selected from an anti-CD47 antagonist (e.g., magrolimab), a STING agonist (e.g., GSK3745417), an ARG1/2 inhibitor (e.g., INCB001158), a CD73xTGFβ mAb (e.g., GS-1423), a CD40 agonist (e.g., Selicrelumab), a FLT3 agonist (e.g., GS3583), a CSF/CSF1R inhibitor (e.g., Pexidartinib), an IDO1 inhibitor (e.g., epacadostat), a TLR agonist (e.g., GS9620) and combinations of the same.
15. The method of any one of embodiments 6 through 13, wherein the agent or treatment is selected from low dose radiation, promotion of T cell activity through checkpoint blockade, T cell agonists (e.g., pembrolizumab, lenalidomide, epcoritamab, and utoliumab), and combinations of the same.
16. The method of any one of embodiments 6 through 15, wherein the agent or treatment is administered prior to, during, and/or after immunotherapy.
17. The method of embodiment 16, wherein the immunotherapy is CAR T-cell therapy.
18. The method of embodiment 17, wherein CAR T cell expansion is increased relative to representative CAR T cell expansion levels without the agent or treatment.
19. A method for quantifying TME myeloid inflammation comprising measuring gene expression of one or more of ARG2, TREM2, IL8, IL13, C8G, CCL20, IFNL2, OSM, IL11RA, CCL11, MCAM PTGDR2, and CCL16 in the tumor, wherein the higher the expression of one or more of these genes, the higher the TME myeloid inflammation level.
20. A method of predicting response/clinical efficacy of immunotherapy of a tumor in a subject in need thereof, comprising measuring gene expression of one or more of ARG2, TREM2, IL8, IL13, C8G, CCL20, IFNL2, OSM, IL11RA, CCL11, MCAM PTGDR2, and CCL16 in the TME, wherein the higher the expression of one or more of these genes the lower the clinical efficacy.
21. A method of predicting response/clinical efficacy to immunotherapy in a patient with high tumor burden, comprising measuring the ratio of activated T cells to suppressive myeloid cells in the TME prior to immunotherapy, the T/M ratio, wherein the higher the ratio of activated T cells index to suppressive myeloid cells index in the TME, the better the response.
22. The method of embodiment 21, wherein T cell activation is measured by measuring the gene expression levels of one or more of CD3D, CD8A, CTLA4, and TIGIT in the TME, preferably wherein the activated T cell index is estimated as the root mean square of CD3D, CD8A, CTLA4, TIGIT gene expression levels, preferably by NanoString.
23. The method of embodiment 21 or 22, wherein the myeloid index is estimated as root mean square of ARG2, TREM2 gene expression levels, preferably by NanoString.
24. The method of embodiment 22 or 23, wherein the T/M ratio is estimated as Log 2((T-cell Index+1)/(Myeloid Index+1)).
25. The method of any one of embodiments 21 through 24, wherein when the ratio of activated T cells to suppressive myeloid cells in the TME is low, the patient is administered myeloid conditioning prior to immunotherapy, preferably wherein low means below the median for a representative tumor population.
26. The method of embodiment 25, wherein a low TME ratio of activated T cells to suppressive myeloid cells (T/M) is a ratio within 1-4.
27. The method of embodiments 25 or 26, wherein myeloid conditioning comprises inhibition of suppressive myeloid TME.
28. The method of embodiment 27, wherein myeloid conditioning is achieved by administration of an anti-CD47 antagonist (e.g., magrolimab), a STING agonist (e.g., GSK3745417), an ARG1/2 inhibitor (e.g., INCB001158), a CD73xTGFβ mAb (e.g., GS-1423), a CD40 agonist (e.g., Selicrelumab), a FLT3 agonist (e.g., GS3583), a CSF/CSF1R inhibitor (e.g., Pexidartinib), an IDO1 inhibitor (e.g., epacadostat), a TLR agonist (e.g., GS9620), or combinations of the same.
29. The method of any one of embodiments 21 through 28, wherein the tumor burden is high if the baseline tumor burden (SPD) is above the median for a representative tumor population, optionally from 2000 to 3700 mm².
30. A method of predicting CAR or TCR peak T cell expansion and or CAR or TCR peak T cell expansion normalized by tumor burden, the method comprising measuring T/M, wherein the higher the T/M ratio the higher the CAR or TCR peak T cell expansion normalized by tumor burden.
31. The method of any one of embodiments 1 through 30, wherein the response/clinical efficacy is assessed by complete response rates, objective response rates, ongoing response rates, median durability of response, median PFS, and/or median OS.
32. The method of any one of embodiments 1 through 31, wherein the immunotherapy is CAR T cell therapy, TCR T cell therapy, tumor infiltrating lymphocytes (TIL) cell therapy, and/or administration of immune checkpoint inhibitors.
33. The method of embodiment 32, wherein the immune checkpoint inhibitor is selected from agents that block immune checkpoint receptors on the surface of T cells, such as cytotoxic T lymphocyte antigen 4 (CTLA-4), lymphocyte activation gene-3 (LAG-3), T-cell immunoglobulin mucin domain 3 (TIM-3), B- and T-lymphocyte attenuator (BTLA), T-cell immunoglobulin and T-cell immunoreceptor tyrosine-based inhibitory motif (ITIM) domain, and programmed cell death 1 (PD-1/PDL-1).
34. The method of embodiment 33, comprising administering to the patient an agonist of 41BB, OX40, and/or TLR.
35. The method of any one of embodiments 1 through 34, wherein the agent, combination agent and/or treatment, are administered before, during, and/or after immunotherapy.
36. The method of any one of embodiments 1 through 35, wherein the immunotherapy is autologous or allogeneic.
37. The method of any one of embodiments 1 through 36, wherein the immunotherapy is CAR T or TCR T cell therapy that recognizes a target antigen.
38. The method of embodiment 37, wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gp120), GPC3 (Glypican 3), as well as any derivate or variant of these antigens.
39. The method of any one of embodiments 1 through 38, wherein the cancer/tumor is selected from a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof
40. The method of embodiment 39, wherein the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.
41. The method of any one of embodiments 1 through 40, wherein the immunotherapy is selected from axicabtagene ciloleucel, brexucabtagene autoleucel, tisagenlecleucel, lisocabtagene maraleucel, and bb2121.

### BRIEF DESCRIPTION OF THE DRAWINGS

FIG. 1. Volcano plot of differentially expressed genes comparing ongoing responders with relapsed and nonresponders. Fold change was determined by the ratio of median value in each ongoing response group, and the p-value was derived from Wilcoxon test. A small constant, 1, was added to the medians to avoid zero in logarithmic transformation. Top differentially expressed gene in relapsed and nonresponder group, including ARG2, TREM2, IL8, C8G, and MASP2, are related to myeloid inflammation. Gene counts are normalized using a ratio of the expression value to the geometric mean of all housekeeping genes on the panel. Housekeeper-normalized gene counts are additionally normalized using a panel standard run on the same cartridge as the observed data.

FIG. 2. Overall and progression-free survival curves of CLINICAL TRIAL-1 subjects grouped by ARG2 gene counts. Kaplan-Meier overall and progression-free survival curves with a median cut-off selection for ARG2 gene counts in pretreatment tumor samples with significance determined by the Log-Rank test. The boxplots show ARG2 gene counts by ongoing response groups. Nonparametric Wilcoxon tests and Kruskal-Wallis tests are conducted for comparisons of 2 or 3 groups, respectively.

FIG. 3. Overall and progression-free survival curves of CLINICAL TRIAL-1 subjects grouped by TREM2 gene counts. Kaplan-Meier overall and progression-free survival curves with a median cut-off selection for TREM2 gene counts in pretreatment tumor samples with significance determined by the Log-Rank test. The boxplots show TREM2 gene counts by ongoing response groups. Nonparametric Wilcoxon tests and Kruskal-Wallis tests are conducted for comparisons of 2 or 3 groups, respectively.

FIG. 4. Overall and progression-free survival curves of CLINICAL TRIAL-1 subjects grouped by IL8 gene counts. Kaplan-Meier overall progression-free survival curves with a median cut-off selection for IL8 gene counts in pretreatment tumor samples with significance determined by the Log-Rank test. The boxplots show IL8 gene counts by ongoing response groups. Nonparametric Wilcoxon tests and Kruskal-Wallis tests are conducted for comparisons of 2 or 3 groups, respectively.

FIG. 5. Overall and progression-free survival curves of CLINICAL TRIAL-1 subjects grouped by IL13 gene counts. Kaplan-Meier overall and progression-free survival curves with a median cut-off selection for IL13 gene counts in pretreatment tumor samples with significance determined by the Log-Rank test. The boxplots show IL13 gene counts by ongoing response groups. Nonparametric Wilcoxon tests and Kruskal-Wallis tests are conducted for comparisons of 2 or 3 groups, respectively.

FIG. 6. Overall and progression-free survival curve of CLINICAL TRIAL-1 subjects grouped by CCL20 gene counts. Kaplan-Meier overall and progression-free survival curves with a median cut-off selection for CCL20 gene counts in pretreatment tumor samples with significance determined by the Log-Rank test. The boxplots show CCL20 gene counts by ongoing response groups. Nonparametric Wilcoxon tests and Kruskal-Wallis tests are conducted for comparisons of 2 or 3 groups, respectively.

FIG. 7. Associations between pretreatment T cell and Myeloid cell gene signature with ongoing response within patients with high (SPD^{hi}) (above the median level for a representative tumor population) or low (SPD^{low}) (below the median level for a representative tumor population) baseline tumor burden. Values in red are representative of a value greater the mean expression while those in blue are representative of a value less than mean expression of the corresponding gene. Total number of infused CD8 (NCD8), total number of infused naïve products (NNV), peak level of CAR-T cells and its value relative to baseline tumor burden (CAR-T peak/SPD) are included as a comparison.

FIG. 8. Association between peak CAR-T levels (cells/μL) by ongoing response groups within patients with high (SPD^{hi}) or low (SPD^{low}) baseline tumor burden. Ongoing responders are shown in green, relapsed patients are shown in orange, and non-responders are shown in blue. Nonparametric Kruskal-Wallis tests are conducted for comparisons of 3 groups.

FIG. 9. Ratio of T cell to myeloid inflammation by ongoing response groups within patients with high (SPD^{hi}) or low (SPD^{low}) baseline tumor burden. Selected genes were used to derive T cell (CD3D, CD8A, CTLA4, TIGIT) and myeloid inflammation (ARG2 and TREM2) indices. Ongoing responders are shown in green, relapsed patients are shown in orange, and non-responders are shown in blue. Nonparametric Kruskal-Wallis tests are conducted for comparisons of 3 groups.

FIG. 10. Associations between peak level of CAR-T cells with T cell, myeloid inflammation indices, and ratio of T cell to myeloid inflammation. Spearman rank coefficient (R) and p values are shown.

FIG. 11. Associations between peak levels of CAR-T cells relative to baseline tumor burden with T cell, myeloid inflammation indices, and ratio of T cell to myeloid inflammation. Spearman rank coefficient (R) and p values are shown.

FIG. 12. Genes negatively associated with ongoing response were positively associated with the myeloid population in the TME. Data are included for 12 patients from ZUMA-1 Cohorts 1-3 with evaluable samples for both gene expression analyses and multiplex immunohistochemistry. The genes presented in the heatmap were selected based on findings from FIG. 1; specifically, these genes were upregulated in patients with treatment resistance versus ongoing responders. Cell values represent the Spearman rank correlation value (R) between the covariates shown. Shading indicate positive and negative associations, respectively, between covariates. ARG2, arginase 2; CBG, complement C8 gamma chain; CCL, chemokine ligand; FoxP3, forkhead box protein P3; IL, interleukin; LAG-3, lymphocyte-activation gene 3; LOX-1, lectin-type oxidized low-density lipoprotein receptor 1; max, maximum; min, minimum; M-MDSC, monocyte myeloid-derived suppressor cell; PD-1, programmed cell death protein 1; PMN-MDSC, polymorphonuclear myeloid-derived suppressor cell; S100A9, S100 calcium-binding protein A9; TIM-3, T-cell immunoglobulin and mucin domain-containing protein 3; TME, tumor microenvironment; TREM2, triggering receptor expressed on myeloid cells 2.

FIG. 13. The suppressive myeloid gene signature was positively associated with gene expression of cancer testis antigens. Data are included for 30 patients from ZUMA-1 Cohorts 1-3 with evaluable samples for gene expression analyses. The genes presented in the heatmap were selected based on findings from FIG. 1; specifically, these genes were upregulated in patients with treatment resistance versus ongoing responders. Cell values represent the Spearman rank correlation value (R) between the covariates shown. Shading indicate positive and negative associations, respectively, between covariates. ARG2, arginase 2; BTK, Burton tyrosine kinase; CBG, complement C8 gamma chain; CCL, chemokine ligand; DDX43, DEAD-box helicase 43; IL, interleukin; IRF, interferon-regulatory factor; ITK, interleukin-2-includible T-cell kinase; MAGE, melanoma antigen gene; MAP2K, mitogen-activated protein kinase kinase; MAP3K, mitogen-activated protein kinase kinase kinase; MAPK, mitogen-activated protein kinase; MAPKAPK, mitogen-activated protein kinase-activatedprotein kinase; max, maximum; min, minimum; PRAME, preferentially expressed antigen of melanoma; SPA17, sperm surface protein Sp17; STAT, signal transducer and activator of transcription; SYK, spleen associated tyrosine kinase; TREM2, triggering receptor expressed on myeloid cells 2.

FIG. 14. Protocol-specified AE management in cohorts 1+2 and cohort 4 of CLINICAL TRIAL-1. “Yes” or “No” indicates whether tocilizumab or corticosteroid was or was not administered, respectively. *Only in case of comorbidities or older age. ^{†}Only if no improvement with tocilizumab; use standard dose. ^{‡}If no improvement after 3 days. AE, adverse event; CRS, cytokine release syndrome; HD, high dose; NE, neurologic event; Mgmt, management.

FIG. 15. Patient disposition diagram. The figure summarizes the disposition of patients enrolled in CLINICAL TRIAL-1 cohort 4. A total of 57 patients were screened according to institutional protocols. There were 11 screen failures. *Due to suicide (n=1) and disease progression (n=1). axicabtagene ciloleucel, axicabtagene ciloleucel.

FIGS. 16A and 16B. ORR and duration of response. (16A) ORR of patients in cohort 4 and rates of SD and PD. Response could not be evaluated in 2 patients: 1 patient died of pneumonia before the first assessment, and 1 patient had a positive result from positron emission tomography with suspected inflammation. (16B) Kaplan-Meier curve of duration of response. CR, complete response; NE, not estimable; NR, not reached; ORR, objective response rate; PD, progressive disease; PR, partial response; SD, stable disease.

FIG. 17. Best response by corticosteroid use. The figure shows the percentages of patients who did or did not receive steroids, with corresponding ORR, CR, and ongoing response at 12 months. CR, complete response; ORR, objective response rate.

FIG. 18. Progression-free survival in cohort 4.

FIGS. 19A and 19B. CAR T-cell expansion and key soluble serum biomarker levels over time. (19A) Median (Q1, Q3) blood levels of CART cells over time. (19B) Median (Q1, Q3) levels of key soluble serum inflammatory biomarkers plotted against time. BL, baseline; CAR, chimeric antigen receptor; CRP, C-reactive protein; GM-CSF, granulocyte-macrophage colony-stimulating factor; IFN, interferon; IL, interleukin.

FIG. 20. Selected CSF analysis at baseline and day 5 and association with neurologic events. The figure shows levels of inflammatory markers in CSF samples from cohort 4 at baseline (dots) and day 5 (triangles) by severity of the neurologic event. The grade of the neurologic event (0 to 5) and number of cases are indicated in the upper and lower rows of text, respectively. The middle line represents the median, and the box represents the interquartile range; whiskers show minimum and maximum values. CRP, C-reactive protein; CSF, cerebrospinal fluid; IFN, interferon; IL, interleukin; R, receptor.

FIG. 21. Selected serum analysis at baseline and day 5 and association with neurologic events. The figure shows levels of inflammatory markers in blood serum samples from cohort 4 at baseline (dots) and day 5 (triangles) by severity of the neurologic event. The grade of the neurologic event (0 to 5) and number of cases are indicated in the upper and lower rows of text, respectively. The middle line represents the median, and the box represents the interquartile range; whiskers show minimum and maximum values. CRP, C-reactive protein; IFN, interferon; IL, interleukin; R, receptor.

### DETAILED DESCRIPTION

The present disclosure is based in part on the discovery that pre-infusion attributes (e.g., T cell fitness) of apheresis material and engineered CAR T cells, as well as pre-treatment characteristics of patients' immune factors and tumor burden may be associated with clinical efficacy and toxicity including durable responses, grade A cytokine release syndrome, and grade 3 neurologic events.

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CHL CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In one embodiment, the CAR T cell treatment comprises “axicabtagene ciloleucel treatment”. “Axicabtagene ciloleucel treatment” consists of a single infusion of anti-CD19 CAR transduced autologous T cells administered intravenously at a target dose of 2×106 anti-CD19 CART cells/kg. For subjects weighing greater than 100 kg, a maximum flat dose of 2×108 anti-CD19 CAR T cells may be administered. The anti-CD19 CAR T cells are autologous human T cells that have been engineered to express an extracellular single-chain variable fragment (scFv) with specificity for CD19 linked to an intracellular signaling part comprised of signaling domains from CD28 and CD3 (CD3-zeta) molecules arranged in tandem anti-CD19 CAR vector construct has been designed, optimized and initially tested at the Surgery Branch of the National Cancer Institute (NCI, IND 13871) (Kochenderfer et al, J Immunother. 2009; 32(7):689-702; Kochenderfer et al, Blood. 2010; 116(19):3875-86). The scFv is derived from the variable region of the anti-CD19 monoclonal antibody FMC63 (Nicholson et al, Molecular Immunology. 1997; 34(16-17):1157-65). A portion of the CD28 costimulatory molecule is added, as murine models suggest this is important for the anti-tumor effect and persistence of anti-CD19 CAR T cells (Kowolik et al, Cancer Res. 2006; 66(22):10995-1004). The signaling domain of the CD3-zeta chain is used for T cell activation. These fragments were cloned into the murine stem cell virus-based (MSGV1) vector, utilized to genetically engineer the autologous T cells. The CAR construct is inserted into the T cells' genome by retroviral vector transduction. Briefly, peripheral blood mononuclear cells (PBMCs) are obtained by leukapheresis and Ficoll separation. Peripheral blood mononuclear cells are activated by culturing with an anti-CD3 antibody in the presence of recombinant interleukin 2 (IL-2). Stimulated cells are transduced with a retroviral vector containing an anti-CD19 CAR gene and propagated in culture to generate sufficient engineered T cells for administration. Axicabtagene ciloleucel is a subject-specific product.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, [add other solid tumors] multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, wherein the extracellular domain comprises a hinge domain, which may be truncated.

A “therapeutically effective amount,” “effective dose,” “effective amount,” or “therapeutically effective dosage” of a therapeutic agent, e.g., engineered CAR T cells, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g. animal model) systems.

The term “combination” refers to either a fixed combination in one dosage unit form, or a combined administration where a compound of the present disclosure and a combination partner (e.g. another drug as explained below, also referred to as “therapeutic agent” or “agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The terms “product” or “infusion product” are used interchangeably herein and refer to the T cell composition that is administered to the subject in need thereof. Typically, in CAR T-cell therapy, the T cell composition is administered as an infusion product.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+ (L-selectin), CD27+, CD28+ and IL-7Rα+, but they also express large amounts of CD95, IL-2R13, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

In the context of this disclosure, the term “TN,” “T naïve-like”, and CCR7+CD45RA+ actually refers to cells that are more like stem-like memory cells than like canonical naïve T cells. Accordingly, all references in the Examples and Claims to T_{N} refers to cells that were experimentally selected only by their characterization as CCR7+CD45RA+ cells and should be interpreted as such. Their better name in the context of this disclosure is stem-like memory cells, but they shall be referred to as CCR7+CD45RA+ cells. Further characterization into stem-like memory cells may be done for example using the methods described in Arihara Y, Jacobsen C A, Armand P, et al. Journal for ImmunoTherapy of Cancer. 2019; 7(1):P210.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” or a “subject” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CD S, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In some embodiments, the treatment may be prophylactic, in which case the treatment is administered before any symptoms of the condition are observed. The term “prophylaxis” as used herein means the prevention of or protective treatment for a disease or disease state. Prevention of a symptom, disease, or disease state may include reduction (e.g., mitigation) of one or more symptoms of the disease or disease state, e.g., relative to a reference level (e.g., the symptom(s) in a similar subject not administered the treatment). Prevention may also include delaying onset of one or more symptoms of the disease or disease state, e.g., relative to a reference level (e.g., the onset of the symptom(s) in a similar subject not administered the treatment). In embodiments, a disease is a disease described herein. In some embodiments, the disease is cancer. In some embodiments, the diseased state is CRS or neurotoxicity. In some embodiments, indicators of improvement or successful treatment include determination of the failure to manifest a relevant score on toxicity grading scale (e.g. CRS or neurotoxicity grading scale), such as a score of less than 3, or a change in grading or severity on the grading scale as discussed herein, such as a change from a score of 4 to a score of 3, or a change from a score of 4 to a score of 2, 1 or 0.

As used herein, “myeloid cells” are a subgroup of leukocytes that includes granulocytes, monocytes, macrophages, and dendritic cells.

In one embodiment, the terms “high” and “low” mean “above” and “below” the median value for a representative population of tumors. In some embodiments (for example, in the context of using NanoString for gene expression analysis), the medians may be as follows:

| Parameter | Median | High | Low |
|---|---|---|---|
| ARG2 | 26.77 | Above median | Below median |
| | | (above 27) | (below 27) |
| TREM2 | 10.32 | Above median | Below median |
| | | (above 10) | (below 10) |
| CCL20 | 0 | Above median | equal to 0 |
| | | (above 0) | |
| IL8 | 41.55 | Above median | Below median |
| | | (above 42) | (below 42) |
| IL13 | 8.95 | Above median | Below median |
| | | (above 9) | (below 9) |
| Baseline | 3721 | Above median | Below median |
| Tumor | | (above 3700) | (below 3700) |
| Burden | | | |
| (SPD) | | | |

As used herein, the term “quartile” is a statistical term describing a division of observations into four defined intervals based upon the values of the data and how they compare to the entire set of observations.

As used herein, the term “Study day 0” is defined as the day the subject received the first CAR T cell infusion. The day prior to study day 0 will be study day −1. Any days after enrollment and prior to study day −1 will be sequential and negative integer-valued.

As used herein, the term “durable response” refers to the subjects who were in ongoing response at least by one year follow up post CAR T cell infusion. In one embodiment, “duration of response” is defined as the time from the first objective response to disease progression or to death due to disease relapse.

As used herein, the term “relapse” refers to the subjects who achieved a complete response (CR) or partial response (PR) and subsequently experienced disease progression.

As used herein, the term “non-response” refers to the subjects who had never experienced CR or PR post CAR T cell infusion, including subjects that with stable disease (SD) and progressive disease (PD).

As used herein, the term “objective response” refers to complete response (CR), partial response (PR), or non-response. It may be assessed per revised IWG Response Criteria for Malignant Lymphoma (Cheson et al., J Clin Oncol. 2007; 25(5):579-86).

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution.

As used herein “objective response rate” (ORR) is determine per International Working Group (IWG) 2007 criteria (Cheson et al. J Clin Oncol. 2007; 25(5):579-86).

As used herein “progression-free survival (PFS)” may be defined as the time from the T cell infusion date to the date of disease progression or death from any cause. Progression is defined per investigator's assessment of response as defined by IWG criteria (Cheson et al., J Clin Oncol. 2007; 25(5):579-86).

The term “overall survival (OS)” may be defined as the time from the T cell infusion date to the date of death from any cause.

As used herein, the expansion and persistence of CART cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+ PBMC/μL in serum attained after Day 0.

As used herein, the “time to Peak of CART cell” is defined as the number of days from Day 0 to the day when the peak of CAR T cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Baseline}{Baseline}$$

As used herein, the “peak of cytokine post baseline” is defined as the maximum level of cytokine in serum attained after baseline (Day −5) up to Day 28.

As used herein, the “time to peak of cytokine” post CART cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

As used herein, treatment-emergent adverse events (TEAEs) are defined as adverse events (AE) with onset on or after the first dose of conditioning chemotherapy. Adverse events may be coded with the Medical Dictionary for Regulatory Activities (MedDRA) version 22.0 and graded using the National Cancer Institute (NCI) Common Terminology Criteria for Adverse Events (CTCAE) version 4.03. Cytokine Release Syndrome (CRS) events may be graded on the syndrome level per Lee and colleagues (Lee et al, 2014 Blood. 2014; 124(2):188-95. Individual CRS symptoms may be graded per CTCAE 4.03. Neurologic events may be identified with a search strategy based on known neurologic toxicities associated with CAR T immunotherapy, as described in, for example, Topp, M S et al. Lancet Oncology. 2015; 16(1):57-66.

Various aspects of the disclosure are described in further detail in the following subsections.

### Characterization of the Tumor Microenvironment (TME)

In some embodiments, the present disclosure provides methods to characterize the tumor microenvironment (TME) using gene expression profiling and/or intratumoral T cell density and/or TME myeloid cell density/myeloid inflammation status measurements prior to treatment with immunotherapy. In one embodiment, these measurements are normalized to tumor burden (TB). In one embodiment, immunotherapy is selected from treatment with a chimeric receptor therapy (e.g., YESCARTA™ axicabtagene ciloleucel (axicabtagene ciloleucel), TECARTUS™-brexucabtagene autoleucel/KTE-X19, KYMRIAH™ (tisagenlecleucel), etc), TCR, TIL, immune check point inhibitors, among others. In one embodiment, the immunotherapy product comprises autologous or allogeneic CAR T cells. In one embodiment, the immunotherapy comprises T-Cell Receptor-modified T cells. In one embodiment, the immunotherapy comprises tumor infiltrating lymphocytes (TILs). In one embodiment, the immunotherapy product comprises Induced Pluripotent Stem Cells (iPSCs). As described herein, the TME characteristics utilizing pre-specified gene sets (e.g., Immunosign®21, Pan Cancer) and immune scores (e.g., Immunosign®21), intratumoral T cell density measurements or indices (e.g., Immunoscore®), TME myeloid cell density, and/or TME myeloid inflammation associate with clinical outcomes of chimeric receptor therapy (e.g., axicabtagene ciloleucel (axicabtagene ciloleucel)) may be used to predict clinical outcomes of all immunotherapies (e.g., T cells, non-T cells, TCR-based therapies, CAR-based therapies, bispecific T-cell engagers (BiTEs), and/or immune checkpoint blockade).

Patient tumor biopsies may be used as starting material to analyze the tumor microenvironment using gene expression profiling (e.g., digital gene expression using NanoString™) and immunohistochemistry (IHC). In some embodiments, the patient biopsy is obtained prior to treatment with a chimeric receptor therapy (e.g., axicabtagene ciloleucel (axicabtagene ciloleucel)) or other immunotherapy. In some embodiments, the biopsy is obtained just prior to the beginning of conditioning therapy.

A bioinformatics and/or data science-based methods may be used to generate an immune score or scores to characterize the TME. In some embodiments, the immune score is a measure of immune related genes that provides information regarding adaptive immunity including T cell cytotoxicity, T cell differentiation, T cell attraction, T cell adhesion and immune suppression including immune orientation, angiogenesis suppression, immune co-inhibition, and cancer stem cells. The bioinformatics method may also include T cell-specific (effector T cell, Th1) genes, interferon pathway-related genes, chemokines, and immune checkpoints.

An expression profiling assay (e.g., The Immunosign® Clinical Research assay utilizes the nCounter® technology (NanoString)) may be used to measure the gene expression level of multiple immune genes in a multiplex format. In some embodiments, a high/low immune score (e.g., Immunosign®21 score) cut-off may be defined as the 25th percentile of the observed scores among samples. In some embodiments, the high score indicates expression of immune-related genes potentially associated with tumor response.

In some embodiments, the immune score is a measure of intratumoral T cell density. Intratumoral T cell density may be determined by, for example, detecting and quantifying T cells, such as CD3+ T cells and/or CD8+ T cells, in the tumor microenvironment. For example, tumor biopsies may be sectioned and stained or labeled for T cell markers such as CD3 and/or CD8, and the relative or absolute abundance of T cells may be quantified by a pathologist or determined using dedicated digital pathology software. In some embodiments, a high/low immune score (e.g., Immunoscore®) is assigned based on intratumoral T cell density. A high/low immune score threshold may be defined, for example, as the median score observed among samples. In some embodiments, intratumoral T cell density is determined using flow cytometry and/or protein-based assays such as western blotting and ELISA.

TME myeloid cell density and TME myeloid inflammation levels, expression and tumor-infiltrating T lymphocyte analysis and scoring may be used to examine associations between TME features and response. In some embodiments, objective response (OR) is determined per the revised IWG Response Criteria for Malignant Lymphoma (Cheson, 2007) and determined by IWG Response Criteria for Malignant Lymphoma (Cheson et al. Journal of Clinical Oncology 32, no. 27 (September 2014) 3059-3067). In some embodiments, Duration of Response is assessed. In some embodiments, Progression-Free Survival (PFS) by investigator assessment per Lugano Response Classification Criteria is evaluated.

In some embodiments, CAR T cells are quantified using a TaqMan-based quantitative polymerase chain reaction (qPCR; Thermo Fisher Scientific) as previously described (Locke F L et al. Lancet Oncol. 2019; 20(1):31-42; Neelapu S S et al. N Engl J Med. 2017; 377(26):2531-2544; Locke F L et al. Mol Ther. 2017; 25(1):285-295). To report frequencies of CAR-positive cells in blood, CAR T cells per microliter are calculated by normalizing CAR gene expression to actin expression in peripheral blood mononuclear cells, followed by normalization to absolute lymphocyte counts (Kochenderfer J N et al. J Clin Oncol. 2017; 35(16):1803-1813). Peak CART expansion, defined by maximum level of CAR T, measured per μL of blood is used for analysis.

In one embodiment, gene expression analysis is done by NanoString. In one embodiment, RNA extraction from frozen or fixed biopsies is performed using QIAGEN RNeasy kit and QIAGEN FFPE RNeasy Extraction kit, respectively. Annotations from the pathologist performing H&E staining are used to guide removal of normal tissue from the slides by macrodissection prior to RNA extraction, and after tissue deparaffinization and lysis. After extraction, RNA quantification is performed with Nanodrop and qualification is performed with the Agilent Bioanalyser. One RNA QC sample is included in each testing run as a positive control for extraction. RNA expression profiling is performed using 3 NanoString datasets.

In one embodiment, the results are subjected to statistical analysis. In one embodiment, a volcano plot, heatmap of transcript expression are generated using Spotfire 7.12.0 (TIBCO Software). Kaplan-Meier survival curves (Overall survival and Progression free survival), boxplots and regression curves are plotted using R Studio 3.4.1. In one embodiment,

In some embodiments, the present disclosure provides a predictive tool for clinical efficacy of immunotherapy (e.g., T cell therapy), by analyzing tumor microenvironment prior to treatment (e.g., pre-conditioning) and changes occurring after T cell therapy administration (e.g., two weeks after, four weeks after).

In one aspect, the disclosure provides that pre-treatment immune TME features related to suppressive myeloid-related activity (i.e., myeloid cell activity that reduces the effects of or impairs the effects of treatment, e.g., immunotherapy; reduces response to treatment), most notably (but not solely) ARG2, TREM2, and IL-8 gene expression, were elevated in patients who failed to respond or relapsed without documented loss of CD19 expression. In one aspect, the disclosure provides that ARG2 and TREM2 levels in pre-treatment biopsies were negatively associated with CD8⁺ T-cell density. In one aspect, patients with high TB who achieved durable response had low pre-treatment ARG2 and TREM2 levels in TME and enhanced CAR T-cell expansion after axicabtagene ciloleucel compared with patients with high TB who relapsed. In one aspect, a high ratio of T-cell to suppressive myeloid cell markers (T/M ratio) in pre-treatment biopsies associated positively with CAR T-cell expansion (peak and peak normalized to TB) and durable response in patients with high TB.

Accordingly, in one embodiment, the disclosure provides a method of predicting a suppressive tumor microenvironment (TME) induced by myeloid cells of in a cancer patient and/or the clinical efficacy of immunotherapy for treating the patient's cancer by quantifying myeloid inflammation in the TME, in a tumor of the cancer patient. In one embodiment, the higher the tumor level of myeloid inflammation, the more treatment-suppressive the TME of the cancer patient. In one embodiment, the higher the level of tumor myeloid inflammation the lower the clinical efficacy of the immunotherapy. In one embodiment, the immunotherapy is selected from CAR-T cells, TCR-T cells, tumor infiltrating lymphocytes, checkpoint inhibitors, and combinations thereof. In one embodiment, the TME myeloid inflammation level is estimated by measuring the gene expression of one or more ofARG2, TREM2, IL8, IL13, C8G, CCL20, IFNL2, OSM, IL11RA, CCL11, MCAM PTGDR2, and CCL16 in the tumor. In one embodiment, the higher the expression of one or more of these genes in the TME, the higher the myeloid inflammation level in the TME. In one embodiment, the clinical efficacy is assessed by complete response rates, objective response rates, ongoing response rates, median durability of response, median PFS, and/or median OS.

In another embodiment, the disclosure provides that immunotherapy (e.g., axicabtagene ciloleucel) may overcome high TB in patients with a favorable immune TME (favorable with respect to favorable to respond to treatment, e.g., respond to immunotherapy) alongside robust CAR T-cell expansion. In one embodiment, robust CAR T-cell expansion comprises the median level of CAR T cell expansion in the general CAR T cell treatment population, where the median is between 0-10, 10-20, 20-30, 30-40, 40-50, 50-60, 60-70, 70-80, 80-90, 90-100, preferably between 40-50). Accordingly, the disclosure provides actionable strategies to overcome high TB in the context of CAR T-cell therapy. In one embodiment, a favorable immune TME is characterized by reduced suppressive myeloid cell activity (low ARG2 and TREM2 expression) and increased T/M ratio. In one embodiment, the disclosure provides a method of treating cancer with immunotherapy (e.g., CAR or TCR-T) in a cancer patient in need thereof, wherein the patient is selected for treatment when the level of TME myeloid inflammation is above/within a reference level. In one embodiment, the patient is selected for treatment when the level of TME myeloid inflammation is the following, using the recited genes as a surrogate for TME myeloid inflammation: 0-27 (ARG2), 0-10 (TREM2), 0-42 (IL8), 0-9 (IL13), 0-11 (C8G), 0 (CCL20), 0-11 (IFNL2), 0-8 (OSM), 0-77 (IL11RA), 0-27 (CCL11), 59-132 (MCAM), 0 (PTGDR2), and/or 0 (CCL16), as measured by NanoString unit methods. A table of ranges and quartile distributions is provided below. In one embodiment, ARG2: 0-27, 27-40, 40-75, 75-120, preferably 0-27; TREM2: 0-10, 10-35, 35-100, 100-500, preferably 0-10; IL8: 0-40, 40-100, 100-200, 200-3000, preferably 0-40; IL13: 0-10, 10-40, 40-90, 90-400, preferably 0-10; CCL20: 0-44, 44-100, 100-500, preferably 0-44.

In one embodiment, increased T/M ratio is a ratio above −0.5-0.02, 0.02-1, 1-4, 4-8, 8-15, preferably above 1-4. In one embodiment, the T cell index is estimated as the root mean square of selected genes (CD3D, CD8A, CTLA4, TIGIT), per NanoString. In other embodiments, other equivalent methods may be used by one of ordinary skill in the art. In some embodiments, the myeloid index is estimated as root mean square of selected genes (ARG2, TREM2). In other embodiments, other equivalent methods may be used by one of ordinary skill in the art. In some embodiments, the T/M ratio is estimated as Log 2((T-cell Index+1)/(Myeloid Index+1)). In other embodiments, other equivalent methods may be used by one of ordinary skill in the art.

In one embodiment, the disclosure provides a method to stratify patients having a tumor (with a TME) for combination therapy including immunotherapy (e.g., CAR or TCR-T) and another agent, the method comprising administering immunotherapy (e.g., CAR or TCR-T) in combination with an agent to the patient prior to CAR-T infusion, at the peak of CAR-T expansion, and/or after peak CAR-T expansion. In one embodiment, the peak of CAR-T expansion is Day 7-14 post infusion. In one embodiment, the peak of CAR-T expansion is Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, or Day 20 post-infusion. In one embodiment, the period after peak CAR-T expansion is the period between Day 14-28 post-infusion. In one embodiment, the period after peak CAR-T expansion is Day 1-Day 5, Day 5-Day 10, Day 10-Day 15, Day 15-Day 20, Day 20-Day 25; after Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 25, Day 30, Day 35, Day 40, Day 45, Day 50, any day after peak expansion. In one embodiment, the combination therapy enhances the proliferation of the T cells. In one embodiment, said combination therapy comprises treatment with pembrolizumab, lenalidomide, epcoritamab, and utoliumab. In one embodiment, the combination therapy reduces the suppressive myeloid population in the TME. In one embodiment, said therapy comprises magrolimab (anti-CD47 antagonist), GSK3745417 (STING agonist), INCB001158 (ARG1/2 inhibitor), GS-1423 (CD73xTGFβ mAb), Selicrelumab (CD40 agonist), GS3583 (FLT3 agonist), Pexidartinib (CSF1R inhibitor, epacadostat (IDO1 inhibitor), GS9620 (TLR agonist).

In one embodiment, the disclosure provides a method of treating a tumor in a subject with a high tumor burden, wherein the high tumor burden in the subject is reduced by administering one or more agents that result in a favorable immune TME and/or by increasing CAR T cell expansion. In one embodiment, the subject has a high tumor burden when baseline tumor burden (longest perpendicular diameters, SPD) is greater than 3000 mm². In one embodiment, a high tumor burden is a baseline tumor burden between 100-2000, 2000-3000, 3000-6000, 6000-40000, preferably above 2000-3000 mm²′ In one embodiment, the immune TME is favorable when the TME presents reduced suppressive myeloid cell activity and/or increased T cell/Myeloid cell ratio. In one embodiment, increased T/M ratio is 1-4, 1, 2, 3, or 4. In one embodiment, increased T/M is a ratio between 1-4. In one embodiment, increased T/M is a ratio between 2-5, 3-6, 7-10, 11-14, 15-18, or 19-20. In one embodiment, increased T/M is a ratio between higher than 10, 20, 30, 40, 50, 60, 70, 80, 90, 100. In one embodiment, reduced myeloid cell activity is low ARG2 and/or low TREM2 gene expression. In one embodiment, low ARG2 and/or TREM2 gene expression is when the gene expression levels fall within 0-27, as measured by Nanostring (see EXAMPLES), or an equivalent value as measured by other gene expression measuring method. In one embodiment, the levels are low when they fall within the first quartile of levels among those in a representative tumor population, as assessed by one of ordinary skill in the art. In one embodiment, the agent reduces tumor myeloid suppressive activity and/or reduces tumoral myeloid cell density as assessed by measuring CD14+ cells, CD68+ cells, CD68+CD163+ cells, CD68+CD206+ cells, CD11b+ CD15+ CD14− LOX-1+ cells, and/or CD11b+ CD15− CD14+ S100A9+ CD68− cells by immunohistochemistry. In one embodiment, the agent is selected from anti-CD47 antagonists, CSF/CSF-1R inhibitors, TLR agonists, CD40 agonists, arginase inhibitors, IDO inhibitors, and TGF-beta inhibitors. In one embodiment, the agent is selected from magrolimab (anti-CD47 antagonist), GSK3745417 (STING agonist), INCB001158 (ARG1/2 inhibitor), GS-1423 (CD73xTGFβ mAb), Selicrelumab (CD40 agonist), GS3583 (FLT3 agonist), Pexidartinib (CSF1R inhibitor), epacadostat (IDO1 inhibitor), and/or GS9620 (TLR agonist).

In one embodiment, the agent is selected from (i) a GM-CSF inhibitor selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule; (ii) a CSF1 inhibitor selected from RG7155, PD-0360324, MCS110/lacnotuzumab), or a biosimilar version of any one of the same; and a small molecule; and/or (iii) a GM-CSFR inhibitor and the CSF1R inhibitor selected from Mavrilimumab (formerly CAM-3001; MedImmune, Inc.); cabiralizumab (Five Prime Therapeutics); LY3022855 (IMC-CS4) (Eli Lilly), Emactuzumab, also known as RG7155 or R05509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule.

In one embodiment, the immunotherapy is combined with low dose radiation, promotion of T cell activity through immune checkpoint blockade, and/or T cell agonists. In one embodiment, the T cell agonist is selected from pembrolizumab, lenalidomide, epcoritamab, and utoliumab. In one embodiment, the combination agent is selected from check-point inhibitors (e.g., anti-PD1 antibodies, pembrolizumab (Keytruda), Cemiplimab (Libtayo), nivolumab (Opdivo); anti-PD-L1 antibodies, Atezolizumab (Tecentriq), Avelumab (Bavencio), Durvalumab (Imfinzi); and/or anti-CTLA-4 antibodies, Ipilimumab (Yervoy)).

In one embodiment, the disclosure provides a method for quantifying TME myeloid inflammation comprising measuring gene expression of one or more of ARG2, TREM2, IL8, IL13, C8G, CCL20, IFNL2, OSM, ILIIRA, CCL11, MCAM, PTGDR2, and CCL16 in the tumor. In one embodiment, the higher the expression of one or more of these genes, the higher the TME myeloid inflammation level.

In one embodiment, the disclosure provides a method of predicting clinical efficacy of immunotherapy (e.g., CAR or TCR-T) of a tumor in a subject in need thereof, comprising measuring gene expression of one or more of ARG2, TREM2, IL8, IL13, C8G, CCL20, IFNL2, OSM, ILIIRA, CCL11, MCAM PTGDR2, and CCL16 in the TME, wherein the higher the expression of one or more of these genes the lower the clinical efficacy. In one embodiment, clinical efficacy is measured by PFS and/or OS, ongoing response rates, complete response rates, and/or objective response rates. In one embodiment, the T/M ratio may be used to differentiate between high and low tumor burden subjects, based on its influence on ongoing response rate.

In one embodiment, the disclosure provides a method of predicting response to immunotherapy (e.g., CAR or TCR-T) in a patient with large tumor burden, comprising measuring the ratio of activated T cells to suppressive myeloid cells in the TME. In one embodiment, the higher the ratio of activated T cells to suppressive myeloid cells in the TME, the better the response. In one embodiment, T cell activation is measured by measuring the gene expression levels of one or more of CD3D, CD8A, CTLA4, and TIGIT in the TME. In one embodiment, the level of suppressive myeloid cells in the TME is measured by measuring the ratio of T cell to myeloid cell index (root mean square of selected genes) with log 2 transformation. In one embodiment, the level of suppressive myeloid cells is measured by measuring the gene expression levels ofARG2 and/or TREM2 in the TME. In one embodiment, the disclosure provides a method of selecting cancer patients for treatment, wherein when the ratio of activated T cells to suppressive myeloid cells in the TME is low, the patient is administered myeloid conditioning prior to immunotherapy. In some embodiments, myeloid conditioning comprises inhibition of suppressive myeloid TME. In one embodiment, myeloid conditioning therapy is selected from agents that target specific myeloid genes (e.g., ARG2, TREM2, IL8, CD163, MRC1, MSR1) and costimulatory genes/pathways (e.g. TLRs, CD40, STING) such as magrolimab (anti-CD47 antagonist), GSK3745417 (STING agonist), INCB001158 (ARG1/2 inhibitor), GS-1423 (CD73xTGFβ mAb), Selicrelumab (CD40 agonist), GS3583 (FLT3 agonist), Pexidartinib (CSF1R inhibitor), epacadostat (IDO1 inhibitor), and/or GS9620 (TLR agonist). Other useful CSF/CSF1R inhibitors are mentioned above. In some embodiments, large tumor burden (longest perpendicular diameters, SPD) is a tumor burden within 3000-40000 mm². In some embodiments, a low T/M ratio within −0.5-4 of activated T cells to suppressive myeloid cells is a ratio within −0.5-4. In one embodiment, increased T/M ratio is above 1-4. In one embodiment, increased T/M is a ratio between 2-5, 3-6, 7-10, 11-14, 15-18, or 19-20. In one embodiment, increased T/M is a ratio between higher than 10, 20, 30, 40, 50, 60, 70, 80, 90, 100. In one embodiment, response is objective response rates, complete response rates, ongoing response rates, median durability of response, median PFS, or median OS.

In one embodiment, the terms low, high, increased, decreased and other relative terms in the previous embodiments are relative to the general distribution in a representative group of tumors of the same kind. In one embodiment, the terms are relative to the distribution of quartiles, median, average, min, max, and range values of the table below.

In one embodiment, the T/M ratio, myeloid signature, baseline tumor burden (SPD), and biomarker gene expression in the TME has a distribution as follows

| Parameter | Min | P10 | Q1 | Median | Q3 | P90 | Max | Range 1 |
|---|---|---|---|---|---|---|---|---|
| ARG2 | 0 | 0 | 0 | 26.77 | 39.57 | 73.88 | 101.14 | 0-0 |
| TREM2 | 0 | 0 | 0 | 10.32 | 34.11 | 101.15 | 195.69 | 0-0 |
| CCL20 | 0 | 0 | 0 | 0 | 44.11 | 100.89 | 390.6 | 0-0 |
| IL8 | 0 | 0 | 0 | 41.55 | 97.93 | 203.99 | 2637.78 | 0-0 |
| IL13 | 0 | 0 | 0 | 8.95 | 39.18 | 88.17 | 193.07 | 0-0 |
| IFNL2 | 0 | 0 | 0 | 10.71 | 72.36 | 152.45 | 633.04 | 0-0 |
| OSM | 0 | 0 | 0 | 7.93 | 38.52 | 121.9 | 354.61 | 0-0 |
| IL11RA | 0 | 0 | 0 | 76.56 | 96.36 | 121.57 | 172.05 | 0-0 |
| CCL11 | 0 | 0 | 0 | 26.67 | 85.47 | 201.78 | 317.84 | 0-0 |
| MCAM | 0 | 0 | 59.37 | 132.31 | 201.27 | 313.65 | 409.77 | 0-0 |
| PTGDR2 | 0 | 0 | 0 | 0 | 21.58 | 39.29 | 181.25 | 0-0 |
| CCL16 | 0 | 0 | 0 | 0 | 19.17 | 49.22 | 194.38 | 0-0 |
| C8G | 0 | 0 | 0 | 11.35 | 48.58 | 102.64 | 130.02 | 0-0 |
| Myeloid | 0 | 0 | 0 | 27.45 | 48.38 | 87.29 | 152.49 | 0-0 |
| Signature | | | | | | | | |
| T Cell/ | −0.47 | −0.02 | 0.86 | 4 | 7.78 | 9.25 | 10.68 | −0.47--0.02 |
| Myeloid | | | | | | | | |
| Ratio | | | | | | | | |
| Baseline | 171 | 485 | 1922 | 3689 | 6533 | 9940 | 39658 | 171-485 |
| Tumor | | | | | | | | |
| Burden | | | | | | | | |
| (SPD) | | | | | | | | |

| Parameter | Range 2 | Range 3 | Range 4 | Range 5 | Range 6 |
|---|---|---|---|---|---|
| ARG2 | 0-0 | 0-26.77 | 26.77-39.57 | 39.57-73.88 | 73.88-101.14 |
| TREM2 | 0-0 | 0-10.32 | 10.32-34.11 | 34.11-101.15 | 101.15-195.69 |
| CCL20 | 0-0 | 0-0 | 0-44.11 | 44.11-100.89 | 100.89-390.6 |
| IL8 | 0-0 | 0-41.55 | 41.55-97.93 | 97.93-203.99 | 203.99-2637.78 |
| IL13 | 0-0 | 0-8.95 | 8.95-39.18 | 39.18-88.17 | 88.17-193.07 |
| IFNL2 | 0-0 | 0-10.71 | 10.71-72.36 | 72.36-152.45 | 152.45-633.04 |
| OSM | 0-0 | 0-7.93 | 7.93-38.52 | 38.52-121.9 | 121.9-354.61 |
| IL11RA | 0-0 | 0-76.56 | 76.56-96.36 | 96.36-121.57 | 121.57-172.05 |
| CCL11 | 0-0 | 0-26.67 | 26.67-85.47 | 85.47-201.78 | 201.78-317.84 |
| MCAM | 0-59.37 | 59.37-132.31 | 132.31-201.27 | 201.27-313.65 | 313.65-409.77 |
| PTGDR2 | 0-0 | 0-0 | 0-21.58 | 21.58-39.29 | 39.29-181.25 |
| CCL16 | 0-0 | 0-0 | 0-19.17 | 19.17-49.22 | 49.22-194.38 |
| C8G | 0-0 | 0-11.35 | 11.35-48.58 | 48.58-102.64 | 102.64-130.02 |
| Myeloid | 0-0 | 0-27.45 | 27.45-48.38 | 48.38-87.29 | 87.29-152.49 |
| Signature | | | | | |
| T Cell/ | −0.02-0.86 | 0.86-4 | 4-7.78 | 7.78-9.25 | 9.25-10.68 |
| Myeloid | | | | | |
| Ratio | | | | | |
| Baseline | 485-1922 | 1922-3689 | 3689-6533 | 6533-9940 | 9940-39658 |
| Tumor | | | | | |
| Burden | | | | | |
| (SPD) | | | | | |

Q1 refers to the data point at the mark of 25% percentile. Five values may be used (min, Q1, median, Q3, max) to find the 4 interquartile ranges. Min - Q1: first quartile; Q1 - median: 2^{nd} quartile; median - Q3, 3^{rd} quartile; Q3 - Max: last quartile.

The disclosure provides that the ratio of activated T cell to suppressive myeloid cell signature is positively associated with response and also positively associated with CAR-T peak cell expansion/tumor burden. Accordingly, the disclosure provides a method to estimate CAR-T peak cell expansion/tumor burden comprising measuring T/M. Patients who have a lower activated T/myeloid ratio may benefit from myeloid conditioning (inhibition of suppressive myeloid TME by targeting specific myeloid genes for example Arg2) before treatment with immunotherapy.

In one embodiment, these methods are applied in immunotherapy, wherein immunotherapy is CAR-T cell therapy. In one embodiment, immunotherapy is selected from TCR-T cells, iPSCs, tumor infiltrating lymphocytes, and checkpoint inhibitors. In one embodiment, the immunotherapy is autologous immunotherapy. In one embodiment, the immunotherapy is allogeneic. Examples of target tumor antigens are listed elsewhere in the specification. Examples of cancers that may be treated by the methods of the disclosure are also provided elsewhere in the specification.

Methods of the present disclosure may also be used in companion testing to inform on whether additional therapies, in combination or used sequentially, will be more effective in subjects with certain tumor microenvironment characteristics. In some embodiments, additional treatments may be cytokines (e.g., IL-2, IL-15), stimulating antibodies (e.g., anti-41BB, OX-40), checkpoint blockade (e.g., CTLA4, PD-1), or innate immune stimulators (e.g., TLR, STING agonists). In some embodiments, additional treatments may be T cell-recruiting chemokines (e.g., CCL2, CCL1, CCL22, CCL17, and combinations thereof) and/or T cells. In some embodiments, the additional therapy or therapies are administered systemically or intratumorally.

One aspect of the present disclosure relates to methods of treating malignancy comprising measuring immune-related gene expression and/or T cell density at one or more site(s) of malignancy (i.e., the tumor microenvironment) prior to administration (e.g., at least one infusion) of CAR-T cells or T cells expressing an exogenous TCR. In some embodiments, said measurement is performed prior to chemotherapeutic conditioning and engineered T cell (e.g., CAR-T cell) administration.

In some embodiments, said measurement comprises determining a composite immune score based on immune-related gene expression, such as an ImmunoSign®21 or Immunosign®15 score. In some embodiments, said measurement comprises determining an immune score based on intratumoral density of T cells, including CD3+ and/or CD8+ T cells, such as Immunoscore®. In some embodiments, said measurement further comprises determining and assigning relative score(s), such as High or Low, based on comparison of a subject's immune score(s) to a predetermined threshold. In some embodiments, such predetermined threshold is or has been determined to have prognostic value with respect to the treatment of the malignancy with the engineered T cell.

In some embodiments, the disclosed methods further comprise a step of treatment optimization based on said measurement(s). For example, in some embodiments, the dose and/or schedule of engineered T cell (e.g., CAR-T cell) administration is optimized based on the myeloid activity/inflammation and the T/M ratio in the TME. In one embodiment, a favorable immune TME is characterized by reduced suppressive myeloid cell activity (low ARG2 and TREM2 expression) and increased T/M ratio. In exemplary embodiments, a subject with higher level of suppressive myeloid activity and/or decreased T/M ratio, is administered a higher dose of CAR-T cells than a subject with a lower level of suppressive myeloid activity and/or increased T/M ratio. In some embodiments, a subject with a higher level of suppressive myeloid activity and/or decreased T/M ratio is administered a dose that is about 25% higher, or about 50% higher, or about 100% higher, than a subject with a subject with a lower level of suppressive myeloid activity and/or increased T/M ratio. In additional and alternative exemplary embodiments, a subject with a subject with higher level of suppressive myeloid activity and/or decreased T/M ratio receives one or more additional CAR-T cell infusions. In some embodiments, a subject with higher level of suppressive myeloid activity and/or decreased T/M ratio is administered a first dose of immunotherapy (e.g., CAR-T cells), the treatment response is assessed, and, if incomplete response is observed, an additional measurement of the level of suppressive myeloid activity and/or T/M ratio is conducted. In some embodiments, an additional administration of immunotherapy (e.g., CAR-T cells) is performed if the subject still has a higher level of suppressive myeloid activity and/or decreased T/M ratio following the first administration.

In some embodiments, the disclosed methods additionally or alternatively comprise a ‘pre-treatment’ step in which subjects with higher level of suppressive myeloid activity and/or decreased T/M ratio are treated with the objective of improving their TME prior to CAR-T administration. For example, in some embodiments, a subject with higher level of suppressive myeloid activity and/or decreased T/M ratio is administered one or more immunostimulants, such as cytokines, chemokines, immune agonists, or immune checkpoint inhibitors. In some embodiments, an additional measurement of suppressive myeloid activity and/or T/M ratio is performed prior to treatment.

In some embodiments, the prognostic value of the suppressive myeloid activity and/or T/M ratio with respect to complete response based on immunotherapy (e.g., CAR-T therapy) is considered when evaluating treatment options. For example, in some embodiments, a subject with a higher suppressive myeloid activity and/or decreased T/M ratio receives CAR-T administration as an earlier line of therapy than a subject with a lower suppressive myeloid activity and/or higher T/M ratio.

In one embodiment, the disclosure provides a method of decreasing primary resistance to immunotherapy (e.g., CAR-T cell treatment) comprising administering to a subject having a tumor in need thereof myeloid conditioning prior to the immunotherapy. In some embodiments, myeloid conditioning comprises inhibition of suppressive myeloid TME. In one embodiment, myeloid conditioning therapy is selected from agents that target specific myeloid genes (e.g., ARG2, TREM2, IL8, CD163, MRC1, MSR1) and costimulatory genes/pathways (e.g. TLRs, CD40, STING) such as magrolimab (anti-CD47 antagonist), GSK3745417 (STING agonist), INCB001158 (ARG1/2 inhibitor), GS-1423 (CD73xTGFβ mAb), Selicrelumab (CD40 agonist), GS3583 (FLT3 agonist), Pexidartinib (CSF1R inhibitor), epacadostat (IDO1 inhibitor), and/or GS9620 (TLR agonist). Other useful CSF/CSF1R inhibitors are mentioned above. In one embodiment, the subject has a high tumor burden.

In one embodiment, the disclosure provides a method of decreasing primary resistance to immunotherapy (e.g.CAR T cell treatment) comprising administering to a subject having a tumor in need thereof an agent that modulates the methylation state of the tumor (e.g. DNA demethylating inhibitors (DDMTi) 5-aza-2′-deoxycytidine (decitabine) and 5-azacytidine or other cytosine analogs), and/or the acetylation state of the tumor (e.g., HDAC inhibitors) prior to, during, or after administration of CAR T cell treatment.

In one embodiment, the disclosure provides a method of decreasing primary resistance to immunotherapy (e.g.CAR T cell treatment) comprising administering to a subject having a tumor in need thereof a checkpoint blocking agent such as agents that block immune checkpoint receptors on the surface of T cells, such as cytotoxic T lymphocyte antigen 4 (CTLA-4), lymphocyte activation gene-3 (LAG-3), T-cell immunoglobulin mucin domain 3 (TIM-3), B- and T-lymphocyte attenuator (BTLA), T-cell immunoglobulin and T-cell immunoreceptor tyrosine-based inhibitory motif (ITIM) domain, and programmed cell death 1 (PD-1/PDL-1) prior to, during, or after administration of CAR T cell treatment. In one embodiment, the checkpoint inhibitor is selected from Pembrolizumab (Keytruda), Nivolumab (Opdivo), Cemiplimab (Libtayo), Atezolizumab (Tecentriq), Avelumab (Bavencio), Durvalumab (Imfinzi), and Ipilimumab (Yervoy). In one embodiment, the disclosure provides a method of decreasing primary resistance to CAR T cell treatment comprising administering to a subject having a tumor in need thereof an agonist of 41BB, OX40, and/or TLR prior to, during, or after administration of CAR T cell treatment.

In one embodiment, the disclosure provides a method of decreasing or overcoming primary resistance to immunotherapy (e.g.CAR T cell treatment) comprising improving CAR T cells by co-expressing gamma chain receptor cytokines under constitutive or inducible promoters.

In one embodiment, the disclosure provides a method of improving immunotherapy (e.g.CAR T cell treatment) by optimization of bridging therapy to modulate the tumor microenvironment to a more favorable immune permissive state. In one embodiment, the optimization comprises administering bridging therapy with Immunomodulatory imide drugs (IMIDs)/cereblon modulators (e.g., lenoalidomide, pomalidomide, iberdomide, and apremilast). In one embodiment, the optimization comprises administering bridging therapy with local radiation.

In one embodiment, the disclosure provides a method of improving immunotherapy (e.g.CAR T cell treatment) by optimization of bridging therapy to diminish tumor burden prior immunotherapy (e.g.CAR T cell treatment) administration. In one embodiment, the optimization comprises administering bridging therapy with R-CHOP, bendamustine, alkylating agents, and/or platinum-based agents. Other exemplary bridging therapies are described elsewhere in this application.

In one embodiment, the disclosure provides a method of improving immunotherapy (e.g.CAR T cell treatment) by optimization of conditioning treatment to modulate the tumor microenvironment to a more favorable immune permissive state (e.g., less myeloid inflammation in the TME). In one embodiment, the optimization comprises addition of local irradiation to cyclophosphamide/fludarabine conditioning. In one embodiment, the optimization comprises administration of platinum-based agents as conditioning agents.

In one embodiment, the disclosure provides a method of improving immunotherapy (e.g.CAR T cell treatment) by coadministration of biological response modifiers together or post-immunotherapy (e.g.CAR T cell treatment) administration to enable CAR T cell activity. In one embodiment, the method comprises administration of gamma chain cytokines (e.g., IL-2, IL-4, IL-7, IL-9, IL-15, and IL-21). In one embodiment, the method comprises administration of checkpoint blocking agents (e.g. anti-CTLA-4).

In one embodiment, the disclosure provides a method of improving immunotherapy (e.g.CAR T cell treatment) by reprogramming of T cells to overcome detrimental tumor microenvironments, including low T/M ratio, high tumor burden, high TME myeloid cell density and/or high TME myeloid inflammation levels. In one embodiment, the T cells are engineered to express gamma chain receptor cytokines. In one embodiment, the gamma chain receptor cytokines are expressed under constitutive or inducible promoters.

In one embodiment, the disclosure provides a method of improving CAR T cell treatment by optimizing T cell manufacturing to help CAR T cells overcome detrimental tumor microenvironments, wherein the characteristics of the tumor microenvironment that may be detrimental comprise low T/M ratio, high tumor burden, high TME myeloid cell density and/or high TME myeloid inflammation levels. In one embodiment, the characteristics of the TME that may be detrimental comprise low T/M ratio (within −0.5-4), high tumor burden (within 3000-40000 mm²), high myeloid cell density (within 1000-4000 cells/mm²) and/or high TME myeloid inflammation levels (within 27-2000). In one embodiment, the method comprises engineering CAR T cells to express gamma chain receptor cytokines. In one embodiment, the gamma chain receptor cytokines are expressed under constitutive or inducible promoters. In one embodiment, the method comprises growing the T cells in the presence of gamma chain cytokines such as IL-15.

In one embodiment, the disclosure provides a method of treating a malignancy in a patient comprising:
(a) analyzing a tumor biopsy from the patient to characterize the tumor microenvironment; and
(b) administering an effective dose of T cells comprising one or more chimeric receptors to the patient, wherein the effective dose is determined using the characteristics of the tumor microenvironment, wherein the characteristics of the tumor microenvironment comprise T/M ratio, tumor burden, TME myeloid cell density and/or TME myeloid inflammation levels, such as low T/M ratio (within −0.5-4), high tumor burden (within 3000-40000 mm²), high myeloid cell density (within 1000-4000 cells/mm²) and/or high myeloid inflammation levels (within 27-2000).

In one embodiment, the tumor microenvironment is characterized using gene expression profiling, intratumoral T cell density measurement, or a combination thereof.

In one embodiment, the gene expression profiling comprises determining the expression level of a specified panel of genes (herein used as biomarkers) and/or a specific subset of T cells, many of which are exemplified in this section of the disclosure and in the Examples.

In one embodiment, the disclosure provides method of determining whether a patient will respond to chimeric receptor treatment comprising:
(a) analyzing a tumor biopsy (before and/or after treatment) from the patient to characterize the tumor microenvironment using a gene expression profile or a T cell profile that is reflective of T/M ratio, tumor burden, TME myeloid cell density and/or TME myeloid inflammation levels, such as low T/M ratio (within −0.5-4), high tumor burden (within 3000-40000 mm²), high TME myeloid cell density (within 1000-4000 cells/mm²) and/or high TME myeloid inflammation levels (within 27-2000);
(b) determining an immune score based on the gene expression profile; and
(c) determining if the patient will respond to chimeric receptor treatment based on the immune score.

In one embodiment, the disclosure provides a method of determining whether a patient will respond to chimeric receptor treatment comprising:
(a) obtaining a tumor biopsy from a patient prior to treatment and after treatment;
(b) analyzing the tumor biopsy to characterize the tumor microenvironment; and
(c) determining if the patient will respond to chimeric receptor treatment based on the characteristics of the tumor microenvironment, wherein the characteristics of the tumor microenvironment comprise T/M ratio, tumor burden, TME myeloid cell density and/or TME myeloid inflammation levels, such as low T/M ratio (within −0.5-4), high tumor burden (within 3000-40000 mm²), high TME myeloid cell density (within 1000-4000 cells/mm²) and/or high TME myeloid inflammation levels (within 27-2000).

In one embodiment, the disclosure provides a method of treating a malignancy in a patient comprising:
(a) analyzing a tumor biopsy from the patient prior to chimeric receptor treatment to characterize the tumor microenvironment;
(b) determining if the patient will respond to chimeric receptor treatment based on the characteristics of the tumor microenvironment; and
(c) administering an effective dose of T cells comprising one or more chimeric receptors to the patient, wherein the effective dose is determined using the characteristics of the tumor microenvironment, wherein the characteristics of the tumor microenvironment comprise T/M ratio, tumor burden, TME myeloid cell density and/or high TME myeloid inflammation levels, such as low T/M ratio (within −0.5-4), high tumor burden (within 3000-40000 mm²), high TME myeloid cell density (within 1000-4000 cells/mm²) and/or high TME myeloid inflammation levels (within 27-2000).

In one embodiment, the characteristics of the tumor microenvironment are any of the characteristics analyzed and described in the Examples and in this section of the disclosure.

Combination of Methods of Treatment that are Adjusted Based on T/M Ratio, Tumor Burden, TME Myeloid Cell Density and/or High TIME Myeloid Inflammation Levels with Measures of Pre-Treatment Attributes

Pre-treatment attributes of the apheresis and engineered cells (T cell attributes) and patient immune factors measured from a patient sample may be used to assess the probability of clinical outcomes including response and toxicity. Attributes associated with clinical outcomes may be tumor related parameters (e.g., tumor burden, serum LDH as hypoxic/cell death marker, inflammatory markers associated with tumor burden and myeloid cell activity), T cell attributes (e.g., T cell fitness, functionality especially T1 related IFNgamma production, and the total number of CD8 T cells infused) and CART cell engraftment measured by peak CAR T cell levels in blood at early time points.

Information extrapolated from T cell attributes and patient pre-treatment attributes may be used to determine, refine or prepare a therapeutically effective dose suitable for treating a malignancy (e.g., cancer). Furthermore, some T cell attributes and patient pre-treatment attributes may be used to determine whether a patient will develop adverse events after treatment with an engineered chimeric antigen receptor (CAR) immunotherapy (e.g., neurotoxicity (NT), cytokine release syndrome (CRS)). Accordingly, an effective adverse event management strategy may be determined (e.g., administration of tocilizumab, a corticosteroid therapy, or an anti-seizure medicine for toxicity prophylaxis based on the measured levels of the one or more attributes).

In some embodiments, the pre-treatment attributes are attributes of the engineered T cells comprising one or more chimeric antigen receptors. In some embodiments, the pre-treatment attributes are T cell transduction rate, major T cell phenotype, numbers of CAR T cells and T cell subsets, fitness of CAR T cells, T cell functionality, T cell polyfunctionality, number of differentiated CAR+CD8+ T cells.

In some embodiments, the pre-treatment attributes are measured from a sample obtained from the patient (e.g., cerebrospinal fluid (CSF), blood, serum, or tissue biopsy). In some embodiments, the one or more pre-treatment attributes is tumor burden, levels of IL-6, or levels of LDH.

### T Cell Phenotypes

As described herein, the T cell phenotypes in manufacturing starting material (apheresis) may be associated with T cell fitness (DT). Total % of Tn-like and Tcm cells (CCR7+ cells) is inversely related to DT. The % of Tem (CCR7− CD45RA−) cells is directly associated with DT. Accordingly, in some embodiments, the pre-treatment attribute is the % of Tn-like and Tcm cells. In some embodiments, the % of Tn-like and Tcm cells is determined by the percentage of CCR7+ cells. In some embodiments, the percentage of CCR7+ cells is measured by flow cytometry.

In some embodiments, the pre-treatment attribute is the % of Tem (CCR7− CD45RA−) cells. In some embodiments, the % of Tem cells is determined by the percentage of CCR7− CD45RA− cells. In some embodiments, the percentage of CCR7− CD45RA− cells is measured by flow cytometry.

As described herein, manufacturing doubling time and product T-cell fitness associate directly with the differentiation state of patients' T cells prior to enrollment in CAR T cell treatment. Accordingly, the disclosure provides a method of predicting the T-cell fitness of the manufactured product comprising determining the differentiation state of the patients' T cells prior to CAR T cell treatment (e.g., in the apheresis product) and predicting T-cell fitness during manufacturing based on the differentiation state.

As described herein, the greater the proportions of effector memory T cells in the apheresis product, within total CD3+ T cells or CD4 and CD8 subsets, the higher the product doubling time. As described herein, the more juvenile the T-cell phenotype in the starting material but better the product T-cell fitness. As described herein, CD27+CD28+ T_{N} cells, which represent immunologically competent subset of T_{N} cells that express key costimulatory molecules, associate positively with product doubling time. As described herein, there is a direct association across all major phenotypic groups, including proportions of T-cell subsets defined by differentiation markers in CD3, CD4, and CD8 subpopulations, in the apheresis product relative to the final product phenotype. As described herein, the proportion of T cells with CD25^{hi} CD4 expression, possibly representing regulatory T cells in the apheresis material, negatively correlates with the CD8 T-cell output in the product. As described herein, tumor burden after CAR T cell treatment is positively associated with the differentiation phenotype of the final product.

As described herein, the number of infused CD8+ T cells normalized to tumor burden is associated with durable response and expansion of CART cells relative to tumor burden. More specifically, quartile analysis of the number of infused CD8 T cells/pretreatment tumor burden, showed a durable response rate of 16% in the lowest quartile vs. 58% in the top quartile.

As described herein, the number of infused specialized T cells, primarily the CD8+T_{N}-cell population, has a positive influence on durable clinical efficacy with CAR T-cell therapy. As described herein, higher numbers of product CD8+ T cells are needed to achieve complete tumor resolution and establish a durable response in patients with higher tumor burden. As described herein, in patients with high tumor burden, durable response is associated with significantly higher number of infused CD8 T cells compared with patients who respond and then relapse. As described herein, the number of infused TN cells normalized to tumor burden positively associates with durable response. As described herein, the CD4:CD8 ratio positively associates with durable response. As described herein, the total number of CD8 T cells in the product normalized to pretreatment tumor burden positively associates with durable response. Among CD8 T cells, the number of T_{N} cells is most significantly associated with durable response. In one embodiment (e.g., axicabtagene ciloleucel), the T_{N} cells that are identified as CCR7+CD45RA+ cells are actually stem-like memory cells and not canonical naïve T cells. The disclosure provides some additional associations, which may be used for one or more of methods of improvement of CART cell infusion product, determination of effective dose, and/or predicting durable response based on one or more of these associations. See Table 1.

TABLE 1
Association between product phenotypes and ongoing response
or peak CAR T-cell levels. P values were calculated
using logistic regression for durable response and
by Spearman correlation for CAR T-cell levels.

| | Association With | | Association With | |
|---|---|---|---|---|
| | Durable Response | | Peak CAR T-cell Levels | |
| | P | Direction of | P | Direction of |
| Parameter | value | association | value | association |
| CD3 infused (%) | 0.201 | Negative | 0.762 | Positive |
| Number of CD3 infused^{a} | 0.654 | Positive | 0.441 | Positive |
| Number of CD3 infused/ | 0.030 | Positive | 0.443 | Positive |
| tumor burden^{a} | | | | |
| ⁺Tₙ infused (%) | 0.454 | Positive | 0.099 | Positive |
| Number of ⁺Tn infused^{a} | 0.182 | Positive | 0.091 | Positive |
| Number of ⁺Tn infused/ | 0.025 | Positive | 0.114 | Positive |
| tumor burden^{a} | | | | |
| % CD8 infused | 0.21 | Positive | 0.126 | Positive |
| Number of CD8^{a} | 0.116 | Positive | 0.154 | Positive |
| Number of CD8 infused/ | 0.009 | Positive | 0.273 | Positive |
| tumor burden^{a} | | | | |
| CD4 infused (%) | 0.21 | Negative | 0.124 | Negative |
| Number of CD4 infused^{a} | 0.930 | Negative | 0.257 | Negative |
| Number of CD4 infused/ | 0.059 | Positive | 0.841 | Positive |
| tumor burden^{a} | | | | |

^{a}Denote analytes in LOG2 transformation.
⁺The cells referred to as T_{N} in the EXAMPLES were identified simply as CCR7+ CD45RA+ T-cells and have been further characterized as stem-like memory cells.

Accordingly, the disclosure provides a method of improving durable clinical efficacy (e.g., durable response) of CAR T-cell therapy in a patient comprising preparing and/or administering to the patient an effective dose of CAR T cell treatment, wherein the effective dose is determined based on a combination of T/M ratio, tumor burden, TME myeloid cell density and/or high TME myeloid inflammation levels and the number of specialized T cells in the infusion product and/or the CD4:CD8 ratio. In some embodiments, the specialized T cells are CD8+ T cells, preferably T_{N} cells. In one embodiment (e.g., axicabtagene ciloleucel), the cells referred to as T_{N} are identified as CCR7+CD45RA+ T-cells and have been further characterized as stem-like memory cells.

In another embodiment, the disclosure provides a method of determining how a patient will respond to treatment comprising (a) characterizing T/M ratio, tumor burden, TME myeloid cell density and/or high TME myeloid inflammation levels and the number of specialized T cells in the infusion product to obtain one or more values and (b) determining how the patient will respond based on the one or more values. In another embodiment, the present disclosure provides a method of treating a malignancy in a patient comprising measuring the T cell phenotypes in a population of T cells obtained from a patient (e.g., apheresis material) in combination with measurements of T/M ratio, tumor burden, TME myeloid cell density and/or high TME myeloid inflammation levels and. In some embodiments, the method further comprises determining whether the patient will respond to chimeric antigen receptor treatment based on the measured percentage of specific T cell types. In some embodiments, the T cell phenotype is measured prior to engineering the cells to express a chimeric antigen receptor (CAR) (e.g., apheresis material). In some embodiments, the T cell phenotype is measured after engineering the cells to express a chimeric antigen receptor (CAR) (e.g., engineered T cells comprising a CAR).

As described herein, the number of CCR7+CD45RA+ cells in the product infusion bag associates positively with a (“rapid”) response (approximately two weeks) to axicabtagene ciloleucel treatment. Accordingly, the percentage or total number of these cells in the T cell product may be manipulated to improve response to T cell therapy.

As described herein, the higher the frequency of CCR7+CD45RA+ T cells in the product infusion bag, the higher the product T-cell fitness. As described herein, the higher the frequency of CCR7+CD45RA+ T cells in the product infusion bag, the lower the product doubling time. Accordingly, the percentage or total number of these cells in the T cell product may be manipulated to decrease DT and improve response to T cell therapy.

As described herein, the majority of CCR7+ CD45RA+ T cells in the axicabtagene ciloleucel product infusion bag were stem-like memory cells, not canonical naïve T cells. As described herein, CCR7+ CD45RA+ T cells from peripheral blood may differentiate in vitro into stem-like memory cells.

As described herein, the T cell subpopulation that best associates with DT was CCR7+CD45RA+CD27+CD28+ T cells. Accordingly, the percentage or total number of these cells in the T cell product may be manipulated to decrease DT and improve response to T cell therapy.

As described herein, CCR7+ CD45RA+ T cells are drivers of anti-tumor activity in the context of T-cell therapies. Accordingly, the percentage or total number of these cells in the T cell product may be manipulated to improve response to T cell therapy.

As described herein, the total number of specialized T cells normalized to pretreatment tumor burden associates better with clinical efficacy than the number of product T cells of CAR T cells. Accordingly, the percentage or total number of these cells in the T cell product may be manipulated to improve response to T cell therapy.

### T1 Functionality

Engineered T cells may be characterized by their immune function characteristics. Methods of the present disclosure provide measuring T/M ratio, tumor burden, TME myeloid cell density and/or TME myeloid inflammation levels in combination with levels of cytokine production ex vivo. In some embodiments, the cytokines are selected from the group consisting of IFNgamma, TNFa, IL-12, MIP1β, MIP1α, IL-2, IL-4, IL-5, and IL-13. In some embodiments, the T cell functionality is measured by levels of Th1 cytokines.

In some embodiments, the Th1 cytokines are selected from the group consisting of IFNgamma, TNFa, and IL-12. In some embodiments, T cell functionality is measured by levels of IFNgamma production. In some embodiments, excess T cell IFNgamma (pre-treatment attribute), and post-treatment T1 activity, are attributes that may be used to determine whether a patient will develop adverse events (e.g., neurotoxicity). In some embodiments, IFNgamma levels produced by engineered CAR T cells are measured by co-culture prior to administration of engineered CAR T cells.

In some embodiments, engineered CAR T cells with lower co-culture IFNgamma result in positive clinical efficacy outcome and reduced grade 3+ neurotoxicity. In one aspect, the present disclosure provides a method of treating a malignancy in a patient comprising measuring the levels of IFNgamma produced by a population of engineered T cells comprising a chimeric antigen receptor (CAR). In some embodiments, the method further comprises determining whether the patient will respond to chimeric antigen receptor treatment based on the measured levels of IFNgamma compared to a reference level. In some embodiments, the reference level is less than about 1 ng/ml, about 2 ng/ml, about 3 ng/ml, about 4 ng/ml, about 5 ng/ml, about 6 ng/ml, about 7 ng/ml, or about 8 ng/ml.

In some embodiments, engineered CAR T cells with excess IFNgamma production show rapidly elevating rate of grade 3+ neurotoxicity and diminution of objective response rate. In one aspect, the present disclosure provides a method of treating a malignancy in a patient comprising measuring the levels of IFNgamma produced by a population of engineered T cells comprising a chimeric antigen receptor (CAR). In some embodiments, the method further comprises determining whether the patient will develop an adverse event to chimeric antigen receptor treatment based on the measured levels of IFNgamma compared to a reference level. In some embodiments, the reference level is greater than about 5 ng/ml, about 6 ng/ml, about 7 ng/ml, or about 8 ng/ml, about 9 ng/ml, about 10 ng/ml, or about 11 ng/ml.

As described herein, there is a direct association of early elevation of IFNgamma in serum after CAR T cell infusion and rate of grade 3+ toxicities. In some embodiments, IFNgamma elevation in serum post CAR T cell infusion (day 1/day 0 fold change) is measured. In some embodiments, day 1/day 0 serum IFNgamma fold change greater than about 25 results in grade 3+ neurotoxicity. In some embodiments, day 1/day 0 serum IFNgamma fold change greater than about 30, about 35, about 40, about 45, or about 50 results in grade 3+ neurotoxicity.

There is a direct association of early elevation of IFNgamma related CXCL10 (IP-10) elevation in serum after CAR T cell infusion and rate of grade 3+ toxicities. In some embodiments, IFNgamma related CXCL10 (IP-10) elevation in serum post CAR T cell infusion (day 1/day 0 fold change) is measured. In some embodiments, day 1/day 0 serum IFNgamma related CXCL10 (IP-10) fold change a greater than about 2.5 results in grade 3+ neurotoxicity. In some embodiments, day 1/day 0 serum IFNgamma related CXCL10 (IP-10) fold change greater than about 3.0, about 3.5, about 4.0, about 4.5, or about 5.0 results in grade 3+ neurotoxicity.

As described herein, pretreatment product T-cell IFNγ production is linked to the more differentiated T cells in the infusion bag and associated positively with severe neurologic toxicities and to a lesser degree with decreased efficacy. Accordingly, in one embodiment, the disclosure provides a method of predicting neurologic toxicities comprising measuring the pretreatment product T-cell IFNγ production level and predicting neurologic toxicities based on that level. In one embodiment, the method further comprises modulating the pretreatment product T-cell IFNγ production level to improve the effectiveness and/or toxicity of the CAR T cell treatment. In some embodiments, the method further comprises administering an effective dose of CAR T cell treatment wherein the effective dose is determined based on the product T-cell IFNγ production level.

Systemic inflammatory conditions have been associated with elevated serum ferritin, C-reactive protein (CRP), IL6, IL8, CCL2, as well as decreased serum albumin and indicate a generalized myeloid activation state. Myeloid-derived suppressor cells are known to be induced by IL8 and CCL2 within tumors and mobilized by IL6 from the bone marrow.

As described herein, low T/M ratio, high tumor burden, high TME myeloid cell density and/or high TME myeloid inflammation levels in combination with pro-inflammatory and myeloid activation markers (e.g., IL6, ferritin, CCL2) in the serum measured prior to conditioning (at baseline) correlate with impaired in vivo CAR T-cell expansion and decreased rate of durable response. Accordingly, in one embodiment, the disclosure provides a method of increasing the rate of durable response after CAR T cell treatment comprising decreasing the baseline levels of pro-inflammatory and myeloid activation markers in the patient serum and/or TME prior to CAR T cell treatment administration. The disclosure also provides a method of determining whether or not a patient will have a durable response to CAR T cell treatment comprising measuring T/M ratio, tumor burden, TME myeloid cell density and/or TME myeloid inflammation levels in combination with the baseline levels of pro-inflammatory and myeloid activation markers and making the determination based on those levels. In some embodiments, the method further comprises administering an effective dose of CAR T cell treatment wherein the effective dose is determined based on the baseline levels of pro-inflammatory and myeloid activation markers. As described herein, persisting systemic inflammation after CAR T-cell infusion associates with a failure of the CAR T cells to completely eliminate the tumor.

As described herein, pretreatment levels measured prior conditioning (at baseline) of pro-inflammatory markers associated positively with each other and negatively with hemoglobin and platelet levels. As described herein, pretreatment tumor burden correlates with baseline serum LDH, ferritin, and IL6 but not with CCL2. As described herein, pretreatment ferritin and LDH negatively associate with CAR T-cell expansion normalized to pretreatment tumor burden (peak CAR T-cell expansion/tumor burden). As described herein, pretreatment tumor burden and systemic inflammation negatively associate with the rate of durable responses; this effect may be mediated by decreased CAR-T-cell expansion relative to the pretreatment tumor burden. Accordingly, in one embodiment, the disclosure provides a method of increasing the rate of durable response after CAR T cell treatment comprising decreasing the systemic inflammation in the patient prior to CAR T cell treatment administration. The disclosure also provides a method of determining whether or not a patient will have a durable response to CAR T cell treatment comprising measuring pretreatment tumor burden and inflammation to obtain their levels and making the determination based on those levels. In some embodiments, the method further comprises administering an effective dose of CAR T cell treatment wherein the effective dose is calculated based on those levels.

As described herein, elevated LDH associates with decreased durable response. Accordingly, the disclosure also provides a method of determining whether or not a patient will have a durable response to CAR T cell treatment comprising measuring the baseline level of LDH and making the determination based on those levels. In some embodiments, the method further comprises administering an effective dose of CAR T cell treatment wherein the effective dose is determined based on the baseline levels of LDH.

As described herein, baseline IL6 elevation associates with both decreased response rates and durable response rates. Accordingly, the disclosure provides a method of increasing the response and durable response after CAR T cell treatment comprising decreasing the baseline levels of IL6 prior to CAR T cell treatment administration. The disclosure also provides a method of determining whether or not a patient will have a durable response to CAR T cell treatment comprising measuring the baseline levels of IL6 and making the determination based on those levels. In some embodiments, the method further comprises administering an effective dose of CAR T cell treatment wherein the effective dose is determined based on the baseline levels of IL6. In one embodiment, baseline IL6 activation or levels are decreased with an agent like tocilizumab (or another anti-IL6/IL6R agent/antagonist).

As described herein, high peak and cumulative ferritin levels within the first 28 days after infusion associate with lower in vivo CAR T-cell expansion and lower rates of durable response. Accordingly, the disclosure provides a method of increasing the response and durable response after CAR T cell treatment comprising decreasing the high peak and cumulative ferritin levels after CAR T cell treatment administration during the first 28 days. The disclosure also provides a method of determining whether or not a patient will have a durable response to CAR T cell treatment comprising measuring the high peak and cumulative ferritin levels within the first 28 days after infusion and making the determination based on those levels.

As described herein, there is an association between ferritin levels over the first 28 days, and peak CAR T-cell levels normalized to tumor burden. As described herein, higher levels of serum ferritin at most time points after CAR T-cell infusion are seen in patients who relapse or have no response compared with those who have durable response. Accordingly, the disclosure also provides a method of determining whether or not a patient will relapse or have no response to CAR T cell treatment comprising measuring the levels of serum ferritin at a time point after CAR T-cell infusion and making the determination based on those levels (e.g., relative to a reference value).

As described herein, elevated pretreatment or posttreatment pro-inflammatory, myeloid-related cytokines (IL6, ferritin, CCL2), as well as LDH, are positively associated with grade ≥3 NE or CRS. Accordingly, the disclosure provides a method of decreasing grade ≥3 NE and/or CRS comprising decreasing the pretreatment and/or posttreatment levels of one or more pro-inflammatory, myeloid-related cytokines (e.g., IL6, ferritin, CCL2) and/or LDH. The disclosure also provides a method of determining whether or not a patient will have ≥3 NE or CRS after administration of CAR T cell treatment comprising measuring the baseline levels of pro-inflammatory, myeloid-related cytokines (IL6, ferritin, CCL2), and/or LDH and making the determination based on those levels. In some embodiments, the method further comprises administering an effective dose of CAR T cell treatment wherein the effective dose is determined based on the baseline levels of pro-inflammatory, myeloid-related cytokines (IL6, ferritin, CCL2), as well as LDH.

As described herein, serum levels of IFNγ, CXCL10, and IL15, measured early posttreatment, associate positively with neurotoxicity but are not associated with durable response rate. Accordingly, the disclosure provides a method of decreasing neurotoxicity comprising decreasing the early posttreatment serum levels of IFNγ, CXCL10, and/or IL15. As described herein, day 0 IL15 serum levels significantly associate with day 1 IFNγ serum levels, rather than product co-culture IFNγ.

The disclosure also provides a method of determining whether or not a patient will show neurotoxicity after administration of CAR T cell treatment comprising measuring the serum levels of IFNγ, CXCL10, and IL15, measured early posttreatment and making the determination based on those levels. In some embodiments, the method further comprises administering an effective dose of agents that decrease neurotoxicity wherein the effective dose is determined based on the baseline levels of IFNγ, CXCL10, and IL15. In some embodiments, the levels are measured at day 0 and/or day 1, posttreatment. In some embodiments, the agents are selected from agents that decrease the levels or activity of IFNγ, CXCL10, and IL15 and/or other cytokines.

Tumor related parameters (e.g., tumor burden, serum LDH as hypoxic/cell death marker, inflammatory markers associated with tumor burden and myeloid cell activity) may be associated with clinical outcomes. In one aspect, the present disclosure provides a method of treating a malignancy in a patient comprising measuring the tumor burden in a patient prior to administration of a CART cell treatment, in combination with measuring T/M ratio, TME myeloid cell density and/or TME myeloid inflammation levels. In some embodiments, the method further comprises determining whether the patient will respond to CAR T cell treatment based on the levels of tumor burden compared to a reference level. In some embodiments, the reference level is less than about 1,000 mm², about 2,000 mm², about 3,000 mm², about 4,000 mm².

As described herein, the higher the tumor burden, the higher the probability of relapse within 1 year post treatment in subjects who achieved an OR, and the higher the probability of grade 3+ neurotoxicity. In some embodiments, tumor burden may be used to assess the probability of relapse in patients who respond, if the pre-treatment tumor burden is greater than about 4,000 mm², about 5,000 mm², about 6,000 mm², about 7,000 mm², or about 8,000 mm².

As described herein, low tumor burden pre-CAR T-cell therapy is a positive predictor of durable response. As described herein, in the highest tumor burden quartile, patients who achieved a durable response had a greater than 3-fold higher peak CAR T-cell expansion compared with patients who relapsed or had no response. As described herein, there is a lower durable response rate at comparable peak CAR T-cell levels in patients with higher tumor burden compared with patients who had lower tumor burden. As described herein, durable responders had a higher peak CAR T-cell/tumor burden ratio compared with nonresponders or responders who subsequently relapsed within one year posttreatment. As described herein, complete responders had a higher peak CAR T-cell/tumor burden ratio compared with partial responders or nonresponders. Accordingly, the disclosure also provides a method of determining whether or not a patient will be a nonresponder, have a durable response, or relapse within one year after administration of CAR T cell treatment comprising measuring the peak CAR T-cell/tumor burden ratio and making the determination based on those levels. As described herein, objective and durable response rate correlate with increasing peak CAR T-cell levels. As described herein, there is a lower durable response rate (12%) in patients within the lowest quartile of peak CAR T-cell/tumor burden ratio than in the top quartiles (>50%). As described herein, durable response in refractory large cell lymphoma treated with anti-CD19 CAR T-cell therapy containing a CD28 costimulatory domain, benefits from early CAR T cell expansion, commensurate with tumor burden.

As described herein, tumor burden positively associates with severe neurotoxicity: while rates increase from quartile 1 to quartile 3, they decline in the highest quartile, generally mirroring the association between CAR T-cell expansion and tumor burden in the overall population.

As described herein, peak CAR T-cell levels that are normalized to either pretreatment tumor burden or body weight associate strongly with efficacy, and the latter associate with grade ≥3 NE. Accordingly, the disclosure also provides a method of determining whether or not a patient will show durable response after administration of CAR T cell treatment comprising measuring the peak CAR T-cell levels normalized to either pretreatment tumor burden or body weight and making the determination based on those levels. Also, the disclosure also provides a method of determining whether or not a patient will show grade ≥3 NE after administration of CAR T cell treatment comprising measuring the peak CAR T-cell levels normalized to pretreatment tumor body weight and making the determination based on those levels.

As described herein, in vivo CAR T-cell expansion commensurate with pretreatment tumor burden and influenced by intrinsic product T-cell fitness, dose of specialized T-cell subsets, and host systemic inflammation, were determining factors for durable response. Accordingly, these parameters may be used as biomarkers for durable response and may also be manipulated experimentally to improve response to T cell therapy.

As described herein, suboptimal product T-cell fitness was a major factor related to primary treatment resistance, and limited numbers of CCR7+CD45RA+ or CD8 T cells in proportion to tumor burden were associated with a failure to achieve durable response. Accordingly, these parameters may be used as biomarkers for durable response and may also be manipulated experimentally to improve response to T cell therapy.

As described herein, high tumor burden, pronounced inflammatory status (reflected by myeloid activation markers pre- and post-CAR T-cell infusion), and excess type-1 cytokines associated negatively with durable efficacy and positively with severe toxicities. Accordingly, these parameters may be used as biomarkers for durable response and may also be manipulated experimentally to improve response to T cell therapy.

### Clinical Outcomes

In some embodiments, the clinical outcome is complete response. In some embodiments, the clinical outcome is durable response. In some embodiments, the clinical outcome is complete response. In some embodiments, the clinical outcome is no response. In some embodiments, the clinical outcome is partial response. In some embodiments, the clinical outcome is objective response. In some embodiments, the clinical outcome is survival. In some embodiments, the clinical outcome is relapse.

In some embodiments, objective response (OR) is determined per the revised IWG Response Criteria for Malignant Lymphoma (Cheson, 2007) and determined by IWG Response Criteria for Malignant Lymphoma (Cheson et al. Journal of Clinical Oncology 32, no. 27 (September 2014) 3059-3067). Duration of Response is assessed. The Progression-Free Survival (PFS) by investigator assessment per Lugano Response Classification Criteria is evaluated.

In some embodiments, response, levels of CART cells in blood, or immune related factors is determined by follow up at about 1 day, about 2 days, about 3 days, about 4 days, about 5 days, about 6 days, or about 7 days after administration of engineered CAR T cells. In some embodiments, response, levels of CAR T cells in blood, or immune related factors is determined by follow up at about 1 week, about 2 weeks, about 3 weeks, or about 4 weeks after administration of engineered CART cells. In some embodiments, response, levels of CART cells in blood and/or immune related factors are determined by follow up at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administration of a engineered CAR T cells. In some embodiments, response, levels of CAR T cells in blood and/or immune related factors are determined by follow up at about 1 year, about 1.5 years, about 2 years, about 2.5 years, about 3 years, about 4 years, or about 5 years after administration of engineered CAR T cells.

In some embodiments, methods described herein may provide a clinical benefit to a subject. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, approximately 1%, 2%, 3%, 4%, 5%, 6%, 7%, 8%, 9%, 10%, 15%, 20%, 25%, 30%, 35%, 40%, 45%, 0%, 55%, 60%, 65%, 70%, 75%, 80%, 85%, 90% or 95% and any unenumerated % in between of patients achieve a clinical benefit. In some embodiments, the response rate is 1%, 2%, 3%, 4%, 5%, 6%, 7%, 8%, 9%, 9.5%, 10.5%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 25 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81, 82, 83, 84, 85, 86, 87, 88, 89, 90, 91, 92, 93, 94, 95, 96, 97, 98, 99, or 100% or some other unenumerated percentage and range in between 1% and 100%. In some embodiments, the response rate is between 0%-10%, 10%-20%, 20%-30%, 30%-40%, 40%-50%, 50%-60%, 60%-70%, 70%-80%, 80%-90%, or 90%-100%. In some embodiments, the response rate is between 0%-1.%, 1%-1.5%, 1.5%-2%, 2%-3%, 3%-4%, 4%-5%, 5%-6%, 6%-7%, 7%-8%, 8%-9%, 9%-10%, 10%-15%, 15%-20%, 20-25%, 25%-30%, 35-40%, and so one and so forth, through 95%-100%.

In one embodiment, the immunotherapy is CAR-T cell immunotherapy. Chimeric antigen receptors (CARs) are genetically engineered receptors. These engineered receptors may be inserted into and expressed by immune cells, including T cells and other lymphocytes in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016).

In some embodiments, a costimulatory domain which includes a truncated hinge domain (“THD”) further comprises some or all of a member of the immunoglobulin family such as IgG1, IgG2, IgG3, IgG4, IgA, IgD, IgE, IgM, or fragment thereof.

In some embodiments, the THD is derived from a human complete hinge domain (“CHD”). In other embodiments, the THD is derived from a rodent, murine, or primate (e.g., non-human primate) CHD of a costimulatory protein. In some embodiments, the THD is derived from a chimeric CHD of a costimulatory protein.

The costimulatory domain for the CAR of the disclosure may further comprise a transmembrane domain and/or an intracellular signaling domain. The transmembrane domain may be fused to the extracellular domain of the CAR. The costimulatory domain may similarly be fused to the intracellular domain of the CAR. In some embodiments, the transmembrane domain that naturally is associated with one of the domains in a CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this disclosure may be derived from (i.e., comprise) 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CD S, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Optionally, short linkers may form linkages between any or some of the extracellular, transmembrane, and intracellular domains of the CAR. In some embodiments, the linker may be derived from repeats of glycine-glycine-glycine-glycine-serine (SEQ ID NO: 2) (G4S)n or GSTSGSGKPGSGEGSTKG (SEQ ID NO: 1). In some embodiments, the linker comprises 3-20 amino acids and an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to GSTSGSGKPGSGEGSTKG (SEQ ID NO: 1).

The linkers described herein, may also be used as a peptide tag. The linker peptide sequence may be of any appropriate length to connect one or more proteins of interest and is preferably designed to be sufficiently flexible so as to allow the proper folding and/or function and/or activity of one or both of the peptides it connects. Thus, the linker peptide may have a length of no more than 10, no more than 11, no more than 12, no more than 13, no more than 14, no more than 15, no more than 16, no more than 17, no more than 18, no more than 19, or no more than 20 amino acids. In some embodiments, the linker peptide comprises a length of at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the linker comprises at least 7 and no more than 20 amino acids, at least 7 and no more than 19 amino acids, at least 7 and no more than 18 amino acids, at least 7 and no more than 17 amino acids, at least 7 and no more than 16 amino acids, at least 7 and no more 15 amino acids, at least 7 and no more than 14 amino acids, at least 7 and no more than 13 amino acids, at least 7 and no more than 12 amino acids or at least 7 and no more than 11 amino acids. In certain embodiments, the linker comprises 15-17 amino acids, and in particular embodiments, comprises 16 amino acids. In some embodiments, the linker comprises 10-20 amino acids. In some embodiments, the linker comprises 14-19 amino acids. In some embodiments, the linker comprises 15-17 amino acids. In some embodiments, the linker comprises 15-16 amino acids. In some embodiments, the linker comprises 16 amino acids. In some embodiments, the linker comprises 3, 4, 5, 6, 7, 8, 9, 10, 11, 12, 13, 14, 15, 16, 17, 18, 19 or 20 amino acids.

In some embodiments, a spacer domain is used. In some embodiments, the spacer domain is derived from CD4, CD8a, CD8b, CD28, CD28T, 4-1BB, or other molecule described herein. In some embodiments, the spacer domains may include a chemically induced dimerizer to control expression upon addition of a small molecule. In some embodiments, a spacer is not used.

The intracellular (signaling) domain of the engineered T cells of the disclosure may provide signaling to an activating domain, which then activates at least one of the normal effector functions of the immune cell. Effector function of a T cell, for example, may be cytolytic activity or helper activity including the secretion of cytokines.

In certain embodiments, suitable intracellular signaling domain include (i.e., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Ly108), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

### Antigen Binding Molecules

Suitable CARs and TCRs may bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (“scFv”). A scFv is a single chain antibody fragment having the variable regions of the heavy and light chains of an antibody linked together. See U.S. Pat. Nos. 7,741,465 and 6,319,494, as well as Eshhar et al., Cancer Immunol Immunotherapy (1997) 45: 131-136. A scFv retains the parent antibody's ability to interact specifically with target antigen. scFv's are useful in chimeric antigen receptors because they may be engineered to be expressed as part of a single chain along with the other CAR components. Id. See also Krause et al., J. Exp. Med., Volume 188, No. 4,1998 (619-626); Finney et al., Journal of Immunology, 1998, 161: 2791-2797. It will be appreciated that the antigen binding molecule is typically contained within the extracellular portion of the CAR or TCR such that it is capable of recognizing and binding to the antigen of interest. Bispecific and multi specific CARs and TCRs are contemplated within the scope of the disclosure, with specificity to more than one target of interest.

In some embodiments, the polynucleotide encodes a CAR or TCR comprising a (truncated) hinge domain and an antigen binding molecule that specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface antigens.

In one embodiment, the immunotherapy is T cell therapy. In one embodiment, the cells from a subject. In one embodiment, the cells are Induced Pluripotent Stem Cells (iPSCs). T cells may be obtained from, e.g., peripheral blood mononuclear cells, bone marrow, lymph node tissue, cord blood, thymus tissue, tissue from a site of infection, ascites, pleural effusion, spleen tissue, tumors, or differentiated in vitro. In addition, the T cells may be derived from one or more T cell lines available in the art. T cells may also be obtained from a unit of blood collected from a subject using any number of techniques known to the skilled artisan, such as FICOLL™ separation and/or apheresis. In some embodiments, the cells collected by apheresis are washed to remove the plasma fraction, and placed in an appropriate buffer or media for subsequent processing. In some embodiments, the cells are washed with PBS. As will be appreciated, a washing step may be used, such as by using a semi-automated flow through centrifuge, e.g., the Cobe™ 2991 cell processor, the Baxter CytoMate™, or the like. In some embodiments, the washed cells are resuspended in one or more biocompatible buffers, or other saline solution with or without buffer. In some embodiments, the undesired components of the apheresis sample are removed. Additional methods of isolating T cells for a T cell therapy are disclosed in U.S. Patent Pub. No. 2013/0287748, which is herein incorporated by references in its entirety.

In some embodiments, T cells are isolated from PBMCs by lysing the red blood cells and depleting the monocytes, e.g., by using centrifugation through a PERCOLL′ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8+ cells are further sorted into naive, central memory, and effector cells by identifying cell surface antigens that are associated with each of these types of CD8+ cells. In some embodiments, the expression of phenotypic markers of central memory T cells includes expression of CCR7, CD3, CD28, CD45RO, CD62L, and CD127 and negative for granzyme B. In some embodiments, central memory T cells are CD8+, CD45RO+, and CD62L+ T cells. In some embodiments, effector T cells are negative for CCR7, CD28, CD62L, and CD127 and positive for granzyme B and perforin. In some embodiments, CD4+ T cells are further sorted into subpopulations. For example, CD4+ T helper cells may be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, the T cells are obtained from a donor subject. In some embodiments, the donor subject is human patient afflicted with a cancer or a tumor. In some embodiments, the donor subject is a human patient not afflicted with a cancer or a tumor.

In some embodiments, a composition comprising engineered T cells comprises a pharmaceutically acceptable carrier, diluent, solubilizer, emulsifier, preservative and/or adjuvant. In some embodiments, the composition comprises an excipient. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the composition is selected for parenteral delivery, for inhalation, or for delivery through the digestive tract, such as orally. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In some embodiments, the vehicle for parenteral injection is sterile distilled water in which composition described herein, with or without at least one additional therapeutic agent, is formulated as a sterile, isotonic solution, properly preserved. In some embodiments, the preparation involves the formulation of the desired molecule with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the methods of treating a cancer in a subject in need thereof comprise a T cell therapy. In some embodiments, the T cell therapy disclosed herein is engineered Autologous Cell Therapy (eACT™). According to this embodiment, the method may include collecting blood cells from the patient. The isolated blood cells (e.g., T cells) may then be engineered to express a CAR disclosed herein. In a particular embodiment, the CAR T cells are administered to the patient. In some embodiments, the CAR T cells treat a tumor or a cancer in the patient. In some embodiments the CAR T cells reduce the size of a tumor or a cancer.

In some embodiments, the donor T cells for use in the T cell therapy are obtained from the patient (e.g., for an autologous T cell therapy). In other embodiments, the donor T cells for use in the T cell therapy are obtained from a subject that is not the patient. In certain embodiments, the T cell is a tumor-infiltrating lymphocyte (TIL), engineered autologous T cell (eACT™), an allogeneic T cell, a heterologous T cell, or any combination thereof.

In some embodiments, the engineered T cells are administered at a therapeutically effective amount. For example, a therapeutically effective amount of the engineered T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the therapeutically effective amount of the engineered viable T cells is between about 1×10⁶ and about 2×10⁶ engineered viable T cells per kg body weight up to a maximum dose of about 1×10⁸ engineered viable T cells.

In some embodiments, the engineered T cells are anti-CD19 CART T cells. In some embodiments, the anti-CD19 CAR T cells are the axicabtagene ciloleucel product, YESCARTA™ axicabtagene ciloleucel (axicabtagene ciloleucel), TECARTUS™-brexucabtagene autoleucel/KTE-X19, KYMRIAH™ (tisagenlecleucel), etc, In some embodiments, the product meets commercial specifications. In some embodiments, the product does not meet commercial specifications (out-of-specification product, OOS). In some embodiments, the OOS product comprises fewer, less differentiated CCR7+ T_{N} and T_{CM} and a greater proportion of more differentiated CCR7− T_{EM}+ T_{EFF} cells than the axicabtagene ciloleucel product that meets commercial specifications. In some embodiments, the OOS product results in a median peak CAR T cell level after administration that is lower than that of the commercial product. In some embodiments, the OOS product still showed a manageable safety profile and meaningful clinical benefit.

The methods disclosed herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In some embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, Waldenstrom macroglobulinemia, or a combination thereof.

In some embodiments, the cancer is a myeloma. In some embodiments, the cancer is multiple myeloma. In some embodiments, the cancer is leukemia. In some embodiments, the cancer is acute myeloid leukemia.

In some embodiments, the cancer is Non-Hodgking lymphoma. In some embodiments, the cancer is relapsed/refractory NHL. In some embodiments, the cancer is mantle cell lymphoma.

In some embodiments, the cancer is advanced-stage indolent non-Hodgkin lymphoma (iNHL), including follicular lymphoma (FL) and marginal zone lymphoma (MZL). In some embodiments, the patient has had relapsed/refractory disease after ≥2 prior lines of therapy, including an anti-CD20 monoclonal antibody with an alkylating agent. In some embodiments, the patient may have received a PI3K inhibitor. In some embodiments, the patient may (also) have received autologous stem cell transplantation. In some embodiments, the patient undergoes leukapheresis to obtain T cells for CAR T cell manufacturing, followed by conditioning chemotherapy with cyclophosphamide at 500 mg/m²/day and fludarabine at 30 mg/m²/day administered on days −5, −4, and −3; on day 0, the patient may receive a single intravenous infusion of CAR T cell therapy (e.g., axicabtagene ciloleucel) at a target dose of 2×10⁶ CAR T cells/kg. In some embodiments, additional infusions may be given at a later period. In some embodiments, if the patient progresses after responding at the month 3 assessment after initial administration, the patient may receive retreatment with CAR T cell treatment (e.g., axicabtagene ciloleucel). In some embodiments, the patient may receive bridging therapy. Examples of bridging therapies are provided elsewhere in the specification, including the Examples. In some embodiments, the patient experiences CRS. In some embodiments, CRS is managed using any one of the protocols described in this application, including the Examples. In some embodiments, CRS is managed with tocilizumab, corticosteroids and/or vasopressor.

In some embodiments, the cancer is relapsed/refractory indolent Non-Hodgkin Lymphoma and the method of treating a subject in need thereof comprises administering to the subject a therapeutically effective amount of CAR T cells as a retreatment, wherein the subject has previously received a first treatment with CAR T cells. In some embodiments, the first treatment with CAR T cells may have been administered as a first line therapy or a second line therapy, optionally wherein the lymphoma is R/R follicular lymphoma (FL) or marginal zone lymphoma (MZL) and optionally wherein the previous prior lines of therapy included anti-CD20 monoclonal antibody combined with an alkylating agent. In some embodiments, the conditioning therapy comprises fludarabine 30 mg/m² IV and cyclophosphamide 500 mg/m² IV on Days −5, −4, and −3. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cells are axicabtagene ciloleucel CAR T cells. In some embodiments, the retreatment eligibility criteria include response of a CR or PR at the month 3 disease assessment with subsequent progression; no evidence of CD19 loss in progression biopsy by local review; and/or no Grade 4 CRS or neurologic events, or life-threatening toxicities with the first treatment with CAR T cells. In some embodiments, the method of treatment is that followed by the CLINICAL TRIAL-5 clinical trial (NCT03105336).

In some embodiments, the cancer is NHL and the immunotherapy (e.g, CAR T or TCR T cell treatment) is administered as a first line therapy. In some embodiments, the cancer is LBCL. In some embodiments, the LBCL is high risk/high grade LBCL with MYC and BCL2 and/or BCL6 translocations or DLBCL with IPI score ≥3 any time before enrollment. In some embodiments, the first line therapy comprises CAR T cell treatment in combination with an anti-CD20 monoclonal antibody and anthracycline-containing regimen. In some embodiments, the CAR T cell treatment is administered first. In some embodiments, the anti-CD20 monoclonal antibody/anthracycline-containing regimen is administered first. In some embodiments, the treatments are administered at least 2 weeks, at least 4 weeks, at least 6 weeks, at least 1 month, at least 2 months, at least 3 months, at least 4 months, at least 5 months, less than a year apart, etc. In some embodiments, the method further comprises bridging therapy administered after leukapheresis and completed prior to initiating conditioning chemotherapy. In some embodiments, additional inclusion criteria include age ≥18 years and ECOG PS 0-1. In some embodiments, the conditioning therapy comprises fludarabine 30 mg/m² IV and cyclophosphamide 500 mg/m² IV on Days −5, −4, and −3. Other exemplary beneficial preconditioning treatment regimens are described in U.S. Provisional Patent Applications 62/262,143 and 62/167,750 and U.S. Pat. Nos. 9,855,298 and 10,322,146, which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. Another embodiment comprises serum cyclophosphamide and fludarabine at days −4, −3, and −2 prior to T cell administration at a dose of 500 mg/m² of body surface area of cyclophosphamide per day and a dose of 30 mg/m² of body surface area per day of fludarabine during that period of time. Another embodiment comprises cyclophosphamide at day −2 and fludarabine at days −4, −3, and −2 prior to T cell administration, at a dose of 900 mg/m² of body surface area of cyclophosphamide and a dose of 25 mg/m² of body surface area per day of fludarabine during that period of time. In another embodiment, the conditioning comprises cyclophosphamide and fludarabine at days −5, −4 and −3 prior to T cell administration at a dose of 500 mg/m² of body surface area of cyclophosphamide per day and a dose of 30 mg/m² of body surface area of fludarabine per day during that period of time, Other preconditioning embodiments comprise 200-300 mg/m² of body surface area of cyclophosphamide per day and a dose of 20-50 mg/m² of body surface area per day of fludarabine for three days. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cell treatment comprises anti-CD19 CAR T cells. In some embodiments, the CAR T cell treatment comprises axicabtagene ciloleucel or YESCARTA™. In some embodiments, the CAR T cell treatment comprises TECARTUS™-brexucabtagene autoleucel/KTE-X19 or KYMRIAH™ (tisagenlecleucel), etc), In some embodiments, the method of treatment is the method used in any one of the ZUMA-1 through ZUMA-19, KITE-585, KITE-222, KITE-037, KITE-363, KITE-439, or KITE-718 clinical trials, which are well-described in the art.

In another embodiment, the disclosure provides a method of treating cancer in a subject in need thereof, comprising administering a therapeutically effective amount of CD19 CAR-T treatment to a subject in which the number of lines of prior therapy are 1-2; 3; 4; or 5. In one embodiment, the disclosure provides a method of treating cancer in a subject in need thereof, comprising administering a therapeutically effective amount of CD19 CAR-T treatment to a subject in which the number of lines of prior therapy are 1-2. The cancer may be any one of the above listed cancers. The CD19 CAR-T treatment may be any one of the above listed CD19 CAR-T treatments. In some embodiments, the CD19 CAR-T treatment is used as first line of treatment. In some embodiments, the CD19 CAR-T treatment is used as a second line of treatment.

In one embodiment, the CD19 CAR-T treatment is any of the of CD19 CAR-T treatments described above. In one embodiment, the CD19 CAR-T treatment comprises axicabtagene ciloleucel treatment. In embodiments, the cancer is refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In one embodiment, a subject selected for axicabtagene ciloleucel treatment has refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In some embodiments, axicabtagene ciloleucel treatment is used as a second line of treatment, where the first line therapy is CHOP, i.e., Cyclophosphamide (Cytoxan®), Doxorubicin (hydroxydoxorubicin), Vincristine (Oncovin®), and Prednisone. In some embodiments, axicabtagene ciloleucel treatment is used as a second line of treatment, where the first line therapy is R-CHOP (CHOP plus Rituximab).

In embodiments, a patient is selected for second-line axicabtagene ciloleucel treatment that has relapsed or refractory disease after first-line chemoimmunotherapy. In embodiments, refractory disease defined as no complete remission to first-line therapy; individuals who are intolerant to first-line therapy are excluded. progressive disease (PD) as best response to first-line therapy, stable disease (SD) as best response after at least 4 cycles of first-line therapy (eg, 4 cycles of R-CHOP), partial response (PR) as best response after at least 6 cycles and biopsy-proven residual disease or disease progression ≤12 months of therapy, and/or relapsed disease defined as complete remission to first-line therapy followed by biopsy-proven relapse ≤12 months of first-line therapy. In some embodiments, first-line therapy comprises R-GDP (Rituximab 375 mg/m2 day 1 (or day 8), Gemcitabine 1 g/m2 on days 1 and 8, Dexamethasone 40 mg on days 1-4, Cisplatin 75 mg/m2 on day 1 (or carboplatin AUC=5)), R-ICE (Rituximab 375 mg/m2 before chemotherapy, Ifosfamide 5 g/m2 24h-CI on day 2 with mesna, Carboplatin AUC=5 on day 2, maximum dose 800 mg, Etoposide 100 mg/m2/d on days 1-3), or R-ESHAP (Rituximab 375 mg/m2 day 1, Etoposide 40 mg/m2/d IV on days 1-4, Methylprednisolone 500 mg/d IV on days 1˜4 or 5, Cisplatin at 25 mg/m2/d CI days 1-4, Cytarabine 2 g/m2 on day 5).

In some embodiments, a patient selected for second-line axicabtagene ciloleucel treatment is provided conditioning therapy comprising fludarabine 30 mg/m² IV and cyclophosphamide 500 mg/m² IV on Days −5, −4, and −3. In some embodiments, axicabtagene ciloleucel treatment is used as a second line of treatment, where the first line therapy embodiments, compositions comprising CAR-expressing immune effector cells disclosed herein may be administered in conjunction (before, after, and/or concurrently with T cell administration) with any number of chemotherapeutic agents. In some embodiments, the antigen binding molecule, transduced (or otherwise engineered) cells (such as CARs), and the chemotherapeutic agent are administered each in an amount effective to treat the disease or condition in the subject. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylol melamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; Polysaccharide K (PSK); razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone, R-CHOP (CHOP plus Rituximab), and G-CHOP (CHOP plus obinutuzumab).

In some embodiments, the chemotherapeutic agent is administered at the same time or within one week after the administration of the engineered cell. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein (before, after, and/or concurrently with T cell administration). For example, potentially useful additional therapeutic agents include PD-1 inhibitors such as nivolumab (OPDIVO®), pembrolizumab (KEYTRUDA®), Cemiplimab (Libtayo), pidilizumab (CureTech), and atezolizumab (Roche), and PD-L1 inhibitors such as atezolizumab, durvalumab, and avelumab.

Additional therapeutic agents suitable for use in combination (before, after, and/or concurrently with T cell administration) with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib), inhibitors of GM-CSF, CSF1, GM-CSFR, or CSF1R, in addition to anti-thymocyte globulin, lenzilumab and mavrilimumab.

In one embodiment, the GM-CSF inhibitor is selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule. In one embodiment, the CSF1 inhibitor is selected from RG7155, PD-0360324, MCS110/lacnotuzumab), or a biosimilar version of any one of the same; and a small molecule. In one embodiment, the GM-CSFR inhibitor and the CSF1R inhibitor is/are selected from Mavrilimumab (formerly CAM-3001; MedImmune, Inc.); cabiralizumab (Five Prime Therapeutics); LY3022855 (IMC-CS4) (Eli Lilly), Emactuzumab, also known as RG7155 or R05509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule.

In some embodiments, the agent is administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, the treatment further comprises therapy, which is therapy between conditioning and the compositions disclosed herein or therapy administered after leukapheresis and completed prior to initiating conditioning chemotherapy. In some embodiments, the bridging therapy comprises, CHOP, G-CHOP, R-CHOP (rituximab, cyclophosphamide, doxorubicin, vincristine, and prednisolone), corticosteroids, bendamustine, platinum compounds, anthracyclines, and/or phosphoinositide 3-kinase (PI3K) inhibitors. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032). In some embodiments, the AKT inhibitor is perifosine, MK-2206. In one embodiment, the mTOR inhibitor is selected from everolimus, sirolimus, temsirolimus, ridaforolimus. In some embodiments, the dual PI3K/mTOR inhibitor is selected from BEZ235, XL765, and GDC-0980. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032).

In some embodiments, the bridging therapy comprises acalabrutinib, brentuximab vedotin, copanlisib hydrochloride, nelarabine, belinostat, bendamustine hydrochloride, carmustine, bleomycin sulfate, bortezomib, zanubrutinib, carmustine, chlorambucil, copanlisib hydrochloride, denileukin diftitox, dexamethasone, doxorubicin hydrochloride, duvelisib, pralatrexate, obinutuzumab, ibritumomab tiuxetan, ibrutinib, idelalisib, recombinant interferon alfa-2b, romidepsin, lenalidomide, mechloretamine hydrochloride, methotrexate, mogamulizumab-kpc, prerixafor, nelarabine, obinutuzumab, denileukin diftitox, pembrolizumab, plerixafor, polatuzumab vedotin-piiq, mogamulizumab-kpc, prednisone, rituximab, hyaluronidase, romidepsin, bortezomib, venetoclax, vinblastine sulfate, vorinostat, zanubrutinib, CHOP, COPP, CVP, EPOCH, R-EPOCH, HYPER-CVAD, ICE, R-ICE, R-CHOP, R-CVP, and combinations of the same.

In some embodiments, the cell immunotherapy is administered in conjunction with debulking therapy, which is used with the aim of reducing tumor burden. In one embodiment, debulking therapy is to be administered after leukapheresis and prior to administration of conditioning chemotherapy or cell infusion. Examples of debulking therapy include the following:

| Type | Proposed Regimen^{a} | Timing/Washout |
|---|---|---|
| R-CHOP | Rituximab 375 mg/m2 Day 1 | Should be administered after |
| | Doxorubicin 50 mg/m2 Day 1 | leukapheresis/enrollment and |
| | Prednisone 100 mg Day 1 through Day 5 | should be completed at least |
| | Cyclophosphamide 750 mg/m2 Day 1 | 14 days prior to the start |
| | Vincristine 1.4 mg/m2 Day 1 | of conditioning chemotherapy |
| R-ICE | Rituximab 375 mg/m2 Day 1 | |
| | Ifosfamide 5 g/m2 24 h-CI Day 2 | |
| | Carboplatin AUC5 Day 2 maximum dose 800 mg | |
| | Etoposide 100 mg/m2/d Days 1 through Day 3 | |
| R-GEMOX | Rituximab 375 mg/m2 Day 1 | |
| | Gemcitabine 1000 mg/m2 Day 2 | |
| | Oxaliplatin 100 mg/m2 Day 2 | |
| R-GDP | Rituximab 375 mg/m2 Day 1 (or Day 8) | |
| | Gemcitabine 1 g/m2 on Day 1 and Day 8 | |
| | Dexamethasone 40 mg on Day 1 through Day 4 | |
| | Cisplatin 75 mg/m2 on Day 1 | |
| | (or carboplatin AUC5 on Day 1) | |
| RADIOTHERAPY^{b} | Per local standard up to 20 to 30 Gy | Should be administered after |
| | | leukapheresis/enrollment and |
| | | should be completed at least |
| | | 5 days prior to the start |
| | | of conditioning chemotherapy |

Abbreviations: AUC, area under the curve
^{a}Other debulking treatment options may be used, to be discussed with the medical monitor. Supportive care with hydration, anti-emesis, mesna, growth factor support, and tumor lysis prophylaxis according to local standard may be used. More than 1 cycle allowed.
^{b}At least 1 target lesion should remain outside of the radiation field to allow for tumor measurements

In some embodiments, a composition comprising an immunotherapy (e.g., engineered CAR T cells) is administered with an anti-inflammatory agent (before, after, and/or concurrently with T cell administration). Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the compositions described herein are administered in conjunction with a cytokine (before, after, or concurrently with T cell administration). Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the administration of the cells and the administration of the additional therapeutic agent are carried out on the same day, are carried out no more than 36 hours apart, no more than 24 hours apart, no more than 12 hours apart, no more than 6 hours apart, no more than 4 hours apart, no more than 2 hours apart, or no more than 1 hour apart or no more than 30 minutes apart. In some embodiments, the administration of the cells and the administration of the additional therapeutic agent are carried out between at or about 0 and at or about 48 hours, between at or about 0 and at or about 36 hours, between at or about 0 and at or about 24 hours, between at or about 0 and at or about 12 hours, between at or about 0 and at or about 6 hours, between at or about 0 and at or about 2 hours, between at or about 0 and at or about 1 hours, between at or about 0 and at or about 30 minutes, between at or about 30 minutes and at or about 48 hours, between at or about 30 minutes and at or about 36 hours, between at or about 30 minutes and at or about 24 hours, between at or about 30 minutes and at or about 12 hours, between at or about 30 minutes and at or about 6 hours, between at or about 30 minutes and at or about 4 hours, between at or about 30 minutes and at or about 2 hours, between at or about 30 minutes and at or about 1 hour, between at or about 1 hours and at or about 48 hours, between at or about 1 hour and at or about 36 hours, between at or about 1 hour and at or about 24 hours, between at or about 1 hour and at or about 12 hours, between at or about 1 hour and at or about 6 hours, between at or about 1 hour and at or about 4 hours, between at or about 1 hour and at or about 2 hours, between at or about 2 hours and at or about 48 hours, between at or about 2 hours and at or about 36 hours, between at or about 2 hours and at or about 24 hours, between at or about 2 hours and at or about 12 hours, between at or about 2 hours and at or about 6 hours, between at or about 2 hours and at or about 4 hours, between at or about 4 hours and at or about 48 hours, between at or about 4 hours and at or about 36 hours, between at or about 4 hours and at or about 24 hours, between at or about 4 hours and at or about 12 hours, between at or about 4 hours and at or about 6 hours, between at or about 6 hours and at or about 48 hours, between at or about 6 hours and at or about 36 hours, between at or about 6 hours and at or about 24 hours, between at or about 6 hours and at or about 12 hours, between at or about 12 hours and at or about 48 hours, between at or about 12 hours and at or about 36 hours, between at or about 12 hours and at or about 24 hours, between at or about 24 hours and at or about 48 hours, between at or about 24 hours and at or about 36 hours or between at or about 36 hours and at or about 48 hours. In some embodiments, the cells and the additional therapeutic agent are administered at the same time.

In some embodiments, the agent is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg.

In some embodiments, the agent is administered in a dosage amount from 0.5 mg/kg to 100 mg/kg, 1 mg/kg to 50 mg/kg, 1 mg kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount from 1 mg/kg to 10 mg/kg, 2 mg kg/to 8 mg/kg, 2 mg/kg to 6 mg/kg, 2 mg/kg to 4 mg/kg or 6 mg/kg to 8 mg/kg, each In some aspects, the agent is administered in a dosage amount of at least 1 mg/kg, 2 mg/kg, 4 mg/kg, 6 mg/kg, 8 mg/kg, 10 mg/kg or more.

In some embodiments, administration of chimeric receptor T cell immunotherapy occurs at a certified healthcare facility.

In some embodiments, the methods disclosed herein comprise monitoring patients at least daily for 7 days at the certified healthcare facility following infusion for signs and symptoms of CRS and neurologic toxicities and other adverse reactions to CAR T cell treatment. In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

In some embodiments, the present disclosure provides methods of preventing the development or reducing the severity of adverse reactions based on the levels of one or more attributes. In some embodiments, the cell therapy is administered in with one or more agents that prevents, delays the onset of, reduces the symptoms of, treats the adverse events, which include cytokine release syndromes and neurologic toxicity. In one embodiment, the agent has been described above. In other embodiments, the agent is described below. In some embodiments, the agent is administered by one of the methods and doses described elsewhere in the specification, before, after, or concurrently with the administration of the cells. In one embodiment, the agent(s) are administered to a subject that may be predisposed to the disease but has not yet been diagnosed with the disease.

In this respect, the disclosed method may comprise administering a “prophylactically effective amount” of tocilizumab, of a corticosteroid therapy, and/or of an anti-seizure medicine for toxicity prophylaxis. In some embodiments, the method comprises administering inhibitors of GM-CSF, CSF1, GM-CSFR, or CSF1R, lenzilumab, mavrilimumab, cytokines, and/or anti-inflammatory agents. The pharmacologic and/or physiologic effect may be prophylactic, i.e., the effect completely or partially prevents a disease or symptom thereof. A “prophylactically effective amount” may refer to an amount effective, at dosages and for periods of time necessary, to achieve a desired prophylactic result (e.g., prevention of onset of adverse reactions).

In some embodiments, the method comprises management of adverse reactions in any subject. In some embodiments, the adverse reaction is selected from the group consisting of cytokine release syndrome (CRS), a neurologic toxicity, a hypersensitivity reaction, a serious infection, a cytopenia and hypogammaglobulinemia.

In some embodiments, the signs and symptoms of adverse reactions are selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia.

In some embodiments, the patient has been identified and selected based on one or more of the biomarkers described in this application. In some embodiments, the patient has been identified and selected simply by the clinical presentation (e.g., presence and grade of toxicity symptom).

In some embodiments, the method comprises preventing or reducing the severity of CRS in a chimeric receptor treatment. In some embodiments, the engineered CAR T cells are deactivated after administration to the patient.

In some embodiments, the method comprises identifying CRS based on clinical presentation. In some embodiments, the method comprises evaluating for and treating other causes of fever, hypoxia, and hypotension. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered.

In some embodiments, the method comprises monitoring patients at least daily for 7 days at the certified healthcare facility following infusion for signs and symptoms of CRS. In some embodiments, the method comprises monitoring patients for signs or symptoms of CRS for 4 weeks after infusion. In some embodiments, the method comprises counseling patients to seek immediate medical attention should signs or symptoms of CRS occur at any time. In some embodiments, the method comprises instituting treatment with supportive care, tocilizumab or tocilizumab and corticosteroids as indicated at the first sign of CRS.

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the cell treatment is administered before, during/concurrently, and/or after the administration of one or more agents (e.g., steroids) or treatments (e.g., debulking) that treat and or prevent (are prophylactic) one or more symptoms of adverse events. A “prophylactically effective amount” refers to an amount effective, at dosages and for periods of time necessary, to achieve the desired prophylactic result. In one embodiment, a prophylactically effective amount is used in subjects prior to or at an earlier stage of disease. In one embodiment, the prophylactically effective amount will be less than the therapeutically effective amount. In some embodiments, the patient is selected for management of adverse events based on the expression of one of more of the markers described herein in this specification. In one embodiment, the adverse event treatment or prophylaxis is administered to any patient that will receive, is receiving, or has received cell therapy.

In some embodiments, the method of managing adverse events comprises monitoring patients at least daily for 7 days at the certified healthcare facility following infusion for signs and symptoms of neurologic toxicities. In some embodiments, the method comprises monitoring patients for signs or symptoms of neurologic toxicities and/or CRS for 4 weeks after infusion.

In some embodiments, the disclosure provides two methods of managing adverse events in subjects receiving CAR T cell treatment with steroids and anti-IL6/anti-IL-6R antibody/ies. In one embodiment, the disclosure provides that early steroid intervention in Cohort 4 is associated with lower rates of severe CRS and neurologic events than what was observed in Cohorts 1+2. In one embodiment, the disclosure provides that earlier use of steroids in Cohort 4 was associated with a median cumulative cortisone-equivalent dose approximately 15% of that in Cohorts 1+2, suggesting that earlier steroid use may allow reduction of overall steroid exposure. Accordingly, in one embodiment, the disclosure provides a method of adverse event management whereby corticosteroid therapy is initiated for management of all cases of grade 1 CRS if there was no improvement after 3 days and for all grade ≥1 neurologic events. In one embodiment, tocilizumab is initiated for all cases of grade 1 CRS if there is no improvement after 3 days and for all grade ≥2 neurologic events. In one embodiment, the disclosure provides a method of reducing overall steroid exposure in patients receiving adverse event management after CAR T cell administration, the method comprising initiation of corticosteroid therapy for management of all cases of grade 1 CRS if there was no improvement after 3 days and for all grade ≥1 neurologic events and/or initiation of tocilizumab for all cases of grade 1 CRS if there is no improvement after 3 days and for all grade ≥2 neurologic events. In one embodiment, the corticosteroid and tocilizumab are administering in a regimen selected from those exemplified in protocols A through C. In one embodiment, the disclosure provides that earlier steroid use is not associated with increased risk for severe infection, decreased CAR T-cell expansion, or decreased tumor response.

In one embodiment, the disclosure supports the safety of levetiracetam prophylaxis in CAR T cell cancer treatment. In one embodiment, the cancer is NHL. In one embodiment, the cancer is R/R LBCL and the patients receive axicabtagene ciloleucel. Accordingly, in one embodiment, the disclosure provides a method of managing adverse events in patients treated with CAR T cells comprising administering to the patient a prophylactic dosage of an anti-seizure medication. In some embodiments, the patients receive levetiracetam (for example, 750 mg orally or intravenous twice daily) starting on day 0 of the CAR T cell treatment (after conditioning) and also at the onset of grade ≥2 neurologic toxicities, if neurologic events occur after the discontinuation of prophylactic levetiracetam. In one embodiment, if a patient does not experience any grade ≥2 neurologic toxicities, levetiracetam is tapered and discontinued as clinically indicated. In one embodiment, levetiracetam prophylaxis is combined with any other adverse event management protocol.

In one embodiment, the disclosure provides that CAR T-cell levels in the patients subject to the adverse management protocol of Cohort 4 were comparable to those of Cohorts 1+2. In one embodiment, the disclosure provides that the numerical levels of key inflammatory cytokines associated with CAR-related inflammatory events (e.g, IFNγ, IL-2 and GM-CSF) are lower in Cohort 4 than in Cohorts 1+2. Accordingly, the disclosure provides a method of reducing CAR T cell treatment-related inflammatory events without impact on CAR T cell levels comprising administering to the patient the adverse event management protocol of Cohort 4. The disclosure also provides a method of reducing cytokine production by immune cells after CAR T cell therapy comprising administering to the patient the adverse event management protocol of Cohort 4. In one embodiment, this effect is obtained without affecting CAR T-cell expansion and response rates. In one embodiment, the patient has R/R LBCL. In one embodiment, the CAR T cell treatment is anti-CD19 CAR T cell treatment. In one embodiment, the CAR T cell treatment comprises axicabtagene ciloleucel.

In one embodiment, the disclosure provides that early or prophylactic use of tocilizumab following axicabtagene ciloleucel for adverse event management decreased grade ≥3 cytokine release syndrome but increased grade ≥3 neurologic events. Accordingly, the disclosure provides a method for adverse event management in CAR T-cell therapy. In one embodiment, patients receive levetiracetam (750 mg oral or intravenous twice daily) starting on day 0. At the onset of grade ≥2 neurologic events, levetiracetam dose is increased to 1000 mg twice daily. If a patient did not experience any grade ≥2 neurologic event, levetiracetam is tapered and discontinued as clinically indicated. Patients also receive tocilizumab (8 mg/kg IV over 1 hour [not to exceed 800 mg]) on day 2. Further tocilizumab (±corticosteroids) may be recommended at the onset of grade 2 CRS in patients with comorbidities or older age, or otherwise in case of grade ≥3 CRS. For patients experiencing grade ≥2 neurologic events, tocilizumab is initiated, and corticosteroids are added for patients with comorbidities or older age, or if there is any occurrence of a grade ≥3 neurologic event with worsening symptoms despite tocilizumab use.

In one embodiment, the disclosure provides that prophylactic steroid use appears to reduce the rate of severe CRS and NEs to a similar extent as early steroid use following axicabtagene ciloleucel administration. Accordingly, the disclosure provides a method for adverse event management in CAR T-cell therapy wherein patients receive dexamethasone 10 mg PO on Days 0 (prior to axicabtagene ciloleucel infusion), 1, and 2. Steroids are also administered starting at Grade 1 NE, and for Grade 1 CRS when no improvement is observed after 3 days of supportive care. Tocilizumab is also administered for Grade ≥1 CRS if no improvement is observed after 24 hours of supportive care.

In one embodiment, the disclosure provides that adverse event management of CAR T-cell therapy with an antibody that neutralizes and/or depletes GM-CSF prevents or reduces treatment-related CRS and/or NEs in treated patients. In one embodiment, the antibody is lenzilumab.

In some embodiments, the adverse events are managed by the administration of an agent/agents that is/are an antagonist or inhibitor of IL-6 or the IL-6 receptor (IL-6R). In some embodiments, the agent is an antibody that neutralizes IL-6 activity, such as an antibody or antigen-binding fragment that binds to IL-6 or IL-6R. For example, in some embodiments, the agent is or comprises tocilizumab (atlizumab) or sarilumab, anti-IL-6R antibodies. In some embodiments, the agent is an anti-IL-6R antibody described in U.S. Pat. No. 8,562,991. In some cases, the agent that targets IL-6 is an anti-TL-6 antibody, such as siltuximab, elsilimomab, ALD518/BMS-945429, sirukumab (CNTO 136), CPSI-2634, ARGX 109, FE301, FM101, or olokizumab (CDP6038), and combinations thereof. In some embodiments, the agent may neutralize IL-6 activity by inhibiting the ligand-receptor interactions. In some embodiments, the IL-6/IL-6R antagonist or inhibitor is an IL-6 mutein, such as one described in U.S. Pat. No. 5,591,827. In some embodiments, the agent that is an antagonist or inhibitor of IL-6/IL-6R is a small molecule, a protein or peptide, or a nucleic acid.

In some embodiments, other agents that may be used to manage adverse reactions and their symptoms include an antagonist or inhibitor of a cytokine receptor or cytokine. In some embodiments, the cytokine or receptor is IL-10, TL-6, TL-6 receptor, IFNγ, IFNGR, IL-2, IL-2R/CD25, MCP-1, CCR2, CCR4, MIP13, CCR5, TNFalpha, TNFR1, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R), IL-1, and IL-1Ralpha/IL-1beta. In some embodiments, the agent comprises situximab, sarilumab, olokizumab (CDP6038), elsilimomab, ALD518/BMS-945429, sirukumab (CNTO 136), CPSI-2634, ARGX 109, FE301, or FM101. In some embodiments, the agent, is an antagonist or inhibitor of a cytokine, such as transforming growth factor beta (TGF-beta), interleukin 6 (TL-6), interleukin 10 (IL-10), IL-2, MIP13 (CCL4), TNF alpha, IL-1, interferon gamma (IFN-gamma), or monocyte chemoattractant protein-I (MCP-1). In some embodiments, the is one that targets (e.g. inhibits or is an antagonist of) a cytokine receptor, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ran-1RP), or IL-10 receptor (IL-10R) and combinations thereof. In some embodiments, the agent is administered by one of the methods and doses described elsewhere in the specification, before, after, or concurrently with the administration of the cells.

In some embodiments, the agent is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the agent is administered in a dosage amount of at least or at least about or about 2 mg/kg, 4 mg/kg, 6 mg/kg or 8 mg/kg. In some embodiments, is administered in a dosage amount from about 1 mg/kg to 12 mg/kg, such as at or about 10 mg/kg. In some embodiments, the agent is administered by intravenous infusion. In one embodiment, the agent is tocilizumab. In some embodiments, the (agent(s), e.g, specifically tocilizumab) is/are administered by one of the methods and doses described elsewhere in the specification, before, after, or concurrently with the administration of the cells.

In some embodiments, the method comprises identifying CRS based on clinical presentation. In some embodiments, the method comprises evaluating for and treating other causes of fever, hypoxia, and hypotension. If CRS is observed or suspected, it may be managed according to the recommendations in protocol A, which may also be used in combination with the other treatments of this disclosure, including Neutralization or Reduction of the CSF/CSFR1 Axis. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered. In some embodiments, a biosimilar or equivalent of tocilizumab may be used instead of tocilizumab in the methods disclosed herein. In other embodiments, another anti-IL6R may be used instead of tocilizumab.

In some embodiments, adverse events are managed according to the following protocol (protocol A):

| CRS Grade (a) | Tocilizumab | Corticosteroids |
|---|---|---|
| Grade 1 | N/A | N/A |
| Symptoms require | | |
| symptomatic treatment only | | |
| (e.g., fever, nausea, fatigue, | | |
| headache, myalgia, malaise). | | |
| Grade 2 | Administer tocilizumab (c) 8 | Manage per Grade 3 if no |
| Symptoms require and | mg/kg IV over 1 hour (not to | improvement within 24 hours |
| respond to moderate | exceed 800 mg). | after starting tocilizumab. |
| intervention. | Repeat tocilizumab every 8 | |
| Oxygen requirement less | hours as needed if not | |
| than 40% FiO₂ or | responsive to IV fluids or | |
| hypotension responsive to | increasing supplemental oxygen. | |
| fluids or low-dose of one | Limit to a maximum of 3 | |
| vasopressor or Grade 2 organ | doses in a 24-hour period; | |
| toxicity (b). | maximum total of 4 doses if | |
| | no clinical improvement in | |
| | the signs and symptoms of CRS. | |
| Grade 3 | Per Grade 2 | Administer |
| Symptoms require and | | methylprednisolone 1 mg/kg |
| respond to aggressive | | IV twice daily or equivalent |
| intervention. | | dexamethasone (e.g., 10 mg |
| Oxygen requirement greater | | IV every 6 hours). |
| than or equal to 40% FiO₂ or | | Continue corticosteroids use |
| hypotension requiring high- | | until the event is Grade 1 or |
| dose or multiple vasopressors | | less, then taper over 3 days. |
| or Grade 3 organ toxicity or | | If not improving, manage as |
| Grade 4 transaminitis. | | Grade 4. |
| Grade 4 | Per Grade 2 | Administer |
| Life-threatening symptoms. | | methylprednisolone 1000 mg |
| Requirements for ventilator | | IV per day for 3 days; if |
| support, continuous veno- | | improves, then manage as above. |
| venous hemodialysis (CVVHD) | | Consider alternate |
| or Grade 4 organ toxicity | | immunosuppressants if no |
| (excluding transaminitis). | | improvement or if condition |
| | | worsens. |

(a) Lee D W et al., (2014). Current concepts in the diagnosis and management of cytokine release syndrome. Blood. 2014 Jul. 10; 124(2): 188-195.
(b) Refer to Procotocol B for management of neurologic toxicity.
(c) Refer to ACEMTRA ® (tocilizumab) Prescribing Information for details, https://www.gene.com/download/pdf/actemra_prescribing.pdf (last accessed Oct. 18, 2017). Initial U.S. approval is indicated to be in 2010.

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. Consider non-sedating, anti-seizure medicines (e.g., levetiracetam) for seizure prophylaxis for any ≥Grade 2 neurologic toxicities. The following treatments may be used in combination with the other treatments of this disclosure, including Neutralization or Reduction of the CSF/CSFR1 Axis.

In some embodiments, adverse events are managed according to the following protocol (protocol B):

| Grading | | |
|---|---|---|
| Assessment | Concurrent CRS | No concurrent CRS |
| Grade 2 | Administer tocilizumab per table above | Administer dexamethasone |
| | (protocol A) for management of Grade 2 CRS. | 10 mg IV every 6 hours. |
| | If no improvement within 24 hours after | Continue dexamethasone use |
| | starting tocilizumab, administer | until the event is Grade 1 or |
| | dexamethasone 10 mg IV every 6 hours if | less, then taper over 3 days. |
| | not already taking other steroids. | |
| | Continue dexamethasone use until the event | |
| | is Grade 1 or less, then taper over 3 days. | |
| | Consider non-sedating, anti-seizure medicines | |
| | (e.g., levetiracetam) for seizure prophylaxis. | |
| Grade 3 | Administer tocilizumab per (protocol A) | Administer dexamethasone |
| | for management of Grade 2 CRS. | 10 mg IV every 6 hours. |
| | In addition, administer dexamethasone 10 mg | Continue dexamethasone use |
| | IV with the first dose of tocilizumab | until the event is Grade 1 or |
| | and repeat dose every 6 hours. Continue | less, then taper over 3 days. |
| | dexamethasone use until the event is | |
| | Grade 1 or less, then taper over 3 days. | |
| | Consider non-sedating, anti-seizure medicines | |
| | (e.g., levetiracetam) for seizure prophylaxis. | |
| Grade 4 | Administer tocilizumab per (protocol A) | Administer methylprednisolone |
| | for management of Grade 2 CRS. | 1000 mg IV per day for 3 days; |
| | Administer methylprednisolone 1000 mg | if improves, then manage as |
| | IV per day with first dose of tocilizumab | above. |
| | and continue methylprednisolone 1000 | |
| | mg IV per day for 2 more days; if | |
| | improves, then manage as above. | |

Consider non-sedating, anti-seizure medicines
(e.g., levetiracetam) for seizure prophylaxis.

Additional Safety Management Strategies with Corticosteroids

Administration of corticosteroids and/or tocilizumab at Grade 1 may be considered prophylactic. Supportive care may be provided in all protocols at all CRS and NE severity grades.

In one embodiment of a protocol for management of adverse events related to CRS, tocilizumab and/or corticosteroids are administered as follows: Grade 1 CRS: no tocilizumab; no corticosteroids; Grade 2 CRS: tocilizumab (only in case of comorbidities or older age); and/or corticosteroids (only in case of comorbidities or older age); Grade 3 CRS: tocilizumab; and/or corticosteroids; Grade 4 CRS: tocilizumab; and/or corticosteroids. In another embodiment of a protocol for management of adverse events related to CRS, tocilizumab and/or corticosteroids are administered as follows: Grade 1 CRS: tocilizumab (if no improvement after 3 days); and/or corticosteroids (if no improvement after 3 days); Grade 2 CRS: tocilizumab; and/or corticosteroids; Grade 3 CRS: tocilizumab; and/or corticosteroids; Grade 4 CRS: tocilizumab; and/or corticosteroids, high dose.

In one embodiment of a protocol for management of adverse events related to NE, tocilizumab and/or corticosteroids are administered as follows: Grade 1 NE: no tocilizumab; no corticosteroids;

Grade 2 NE: no tocilizumab; no corticosteroids; Grade 3 NE: tocilizumab; and/or corticosteroids (only if no improvement to tocilizumab, standard dose); Grade 4 NE: tocilizumab; and/or corticosteroids.

In another embodiment of a protocol for management of adverse events related to NE, tocilizumab and/or corticosteroids are administered as follows: Grade 1 NE: no tocilizumab; and/or corticosteroids; Grade 2 NE: tocilizumab; and/or corticosteroids; Grade 3 NE: tocilizumab; and/or corticosteroids, high dose; Grade 4 NE: tocilizumab; and/or corticosteroids, high dose.

In one embodiment, corticosteroid treatment is initiated at CRS grade ≥2 and tocilizumab is initiated at CRS grade ≥2. In one embodiment, corticosteroid treatment is initiated at CRS grade ≥1 and tocilizumab is initiated at CRS grade ≥1. In one embodiment, corticosteroid treatment is initiated at NE grade ≥3 and tocilizumab is initiated at CRS grade ≥3. In one embodiment, corticosteroid treatment is initiated at CRS grade ≥1 and tocilizumab is initiated at CRS grade ≥2. In some embodiments, prophylactic use of tocilizumab administered on Day 2 may decrease the rates of Grade ≥3 CRS.

In one embodiment, the protocol for treatment of adverse events comprises Protocol C, as follows:

| CRS | | |
|---|---|---|
| Grade | Tocilizumab Dose^{a} | Corticosteroid Dose^{a} |
| 1 | 8 mg/kg over 1 hour^{b} if no | Dexamethasone 10 mg × 1 |
| | improvement after 24 hours of | if no improvement after 3 days |
| | supportive care; repeat every | |
| | 4-6 hours as needed | |
| 2 | 8 mg/kg over 1 hour^{b}; repeat | Dexamethasone 10 mg × 1 |
| | every 4-6 hours as needed | |
| 3 | Per Grade 2 | Methylprednisolone 1 mg/kg |
| | | IV twice daily or equivalent |
| | | dexamethasone dose |
| 4 | Per Grade 2 | Methylprednisolone 1000 |
| | | mg/d IV for 3 days |
| NE | | |
| Grade | Tocilizumab Dose | Corticosteroid Dose |
| 1 | N/A | Dexamethasone 10 mg × 1 |
| 2 | Only in the case of | Dexamethasone 10 mg 4×/day |
| | concurrent CRS; | |
| | 8 mg/kg over 1 hour; repeat | |
| | every 4-6 hours as needed | |
| 3 | Per Grade 2 | Methylprednisolone 1 g once |
| | | daily |
| 4 | Per Grade 2 | Methylprednisolone 1 g twice |
| | | daily |

^{a}Therapy to be tapered on improvement of symptoms at investigator's discretion;
^{b}Not to exceed 800 mg;
AE, adverse event;
CRS, cytokine release syndrome;
IV, intravenous;
N/A, not applicable;
NE, neurologic event

Any corticosteroid may be appropriate for this use. In one embodiment, the corticosteroid is dexamethasone. In some embodiments, the corticosteroid is methylprednisolone. In some embodiments, the two are administered in combination. In some embodiments, glucocorticoids include synthetic and non-synthetic glucocorticoids. Exemplary glucocorticoids include, but are not limited to: alclomethasones, algestones, beclomethasones (e.g. beclomethasone dipropionate), betamethasones (e.g. betamethasone 17 valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, flupredni solones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylpredni solones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21 palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980) and Remington: The Science and Practice of Pharmacy, 22nd Edition, Lippincott Williams & Wilkins, Philadelphia, Pa. (2013) and any other editions, which are hereby incorporated by reference. In some embodiments, the glucocorticoid is selected from among cortisones, dexamethasones, hydrocortisones, methylprednisolones, prednisolones and prednisones. In an embodiment, the glucocorticoid is dexamethasone. In other embodiments, the steroid is a mineralcorticoid. Any other steroid may be used in the methods provided herein.

The one or more corticosteroids may be administered at any dose and frequency of administration, which may be adjusted to the severity/grade of the adverse event (e.g., CRS and NE). Tables 1 and 2 provide examples of dosage regimens for management of CRS and NE, respectively. In another embodiment, corticosteroid administration comprises oral or IV dexamethasone 10 mg, 1-4 times per day. Another embodiment, sometimes referred to as “high-dose” corticosteroids, comprises administration of IV methylprednisone 1 g per day alone, or in combination with dexamethasone. In some embodiments, the one or more cortico steroids are administered at doses of 1-2 mg/kg per day.

The corticosteroid may be administered in any amount that is effective to ameliorate one or more symptoms associated with the adverse events, such as with the CRS or neurotoxicity. The corticosteroid, e.g., glucocorticoid, may be administered, for example, at an amount between at or about 0.1 and 100 mg, per dose, 0.1 to 80 mg, 0.1 to 60 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject. Typically, the corticosteroid, such as a glucocorticoid is administered at an amount between at or about 0.4 and 20 mg, for example, at or about 0.4 mg, 0.5 mg, 0.6 mg, 0.7 mg, 0.75 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg or 20 mg per dose, to an average adult human subject.

In some embodiments, the corticosteroid may be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

Generally, the dose of corticosteroid administered is dependent upon the specific corticosteroid, as a difference in potency exists between different corticosteroids. It is typically understood that drugs vary in potency, and that doses may therefore vary, in order to obtain equivalent effects. Equivalence in terms of potency for various glucocorticoids and routes of administration. is well known. Information relating to equivalent steroid dosing (in a non-chronotherapeutic manner) may be found in the British National Formulary (BNF) 37, March 1999.

In some embodiments, the adverse events are managed by the following protocol: patients receive levetiracetam (750 mg oral or intravenous twice daily) starting on day 0 of administration of T cell therapy; at the onset of grade ≥2 neurologic events, levetiracetam dose is increased to 1000 mg twice daily; if a patient did not experience any grade ≥2 neurologic event, levetiracetam is tapered and discontinued as clinically indicated; patients also receive tocilizumab (8 mg/kg IV over 1 hour [not to exceed 800 mg]) on day 2; further tocilizumab (±corticosteroids) may be recommended at the onset of grade 2 CRS in patients with comorbidities or older age, or otherwise in case of grade ≥3 CRS; for patients experiencing grade ≥2 neurologic events, tocilizumab is initiated, and corticosteroids are added for patients with comorbidities or older age, or if there is any occurrence of a grade ≥3 neurologic event with worsening symptoms despite tocilizumab use. In some embodiments, levetiracetam is administered for prophylaxis and at the onset of grade ≥2 neurologic toxicities, if neurologic events occur after the discontinuation of prophylactic levetiracetam and/or levetiracetam is tapered and discontinued if the patient does not experience any grade ≥2 neurologic toxicities.

In some embodiments, the adverse events are managed by the following protocol: patients receive dexamethasone 10 mg PO on Days 0 (prior to T cell therapy infusion), 1, and 2; steroids are also administered starting at Grade 1 NE, and for Grade 1 CRS when no improvement is observed after 3 days of supportive care; tocilizumab is also administered for Grade ≥1 CRS if no improvement is observed after 24 hours of supportive care.

In some embodiments, patients treated with CAR T cells (e.g., CD19-directed) or other genetically modified autologous T cell immunotherapy may develop secondary malignancies. In certain embodiments, patients treated with CAR T cells (e.g., CD19-directed) or other genetically modified allogeneic T cell immunotherapy may develop secondary malignancies. In some embodiments, the method comprises monitoring life-long for secondary malignancies.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

The present disclosure is further illustrated by the following examples, which should not be construed as further limiting. The contents of all references cited throughout this application are expressly incorporated herein by reference.

The disclosures provided by this application may be used in a variety of methods in additional to, or as a combination of, the methods described above. The following is a compilation of exemplary methods that may be derived from the disclosures provided in this application.

In one embodiment, the disclosure provides a method of manufacturing an immunotherapy product with improved clinical efficacy and/or decreased toxicity. In some embodiments, the immunotherapy product comprises blood cells. In some embodiments, blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some embodiments, a washing step is accomplished a semi-automated“flow-through” centrifuge (for example, the Cobe 2991 cell processor, Baxter) according to the manufacturer's instructions. In some embodiments, a washing step is accomplished by tangential flow filtration (TFF) according to the manufacturer's instructions. In some embodiments, the cells are resuspended in a variety of biocompatible buffers after washing, such as, for example, Ca++Mg++free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient. In some embodiments, the methods include leukapheresis.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some embodiments includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some embodiments of such processes, a volume of cells is mixed with an amount of a desired affinity-based selection reagent. The immunoaffinity-based selection may be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) may be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a chamber, for example, under centrifugal rotation. In some embodiments, incubation of cells with selection reagents, such as immunoaffinity-based selection reagents, is performed in a chamber.

In some embodiments, by conducting such selection steps or portions thereof (e.g., incubation with antibody-coated particles, e.g., magnetic beads) in the cavity of a chamber, the user is able to control certain parameters, such as volume of various solutions, addition of solution during processing and timing thereof, which may provide advantages compared to other available methods. For example, the ability to decrease the liquid volume in the cavity during the incubation may increase the concentration of the particles (e.g. bead reagent) used in the selection, and thus the chemical potential of the solution, without affecting the total number of cells in the cavity. This in turn may enhance the pairwise interactions between the cells being processed and the particles used for selection.

In some embodiments, carrying out the incubation step in the chamber, e.g., when associated with the systems, circuitry, and control as described herein, permits the user to effect agitation of the solution at desired time(s) during the incubation, which also may improve the interaction.

In some embodiments, at least a portion of the selection step is performed in a chamber, which includes incubation of cells with a selection reagent. In some embodiments of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which may aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some embodiments also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps may be based on positive selection, in which the cells having bound the reagents are retained for further use, and/or negative selection, in which the cells having not bound to the antibody or binding partner are retained. In some examples, both fractions are retained for further use.

In some embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+, CD28+ T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+ CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhlgh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations may be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long term survival, expansion, and/or engraftment following administration, which in some embodiments is particularly robust in such sub-populations. In some embodiments, combining TcM-enriched CD8+ T cells and CD4+ T cells further enhances efficacy. In some embodiments, enriching for T cells with naïve phenotype (CD45RA+ CCR7+) enhances efficacy.

In embodiments, memory T cells are present in both CD62L+ and CD62L subsets of CD8+ peripheral blood lymphocytes. PBMC may be enriched for or depleted of CD62L CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some embodiments, it is based on negative selection for cells expressing or highly expressing CD45RA and/or granzyme B. In some embodiments, isolation of a CD8+ population enriched for TCM cells is carried out by depletion of cells expressing CD4, CD 14, CD45RA, and positive selection or enrichment for cells expressing CD62L. In one embodiment, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some embodiments are carried out simultaneously and in other embodiments are carried out sequentially, in either order. In some embodiments, the same CD4 expression-based selection step used in preparing the CD8+ cell population or subpopulation, also is used to generate the CD4+ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naive, central memory, and effector cells by identifying cell populations that have cell surface antigens. CD4+ lymphocytes may be obtained by standard methods. In some embodiments, naive CD4+ T lymphocytes are CD45RO, CD45RA+, CD62L+, CD4+ T cells. In some embodiments, central memory CD4+ cells are CD62L+ and CD45RO+. In some embodiments, effector CD4+ cells are CD62L and CD45RO. In some embodiments, T cells with naïve phenotype are CD45RA+ CCR7+.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques.

In some embodiments, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps.

In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., ah, of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps.

In some embodiments, the separation and/or other steps is carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls ah components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some embodiments is equipped with a cell processing unity that permits automated washing and fractionation of cells by centrifugation. The CliniMACS Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some embodiments may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In one embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the cells are incubated and/or cultured prior to or in connection with genetic engineering. The incubation steps may include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions may include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some embodiments, the agent turns on or initiates TCR/CD3 intracellular signaling cascade in a T cell. Such agents may include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some embodiments, the IL-2 concentration is at least about 10 units/mL. In some embodiments, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some embodiments, the non-dividing feeder cells may comprise gamma-irradiated PBMC feeder cells. In some embodiments, the PBMC are irradiated with gamma rays in the range of about 3000 to 3600 rads to prevent cell division. In some embodiments, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL may be irradiated with gamma rays in the range of about 6000 to 10,000 rads. The LCL feeder cells in some embodiments is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones may be generated to cytomegalovirus antigens by isolating T cells from infected subjects and stimulating the cells in vitro with the same antigen.

In some embodiments, at least a portion of the incubation in the presence of one or more stimulating conditions or stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some embodiments of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which may aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the stimulation results in activation and/or proliferation of the cells, for example, prior to transduction.

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g. retroviral or lentiviral, transduction, transposons, and electroporation. In some embodiments, the engineering produces one or more engineered compositions of enriched T cells.

In certain embodiments, the one or more compositions of stimulated T cells are or include two separate stimulated compositions of enriched T cells. In some embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In some embodiments, the two separate compositions include a composition of enriched CD8+ T cells. In some embodiments, two separate compositions of enriched CD4+ T cells and enriched CD8+ T cells are genetically engineered separately. In some embodiments, the same composition is enriched for both CD4+ T cells and CD8+ T cells and these are genetically engineered together.

In one embodiment, the sample of T lymphocytes is prepared by leukapheresis of PBMCs from the subject. In one embodiment, the leukapheresis sample is further subject to T lymphocyte enrichment through positive selection for CD4+ and/or CD8+ cells. In one embodiment, the lymphocytes are further engineered to comprise a CAR or an exogenous TCR. Examples of CARs and TCRs and methods of engineering lymphocytes are described elsewhere in the disclosure. In one embodiment, the method comprises expanding the engineered lymphocytes to produce a T cell infusion product in the presence of IL-2. In one embodiment, the engineered lymphocytes are expanded for about 2-7 days in the presence of IL-2.

Under circumstances where subjects initially respond and subsequently relapse, subjects may be eligible for a second course of conditioning chemotherapy and axicabtagene ciloleucel. Retreatment may be administered under conditions such as: subject has a PR or CR; subject's disease subsequently progresses; CD19 tumor expression confirmed locally by biopsy after disease progression and prior to re-treatment; Subject continues to meet the original study eligibility criteria with exception of prior axicabtagene ciloleucel use. Screening assessments should be repeated if clinically indicated, as determined by the investigator, to confirm eligibility; Subject has not received subsequent therapy for the treatment of lymphoma; Toxicities related to conditioning chemotherapy (fludarabine and cyclophosphamide), with the exception of alopecia, have resolved to ≤Grade 1 or returned to baseline prior to retreatment; and Subject does not have known neutralizing antibodies (exception: if a non-neutralizing antibody develops subject may be retreated if they meet the original study eligibility criteria).

### EXAMPLES

### Example 1

CLINICAL TRIAL-1 is a clinical study wherein patients with relapsed/refractory NHL have been treated with axicabtagene ciloleucel. Axicabtagene ciloleucel is a CD19-directed genetically modified autologous T cell immunotherapy, comprising the patient's own T cells harvested and genetically modified ex vivo by retroviral transduction to express a chimeric antigen receptor (CAR) comprising an anti-CD19 single chain variable fragment (scFv) linked to CD28 and CD3-zeta co-stimulatory domains. Patients may have had diffuse large B-cell lymphoma, primary mediastinal B-cell lymphoma, or transformed follicular lymphoma with refractory disease despite undergoing recommended prior therapy. Patients received a target dose of 2×10⁶ anti-CD19 CAR T cells per kilogram of body weight after receiving a conditioning regimen of low-dose cyclophosphamide and fludarabine. (Neelapu, S S et al. 2017, N Engl J Med 2017; 377(26):2531-44.

Biomarker data from CLINICAL TRIAL-1 patients were analyzed according to an expanded statistical analysis plan for correlates of response and parameters differentially associated with treatment efficacy and toxicities, as well as product fitness. Several correlations were revealed. Available samples from patients in CLINICAL TRIAL-1 (NCT02348216) were analyzed. Safety and efficacy results were previously reported. (Neelapu, S S et al. 2017, N Engl J Med 2017; 377(26):2531-44; Locke F L et al. 2019; Lancet Oncol. 2019 January; 20(1):31-42. doi:10.1016/51470-2045(18)30864-7. Epub 2018 Dec. 2). Durable response refers to those patients who were in ongoing response at time of data cut-off. Relapse refers to those patients who achieved a CR or PR and subsequently experienced disease progression. Patients who achieved stable or progressive disease as best response are included in no response category.

While conventional prognostic factors for LBCL were not associated with outcomes in the pivotal CLINICAL TRIAL-1 study (Neelapu et al. NEJM. 2017), other attributes like chimeric antigen receptor (CAR) T-cell fitness and composition (CCR7+CD45RA+ T cells), reduced pretreatment tumor burden, immune tumor microenvironment (TME) with presence of activated CD8+PD-1+LAG-3+/−TIM-3− T cells were associated with efficacy (Locke et al., Blood Advances, 2020 https://doi.org/10.1182/bloodadvances.2020002394 and Galon et al., ASCO, 2020https://ascopubs.org/doi/ab s/10.1200/JCO.2020.38.15 supp1.3022). By further interrogating the tumor immune contexture (TIC) (e.g. density, composition, and function of immune cells) in patients with larger baseline tumor burden (SPD>=3721 mm2) and comparing to those with small baseline tumor burden (SPD<3721 mm2), an association was uncovered between myeloid inflammation in pretreatment TIC and CAR-T expansion that influences durability of response, particularly in the patients that are with larger tumors and noticeably harder to treat.

The analysis of pretreatment TIC was performed by multiplex immunohistochemistry (n=18) and gene expression analysis (N=30) as previously described (Rossi et al, Cancer Res Jul. 1, 2018 (78) (13 Supplement) LB-016; DOI: 10.1158/1538-7445.AM2018-LB-016, Galon et al, Journal of Clinical Oncology 2020 (38) (15 suppl), 3022-3022 DOI: 10.1200/JCO.2020.38.15 supp1.3022 Journal of Clinical Oncology 38, no. 15 suppl (May 20, 2020) 3022-3022. To further interrogate the activated T cell and suppressive myeloid signatures, the indices were derived with root mean square of selected genes for T cell (CD3D, CD8A, CTLA4, TIGIT) and myeloid cell (ARG2, TREM2). The ratio between activated T cell and suppressive myeloid cell index was determined by Log 2((T-cell index+1)/Myeloid Index+1)).

Pretreatment immune TME features related to suppressive myeloid-related activity, most notably ARG2, TREM2, and IL-8 gene expression, were elevated in patients who failed to respond or relapsed without documented loss of CD19 expression. ARG2 and TREM2 levels in pretreatment biopsies were negatively associated with CD8+ T-cell density. Patients with high tumor burden who achieved durable response had low pretreatment ARG2 and TREM2 levels in TME and enhanced CAR T-cell expansion after axicabtagene ciloleucel compared with patients with high tumor burden who relapsed. High ratio of T cell to suppressive myeloid cell markers (T/M ratio) in pretreatment biopsies associated positively with CAR T-cell expansion (peak and peak normalized to tumor burden) and durable response in patients with high tumor burden.

Axicabtagene ciloleucel may overcome high tumor burden in patients with a favorable immune TIC alongside robust CAR T-cell expansion. Favorable immune TME is characterized by reduced suppressive myeloid cell activity (low ARG2 and TREM2 expression) and increased T/M ratio. These data suggest possible actionable strategies to overcome high TB in the context of CAR T-cell therapy.

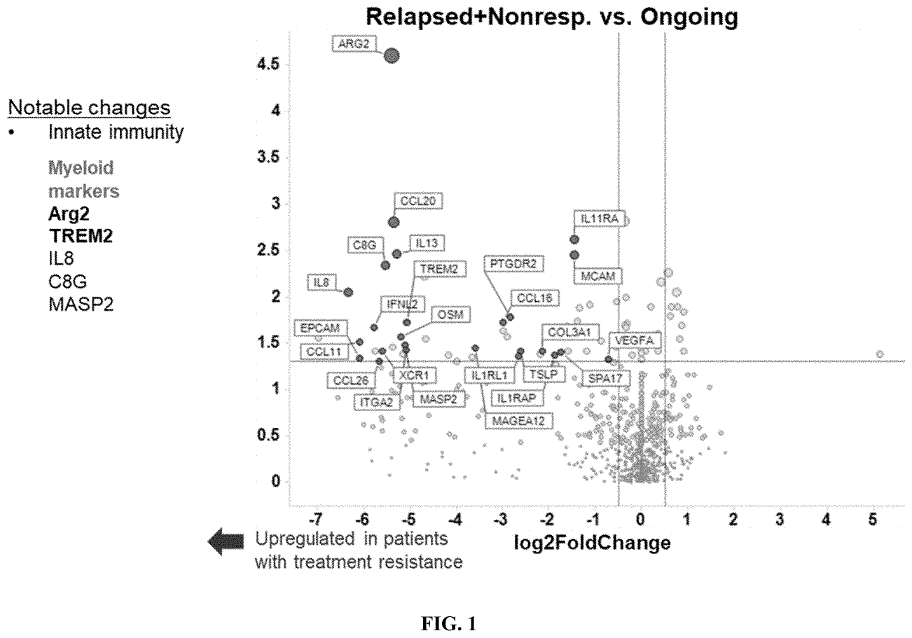

Myeloid associated gene signature is upregulated in relapsed and nonresponders compared with ongoing responders. FIG. 1. Volcano plot of differentially expressed genes comparing ongoing responders with relapsed and nonresponders. Fold change was determined by the ratio of median value in each ongoing response group, and the p-value was derived from Wilcoxon test. A small constant, 1, was added to the medians to avoid zero in logarithmic transformation. Top differentially expressed gene in relapsed and nonresponder group, including ARG2, TREM2, IL8, CBG, and MASP2, are related to TME myeloid inflammation. Gene counts are normalized using a ratio of the expression value to the geometric mean of all housekeeping genes on the panel. Housekeeper-normalized gene counts are additionally normalized using a panel standard run on the same cartridge as the observed data.

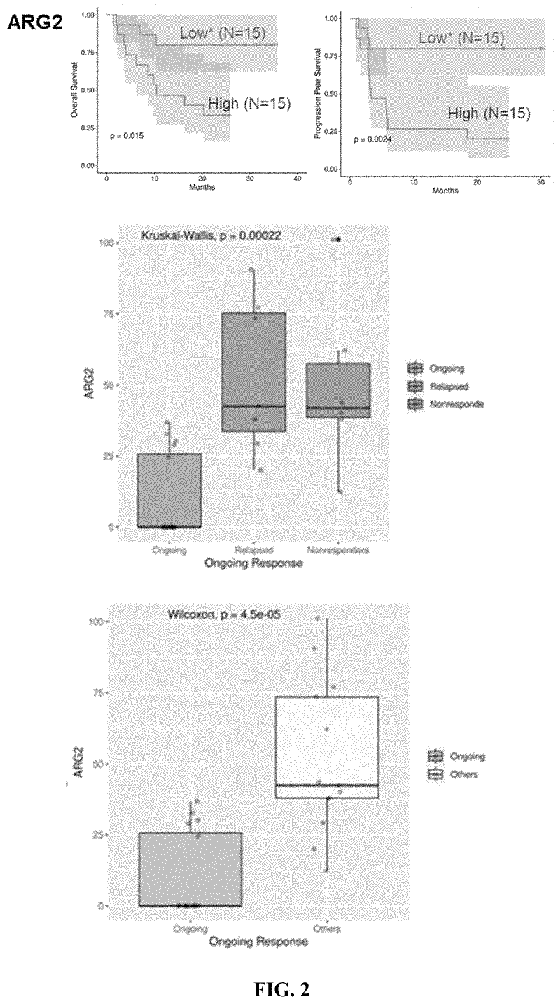

Patients with higher ARG2 expression (determined by the median of 30 patients) in pretreatment tumors have worse overall and progression free survival than those with lower ARG2 expression. The boxplots show ongoing responders expressing lower level of ARG2 in pretreatment tumor than relapsed and/or non-responders. FIG. 2. Overall and progression-free survival curves of CLINICAL TRIAL-1 subjects grouped by ARG2 gene counts. Kaplan-Meier overall and progression-free survival curves with a median cut-off selection for ARG2 gene counts in pretreatment tumor samples with significance determined by the Log-Rank test. The boxplots show ARG2 gene counts by ongoing response groups. Ongoing responders are shown in green, relapsed patients are shown in orange, non-responders are shown in blue, while relapsed with nonresponders (others) are show in yellow. Nonparametric Wilcoxon tests and Kruskal-Wallis tests are conducted for comparisons of 2 or 3 groups, respectively.

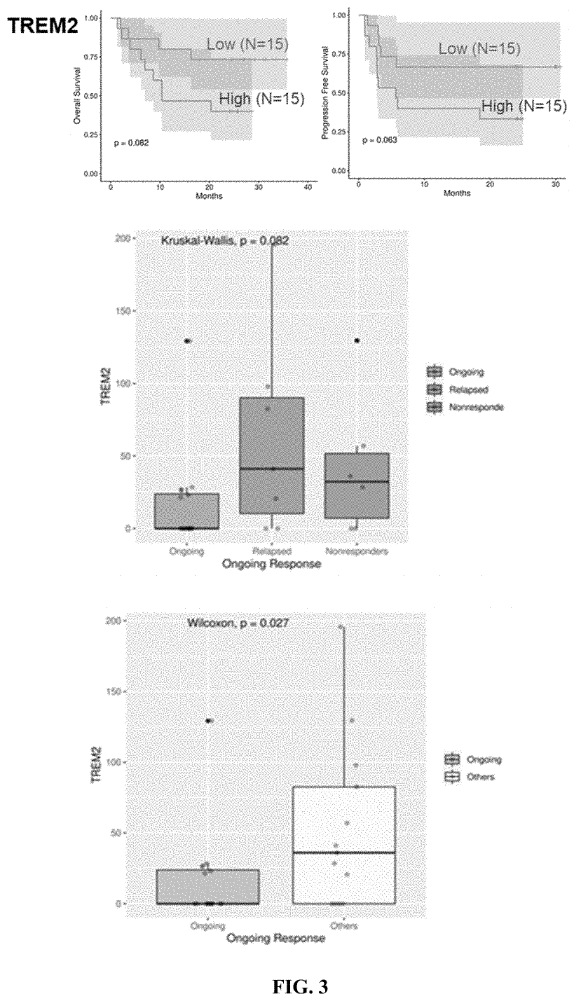

Patients with higher TREM2 expression (determined by the median of 30 patients) in pretreatment tumors have worse overall and progression free survival than those with lower TREM2 expression. The boxplots show ongoing responders expressing lower level of TREM2 in pretreatment tumor than relapsed and/or non-responders. FIG. 3. Overall and progression-free survival curves of CLINICAL TRIAL-1 subjects grouped by TREM2 gene counts. Kaplan-Meier overall and progression-free survival curves with a median cut-off selection for TREM2 gene counts in pretreatment tumor samples with significance determined by the Log-Rank test. The boxplots show TREM2 gene counts by ongoing response groups. Ongoing responders are shown in green, relapsed patients are shown in orange, non-responders are shown in blue, while relapsed with nonresponders (others) are show in yellow. Nonparametric Wilcoxon tests and Kruskal-Wallis tests are conducted for comparisons of 2 or 3 groups, respectively.

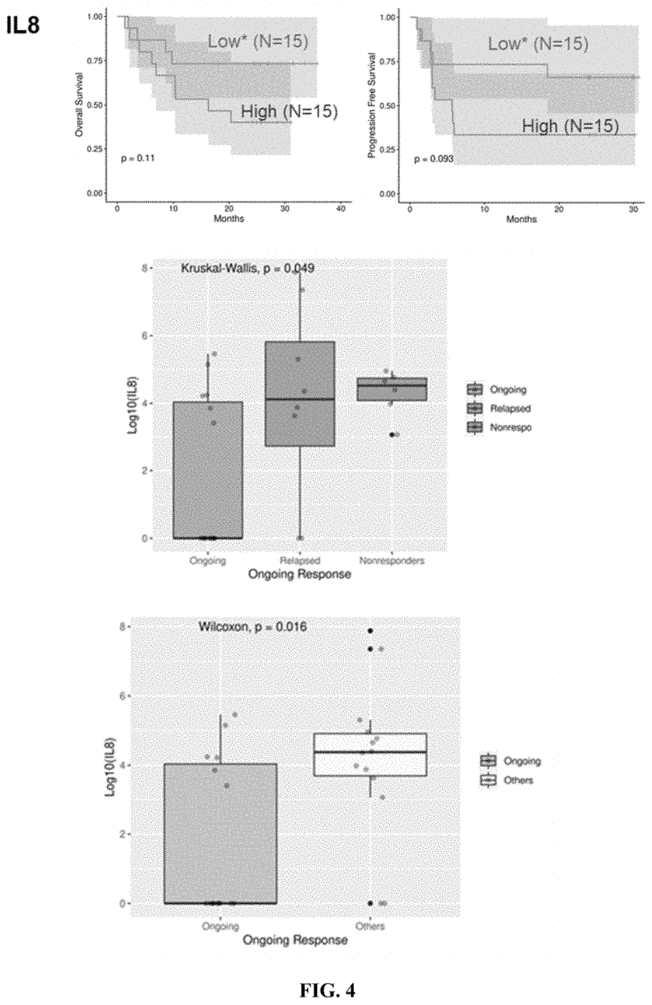

Patients with higher IL8 expression (determined by the median of 30 patients) in pretreatment tumors have worse overall and progression free survival than those with lower IL8 expression. The boxplots show ongoing responders expressing lower level of IL8 pretreatment tumor than relapsed and/or non-responders. FIG. 4. Overall and progression-free survival curves of CLINICAL TRIAL-1 subjects grouped by IL8 gene counts. Kaplan-Meier overall progression-free survival curves with a median cut-off selection for IL8 gene counts in pretreatment tumor samples with significance determined by the Log-Rank test. The boxplots show IL8 gene counts by ongoing response groups. Ongoing responders are shown in green, relapsed patients are shown in orange, non-responders are shown in blue, while relapsed with nonresponders (others) are show in yellow. Nonparametric Wilcoxon tests and Kruskal-Wallis tests are conducted for comparisons of 2 or 3 groups, respectively.

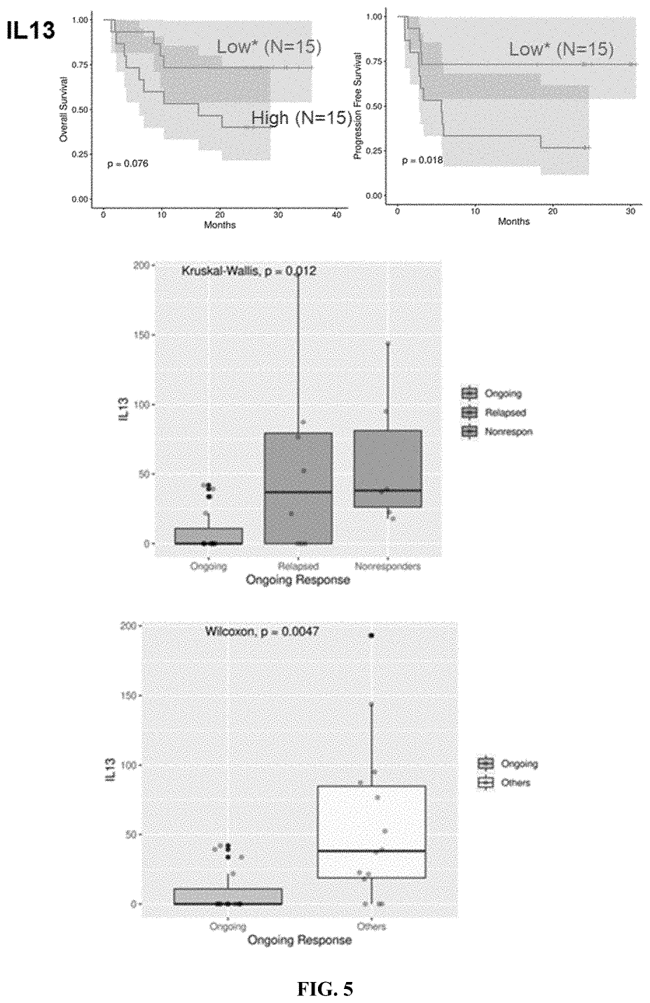

Patients with higher IL13 expression (determined by the median of 30 patients) in pretreatment tumors have worse overall and progression free survival than those with lower IL13 expression. The boxplots show ongoing responders expressing lower level of IL13 pretreatment tumor than relapsed and/or non-responders. FIG. 5. Overall and progression-free survival curves of CLINICAL TRIAL-1 subjects grouped by IL13 gene counts. Kaplan-Meier overall and progression-free survival curves with a median cut-off selection for IL13 gene counts in pretreatment tumor samples with significance determined by the Log-Rank test. The boxplots show IL13 gene counts by ongoing response groups. Ongoing responders are shown in green, relapsed patients are shown in orange, non-responders are shown in blue, while relapsed with nonresponders (others) are show in yellow. Nonparametric Wilcoxon tests and Kruskal-Wallis tests are conducted for comparisons of 2 or 3 groups, respectively.

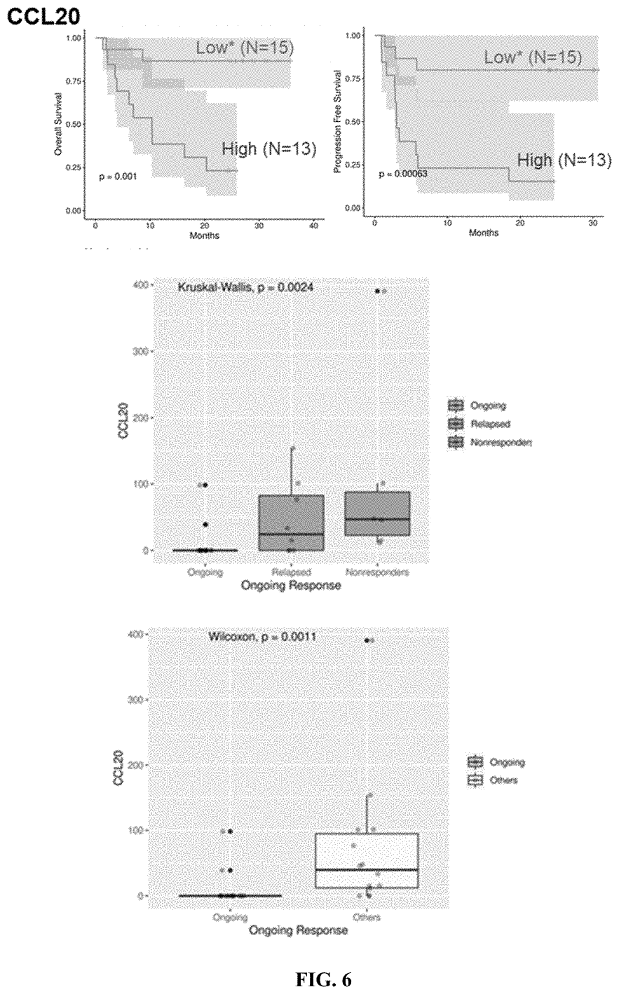

Patients with higher CCL20 expression (determined by the median of 30 patients) in pretreatment tumors have worse overall and progression free survival than those with lower CCL20 expression. The boxplots show ongoing responders expressing lower level of CCL20 in pretreatment tumor than relapsed and/or non-responders. FIG. 6. Overall and progression-free survival curve of CLINICAL TRIAL-1 subjects grouped by CCL20 gene counts. Kaplan-Meier overall and progression-free survival curves with a median cut-off selection for CCL20 gene counts in pretreatment tumor samples with significance determined by the Log-Rank test. The boxplots show CCL20 gene counts by ongoing response groups. Ongoing responders are shown in green, relapsed patients are shown in orange, non-responders are shown in blue, while relapsed with nonresponders (others) are show in yellow. Nonparametric Wilcoxon tests and Kruskal-Wallis tests are conducted for comparisons of 2 or 3 groups, respectively.

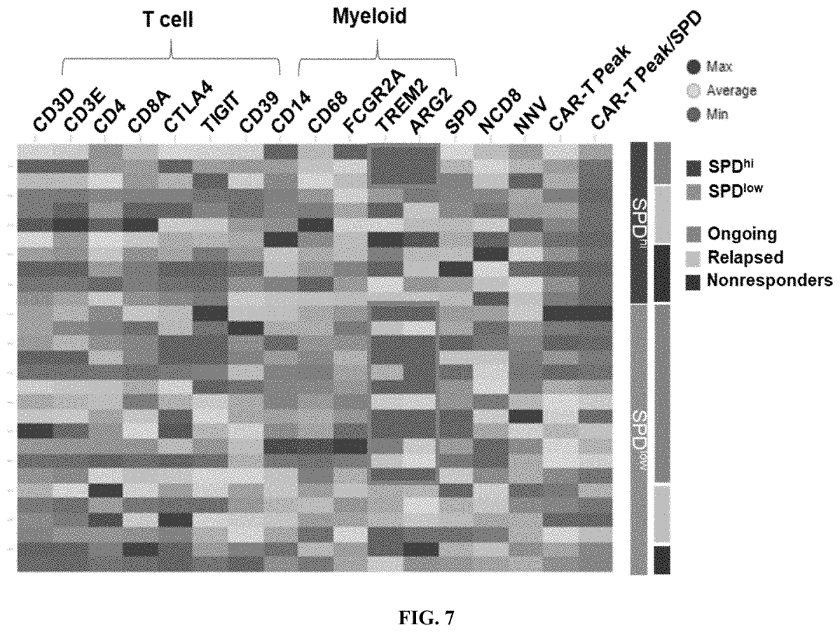

Patients in durable response show lower expression of ARG2 and TREM2 while relapsed and nonresponders show higher expression of ARG2 and TREM2, particularly in patients with higher baseline tumor burden. FIG. 7. Associations between pretreatment T cell and Myeloid cell gene signature with ongoing response within patients with high (SPDhi) or low (SPDlow) baseline tumor burden. Values in red are representative of a value greater the mean expression while those in blue are representative of a value less than mean expression of the corresponding gene. Total number of infused CD8 (NCD8), total number of infused naïve products (NNV), peak level of CAR-T cells and its value relative to baseline tumor burden (CAR-T peak/SPD) are included as a comparison.

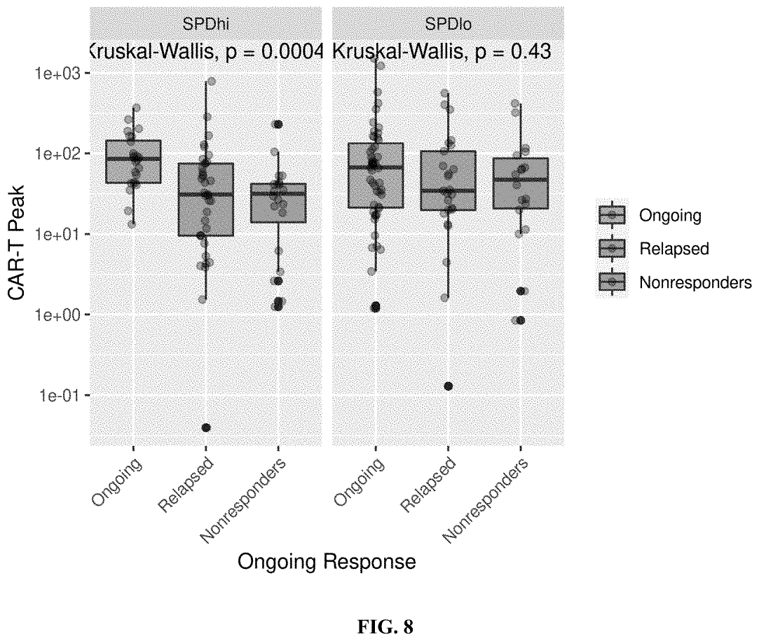

CAR-T peak expansion is positively associated with ongoing response, particularly in patients with large baseline tumor burden. FIG. 8. Association between peak CAR-T levels (cells/μL) by ongoing response groups within patients with high (SPDhi) or low (SPDlow) baseline tumor burden. Ongoing responders are shown in green, relapsed patients are shown in orange, and non-responders are shown in blue. Nonparametric Kruskal-Wallis tests are conducted for comparisons of 3 groups.

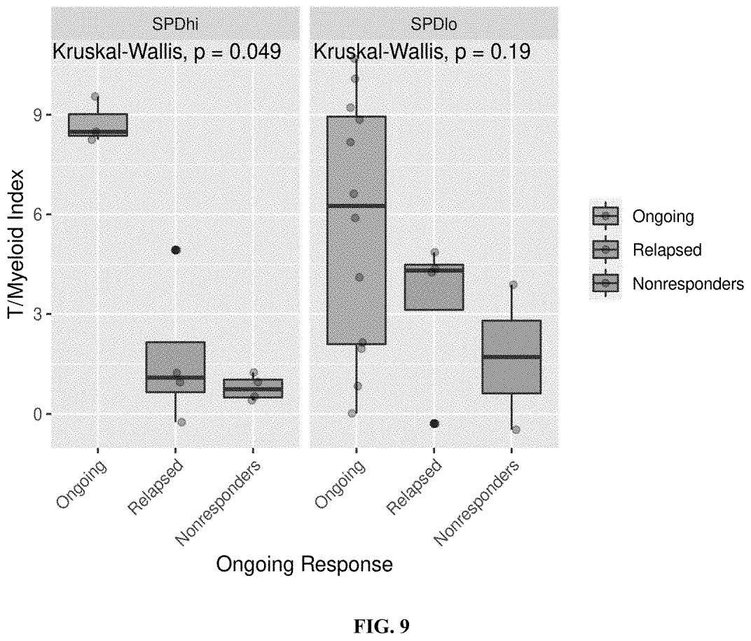

Ratio of T/Myeloid Index is positively associated with ongoing response, particularly in patients with large baseline tumor burden. FIG. 9. Ratio of T cell to TME myeloid inflammation by ongoing response groups within patients with high (SPDhi) or low (SPDlow) baseline tumor burden. Selected genes were used to derive T cell (CD3D, CD8A, CTLA4, TIGIT) and TME myeloid inflammation (ARG2 and TREM2) indices. Ongoing responders are shown in green, relapsed patients are shown in orange, and non-responders are shown in blue. Nonparametric Kruskal-Wallis tests are conducted for comparisons of 3 groups.

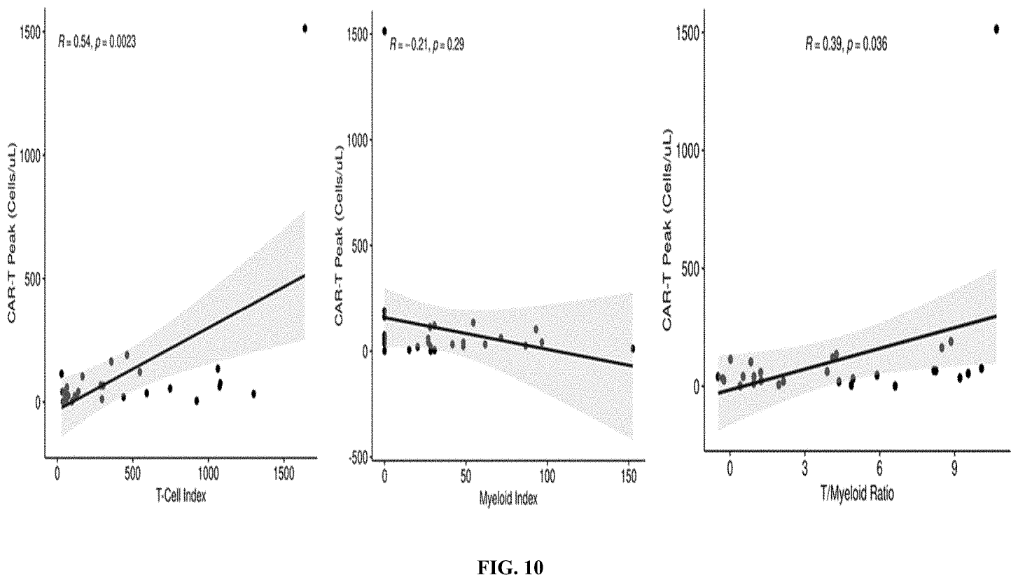

CAR-T peak expansion is positively associated with T cell index and T/Myeloid ratio. FIG. 10. Associations between peak level of CAR-T cells with T cell, TME myeloid inflammation indices, and ratio of T cell to TME myeloid inflammation. Spearman rank coefficient (R) and p values are shown.

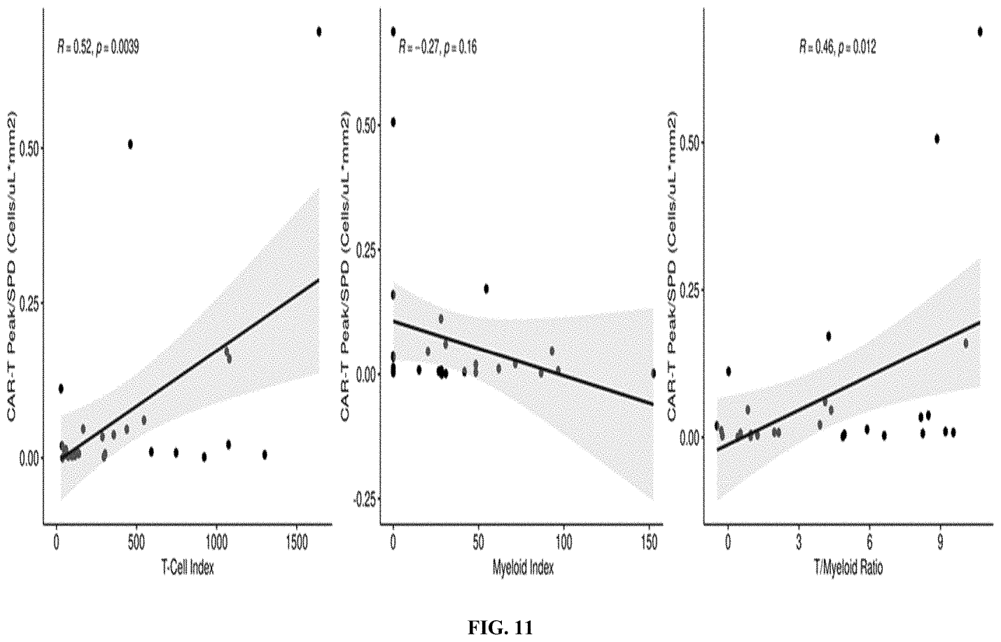

Peak level of CAR-T cells relative to baseline tumor burden is positively associated with T cell index and T/Myeloid ratio. FIG. 11. Associations between peak levels of CAR-T cells relative to baseline tumor burden with T cell, TME myeloid inflammation indices, and ratio of T cell to TME myeloid inflammation. Spearman rank coefficient (R) and p values are shown.

TABLE 2
Representative Results

| Parameter | Min | P10 | Q1 | Median | Q3 | P90 | Max | Range 1 |
|---|---|---|---|---|---|---|---|---|
| ARG2 | 0 | 0 | 0 | 26.77 | 39.57 | 73.88 | 101.14 | 0-0 |
| TREM2 | 0 | 0 | 0 | 10.32 | 34.11 | 101.15 | 195.69 | 0-0 |
| CCL20 | 0 | 0 | 0 | 0 | 44.11 | 100.89 | 390.6 | 0-0 |
| IL8 | 0 | 0 | 0 | 41.55 | 97.93 | 203.99 | 2637.78 | 0-0 |
| IL13 | 0 | 0 | 0 | 8.95 | 39.18 | 88.17 | 193.07 | 0-0 |
| IFNL2 | 0 | 0 | 0 | 10.71 | 72.36 | 152.45 | 633.04 | 0-0 |
| OSM | 0 | 0 | 0 | 7.93 | 38.52 | 121.9 | 354.61 | 0-0 |
| IL11RA | 0 | 0 | 0 | 76.56 | 96.36 | 121.57 | 172.05 | 0-0 |
| CCL11 | 0 | 0 | 0 | 26.67 | 85.47 | 201.78 | 317.84 | 0-0 |
| MCAM | 0 | 0 | 59.37 | 132.31 | 201.27 | 313.65 | 409.77 | 0-0 |
| PTGDR2 | 0 | 0 | 0 | 0 | 21.58 | 39.29 | 181.25 | 0-0 |
| CCL16 | 0 | 0 | 0 | 0 | 19.17 | 49.22 | 194.38 | 0-0 |
| C8G | 0 | 0 | 0 | 11.35 | 48.58 | 102.64 | 130.02 | 0-0 |
| Myeloid | 0 | 0 | 0 | 27.45 | 48.38 | 87.29 | 152.49 | 0-0 |
| Signature | | | | | | | | |
| T Cell/ | −0.47 | −0.02 | 0.86 | 4 | 7.78 | 9.25 | 10.68 | −0.47--0.02 |
| Myeloid | | | | | | | | |
| Ratio | | | | | | | | |
| Baseline | 171 | 485 | 1922 | 3689 | 6533 | 9940 | 39658 | 171-485 |
| Tumor | | | | | | | | |
| Burden | | | | | | | | |
| (SPD) | | | | | | | | |

| Parameter | Range 2 | Range 3 | Range 4 | Range 5 | Range 6 |
|---|---|---|---|---|---|
| ARG2 | 0-0 | 0-26.77 | 26.77-39.57 | 39.57-73.88 | 73.88-101.14 |
| TREM2 | 0-0 | 0-10.32 | 10.32-34.11 | 34.11-101.15 | 101.15-195.69 |
| CCL20 | 0-0 | 0-0 | 0-44.11 | 44.11-100.89 | 100.89-390.6 |
| IL8 | 0-0 | 0-41.55 | 41.55-97.93 | 97.93-203.99 | 203.99-2637.78 |
| IL13 | 0-0 | 0-8.95 | 8.95-39.18 | 39.18-88.17 | 88.17-193.07 |
| IFNL2 | 0-0 | 0-10.71 | 10.71-72.36 | 72.36-152.45 | 152.45-633.04 |
| OSM | 0-0 | 0-7.93 | 7.93-38.52 | 38.52-121.9 | 121.9-354.61 |
| IL11RA | 0-0 | 0-76.56 | 76.56-96.36 | 96.36-121.57 | 121.57-172.05 |
| CCL11 | 0-0 | 0-26.67 | 26.67-85.47 | 85.47-201.78 | 201.78-317.84 |
| MCAM | 0-59.37 | 59.37-132.31 | 132.31-201.27 | 201.27-313.65 | 313.65-409.77 |
| PTGDR2 | 0-0 | 0-0 | 0-21.58 | 21.58-39.29 | 39.29-181.25 |
| CCL16 | 0-0 | 0-0 | 0-19.17 | 19.17-49.22 | 49.22-194.38 |
| C8G | 0-0 | 0-11.35 | 11.35-48.58 | 48.58-102.64 | 102.64-130.02 |
| Myeloid | 0-0 | 0-27.45 | 27.45-48.38 | 48.38-87.29 | 87.29-152.49 |
| Signature | | | | | |
| T Cell/ | −0.02-0.86 | 0.86-4 | 4-7.78 | 7.78-9.25 | 9.25-10.68 |
| Myeloid | | | | | |
| Ratio | | | | | |
| Baseline | 485-1922 | 1922-3689 | 3689-6533 | 6533-9940 | 9940-39658 |
| Tumor | | | | | |
| Burden | | | | | |
| (SPD) | | | | | |

### Example 2

This Example is a continuation of Example 1 and the data were obtained from the same patient populations and by the same methods. The goal was to systematically analyze pretreatment tumor microenvironment (TME) characteristics that may influence CAR T-cell performance in patients with LBCL from Clinical Trial-1, particularly those with higher tumor burden and lower ongoing response rate. In this post-hoc analysis, evaluable samples from patients in clinical trial-1 Phase 1 and Phase 2 Cohorts 1-3 were analyzed. As such, n values vary by assay type Cohorts 1 and 2 represent the pivotal cohorts. (Locke F L, et al. Lancet Oncol. 2019; 20:31-42; Neelapu S S, et al. N Engl J Med. 2017; 377:2531-2544). Cohort 3, one of several exploratory safety management cohorts added to ZUMA-1, evaluated the prophylactic use of the anticonvulsant levetiracetam and the anti-interleukin-6 receptor antibody tocilizumab to minimize CAR T-cell treatment-related toxicities. (Locke F L, et al. Blood. 2017; 130(suppl, abstr):1547). Patients in Phase 1 and Phase 2 Cohorts 1 and 2 had ≥2 years of follow-up (median, 27.1 months). Patients in Cohort 3 had ≥6 months of follow-up (median, 9.8 months). The pretreatment immune TME was analyzed by multiplex immunohistochemistry and gene expression profiling (NanoString), as previously described. (Galon J, et al. J Clin Oncol. 2020; 38(suppl, abstr):3022; Rossi J M, et al. Cancer Res. 2018; 78(suppl, abstr):LB-016). The baseline tumor burden (by SPD) was evaluated as previously described. (Locke F L, et al. Blood Adv. 2020; 4:4898-4911). Correlative analyses of the above covariates with clinical outcomes were performed by Spearman rank correlation or Wilcoxon or Kruskal-Wallis test. The median tumor burden (by SPD) from clinical trial-1 Phase 1 and Phase 2 Cohorts 1+2 was used as a cutoff for high (>3721 mm2) versus low (≤3721 mm2) tumor burden. Response definitions were according to response at the time of data cutoff and were as follows: ongoing/durable responders were patients who achieved a complete or partial response and remained in response; nonresponders were patients who experienced stable or progressive disease as best response; and relapsed were patients who achieved a complete or partial response and subsequently experienced disease progression.

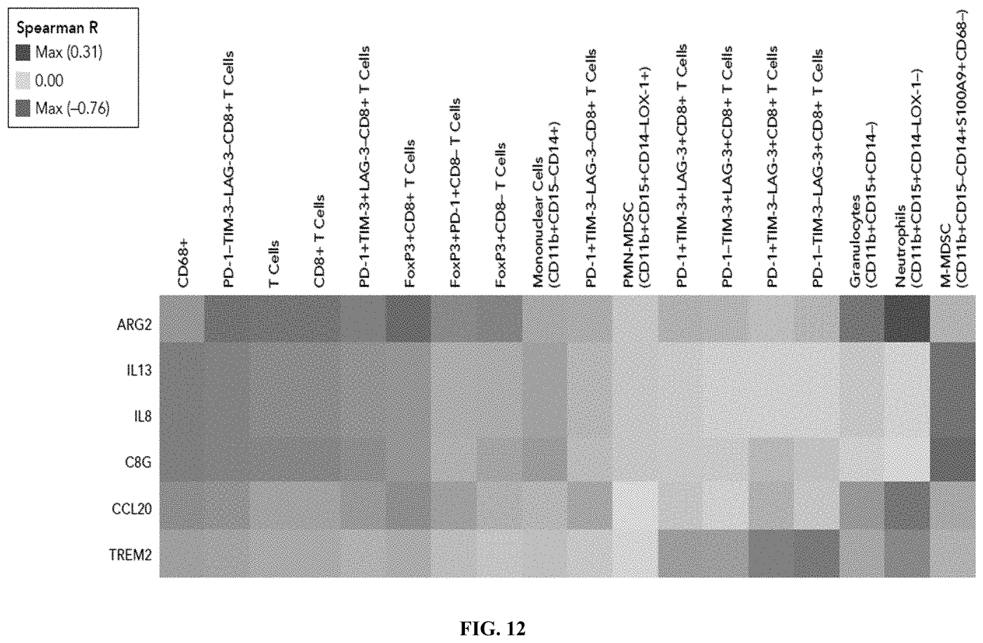

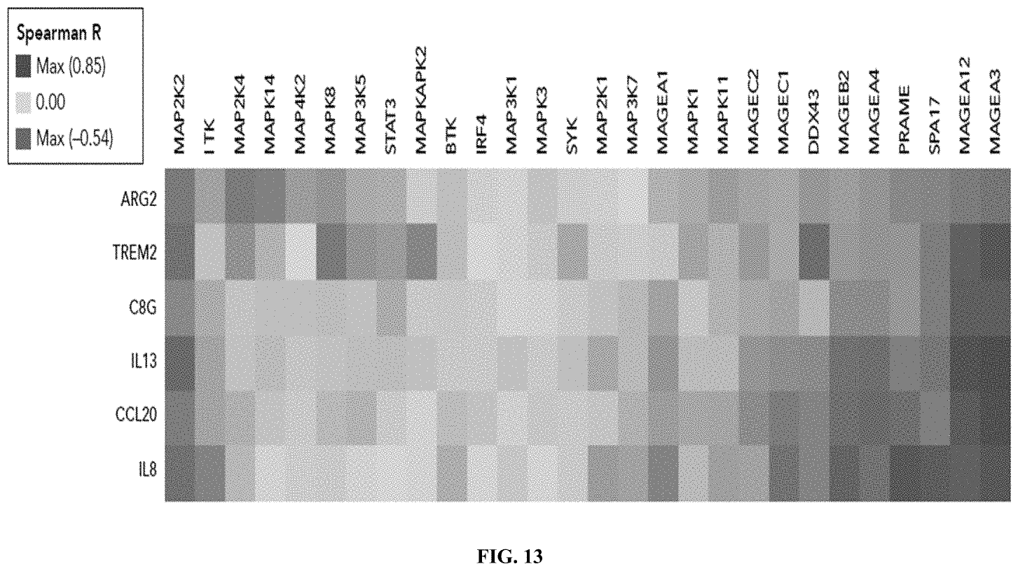

The myeloid signature obtained from FIG. 1 (see Example 1), which was generated by Nanostring, was associated with key TME immune cell subsets, which was shown using data generated utilizing multiplex IHC. FIG. 12. Genes negatively associated with ongoing response (e.g., ARG2, IL13, IL8, CBG, CCL20, and TREM2) were positively associated with the myeloid cell population within the TME. Conversely, top genes differentially expressed in relapsed patients and non-responders showed positive association with myeloid cells (granulocytes, neutrophils, and M-MDSC) and negative association with T cells (e.g., CD8+ T cells; FoxP3+CD9+ T cells) within the TME. FIG. 12. The suppressive myeloid gene signature was also shown to be positively associated with cancer testis antigens (CTA). FIG. 13. CTA genes have previously been shown to be negatively associated with best response (Rossi J M, et al. Cancer Res. 2018; 78(suppl, abstr):LB-016). A favorable immune TME comprised a more pronounced T-cell gene expression signature relative to suppressive myeloid cell gene expression signature. Patients with low ARG2 and TREM2 gene expression in the pretreatment TME who showed relatively higher CAR T-cell expansion commensurate with tumor burden achieved durable response. These data suggest that overcoming a dysregulated myeloid-related TME in conjunction with utilizing highly functional CAR T-cell products maximizing the durable clinical benefit in patients with high tumor burden. Axicabtagene ciloleucel may overcome high pretreatment tumor burden in patients with a favorable immune TME and high CAR T-cell expansion.

### Example 3

Axicabtagene ciloleucel, an autologous anti-CD19 chimeric antigen receptor (CAR) T-cell therapy, is approved for treatment of relapsed/refractory large B-cell lymphoma (R/R LBCL) after ≥2 prior systemic therapies (YESCARTA® (axicbatagene ciloleucel) [summary of product characteristics]. Amsterdam, the Netherlands: Kite Pharma EU B.V.; 2018; YESCARTA® (axicabtagene ciloleucel) [package insert]. Santa Monica, Calif.: Kite Pharma, Inc; 2017). To reduce axicabtagene ciloleucel-related toxicity, several exploratory safety management cohorts were added to CLINICAL TRIAL-1 (NCT02348216), the pivotal phase 1/2 study of axicabtagene ciloleucel in refractory LBCL. Cohort 4 evaluated the rates and severity of cytokine release syndrome (CRS) and neurologic events (NEs) with earlier corticosteroid and tocilizumab use. Primary endpoints were incidence and severity of CRS and NEs. Patients received 2×10⁶ anti-CD19 CAR T cells/kg after conditioning therapy. Forty-one patients received axicabtagene ciloleucel. Incidences of any-grade CRS and NEs were 93% and 61%, respectively (grade ≥3, 2% and 17%). There was no grade 4 or 5 CRS or NE. Despite earlier dosing, the cumulative cortisone-equivalent corticosteroid dose in patients requiring corticosteroid therapy was lower than that reported in the pivotal CLINICAL TRIAL-1 cohorts. With a median follow-up of 14.8 months, objective and complete response rates were 73% and 51%, respectively, and 51% of treated patients were in ongoing response. Earlier and measured use of corticosteroids and/or tocilizumab has the potential to reduce the incidence of grade ≥3 CRS and NEs in patients with R/R LBCL receiving axicabtagene ciloleucel.

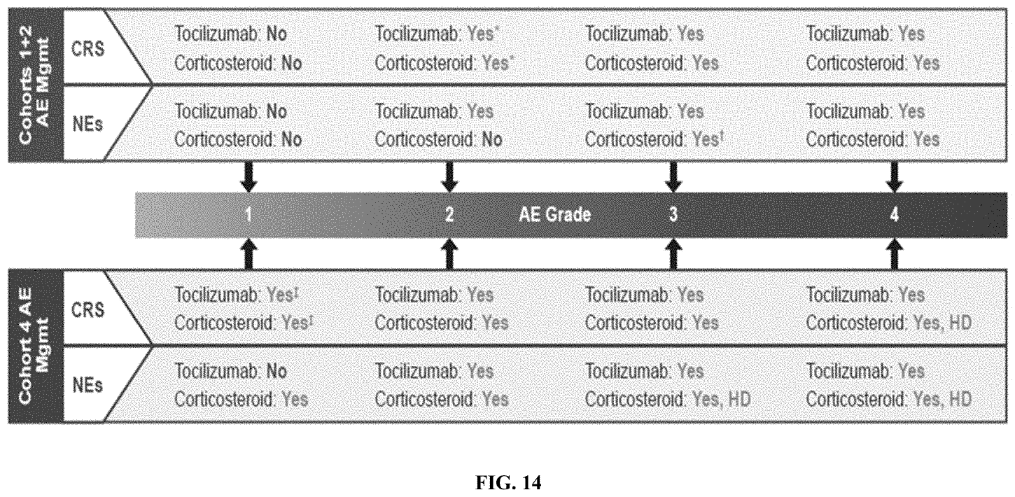

CLINICAL TRIAL-1 is a single-arm, multicenter, registrational study of axicabtagene ciloleucel in R/R LBCL being conducted in the United States, Europe, Canada, and Israel. Cohort 4 procedures were similar to those described for cohorts 1+2. (Neelapu et al., N Engl J Med. 2017; 377(26):2531-44) The primary differences in cohort 4 were use of levetiracetam prophylaxis and earlier corticosteroid and tocilizumab intervention for managing CRS and NEs (FIG. 14).

Eligible patients in cohort 4 had R/R LBCL after ≥2 systemic lines of therapy or were refractory to first-line therapy (i.e., best response of progressive disease (PD) or stable disease (to ≥4 cycles of first-line therapy with stable disease duration no longer than 6 months). Prior therapy must have included an anti-CD20 monoclonal antibody (unless the tumour was CD20-negative) and an anthracycline-containing chemotherapy regimen. Patients were required to have an Eastern Cooperative Oncology Group performance status of 0 or 1. Additional inclusion criteria were absolute neutrophil count >1,000 cells/μL, absolute lymphocyte count >100 cells/μL, platelet count >75,000 cells/μL, adequate organ function, no central nervous system involvement, and no active infection.

Cohort 4 patients received a conditioning regimen of cyclophosphamide (500 mg/m²/day) and fludarabine (30 mg/m²/day) on days −5 to −3, and 1 dose of axicabtagene ciloleucel (target dose, 2×10⁶ CAR T cells/kg) on day 0. Bridging therapy prior to initiation of conditioning chemotherapy (Table 3) was allowed per investigator's discretion (e.g., bulky disease or rapidly progressing disease at screening or baseline).

TABLE 3
Bridging therapy regimens.*

| Type | Therapy regimens^{†} | Timing and washout requirements |
|---|---|---|
| Corticosteroid | Dexamethasone at a dose of | May be administered after |
| | 20 mg to 40 mg or equivalent, | apheresis/enrollment and must be |
| | either PO or IV daily for 1 to 4 days | completed before the start of |
| | Choice of corticosteroid and dose | conditioning chemotherapy |
| | may be adjusted for age/comorbidities | |
| | or per local or institutional guidelines | |
| HDMP + | 1 g/m² of HDMP for 3 days in | May be administered after |
| rituximab | combination with rituximab at | enrollment and completed ≥7 days |
| | 375 mg/m² weekly for 3 weeks | before the start of conditioning |
| | | chemotherapy |
| Combination | B-R: bendamustine (90 mg/m², | May be administered after |
| chemotherapy | days 1 and 2); rituximab | enrollment and completed ≥14 days |
| | (375 mg/m², day 1) | before the start of conditioning |
| | | chemotherapy |

HDMP, high-dose methylprednisolone; IV, intravenously; PET-CT, positron emission tomography-computed tomography; PO, orally.
*A new baseline PET-CT was performed post-bridging therapy.
^{†}The bridging therapy regimen may be chosen at the investigator's discretion.

Patients received levetiracetam (750 mg orally or intravenously twice daily) starting on day 0 and at the onset of grade ≥2 neurologic toxicities if NEs occurred after the discontinuation of prophylactic levetiracetam. If a patient did not experience any grade ≥2 neurologic toxicities, levetiracetam was tapered and discontinued as clinically indicated. Corticosteroid therapy was initiated to manage all grade 1 CRS if there was no improvement after 3 days and for all grade ≥1 NEs (FIG. 14; Table 4). Tocilizumab was initiated at grade 1 CRS if there was no improvement after 3 days, at grade ≥2 CRS, and at grade ≥2 NE (Table 4).

TABLE 4
Tocilizumab and corticosteroid guidelines for adverse
event management in CLINICAL TRIAL-1 cohort 4.

| CRS | | |
|---|---|---|
| grade | Tocilizumab dose* | Corticosteroid dose* |
| 1 | If no improvement after 3 days, | If no improvement after 3 days, |
| | 8 mg/kg over 1 hour^{†}; repeat | dexamethasone 10 mg × 1 |
| | every 4-6 hours as needed | |
| 2 | 8 mg/kg over 1 hour^{†}; repeat | Dexamethasone 10 mg × 1 |
| | every 4-6 hours as needed | |
| 3 | Per grade 2 | Methylprednisolone 1 mg/kg |
| | | IV twice daily or equivalent |
| | | dexamethasone dose |
| 4 | Per grade 2 | Methylprednisolone 1000 |
| | | mg/day IV × 3 days |
| NE | | |
| grade | Tocilizumab dose | Corticosteroid dose |
| 1 | N/A | Dexamethasone 10 mg × 1 |
| 2 | 8 mg/kg over 1 hour; repeat | Dexamethasone 10 mg 4 |
| | every 4-6 hours as needed | times/day |
| 3 | As per grade 2 | Methylprednisolone 1 g once |
| | | daily |
| 4 | As per grade 2 | Methylprednisolone 1 g twice |
| | | daily |

CRS, cytokine release syndrome; IV, intravenously; N/A, not applicable; NE, neurologic event.
*Therapy to be tapered upon improvement of symptoms at investigator's discretion.
^{†}Not to exceed 800 mg.

No formal hypothesis was tested, and all endpoints were analyzed descriptively. The primary endpoint in cohort 4 was the incidence and severity of CRS and NEs. CRS was graded according to modified Lee et al criteria (Lee et al., Blood. 2014; 124(2):188-95) and NEs were graded per Common Terminology Criteria for Adverse Events version 4.03 (U.S. Department of Health and Human Services. Common Terminology Criteria for Adverse Events (CTCAE) Version 4.03. 2010). Key safety-related secondary endpoints included the incidence of other adverse events and clinically significant changes in safety laboratory values. Key efficacy-related secondary endpoints included ORR per investigator assessment, duration of response, PFS, OS, anti-CD19 CAR T-cell levels in the blood, and cytokine levels in the serum.

The modified intent-to-treat population included patients enrolled and treated with an axicabtagene ciloleucel dose of ≥1×10⁶ anti-CD19 CAR T cells/kg. This analysis set was used for all objective response analyses and endpoints based on objective response. The safety analysis set included all patients treated with any dose of axicabtagene ciloleucel. Tumour burden in cohort 4 was measured after bridging and before conditioning chemotherapy. The cumulative corticosteroid dose was calculated by conversion to systemic cortisone-equivalent dose during the initial hospitalization period.

Pharmacokinetic analysis was performed using a validated polymerase chain reaction enumerating the gene-marked CAR T cells in blood (Neelapu et al., N Engl J Med. 2017; 377(26):2531-44; Kochenderfer et al., J Clin Oncol. 2015; 33(6):540-9). Serum was obtained at multiple timepoints for quantification of soluble markers, including cytokines. Cerebrospinal fluid (CSF) was collected after confirmation of eligibility, before conditioning chemotherapy, on day 5 (±3 days) after axicabtagene ciloleucel infusion, and at the Week 4 visit (±3 days). Up to 46 soluble markers were measured in serum and CSF using multiplex assay kits from Meso Scale Discovery or Luminex, the ProteinSimple Simple Plex, or the R&D Systems Quantikine® enzyme-linked immunosorbent assay kit. Product cells were characterized by flow cytometry and coculture with CD19-expressing target cells followed by enzyme-linked immunosorbent assay or Meso Scale Discovery.

Exploratory (Propensity score matching analysis) PSM analysis (Rosenbaum and Rubin, Biometriks. 1983; 70(1):41-55; Austin, Multivariate Behav Res. 2011; 46(3):399-424) was performed to allow descriptive comparison of results for patients in cohort 4 versus cohorts 1+2 (median follow-up, 15.4 months) after balancing for the following baseline characteristics: age, Eastern Cooperative Oncology Group (ECOG) performance status, tumour burden, International Prognostic Index score, number of prior lines of chemotherapy, prior platinum use, disease stage, and lactate dehydrogenase (LDH) level (Supplemental Methods). Standardised mean difference (Austin, Stat Med. 2008; 27(12):2037-49; Imai et al., J R Statist Soc A. 2008; 171:481-502) within ±0.2 between cohort 4 and matched cohorts 1+2 was used as a criterion to assess the balance of covariates after PSM. PSM analysis represents a statistical method to reduces bias in comparisons between two groups by minimizing potential confounding effects of measured or unmeasured baseline characteristics that may be present between groups when using observational data (Rosenbaum and Rubin, Biometriks. 1983; 70(1):41-55; Austin, Multivariate Behav Res. 2011; 46(3):399-424). Using this approach, the effects of treatment on outcomes between two distinct groups may be estimated in the absence of a randomized trial (Rosenbaum and Rubin, Biometriks. 1983; 70(1):41-55; Austin, Multivariate Behav Res. 2011; 46(3):399-424). Here, a post hoc propensity score matching analysis was performed to descriptively compare cohort 4 and pivotal cohorts 1+2 of CLINICAL TRIAL-1. Covariate balance before and after matching was assessed by standardized mean difference (SMD), or the calculated difference in means between the 2 groups divided by the standard deviation (Austin, Stat Med. 2008; 27(12):2037-49; Imai et al., J R Statist Soc A. 2008; 171:481-502). This statistical method is the most widely used diagnostic metric for propensity score matching analysis and is not influenced by factors beyond improved balance (eg, sample size of matched subgroups) (Austin, Stat Med. 2008; 27(12):2037-49; Imai et al., J R Statist Soc A. 2008; 171:481-502). For this reason, the validity of propensity score matching comparisons is established through SMD covariate balance diagnosis after matching.

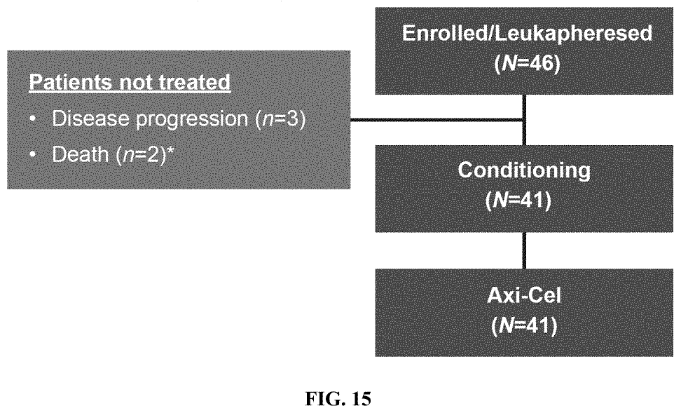

Cohort 4 enrollment commenced in February 2018. Forty-six patients were enrolled and leukapheresed in cohort 4, and 41 patients received the minimum target dose of axicabtagene ciloleucel. The latter group comprised both the modified intent-to-treat and safety analysis sets (FIG. 15). Sixty-eight percent of patients (n=28/41) received bridging therapy before receiving axicabtagene ciloleucel with a median reduction in tumour burden among the 17 evaluable patients of 10%. As of the Nov. 6, 2019 data cutoff, the median follow-up was 14.8 months (range, 8.9-19.9 months). Among treated patients, the median age was 61 years (range, 19-77; Table 5).

TABLE 5
Baseline characteristics

| | Cohort 4 | |
|---|---|---|
| Characteristic | (N = 41) | |
| Disease type, n (%) | | |
| DLBCL | 26 | (63) |
| PMBCL | 2 | (5) |
| TFL | 10 | (24) |
| HGBCL | 3 | (7) |
| Age | | |
| Median (range), years | 61.0 | (19-77) |
| ≥65 y, n (%) | 13 | (32) |
| Male sex, n (%) | 28 | (68) |
| ECOG performance status score of 1, n (%) | 20 | (49) |
| Disease stage, n (%) | | |
| I or II | 11 | (27) |
| III or IV | 29 | (71) |
| IPI score, n (%) | | |
| 0-2 | 21 | (51) |
| 3-4 | 20 | (49) |
| CD19 positivity, n/N (%)* | | |
| Yes | 22/24 | (92) |
| No | 2/24 | (8) |
| Number of prior lines of chemotherapy, n (%) | | |
| 1 | 0 | |
| 2 | 15 | (37) |
| 3 | 15 | (37) |
| 4 | 8 | (20) |
| ≥5 | 3 | (7) |
| Prior SCT, n (%) | 14 | (34) |
| PD as best response to most recent | 15 | (37) |
| chemotherapy, n (%)^{†} | | |
| Median (range) tumour burden by SPD,^{‡} mm² | 2100 | (204-24,758) |
| Median (range) LDH, U/l | 263 | (145-4735) |
| Median (range) ferritin, ng/ml | 393 | (23-3457) |
| Refractory subgroup, n (%) | | |
| Primary refractory | 0 | (0) |
| Refractory ≥2^{nd}-line therapy | 28 | (68) |
| Relapsed ≥2^{nd}-line therapy | 5 | (12) |
| Relapsed post-ASCT | 8 | (20) |

ASCT, autologous stem cell transplant; DLBCL, diffuse large B-cell lymphoma; ECOG, Eastern Cooperative Oncology Group; HGBCL, high-grade B-cell lymphoma; IPI, International Prognostic Index; LDH, lactate dehydrogenase; PD, progressive disease; PMBCL, primary mediastinal B-cell lymphoma; SCT, stem cell transplant; SPD, sum of the products of diameters; TFL, transformed follicular lymphoma.
*Archival and on-study pretreatment tumour biopsy ascertainment rate was 59% (24/41) by central confirmation of diagnosis. Two additional subjects had missing confirmatory diagnosis due to absence of tumour tissue within the biopsy specimen sent for central assessment.
^{†}For patients who had not relapsed post-ASCT.
^{‡}At last observation before conditioning chemotherapy; may have been measured before or after bridging in patients who received bridging.

The most common disease subtype was diffuse LBCL (63%). Most patients (71%) had disease stage III or IV, 63% had ≥3 prior therapies, and 37% had a best response of progressive disease to their most recent chemotherapy. Product characteristics were largely comparable with those previously reported in CLINICAL TRIAL-1 (Table 6).

TABLE 6

| Parameter | Cohort 4 | |
|---|---|---|
| Median (min-max) | (N = 41) | |
| Total number of T cells per μL | 275.4 | (176.4-487.8) |
| Total number of CAR T cells per μL | 155.0 | (100.0-200.0) |
| Percent transduction, % | 55.0 | (33.0-73.0) |
| IFN-γ level, pg/ml | 8141.0 | (1086.0-1.9 × 10⁴) |
| Viability, % | 92.0 | (72.0-96.0) |
| CD4/CD8 ratio | 1.53 | (0.5-7.2) |
| Naive (CCR7+CD45RA+) T cells, % | 20.35 | (2.5-53.5) |
| Central memory (CCR7+CD45RA−) | 35.25 | (16.4-44.9) |
| T cells, % | | |

CAR, chimeric antigen receptor; IFN, interferon; max, maximum; min, minimum.

All patients who received axicabtagene ciloleucel experienced AEs, with 98% experiencing at least 1 grade ≥3 event—most frequently neutropenia (39%), decreased neutrophil count (29%), anemia (24%), and pyrexia (24%; Table 7). Any-grade infection was reported in 25 (61%) patients, with worst grade 3, 4, and 5 occurring in 8 (20%), 1 (2%), and 1 (2%) patient, respectively.

TABLE 7
Incidence and severity of TEAEs.*

| | Cohort 4 | | | | | |
|---|---|---|---|---|---|---|
| | (N = 41) | | | | | |
| | Any | | Worst | | Worst | |
| | grade | | grade 3 | | grade 4 | |
| Any, n (%) | 41 | (100) | 12 | (29) | 22 | (54) |
| Pyrexia | 39 | (95) | 10 | (24) | 0 | (0) |
| Diarrhea | 25 | (61) | 4 | (10) | 0 | (0) |
| Hypotension | 25 | (61) | 4 | (10) | 0 | (0) |
| Anemia | 19 | (46) | 10 | (24) | 0 | (0) |
| Fatigue | 19 | (46) | 3 | (7) | 0 | (0) |
| Headache | 16 | (39) | 1 | (2) | 0 | (0) |
| Neutropenia | 16 | (39) | 4 | (10) | 12 | (29) |
| Nausea | 12 | (29) | 0 | (0) | 0 | (0) |
| Neutrophil count decreased | 12 | (29) | 1 | (2) | 11 | (27) |
| Chills | 11 | (27) | 0 | (0) | 0 | (0) |
| Cough | 10 | (24) | 0 | (0) | 0 | (0) |
| Platelet count decreased | 10 | (24) | 2 | (5) | 2 | (5) |
| Somnolence | 8 | (20) | 3 | (7) | 0 | (0) |
| Dizziness | 7 | (17) | 0 | (0) | 0 | (0) |
| Encephalopathy | 7 | (17) | 2 | (5) | 0 | (0) |
| Leukopenia | 7 | (17) | 1 | (2) | 5 | (12) |
| Tachycardia | 7 | (17) | 1 | (2) | 0 | (0) |
| Thrombocytopenia | 7 | (17) | 4 | (10) | 1 | (2) |
| Back pain | 6 | (15) | 0 | (0) | 0 | (0) |
| Constipation | 6 | (15) | 0 | (0) | 0 | (0) |
| Hypokalemia | 6 | (15) | 1 | (2) | 0 | (0) |
| Hypophosphatemia | 6 | (15) | 4 | (10) | 0 | (0) |
| Hypoxia | 6 | (15) | 3 | (7) | 0 | (0) |
| Tremor | 6 | (15) | 0 | (0) | 0 | (0) |
| Vomiting | 6 | (15) | 1 | (2) | 0 | (0) |
| White blood cell count decreased | 6 | (15) | 1 | (2) | 5 | (12) |

TEAE, treatment-emergent adverse event.
*TEAEs that occurred in ≥15% of patients and includes all grade ≥3 events that occurred in >10% of patients.

There were 2 deaths due to AEs and both were reported as related to conditioning chemotherapy (day 13 pneumonia) or prior chemotherapy (day 354 acute myeloid leukemia; shown by retrospective analysis to have transformed from underlying myelodysplastic syndrome present at leukapheresis). Grade ≥3 cytopenias present on or after day 30 were reported in 39% of patients (Table 8).

TABLE 8
Incidence of worst grade ≥3 neutropenia, thrombocytopenia,
and anemia present on or after day 30 following
axicabtagene ciloleucel infusion

| | Cohort 4 | |
|---|---|---|
| TEAE, n (%) | (N = 41) | |
| Any | 16 | (39) |
| Neutropenia | 13 | (32) |
| Thrombocytopenia | 4 | (10) |
| Anemia | 3 | (7) |

The overall incidence of CRS was 93%, grade 3 CRS occurred in 2% of patients (Table 9), and there were no grade 4 CRS events or deaths in the setting of CRS. The most common grade ≥3 symptoms of CRS were pyrexia (24%), hypotension (8%) and hypoxia (5%). The median time to onset of CRS was 2 days, with a median duration of 6.5 days, and all CRS events resolved by the data cutoff NEs occurred in 61% of patients, with incidences of grade ≥3 NEs of 17% (Table 9).

TABLE 9
Incidence, severity, onset, and duration of CRS and NEs

| | Cohort 4 | |
|---|---|---|
| TEAE | (N = 41) | |
| CRS | | |
| Any, n (%) | 38 | (93) |
| Worst grade 1, n (%) | 13 | (32) |
| Worst grade 2, n (%) | 24 | (59) |
| Worst grade 3, n (%) | 1 | (2) |
| Worst grade 4, n (%) | 0 | |
| Worst grade 5, n (%) | 0 | |
| Median (range) time to onset | 2.0 | (1.0-8.0) |
| of any grade CRS, days | | |
| Median (range) duration, days | 6.5 | (2.0-16.0) |
| NEs | | |
| Any, n (%) | 25 | (61) |
| Worst grade 1, n (%) | 14 | (34) |
| Worst grade 2, n (%) | 4 | (10) |
| Worst grade 3, n (%) | 7 | (17) |
| Worst grade 4, n (%) | 0 | |
| Worst grade 5, n (%) | 0 | |
| Median (range) time to onset | 6.0 | (1.0--93.0) |
| of any grade NE, days | | |
| Median (range) duration, days | 8.0 | (1.0-144.0) |

CRS, cytokine release syndrome; NE, neurologic event; TEAE, treatment-emergent adverse event.

The most common grade ≥3 NEs in cohort 4 were somnolence (7%), confusional state (7%), and encephalopathy (5%). There were no grade 4 or 5 NEs. Notably, grade ≥3 NEs were limited to patients who received bridging therapy. The median time to onset of NEs was 6 days, with a median duration of 8 days. Three patients had ongoing NEs as of the data cutoff (Table 10).

TABLE 10
Summary of neurologic events unresolved at data cutoff.

| | | | Related to | |
|---|---|---|---|---|
| | Neurologic event | | axicabtagene | Duration as of |
| Patient | (preferred term) | Grade | ciloleucel | data cutoff |
| 1 | Memory | 1 | Related | 345 days |
| | impairment | | | |
| 2 | Dysesthesia | 1 | Not related | 77 days |
| 3 | Myelitis | 1 | Related | 252 days |
| 4 | Disorientation | 3 | Not related | N/A* |
| | Somnolence | 2 | Not related | |
| 5 | Disorientation | 1 | Related | N/A^{†} |
| | Somnolence | 1 | Related | |

axicabtagene ciloleucel, axicabtagene ciloleucel; N/A, not applicable.
*Neurologic events were ongoing at time of death due to pneumonia on day 13.
^{†}Neurologic events were ongoing at time of death due to disease progression on day 6.

Bridging therapy did not contribute to a reduction in the incidence of grade ≥3 CRS (bridging, 1/28 [4%]; no bridging, 0/13 [0%]) or NEs (bridging, 7/28 [25%]; no bridging, 0/13 [0%]) in cohort 4. A total of 73% patients received corticosteroids in cohort 4. Among those who received corticosteroids, the cumulative cortisone-equivalent corticosteroid dose was 939 mg, and 43% received ≥5 doses (Table 11). Tocilizumab was administered to 76% of patients.

TABLE 11
Cumulative dose and frequency of corticosteroid use.

| | Cohort 4 | |
|---|---|---|
| | (N = 30) | |
| Patients receiving corticosteroids, n (%)* | | |
| 1 dose | 7 | (23) |
| 2 doses | 7 | (23) |
| 3 doses | 3 | (10) |
| ≥5 doses | 13 | (43) |
| Cumulative corticosteroid dose, mg^{†} | | |
| Median (min-max) | 939 | (313-33,463) |
| Mean (SD) | 5152 | (7654) |

max, maximum; min, minimum.
*Corticosteroid use includes those doses that started on or after the start date of the first dose of axicabtagene ciloleucel but before or on the hospital discharge date.
^{†}Cumlative systemic cortisone-equivalent dose between infusion and hospital discharge date.

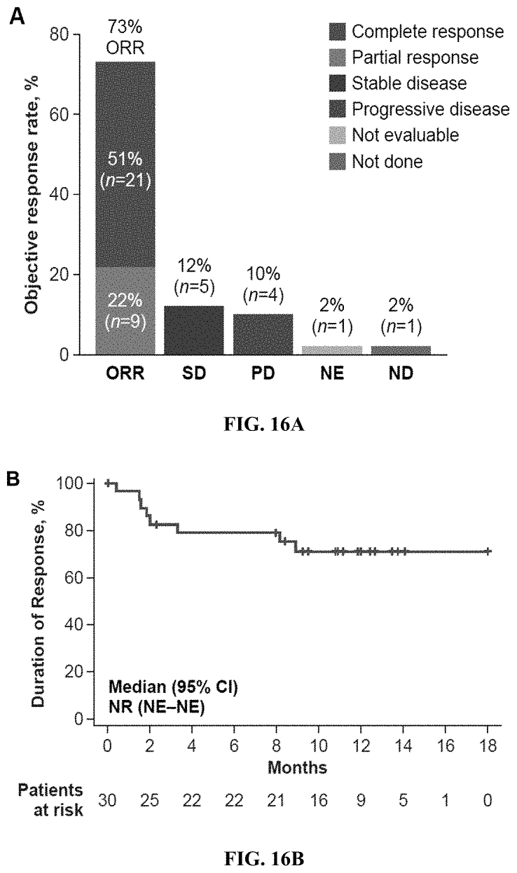

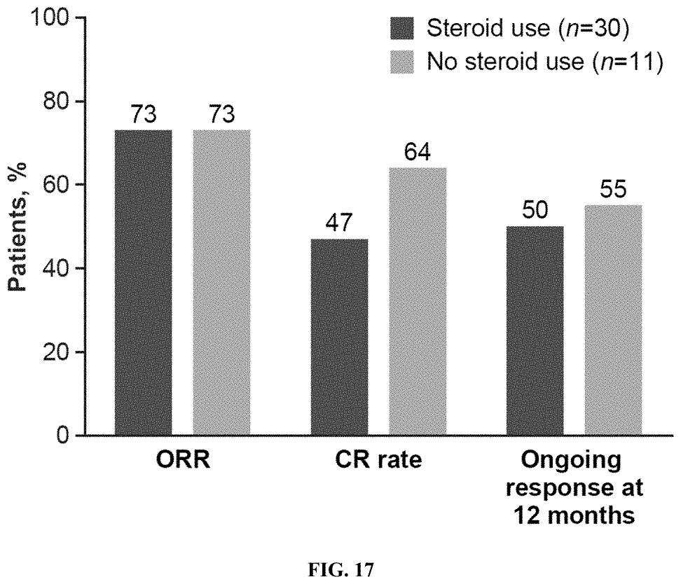

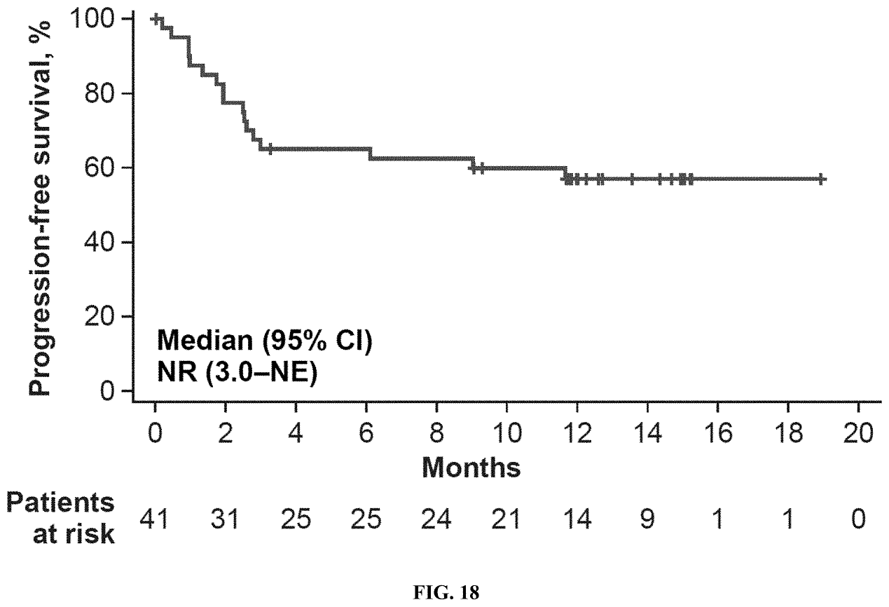

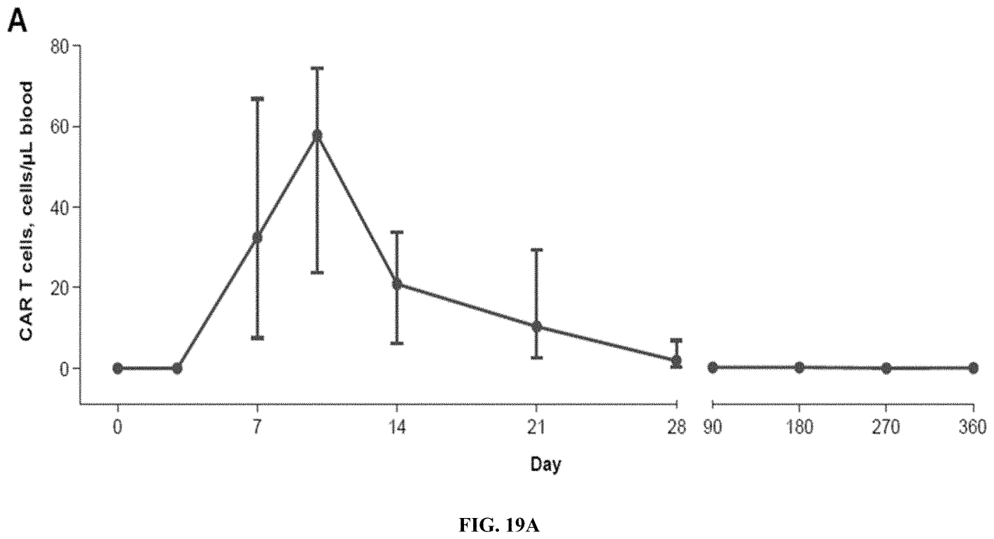

The investigator-assessed objective response rate (ORR) in cohort 4 was 73%, with a CR rate of 51% (FIG. 16). While the study was not designed to evaluate the effect of bridging therapy, comparable ORRs were observed in cohort 4 patients who did and did not receive bridging therapy (71% vs 77%, respectively), although the CR rate was numerically lower in patients who received bridging therapy (46% vs 62%). The KM estimate of the 12-month duration of response rate was 71%, and 51% of treated patients remained in response as of the data cutoff date. Response did not appear to be affected by corticosteroid use (FIG. 17). Neither median PFS (FIG. 18) nor median OS was reached with a minimum of 1 year of follow-up in cohort 4 (PFS: 95% CI, 3.0 months—not estimable; OS: 95% CI, 15.8 months—not estimable). KM estimates of 12-month PFS and OS rates were 57% and 68%, respectively.

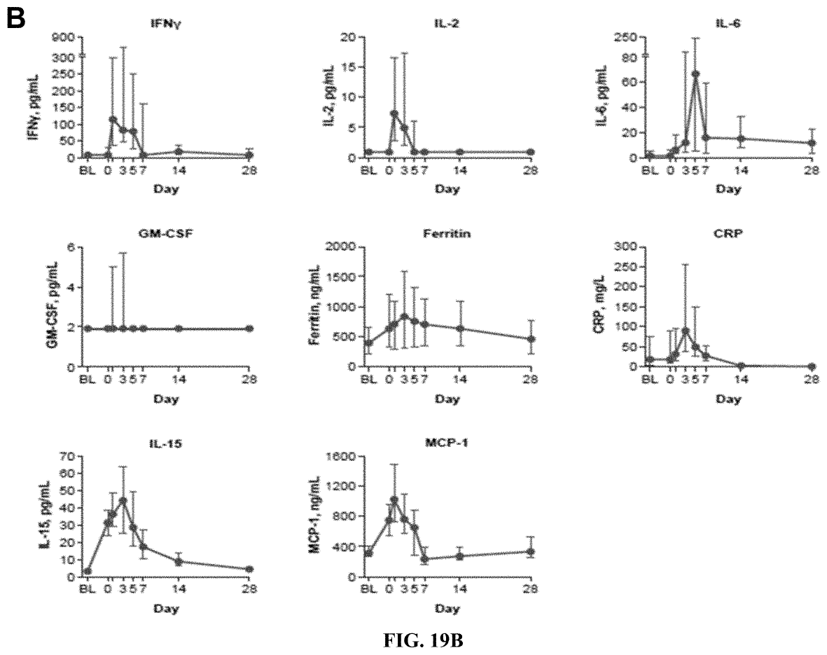

Median peak CAR T-cell expansion for cohort 4 was 52.9 cells/μL blood and was observed within 14 days after axicabtagene ciloleucel infusion (FIG. 19A). Post-treatment median levels of key inflammatory serum biomarkers associated with CRS and/or NEs—including IFN-γ, IL-2, IL-6, IL-15, GM-CSF, and ferritin—peaked during the first week after axicabtagene ciloleucel infusion (FIG. 19B; Table 12).

TABLE 12
Summary of serum biomarkers

| | Cohort 4 | | | |
|---|---|---|---|---|
| | (N = 41)* | | | |
| | Peak | | AUC₀₋₂₈ | |
| Biomarker | Median (min-max), pg/ml^{†} | | Median (min-max), pg/ml × day^{†} | |
| CRP | 126.5 (18.2-496.0) mg/1 | | 852.8 (209.5-5698.2) mg/1 × day | |
| Eotaxin-1 | 206.7 | (93.4-638.1) | 4822.2 | (1047.9-15,619.8) |
| Eotaxin-3 | 10.2 | (10.2-318.7) | 336.6 | (81.6-3884.4) |
| Ferritin | 1086.4 (95.5-23,869.6) ng/ml | | 22.7 (1.3-336.5) × 10³ ng/ml × day | |
| GM-CSF | 4.4 | (1.9-47.0) | 62.7 | (39.9-177.2) |
| Granzyme A | 20.0 | (20.0-3396.4) | 660.0 | (160.0-46,773.3) |
| ICAM-1 | 938.7 (359.5-5141.6) ng/ml | | 20,147.4 (10,002.8-64,670.3) ng/ml × day | |
| IFN-γ | 334.5 | (24.9-1876.0) | 1758.7 | (429.6-16,408.0) |
| IL-1 RA | 1093.7 (193.3-4493.1) (n = 31) | | 16397.4 (3278.4-41,090.6) (n = 27) | |
| IL-1 alpha | 2.9 | (2.9-2.9) | 95.7 | (23.2-95.7) |
| IL-1 beta | 2.1 | (2.1-6.4) | 69.3 | (16.8-69.3) |
| IL-10 | 19.6 | (1.4-466.0) | 142.5 | (25.2-6032.4) |
| IL-12 P40 | 160.5 | (5.7-756.1) | 3425.6 | (218.3-13,023.2) |
| IL-12 P70 | 1.2 | (1.2-6.4) | 39.6 | (9.6-48.7) |
| IL-13 | 4.2 | (4.2-8.5) | 138.6 | (33.6-138.6) |
| IL-15 | 45.8 | (22.3-272.7) | 463.3 | (223.6-2783.9) |
| IL-16 | 216.8 | (98.9-3740.0) | 5309.4 | (2003.9-61,679.4) |
| IL-17 | 9.3 | (9.3-314.1) | 306.9 | (126.5-1193.1) |
| IL-2 | 11.2 | (0.9-79.4) | 56.9 | (29.7-244.3) |
| IL-2 R alpha | 10.8 (2.8-94.6) × 10³ | | 184.5 (70.8-1063.9) × 10³ | |
| IL-4 | 0.5 | (0.5-4.1) | 16.5 | (4.0-40.3) |
| IL-5 | 34.4 | (6.3-853.7) | 274.4 | (178.9-8978.1) |
| IL-6 | 136.7 | (1.6-976.0) | 952.8 | (56.6-9322.4) |
| IL-7 | 33.1 | (18.0-67.5) | 689.8 | (353.6-1307.8) |
| IL-8 | 67.4 | (8.5-750.0) | 687.5 | (214.2-9972.8) |
| CXCL10 | 1571.7 | (469.2-2000.0) | 21961.7 | (4013.2-51,730.6) |
| MCP-1 | 1221.8 | (510.2-1500.0) | 14412.0 | (8259.1-37,739.2) |
| MCP-4 | 129.7 | (47.3-741.6) | 2709.1 | (558.6-14,063.6) |
| MDC | 852.3 | (88.3-18,936.9) | 19171.7 | (1833.7-33,8618.7) |
| MIP-1 alpha | 13.8 | (13.8-434.3) | 455.4 | (262.2-2146.6) |
| MIP-1 beta | 235.4 | (67.3-1689.2) | 3827.8 | (1600.2-7533.5) |
| PDL1 | 163.2 (45.1-1136.6) (n = 27) | | 4248.6 (422.3-8979.7) (n = 22) | |
| Perforin | 17.2 (3.9-44.4) × 10³ | | 348.5 (66.5-744.5) × 10³ | |
| SAA | 408.8 (4.1-1380.0) × 10⁶ | | 1459.4 | (363.5-13,278.9) |
| SFASL | 10.0 | (10.0-543.2) | 330.0 (190.0-1547.4) × 10⁶ | |
| CCL17 (TARC) | 871.8 | (82.7-4480.0) | 18808.2 | (834.6-12,7561.0) |
| TNF alpha | 5.7 | (2.0-54.6) | 92.6 | (35.1-286.1) |
| TNF beta | 1.2 | (1.2-19.5) | 39.6 | (9.6-76.2) |

| | | |
|---|---|---|
| VCAM-1 | 12.6 (5.9-39.3) × 10⁵ | 27.5 (7.1-62.) × 10⁶ |

AUC₀₋₂₈, area under the curve from day 0 to 28; CCL, chemokine (C-C motif) ligand; CRP, C-reactive protein; CXCL, chemokine (C-X-C motif) ligand; GM-CSF, granulocyte-macrophage colony-stimulating factor; ICAM, intercellular adhesion molecule; IFN, interferon; IL, interleukin; max, maximum; MCP, monocyte chemotactic protein; MDC, macrophage-derived chemokine; min, minimum; MIP, macrophage inflammatory protein; N/A, not applicable; PD-L1, programmed death-ligand 1; R, receptor; RA, receptor antagonist; SAA, serum amyloid A; SFASL, serum soluble Fas ligand; TARC, thymus- and activation-regulated chemokine; TNF, tumour necrosis factor; VCAM, vascular cell adhesion molecule.
*N is specified in cell if it differs from that of the overall group.
^{†}Specified units unless otherwise noted.

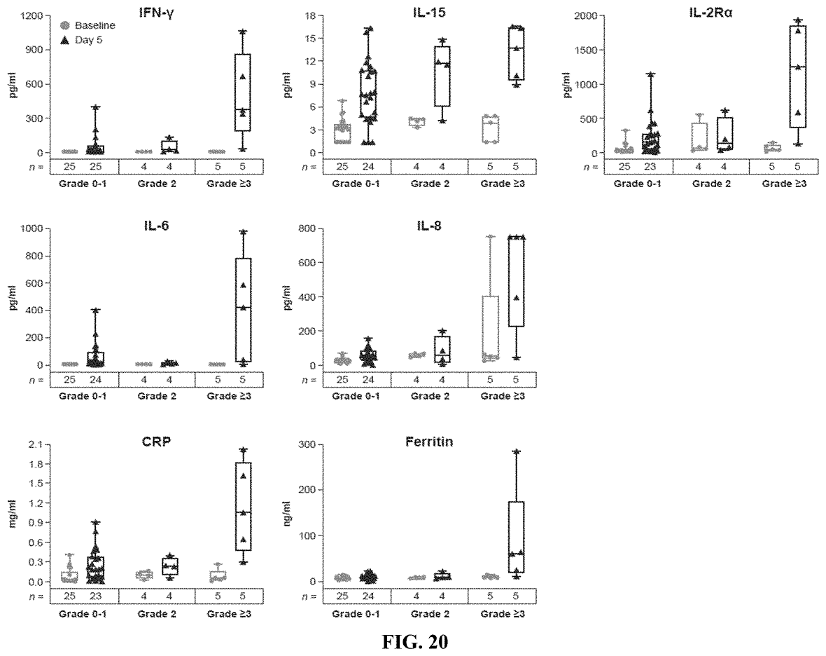

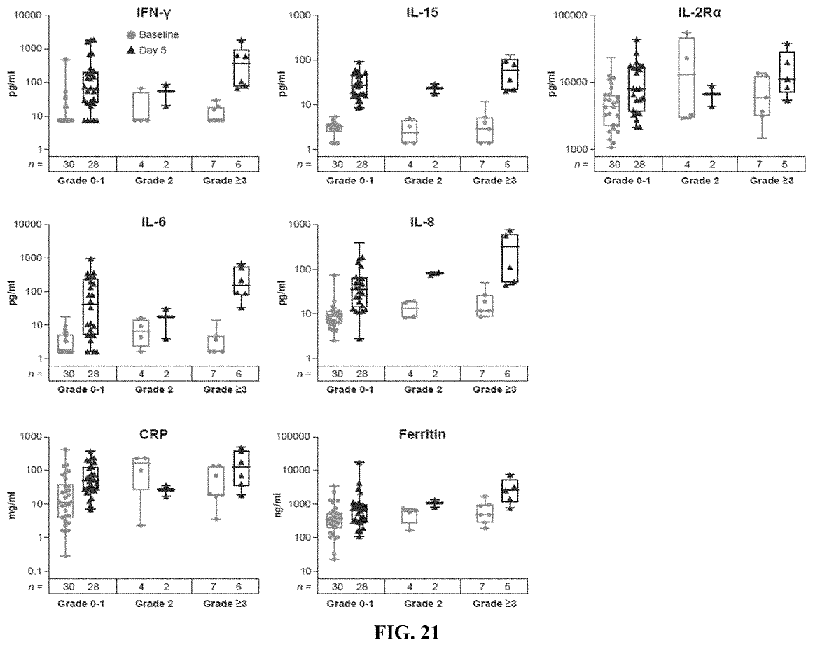

Cohort 4 patients with evaluable samples and grade ≥3 NEs had numerically greater post-infusion (day 5) cerebrospinal fluid levels of IFN-γ, IL-15, IL-2Rα, IL-6, and IL-8 than did those with grade 0 to 1 NEs, despite low and comparable baseline levels across cohort 4 (FIG. 20). A similar pattern was observed for serum biomarkers (FIG. 21).

The incidence of grade ≥3 CRS and grade ≥3 NEs observed in cohort 4 (2% and 17%, respectively) was numerically lower than in cohorts 1+2 (12% and 29%, respectively).³ Because cohort 4 was not designed for statistical comparison with cohorts 1+2, an exploratory PSM analysis was used to matched these cohorts with respect to key baseline characteristics. After PSM, baseline disease and product characteristics were generally similar between patients in cohort 4 and cohorts 1+2, although fewer cohort 4 patients had baseline ECOG performance status of 1 (49% vs 68%; Table 13).

TABLE 13
Comparison of baseline and product characteristics between patients in
cohorts 1 + 2 and cohort 4 before and after propensity score matching.

| | Cohorts 1 + 2 | | Cohorts 1 + 2 | | | |
|---|---|---|---|---|---|---|
| | Overall | | After matching | | Cohort 4 | |
| Characteristic | (N = 101) | | (N = 41) | | (N = 41) | |
| Median tumour burden by | 3723.0 | (2200.0-7138.0) | 2035.0 | (792.0-3719.0) | 2100.0 | (810.0-5526.0) |
| SPD* (Q1-Q3), mm² | | | | | | |
| Median age (Q1-Q3), | 58.0 | (51.0-64.0) | 60.0 | (54.0-68.0) | 61.0 | (52.0-65.0) |
| years | | | | | | |
| Disease stage III or IV, | 86 | (85.1) | 28 | (68.3) | 29 | (70.7) |
| n (%) | | | | | | |
| ECOG performance status | 59 | (58.4) | 28 | (68.3) | 20 | (48.8) |
| of 1, n (%) | | | | | | |
| IPI score 3-4, n (%) | 46 | (45.5) | 16 | (39.0) | 20 | (48.8) |
| Number of prior lines of | | | | | | |
| chemotherapy, % | | | | | | |
| ≤2 | 31 | (30.7) | 15 | (36.6) | 15 | (36.6) |
| 3 | 29 | (28.7) | 19 | (46.3) | 15 | (36.6) |
| ≥4 | 41 | (40.6) | 7 | (17.1) | 11 | (26.8) |
| Prior platinum use, n (%) | 90 | (89.1) | 39 | (95.1) | 39 | (95.1) |
| Median LDH (Q1-Q3), U/l | 356.0 | (219.0-743.0) | 241.0 | (190.0-425.0) | 262.0 | (197.0-401.0) |
| Product characteristics,^{†} median (Q1-Q3) | | | | | | |
| CD8+ T cells, % | 53.6 | (35.0-65.0) | 47.8 | (38.3-65.2) | 40.8 | (31.0-51.4) |
| Naive T cells, % | 13.8 | (7.7-24.3) | 15.8 | (8.1-25.7) | 13.4 | (8.3-22.6) |
| Percent transduction, % | 52.6 | (44.3-63.6) | 52.4 | (37.2-62.4) | 55.0 | (48.0-64.0) |

CAR, chimeric antigen receptor; ECOG, Eastern Cooperative Oncology Group; IPI, International Prognostic Index; LDH, lactate dehydrogenase; Q, quartile; SPD, sum of the products of diameters.
*Measured before conditioning therapy. For cohort 4, who received bridging therapy, baseline tumour burden was measured after bridging but before conditioning therapy.
^{†}Product characteristic parameters were not used for propensity score matching and are presented descriptively here in before matching and after matching subgroups.

Notably, the differences in grade ≥3 CRS and NEs observed between patients in cohorts 1+2 and cohort 4 before PSM were maintained after matching. Although CR rates after PSM were numerically lower in cohort 4 versus cohorts 1+2, ongoing response rates remained comparable. Clinical outcomes were corroborated by lower levels of key inflammatory soluble biomarkers associated with CAR-related inflammatory events (e.g., IFN-γ, IL-2, IL-8, C-reactive protein, ferritin, GM-CSF),^{3, 10} and by generally comparable peak CAR T-cell levels in cohort 4 versus cohorts 1+2 both before and after PSM. The median cumulative cortisone-equivalent corticosteroid dose required to manage CRS or NEs remained lower in cohort 4 (939 mg) than in matched cohorts 1+2 (6886 mg; Table 14).

TABLE 14
Comparison of efficacy and safety outcomes and CAR T-cell and soluble serum biomarker levels
between patients in cohorts 1 + 2 and cohort 4 before and after propensity score matching.

| | Cohorts 1 + 2 | | Cohorts 1 + 2 | | | |
|---|---|---|---|---|---|---|
| | overall | | after matching | | Cohort 4 | |
| Characteristic | (N = 101) | | (N = 41) | | (N = 41) | |
| Efficacy | | | | | | |
| Response | | | | | | |
| Objective | 84 | (83.2) | 38 | (92.7) | 30 | (73.2) |
| response, n (%) | | | | | | |
| Complete | 59 | (58.4) | 31 | (75.6) | 21 | (51.2) |
| response, n (%) | | | | | | |
| Ongoing response | 42 | (41.6) | 21 | (51.2) | 21 | (51.2) |
| at data cutoff, n | | | | | | |
| (%) | | | | | | |
| Safety | | | | | | |
| CRS | | | | | | |
| Worst grade 2 | 45 | (44.6) | 16 | (39.0) | 24 | (58.5) |
| Worst grade ≥3, n | 12 | (11.9) | 6 | (14.6) | 1 | (2-4) |
| (%) | | | | | | |
| Median (Q1-Q3) | 2 | (2-3) | 2 | (2-3) | 2 | (1-4) |
| time to onset of | | | | | | |
| any grade CRS, | | | | | | |
| days | | | | | | |
| NEs | | | | | | |
| Worst grade 2 | 14 | (13.9) | 5 | (12.2) | 4 | (9.8) |
| Worst grade ≥3, n | 29 | (28.7) | 11 | (26.8) | 7 | (17.1) |
| (%) | | | | | | |
| Median (Q1-Q3) | 5 | (3-7) | 6 | (3-7) | 6 | (2-9) |
| time to onset of | | | | | | |
| any grade NE, | | | | | | |
| days | | | | | | |
| Corticosteroid use* | | | | | | |
| Patients receiving | 26 | (25.7) | 8 | (19.5) | 30 | (73.2) |
| corticosteroids, n | | | | | | |
| (%) | | | | | | |
| Median (Q1-Q3) | 6387 | (3051-15,862) | 6886 | (1565-15,963) | 939 | (626-8138) |
| cumulative | | | | | | |
| corticosteroid | | | | | | |
| dose, mg | | | | | | |
| Tocilizumab use | | | | | | |
| Patients receiving | 43 | (42.6) | 12 | (29.3) | 31 | (75.6) |
| tocilizumab, n | | | | | | |
| (%) | | | | | | |
| Pharmacokinetics | | | | | | |
| and | | | | | | |
| pharmacodynamics | | | | | | |
| Peak CAR T-cell | | | | | | |
| levels, median | | | | | | |
| (Q1-Q3) | | | | | | |
| CAR T-cell | 38.3 | (14.7-83.0) | 33.8 | (17.1-106.9) | 52.9 | (27.3-92.8) |
| expansion, | | | | | | |
| cells/μl | | | | | | |
| AUC₀₋₂₈, cells/μl × day | 453.5 | (148.7-920.3) | 450.0 | (231.9-975.6) | 511.2 | (216.0-973.5) |
| Peak cytokine | | | | | | |
| levels, median | | | | | | |
| (Q1-Q3) | | | | | | |
| IFN-γ, pg/ml | 477.4 | (196.3-1096.7) | 452.0 | (137.3-1094.3) | 334.5 | (136.1-737.3) |
| IL-15, pg/ml | 52.9 | (34.7-72.1) | 56.5 | (36.1-74.4) | 45.8 | (31.2-59.5) |
| IL-2, pg/ml | 21.7 | (10.2-37.8) | 29.7 | (10.2-45.9) | 11.2 | (5.2-20.9) |
| IL-6, pg/ml | 83.3 | (23.3-347.5) | 63.90 | (15.9-261.0) | 136.70 | (14.9-366.3) |
| IL-8, pg/ml | 93.6 | (46.6-329.3) | 124.9 | (37.0-329.9) | 67.4 | (31.6-175.2) |
| MCP-1 (CCL2), | 1500.0 | (900.1-1500.0) | 1500.0 | (879.5-1500.0) | 1221.8 | (748.9-1500.0) |
| pg/ml | | | | | | |
| CRP, mg/1 | 214.2 | (141.4-353.4) | 185.2 | (141.4-382.1) | 126.5 | (60.9-275.6) |
| Ferritin, ng/ml | 3001.4 | (1325.6-6683.5) | 2461.1 | (1154.9-5819.1) | 1086.4 | (481.0-1586.6) |
| GM-CSF, pg/ml | 7.3 | (1.9-16.1) | 9.5 | (1.9-22.5) | 4.4 | (1.9-6.9) |

AUC₀₋₂₈, area under the curve from day 0 to day 28; CAR, chimeric antigen receptor; CRP, C-reactive protein; CRS, cytokine release syndrome; GM-CSF, granulocyte-macrophage colony-stimulating factor; IFN, interferon; IL, interleukin; MCP-1, monocyte chemoattractant protein-1; NE, neurologic event; Q, quartile.
*Corticosteroid use includes those doses that started on or after the start date of axicabtagene ciloleucel but before the hospital discharge date.

AE management in CAR T-cell therapy is an evolving field with ongoing efforts to improve the safety profile of this treatment modality without compromising durable clinical benefit. To this end, CLINICAL TRIAL-1 cohort 4 patients received corticosteroid and/or tocilizumab intervention earlier than did the pivotal cohorts 1+2 (Neelapu et al., N Engl J Med. 2017; 377(26):2531-44; Locke F L, Ghobadi A, Jacobson C A, Miklos D B, Lekakis L J, Oluwole O O, et al., Lancet Oncol. 2019; 20(1):31-42). Numerically lower rates of grade ≥3 CRS and NEs were observed in cohort 4 (2% and 17%, respectively) than in cohorts 1+2 (12% and 29%), suggesting that earlier intervention with corticosteroids and/or tocilizumab may have the potential to change the safety profile of axicabtagene ciloleucel in patients with R/R LBCL. In patients treated with corticosteroids, the median cumulative cortisone-equivalent dose was 939 mg in cohort 4 versus 6388 mg reported in cohorts 1+2, suggesting that earlier corticosteroid use does not increase cumulative corticosteroid dose. Furthermore, this revised safety management regimen did not appear to negatively affect the ongoing response rate at 1 year (cohort 4: 51%; cohorts 1+2: 42%).

Differences in baseline characteristics and cohort sizes should be considered when comparing cohort 4 with pivotal cohorts 1+2. Cohort 4 patients had lower levels of inflammatory serum biomarkers (e.g., ferritin or LDH) at baseline, and a lower proportion of patients had progressive disease in response to the most recent line of therapy (Locke et al., Lancet Oncol. 2019; 20(1):31-42; Topp et al., Blood. 2019; 134(Suppl 1):243-) Cohort 4 also had lower tumour burden, which was previously associated with lower rates of NEs, and increased efficacy (Locke et al., Blood Adv. 2020; 4(19):4898-911; Dean et al., Blood Adv. 2020; 4(14):3268-76). To overcome these limitations and reduce bias in the absence of a randomized trial, PSM (Rosenbaum and Rubin, Biometriks. 1983; 70(1):41-55; Austin, Multivariate Behav Res. 2011; 46(3):399-424) was applied to cohorts 1+2 and cohort 4. This statistical method adjusts for potential imbalances in baseline disease characteristics between cohorts, thereby providing a more balanced and robust comparison (Austin, Stat Med. 2008; 27(12):2037-49; Zhang et al., Ann Transl Med. 2019; 7(1):16). Although minor differences in pretreatment characteristics remained after matching, the aforementioned differences in toxicity outcomes observed between patients in cohort 4 and cohorts 1+2 before PSM were maintained after matching, supporting the benefit of earlier corticosteroid and/or tocilizumab. PSM also had little effect on peak CAR T-cell levels, and ongoing response rates at 1 year remained comparable.

The results presented here are consistent with the primary analysis of CLINICAL TRIAL-1 (cohorts 1+2), which suggested no substantial effect of corticosteroid use on ORR (corticosteroid, 78% [58-91%]; no corticosteroid, 84% [73-91%]). Retrospective analyses of real-world data have delivered varying results regarding the impact of corticosteroid use on clinical outcomes after axicabtagene ciloleucel in R/R LBCL (Strati et al., Blood. 2021, Nastoupil et al., J Clin Oncol. 2020:[online ahead of print]). However, in the larger of these 2 studies (N=298), multivariate analysis demonstrated no significant difference in PFS, CR rates, or OS in patients treated with versus without corticosteroids (Nastoupil et al., J Clin Oncol. 2020:[online ahead of print]). It is important to note that the clinical applicability of these studies is unclear given their retrospective nature and potential imbalances in baseline characteristics (e.g., tumour burden) (Locke et al., Blood Adv. 2020; 4(19):4898-911; Dean et al., Blood Adv. 2020; 4(14):3268-76; Gauthier et al., J Clin Oncol. 2018; 36(15 suppl):7567-; Jacobson et al., Blood. 2018; 132:abstract 92) in patients requiring corticosteroids versus not requiring corticosteroids. Although studies of other CAR T-cell products in B-cell acute lymphoblastic leukemia have not been designed to assess the impact of corticosteroid use, published analyses have shown no substantial effect of corticosteroid use on CAR T-cell expansion or tumour response (Gardner et al., Blood. 2019; 134(24):2149-58; Liu et al., Blood Cancer J. 2020; 10(2):15).

### Example 4

An open-label, global, multicenter, Phase 3 study was conducted to evaluate the safety and efficacy of axicabtagene ciloleucel versus current standard of care for second-line therapy (platinum-based salvage combination chemotherapy regimen followed by high-dose therapy and autologous stem cell transplant in those who respond to salvage chemotherapy) in adult patients with relapsed or refractory Diffuse Large B-Cell Lymphoma (DLBCL). In this study, 359 patients were randomized (1:1) to receive a single infusion of axicabtagene ciloleucel or the current standard of care second-line therapy. The primary endpoint was event-free survival (EFS), defined as the time from randomization to the earliest date of disease progression per Lugano Classification (see Cheson et al, J Clin Oncol. 2014 Sep. 20; 32(27):3059-68.), commencement of new lymphoma therapy, or death from any cause. Key secondary endpoints include objective response rate (ORR) and overall survival (OS). Other secondary endpoints include modified event-free survival, progression-free survival (PFS) and duration of response (DOR). Patients enrolled in the study ranged in age from 22 to 81, with 30% of the patients over the age of 65. The study described in this example evaluated a one-time infusion of the cell therapy axicabtagene ciloleucel compared to second-line standard of care (SOC) in adult patients with relapsed or refractory LBCL. The study SOC arm was a 2-step process: following initial relapse, immunochemotherapy was reintroduced and if the patient responded and can tolerate further treatment, then they move on to high-dose chemotherapy plus stem cell transplant.

### Key Inclusion Criteria:

1. Histologically proven large B-cell lymphoma including the following types defined by WHO 2016 (see Swerdlow et al Blood. 2016 May 19; 127(20):2375-90. doi: 10.1182/blood-2016-01-643569. Epub 2016 Mar. 15. Review.)
   DLBCL not otherwise specified (ABC/GCB)
   HGBL with or without MYC and BCL2 and/or BCL6 rearrangement
   DLBCL arising from FL
   T-cell/histiocyte rich large B-cell lymphoma
   DLBCL associated with chronic inflammation
   Primary cutaneous DLBCL, leg type
   Epstein-Barr virus (EBV)+DLBCL
2. Relapsed or refractory disease after first-line chemoimmunotherapy
   Refractory disease defined as no complete remission to first-line therapy; individuals who are intolerant to first-line therapy are excluded.
   Progressive disease (PD) as best response to first-line therapy
   Stable disease (SD) as best response after at least 4 cycles of first-line therapy (eg, 4 cycles of R-CHOP)
   Partial response (PR) as best response after at least 6 cycles and biopsy-proven residual disease or disease progression ≤12 months of therapy
   Relapsed disease defined as complete remission to first-line therapy followed by biopsy-proven relapse ≤12 months of first-line therapy
3. Individuals must have received adequate first-line therapy including at a minimum:
   Anti-CD20 monoclonal antibody unless investigator determines that tumor is CD20 negative, and
   An anthracycline containing chemotherapy regimen
4. No known history or suspicion of central nervous system involvement by lymphoma
5. Eastern cooperative oncology group (ECOG) performance status of 0 or 1
6. Adequate bone marrow function as evidenced by:
   Absolute neutrophil count (ANC) ≥1000/uL
   Platelet ≥75,000/uL
   Absolute lymphocyte count ≥100/uL
7. Adequate renal, hepatic, cardiac, and pulmonary function as evidenced by:
   Creatinine clearance (Cockcroft Gault) ≥60 mL/min
   Serum Alanine aminotransferase/Aspartate aminotransferase (ALT/AST)≤2.5 Upper limit of normal (ULN)
   Total bilirubin ≤1.5 mg/dl
   Cardiac ejection fraction ≥50%, no evidence of pericardial effusion as determined by an Echocardiogram (ECHO), and no clinically significant Electrocardiogram (ECG) findings
   No clinically significant pleural effusion
   Baseline oxygen saturation ≥92% on room air
      Key Exclusion Criteria were:
1. History of malignancy other than nonmelanoma skin cancer or carcinoma in situ (eg cervix, bladder, breast) unless disease free for at least 3 years
2. Received more than one line of therapy for DLBCL
3. History of autologous or allogeneic stem cell transplant
4. Presence of fungal, bacterial, viral, or other infection that is uncontrolled or requiring intravenous antimicrobials for management.
5. Known history of infection with human immunodeficiency virus (HIV) or hepatitis B (HBsAg positive) or hepatitis C virus (anti-HCV positive). If there is a positive history of treated hepatitis B or hepatitis C, the viral load must be undetectable per quantitative polymerase chain reaction (PCR) and/or nucleic acid testing.
6. Individuals with detectable cerebrospinal fluid malignant cells or known brain metastases, or with a history of cerebrospinal fluid malignant cells or brain metastases.
7. History or presence of non-malignant central nervous system (CNS) disorder such as seizure disorder, cerebrovascular ischemia/hemorrhage, dementia, cerebellar disease, or any autoimmune disease with CNS involvement
8. Presence of any indwelling line or drain. Dedicated central venous access catheter such as a Port-a-Cath or Hickman catheter are permitted.
9. History of myocardial infarction, cardiac angioplasty or stenting, unstable angina, New York Heart Association Class II or greater congestive heart failure, or other clinically significant cardiac diseases within 12 months of enrollment
10. History of symptomatic deep vein thrombosis or pulmonary embolism within 6 months of enrollment
11. History of autoimmune disease, requiring systemic immunosuppression and/or systemic disease modifying agents within the last 2 years
12. History of anti-CD19 or CAR-T therapy or history of prior randomization

A primary analysis of the study showed superiority of axicabtagene ciloleucel compared to standard of care (SOC) in second-line relapsed or refractory large B-cell lymphoma (LBCL). The study met the primary endpoint of event free survival (EFS; hazard ratio 0.398, p<0.0001), and the key secondary endpoint of objective response rate (ORR). The interim analysis of overall survival (OS) showed a trend favoring axicabtagene ciloleucel but the data is immature and additional analysis and/or studies may be warranted.

Safety results from the study were consistent with the known safety profile of axicabtagene ciloleucel for the treatment of LBCL in the third-line setting. Six percent of patients experienced CRS grade 3 or higher, and 21% experienced neurological events grade 3 or higher. No new safety concerns were identified in this second-line setting.

### Example 5

This example relates to and expands upon Example 4. An open-label, global, multicenter, Phase 3 study was conducted to evaluate the safety and efficacy of axicabtagene ciloleucel versus current standard of care (SOC) for second-line therapy (platinum-based salvage combination chemotherapy regimen followed by high-dose therapy and autologous stem cell transplant in those who respond to salvage chemotherapy) in adult patients with relapsed or refractory Diffuse Large B-Cell Lymphoma (DLBCL). Common regimens included rituximab+gemcitabine, dexamethasone and cisplatin/carboplatin (R-GDP), rituximab+dexamethasone, high-dose cytarabine and cisplatin (R-DHAP), rituximab+ifosfamide, carboplatin, and etoposide (R-ICE), and rituximab+etoposide, methylprednisolone, cytarabine, cisplatin (R-ESHAP). As no single salvage regimen has demonstrated superiority, (Crump, et al. J Clin Oncol. 2014; 32:3490-6; Gisselbrecht, et al. J Clin Oncol. 2012; 30:4462-9) institutional preference and toxicity profile was considered when selected SOC regimen for patients. Suggested dosing of common regimens for SOC is shown in table 15.

TABLE 15
SOC chemotherapy

| SOC | |
|---|---|
| chemotherapy | Dosing |
| R-GDP | Rituximab 375 mg/m² day 1 (or day 8) |
| | Gemcitabine 1 g/m² on days 1 and 8 |
| | Dexamethasone 40 mg on days 1-4 |
| | Cisplatin 75 mg/m² on day 1 (or |
| | carboplatin AUC = 5) |
| R-DHAP | Rituximab 375 mg/m² before chemotherapy |
| | Dexamethasone 40 mg/day on days 1-4 |
| | High-dose cytarabine 2 g/m² every |
| | 12 hours for 2 doses on day 2 following |
| | platinum |
| | Cisplatin 100 mg/m² 24 h-CI on day 1 |
| | (or oxaliplatin 100 mg/m²) (Lignon, et al. |
| | Clin Lymphoma Myeloma Leuk. 2010; |
| | 10: 262-9.) |
| R-ICE | Rituximab 375 mg/m² before chemotherapy |
| | Ifosfamide 5 g/m² 24 h-CI on day 2 |
| | with mesna |
| | Carboplatin AUC = 5 on day 2, maximum |
| | dose 800 mg |
| | Etoposide 100 mg/m²/d on days 1-3 |
| R-ESHAP | Rituximab 375 mg/m² day 1 |
| | Etoposide 40 mg/m²/d IV on days 1-4 |
| | Methylprednisolone 500 mg/d IV on days |
| | 1-4 or 5 |
| | Cisplatin at 25 mg/m²/d CI days 1-4 |
| | Cytarabine 2 g/m² on day 5 |

24 h-CI, 24 hour continuous infusion; AUC, area under the curve; CI, continuous infusion; IV, intravenous; R-GDP, rituximab + gemcitabine, dexamethasone and cisplatin/carboplatin; R-DHAP, rituximab + dexamethasone, high-dose cytarabine and cisplatin; R-ICE, rituximab + ifosfamide, carboplatin, and etoposide; R-ESHAP, and rituximab + etoposide, methylprednisolone, cytarabine, cisplatin.

This study was conducted at 77 sites worldwide. Eligible patients were aged ≥18 years with histologically confirmed LBCL per World Health Organization 2016 classification criteria (Swerdlow, et al. Blood. 2016; 127:2375-90.) that was R/R ≤12 months of first-line chemoimmunotherapy, including an anti-CD20 monoclonal antibody and anthracycline-containing regimen, and intended to proceed to HDT-ASCT. Refractory disease was defined as no CR to first-line therapy; relapsed disease was defined as CR followed by biopsy-proven disease relapse ≤12 months of first-line therapy. Enrollment was open to any patient deemed eligible by the investigator for inclusion in the study.

### Additional Inclusion Criteria:

Histologically proven large B-cell lymphoma including the following types defined by World Health Organization 2016 (Swerdlow, et al. Blood. 2016; 127:2375-90.)
   Diffuse large B-cell lymphoma (DLBCL) not otherwise specified (including activated B-cell like [ABC]/germinal center B-cell like [GCB])
   High grade B-cell lymphoma with or without MYC Proto-Oncogene, BHLH Transcription Factor (MYC) and BCL2 apoptosis regulator and/or BCL6 transcription repressor rearrangement
   DLBCL arising from follicular lymphoma
   T-cell/histiocyte rich large B-cell lymphoma
   DLBCL associated with chronic inflammation
   Primary cutaneous DLBCL, leg type
   Epstein-Barr virus+DLBCL
Relapsed or refractory disease after first-line chemoimmunotherapy
   Refractory disease defined as no complete remission to first-line therapy; patients who are intolerant to first-line therapy are excluded
      Progressive disease (PD) as best response to first-line therapy
      Stable disease (SD) as best response after at least 4 cycles of first-line therapy (eg, 4 cycles of cyclophosphamide/doxorubicin/prednisone/rituximab/vincristine)
      Partial response (PR) as best response after at least 6 cycles and biopsy-proven residual disease or disease progression ≤12 months of therapy
   Relapsed disease defined as complete remission to first-line therapy followed by biopsy-proven disease relapse ≤12 months of first-line therapy
Patients must have had received adequate first-line therapy including at a minimum:
   Anti-CD20 monoclonal antibody unless investigator determines that tumor is CD20 negative, and
   An anthracycline containing chemotherapy regimen
Intended to proceed to high-dose therapy with autologous stem cell rescue (HDT-ASCT) if response to second-line therapy
Patients must have had radiographically documented disease
No known history or suspicion of central nervous system (CNS) involvement by lymphoma
At least 2 weeks or 5 half-lives, whichever is shorter, must have had elapsed since any prior systemic cancer therapy at the time the patient provides consent
Age 18 years or older at the time of informed consent
Eastern Cooperative Oncology Group (ECOG) performance status of 0 or 1
Adequate bone marrow, renal, hepatic, pulmonary and cardiac function defined as:
   Absolute neutrophil count ≥1000/μL
   Platelet count ≥75,000/μL
   Absolute lymphocyte count ≥100/μL
   Creatinine clearance (as estimated by Cockcroft Gault) ≥60 mL/min
   Serum alanine aminotransferase/aspartate aminotransferase ≤2.5 upper limit of normal
   Total bilirubin ≤1.5 mg/dl, except in patients with Gilbert's syndrome
   Cardiac ejection fraction ≥50%, no evidence of pericardial effusion as determined by an echocardiogram, and no clinically significant electrocardiogram findings
   No clinically significant pleural effusion
   Baseline oxygen saturation >92% on room air
Females of childbearing potential must have had a negative serum or urine pregnancy test (females who have undergone surgical sterilization or who have been postmenopausal for at least 2 years are not considered to be of childbearing potential)

### Additional Exclusion Criteria:

History of malignancy other than nonmelanoma skin cancer or carcinoma in situ (eg, cervix, bladder, breast) unless disease free for at least 3 years
History of Richter's transformation of chronic lymphocytic leukemia or primary mediastinal large B-cell lymphoma
History of autologous or allogeneic stem cell transplant
Received more than one line of therapy for DLBCL
Prior CD19 targeted therapy
Treatment with systemic immunostimulatory agents (including, but not limited to, interferon and IL-2) within 6 weeks or 5 half-lives of the drug, whichever is shorter, prior to the first dose of axicabtagene ciloleucel (axicabtagene ciloleucel) or standard-of-care (SOC)
Prior chimeric antigen receptor (CAR) therapy or other genetically modified T-cell therapy or prior randomization
History of severe, immediate hypersensitivity reaction attributed to aminoglycosides
Presence of fungal, bacterial, viral, or other infection that is uncontrolled or requiring intravenous (IV) antimicrobials for management. Simple urinary tract infection and uncomplicated bacterial pharyngitis are permitted if responding to active treatment
Known history of infection with human immunodeficiency virus (HIV) or hepatitis B (HBsAg positive) or hepatitis C virus (anti-HCV positive). If there is a positive history of treated hepatitis B or hepatitis C, the viral load must be undetectable per quantitative polymerase chain reaction (PCR) and/or nucleic acid testing
Active tuberculosis
Presence of any indwelling line or drain (eg, percutaneous nephrostomy tube, indwelling Foley catheter, biliary drain, or pleural/peritoneal/pericardial catheter). Dedicated central venous access catheters, such as a Port-a-Cath or Hickman catheter, are permitted.
Patients with detectable cerebrospinal fluid malignant cells or known brain metastases or with a history of cerebrospinal fluid malignant cells or brain metastases
History or presence of non-malignant CNS disorder, such as seizure disorder, cerebrovascular ischemia/hemorrhage, dementia, cerebellar disease, or any autoimmune disease with CNS involvement
Patients with cardiac atrial or cardiac ventricular lymphoma involvement
History of myocardial infarction, cardiac angioplasty or stenting, unstable angina, New York Heart Association Class II or greater congestive heart failure, or other clinically significant cardiac disease within 12 months of enrollment
Requirement for urgent therapy due to tumor mass effects, such as bowel obstruction or blood vessel compression
History of autoimmune disease requiring systemic immunosuppression and/or systemic disease modifying agents within the last 2 years
History of idiopathic pulmonary fibrosis, organizing pneumonia (eg, bronchiolitis obliterans), drug-induced pneumonitis, idiopathic pneumonitis, or evidence of active pneumonitis per chest computed tomography (CT) scan at screening. History of radiation pneumonitis in the radiation field (fibrosis) is allowed.
History of symptomatic deep vein thrombosis or pulmonary embolism within 6 months of enrollment
Any medical condition likely to interfere with assessment of safety or efficacy of study treatment
History of severe immediate hypersensitivity reaction to tocilizumab or any of the agents used in this study
Treatment with a live, attenuated vaccine within 6 weeks prior to initiation of study treatment or anticipation of need for such a vaccine during the course of the study
Women of childbearing potential who were pregnant or breastfeeding because of the potentially dangerous effects of chemotherapy on the fetus or infant. Patients of either sex who were not willing to practice birth control from the time of consent and at least 6 months after the last dose of axicabtagene ciloleucel or SOC chemotherapy
In the investigator's judgment, the patient was unlikely to complete all protocol-required study visits or procedures, including follow-up visits, or comply with the study requirements for participation

Per the original protocol, the timeframe for relapsed disease of CR to first-line therapy followed by biopsy-proven disease relapse was ≤12 months of initiating first-line therapy. This was broadened to ≤12 months of first-line therapy. Per the original protocol, randomization was stratified by relapse ≤6 months of initiating first-line therapy and relapse >6 and ≤12 months of initiating first-line therapy. This was broadened to relapse ≤6 months of first-line therapy and relapse >6 and ≤12 months of first-line therapy. Randomization was stratified by response to first-line therapy (primary refractory, versus relapse ≤6 months of first-line therapy, versus relapse >6 and ≤12 months of first-line therapy) and second-line age-adjusted IPI (sAAIPI; 0-1 versus 2-3) as assessed at screening. Patients initiated either leukapheresis (for axicabtagene ciloleucel cohort) or SOC therapy (for SOC cohort) within approximately 5 days of randomization.

Following screening, patients were randomized 1:1 to axicabtagene ciloleucel or investigator-selected SOC chemotherapy, stratified by response to first-line therapy and second-line age-adjusted IPI (sAAIPI) at screening. Axicabtagene ciloleucel patients underwent leukapheresis followed by conditioning chemotherapy. On day 0, patients received a single axicabtagene ciloleucel infusion. Bridging therapy was limited to corticosteroids only per investigator's discretion. SOC patients received 2-3 cycles of a protocol-defined, investigator-selected platinum-based chemoimmunotherapy regimen supplied by the site. Patients who achieved a CR or a partial response (PR) proceeded to HDT-ASCT. Although there was no planned crossover between arms, patients unresponsive to SOC could receive cellular immunotherapy off protocol (treatment switching). Toxicity management followed that of Neelapu, et al. N Engl J Med. 2017; 377:2531-2544. Cytokine release syndrome (CRS) was graded per modified Lee criteria. (Lee, et al. Blood. 2014; 124:188-95.) Adverse events (AEs) and CRS and neurologic event symptoms were graded per National Cancer Institute Common Terminology Criteria for Adverse Events version 4.03.

The primary endpoint was event-free survival (EFS; time from randomization to the earliest date of disease progression per Lugano Classification (Cheson, et al. J Clin Oncol. 2014; 32:3059-68.), commencement of new lymphoma therapy, or death from any cause) by blinded central review. Key secondary endpoints were ORR and OS. Secondary endpoints included investigator-assessed EFS, progression-free survival (PFS), and incidence of AEs.

Disease assessments were evaluated per Lugano Classification Response Criteria. (Cheson, et al. J Clin Oncol. 2014; 32:3059-68.) Screening fluorodeoxyglucose (FDG)-positron emission tomography (PET) from skull base to mid-thighs and diagnostic quality contrasted-enhanced computed tomography (CT) from skull base through lesser trochanters (PET-CT), along with appropriate imaging of all other disease sites were required to confirm eligibility and to establish baseline within 28 days prior to randomization. Patients had their first post-treatment planned PET-CT tumor assessment within the day 50 assessment period (calculated from randomization date). Disease assessments were conducted at day 50, 100, and 150 from randomization. PET-CTs continued through month 9 or until change in lymphoma therapy or disease progression, whichever came first. If the patient's disease did not progress by month 9, disease assessments were evaluated per CT scans where complete response was suspected and per PET-CTs where a PR was suspected. Patients with symptoms suggestive of disease progression were evaluated for progression at time of symptoms. PET-CT could be performed at any time disease progression was suspected. FDG-PET assessment took precedence over CT assessments for time points when both were available. If only CT was available for a time point, assessment may have been affected by the PET-CT assessment at the prior time point. In addition to investigator's assessment, PET-CT scans were submitted to and reviewed by an independent central reviewer blinded to treatment cohort. A patient's bone marrow involvement was confirmed by PET-CT or bone marrow biopsy and aspirate prior to randomization.

Efficacy analyses included all randomized patients on an intent-to-treat basis. Safety analyses included all randomized patients who received ≥1 dose of axicabtagene ciloleucel or SOC on protocol; patients were analyzed by the protocol therapy received. Kaplan-Meier estimates were provided for time-to-event endpoints. Two-sided 95% CIs and estimated hazard ratios (HRs) were calculated from a Cox proportional hazards model stratified by the randomization stratification factors. Stratified log-rank P-values were calculated for time to event endpoints. A stratified Cochran-Mantel-Haenszel test was performed for ORR.

Of 437 patients screened, 359 were randomized to axicabtagene ciloleucel (N=180) or SOC (N=179). The median follow-up time from randomization to data cutoff was 24.9 months. Overall, the median age was 59 years, with 30% aged ≥65 years, 74% of patients had primary refractory disease, 46% had high sAAIPI (2-3), and 19% had HGBL (including double/triple-hit lymphoma) per investigator-assessment (Table 16). Baseline characteristics were balanced between the 2 treatment cohorts.

TABLE 16
Baseline Patient Characteristics in All Treated Patients.

| | Axicabtagene | | | | | |
|---|---|---|---|---|---|---|
| | ciloleucel | | SOC | | Overall | |
| Characteristic | N = 180 | | N = 179 | | N = 359 | |
| Age, median (range), years | 58 | (21-80) | 60 | (26-81) | 59 | (21-81) |
| ≥65 years, n (%) | 51 | (28) | 58 | (32) | 109 | (30) |
| Male sex, n (%) | 110 | (61) | 127 | (71) | 237 | (66) |
| ECOG PS of 1, n (%) | 85 | (47) | 79 | (44) | 164 | (46) |

Disease stage, n (%)

| | | | | | | |
|---|---|---|---|---|---|---|
| I-II | 41 | (23) | 33 | (18) | 74 | (21) |
| III-IV | 139 | (77) | 146 | (82) | 285 | (79) |
| sAAIPI of 2-3, n (%) | 86 | (48) | 79 | (44) | 165 | (46) |

Molecular subgroup percentral laboratory, n (%)*

| | | | | | | |
|---|---|---|---|---|---|---|
| Germinal center B-cell like | 109 | (61) | 99 | (55) | 208 | (58) |
| Activated B-cell like | 16 | (9) | 9 | (5) | 25 | (7) |
| Unclassified | 17 | (9) | 14 | (8) | 31 | (9) |
| Not applicable | 10 | (6) | 16 | (9) | 26 | (7) |
| Missing | 28 | (16) | 41 | (23) | 69 | (19) |

Response to IL therapy at randomization, n (%)

| | | | | | | |
|---|---|---|---|---|---|---|
| Primary refractory | 133 | (74) | 132 | (74) | 265 | (74) |
| Relapse ≤6 months of initiation | 26 | (14) | 22 | (12) | 48 | (13) |
| or completion of | | | | | | |
| IL therapy | | | | | | |
| Relapse >6 and ≤12 months of | 20 | (11) | 24 | (13) | 44 | (12) |
| initiation or completion of | | | | | | |
| IL therapy | | | | | | |
| Missing | 1 | (1) | 1 | (1) | 2 | (1) |

Disease type per central laboratory, n (%)

| | | | | | | |
|---|---|---|---|---|---|---|
| DLBCL^{†} | 126 | (70) | 120 | (67) | 246 | (69) |
| HGBL, NOS | 0 | (0) | 1 | (1) | 1 | (0) |
| HGBL, with MYC/BCL2/BCL6 | 301 | (17) | 25 | (14) | 56 | (16) |
| rearrangement | | | | | | |
| Not confirmed/missing | 18 | (10) | 28 | (16) | 46 | (13) |
| Other | 5 | (3) | 5 | (3) | 10 | (3) |

Disease type per investigator, n (%)

| | | | | | | |
|---|---|---|---|---|---|---|
| LBCL not otherwise specified | 110 | (61) | 116 | (65) | 226 | (63) |
| T cell/histiocyte rich LBCL | 5 | (3) | 6 | (3) | 11 | (3) |
| Epstein-Barr virus + DLBCL | 2 | (1) | 0 | (0) | 2 | (1) |
| Large cell transformation | 19 | (11) | 27 | (15) | 46 | (13) |
| from follicular | | | | | | |
| lymphoma | 43 | (24) | 27 | (15) | 70 | (19) |
| HGBL with or without MYC and BCL2 | 1 | (1) | 0 | (0) | 1 | (0) |
| and/or BCL6 rearrangement | | | | | | |
| Primary cutaneous DLBCL (leg type) | 0 | (0) | 3 | (2) | 3 | (1) |
| Other | | | | | | |

Prognostic marker per central laboratory, n (%)

| | | | | | | |
|---|---|---|---|---|---|---|
| HGBL - double-/triple-hit | 31 | (17) | 25 | (14) | 56 | (16) |
| Double expressor lymphoma | 57 | (32) | 62 | (35) | 119 | (33) |
| MYC rearrangement | 15 | (8) | 7 | (4) | 22 | (6) |
| N/A | 74 | (41) | 70 | (39) | 144 | (40) |
| Missing | 3 | (2) | 15 | (8) | 18 | (5) |
| Positive CD19 status by IHC | 144 | (80) | 134 | (75) | 278 | (77) |
| per central laboratory, n (%)^{‡} | | | | | | |
| Lymphoma present in bone marrow, n (%) | 17 | (9) | 14 | (8) | 31 | (9) |
| Tumor burden per central | 2123 | (181-22,538) | 2069 | (252-20,117) | 2118 | (181-22,538) |
| laboratory^{§}, median | | | | | | |
| (range), mm² | | | | | | |

*Molecular subgroup assessed per investigator (n [%]) was 96 (53%), 84 (47%), and 180 (50%) for germinal center B-cell like; 47 (26%), 54 (30%), and 101 (28%) for non-germinal center B-cell like; and 37 (21%), 41 (23%), and 78 (22%) for not tested in the axicabtagene ciloleucel cohort, SOC cohort, and overall patient population, respectively.
^{†}Definition of DLBCL per central laboratory included cases of incomplete evaluation due to inadequate sample amount or sample type, for which further classification of DLBCL subtype was not possible. DLBCL NOS, per World Health Organization 2016 definition, (Swerdlow, et al. Blood. 2016; 127: 2375-90.) is also included.
^{‡}CD19 staining was not required for participation in the study.
^{§}Tumor burden was measured by sum of product diameters of target lesions per Cheson criteria (Cheson, et al. J Clin Oncol. 2007; 25: 579-586.) and assessed by central laboratory. Data shown are from 180, 179, and 359 patients in the axicabtagene ciloleucel cohort, SOC cohort, and overall patient population, respectively. 1L, first-line; BCL, B-cell lymphoma; DLBCL, diffuse large B-cell lymphoma; ECOG PS, Eastern Cooperative Oncology Group performance status; HGBL, high grade B-cell lymphoma; IHC, immunohistochemistry; LBCL, large B-cell lymphoma; NOS, not otherwise specified; sAAIPI, second-line age-adjusted International Prognostic Index; SOC, standard of care.

Among axicabtagene ciloleucel patients, 178/180 (99%) underwent leukapheresis and 170/180 (94%) received axicabtagene ciloleucel; 60/180 (33%) patients received bridging corticosteroids. Axicabtagene ciloleucel was successfully manufactured for all patients who underwent leukapheresis. The median time from leukapheresis to product release (when product passed quality testing and was made available to investigator) was 13 days (range, 10-24). Among SOC patients, 168/179 (94%) received platinum-based SOC chemotherapy, and 64/179 (36%) received HDT-ASCT (including 2 patients who received ASCT off protocol; Table 17).

TABLE 17
Baseline Characteristics of SOC Patients Who Proceeded to ASCT.

| | SOC | |
|---|---|---|
| Characteristic | n = 62 | |
| ECOG PS of 1, n (%) | 20 | (32) |

Disease stage, n (%)

| | | |
|---|---|---|
| I-II | 11 | (18) |
| III-IV | 51 | (82) |
| sAAIPI of 2-3, n (%) | 23 | (37) |

Molecular subgroup per central laboratory, n (%)

| | | |
|---|---|---|
| Germinal center B-cell like | 39 | (63) |
| Activated B-cell like | 3 | (5) |
| Unclassified | 2 | (3) |
| Not applicable | 7 | (11) |
| Missing | 11 | (18) |

Response to 1L at randomization, n (%)

| | | |
|---|---|---|
| Primary refractory | 38 | (61) |
| Relapse ≤6 months of initiation | 1 | (2) |
| or completion of IL therapy | | |
| Relapse >6 and ≤12 months of | 23 | (37) |
| initiation or completion of IL therapy | | |

Disease type percentral laboratory, n (%)

| | | |
|---|---|---|
| DLBCL* | 47 | (76) |
| HGBL, NOS | 1 | (2) |
| HGBL, with MYC/BCL2/BCL6 rearrangement | 8 | (13) |
| Not confirmed/missing | 3 | (5) |
| Other | 2 | (3) |

Disease type per investigator, n (%)

| | | |
|---|---|---|
| LBCL not otherwise specified | 36 | (58) |
| T cell/histiocyte rich LBCL | 5 | (8) |
| Large cell transformation from | 11 | (18) |
| follicular lymphoma | | |
| HGBL with or without MYC and BCL2 | 10 | (16) |
| and/or BCL6 rearrangement | | |

Prognostic marker percentral laboratory, n (%)^{‡}

| | | |
|---|---|---|
| HGBL - double/triple-hit | 8 | (13) |
| Double expressor lymphoma | 28 | (45) |
| MYC rearrangement | 1 | (2) |
| N/A | 23 | (37) |
| Missing | 2 | (3) |
| Positive CD19 status by IHC per central | 50 | (81) |
| laboratory, n (%) | | |
| Lymphoma present in bone marrow, n (%) | 5 | (8) |

*Definition of DLBCL per central laboratory included cases of incomplete evaluation due to inadequate sample amount or sample type, for which further classification of DLBCL subtype was not possible. DLBCL NOS, per World Health Organization 2016 definition (Swerdlow, et al. Blood. 2016; 127: 2375-90.), is also included.
^{†}CD19 staining was not required for participation in the study.
IL, first-line; ASCT, autologous stem cell transplant; DLBCL, diffuse large B-cell lymphoma; ECOG PS, Eastern Cooperative Oncology Group performance status; HGBL, high grade B-cell lymphoma; IHC, immunohistochemistry; LBCL, large B-cell lymphoma; NOS, not otherwise specified; sAAIPI, second-line age-adjusted International Prognostic Index; SOC, standard of care.

The primary endpoint of EFS was met, demonstrating treatment with axicabtagene ciloleucel was superior to SOC (HR, 0.398; 95% CI, 0.308-0.514; P<0.0001). Median EFS by blinded central review was significantly longer in the axicabtagene ciloleucel versus SOC cohort (8.3 months [95% CI, 4.5-15.8] versus 2.0 [95% CI, 1.6-2.8], respectively). The 24-month estimated EFS rates were 40.5% (95% CI, 33.2-47.7) versus 16.3% (95% CI, 11.1-22.2) in the axicabtagene ciloleucel versus SOC cohorts, respectively (Table 18). EFS improvements with axicabtagene ciloleucel versus SOC were consistent among all key patient subgroups (Table 19). Investigator-assessed EFS was similar to EFS by blinded central review.

TABLE 18
Kaplan-Meier Estimates of Event-free Survival
in axicabtagene ciloleucel and SOC Cohorts.

| | axicabtagene | |
|---|---|---|
| | ciloleucel | SOC |
| % (95% CI) | N = 180 | N = 179 |
| 3 month | 80.6 (74.0, 85.6) | 40.5 (33.2, 47.8) |
| 6 month | 51.1 (43.6, 58.1) | 26.6 (20.2, 33.3) |
| 9 month | 49.4 (42.0, 56.5) | 19.4 (13.8, 25.6) |
| 12 month | 47.2 (39.8, 54.3) | 17.6 (12.3, 23.6) |
| 15 month | 43.9 (36.5, 50.9) | 17.0 (11.8, 23.0) |
| 18 month | 41.5 (34.2, 48.6) | 17.0 (11.8, 23.0) |
| 21 month | 41.5 (34.2, 48.6) | 16.3 (11.1, 22.2) |
| 24 month | 40.5 (33.2, 47.7) | 16.3 (11.1, 22.2) |
| 27 month | 40.5 (33.2, 47.7) | 16.3 (11.1, 22.2) |

Event-free survival was assessed by blinded central review.
SOC, standard of care.

TABLE 19

| | Axi-cel No. of | | SOC No. of | | HR (95% CI) | |
|---|---|---|---|---|---|---|
| | patients with a | | patients with a | | 0.0-1.0 = | |
| | Response/No. | | Response/No. | | Axi-cel Better; | |
| | of Patients | % | of Patients | % | 1.0-5.0 = SOC Better | |
| Overall | 108/180 | 60 | 144/179 | 80 | 0.398 | (0.308-0.514) |
| Age, Years | | | | | | |
| <65 | 81/129 | 63 | 96/121 | 79 | 0.490 | (0.361-0.666) |
| ≥65 | 27/51 | 53 | 48/58 | 83 | 0.276 | (0.164-0.465) |
| Response to 1L therapy at | | | | | | |
| randomization | | | | | | |
| Primary refractory | 85/133 | 64 | 106/131 | 81 | 0.426 | (0.319-0.570) |
| Relapse ≤12 months of | 23/47 | 49 | 38/48 | 79 | 0.342 | (0.202-0.579) |
| initiation or completion of | | | | | | |
| 1L therapy | | | | | | |
| sAAIPI | | | | | | |
| 0-1 | 54/98 | 55 | 73/100 | 73 | 0.407 | (0.285-0.582) |
| 2-3 | 54/82 | 66 | 71/79 | 90 | 0.388 | (0.269-0.561) |
| Prognostic marker per | | | | | | |
| central laboratory | | | | | | |
| HGBL-double/triple hit | 15/31 | 48 | 21/25 | 84 | 0.285 | (0.137-0.593) |
| Double expressor lymphoma | 35/57 | 61 | 50/62 | 81 | 0.424 | (0.268-0.671) |
| Molecular subgroup per | | | | | | |
| central laboratory | | | | | | |
| Germinal center B-cell like | 64/109 | 59 | 80/99 | 81 | 0.407 | (0.290-0.570) |
| Activated B-cell like | 11/16 | 69 | 9/9 | 100 | 0.182 | (0.046-0.720) |
| Unclassified | 8/17 | 47 | 12/14 | 86 | 0.000 | (0.000-NE) |

ORR was significantly greater in axicabtagene ciloleucel versus SOC patients (83% versus 50%, respectively; odds ratio, 5.31 [95% CI, 3.1-8.9; P<0.0001]), with CR rates of 65% versus 32%. The interim analysis of OS favored axicabtagene ciloleucel (median not reached [NR]) versus SOC (median, 35.1 months [HR, 0.730; P=0.0270]). The proportion of SOC patients who received subsequent cellular immunotherapy was 56% (HR, 0.695; 95% CI, 0.461-1.049). A preplanned OS sensitivity analysis, conducted to address the confounding effects of treatment switching to subsequent cellular immunotherapy in the SOC cohort, demonstrated a statistically significant difference in OS in favor of axicabtagene ciloleucel with a stratified HR of 0.580 (95% CI, 0.416-0.809; descriptive log-rank P=0.0006 using the Rank Preserving Structural Failure Time (RPSFT) model. The validated and commonly-used RPSFT model preserves randomization, (Danner and Sarkar. PharmaSUG. 2018; EP-04.) revealing the difference in treatment effect if SOC patients did not receive subsequent cellular immunotherapy.

Median PFS was longer in axicabtagene ciloleucel versus SOC patients (14.7 months [95% CI, 5.4-NE] versus 3.7 months [95% CI, 2.9-5.3]); HR, 0.490; P<0.0001). Estimated 24-month PFS rates were 45.7% (95% CI, 38.1-53.0) in the axicabtagene ciloleucel cohort and 27.4% (95% CI, 20.0-35.3) in the SOC cohort. Median duration of response (DOR) numerically favored axicabtagene ciloleucel over SOC but did not reach statistical significance (26.9 months [95% CI, 13.6-NE] versus 8.9 months [95% CI, 5.7-NE]; HR, 0.769; P=0.0695).

Due to risks associated with axicabtagene ciloleucel treatment, infusion was delayed, and an appropriate assessment performed if a patient had any of the following conditions:
Unresolved serious adverse reactions (especially pulmonary reactions, cardiac reactions, or hypotension), including those from previous chemotherapies
Active uncontrolled infection
Active graft versus host disease

Cytokine release syndrome (CRS) management in anti-CD19 CAR T-cell therapy was intended to prevent life-threatening conditions while preserving the benefits of antitumor effects. Patients were monitored for signs and symptoms of CRS. Diagnosis of CRS required excluding alternate causes of systemic inflammatory response, particularly infection. Patients who experienced grade ≥2 CRS were monitored with continuous cardiac telemetry and pulse oximetry. For patients experiencing severe CRS, an echocardiograph was considered to assess cardiac function. For severe or life-threatening CRS, intensive care supportive therapy was considered. Table 20 outlines the recommended management of CRS associated with treatment with axicabtagene ciloleucel.

TABLE 20
recommended management of CRS associated with
treatment with axicabtagene ciloleucel

| | | | | |
|---|---|---|---|---|
| | Supportive | | | |
| CRS Grade* | Care | Tocilizumab | Corticosteroids | Follow-up |

Grade 1

| | | | | |
|---|---|---|---|---|
| Symptoms | Supportive | N/A | N/A | Not improving after |
| require | care per | | | 24 hours |
| symptomatic | institutional | | | Tocilizumab |
| treatment only | SOC | | | 8 mg/kg IV over 1 |
| (eg, fever, | Closely | | | hour (not to exceed |
| nausea, | monitor | | | 800 mg) |
| fatigue, | neurologic | | | |
| headache, | status | | | |
| myalgia, | | | | |
| malaise) | | | | |

Grade 2

| | | | | |
|---|---|---|---|---|
| Symptoms | Continuous | Tocilizumab | If no improvement | Improving |
| require and | cardiac | 8 mg/kg IV | within 24 hours | Manage as above |
| respond to | telemetry and | over 1 hour | after starting | If corticosteroids |
| moderate | pulse | (not to | tocilizumab, | were started: |
| intervention | oximetry as | exceed 800 | manage per Grade | continue |
| Oxygen | indicated | mg) | 3 | corticosteroids use |
| requirement | IV fluids | Repeat | | until the event is |
| <40% FiO2 or | bolus for | tocilizumab | | Grade 1 or less, then |
| hypotension | hypotension | every 8 | | taper over 3 days |
| responsive to | with 0.5 to 1.0 | hours as | | Not improving |
| fluids or low | L isotonic | needed if not | | Manage as Grade 3 |
| dose of 1 | fluids | responsive to | | (below) |
| vasopressor or | Vasopressor | IV fluids or | | |
| Grade 2 organ | support for | increasing | | |
| toxicity | hypotension | supplemental | | |
| | not responsive | oxygen; | | |
| | to IV fluids | maximum of | | |
| | Supplemental | 3 doses/24 | | |
| | oxygen as | hours. | | |
| | indicated | Maximum | | |
| | | total of 4 | | |
| | | doses if no | | |
| | | clinical | | |
| | | improvement | | |
| | | in the signs | | |
| | | and | | |
| | | symptoms of | | |
| | | CRS | | |

Grade 3

| | | | | |
|---|---|---|---|---|
| Symptoms | Management | Per Grade 2 | Methylprednisolone | Improving |
| require and | in monitored | | 1 mg/kg IV BID or | Manage as Grade 2 |
| respond to | care or | | equivalent (above) | Continue |
| aggressive | intensive care | | dexamethasone (eg, | corticosteroids use |
| intervention | unit | | 10 mg IV every | until the event is |
| Oxygen | | | 6 hours) | Grade 1 or less, then |
| requirement ≥ | | | | taper over 3 days |
| 40% FiO2 or | | | | Not improving |
| hypotension | | | | Manage as Grade 4 |
| requiring high- | | | | (below) |
| dose or | | | | |
| multiple | | | | |
| vasopressors | | | | |
| or Grade 3 | | | | |
| organ toxicity | | | | |
| or Grade 4 | | | | |
| transaminitis | | | | |

Grade 4

| | | | | |
|---|---|---|---|---|
| Life- | Per Grade 3 | Per Grade 2 | High-dose | Improving |
| threatening | | Mechanical | corticosteroids: | Manage as above |
| symptoms | | ventilation | methylprednisolone | Continue |
| Requirements | | and/or renal | 1000 mg/day IVx 3 | corticosteroids use |
| for ventilator | | replacement | days | until the event is |
| support or | | therapy may | | Grade 1 or less, then |
| continuous | | be required | | taper over 3 days |
| veno-venous | | | | Not improving |
| hemodialysis | | | | Consider alternate |
| (CVVHD) | | | | immunosuppressants |
| Grade 4 organ | | | | Contact Medical |
| toxicity | | | | Monitor |
| (excluding | | | | |
| transaminitis) | | | | |

BID, twice daily; IV, intravenous; CRS, cytokine release syndrome; FiO2, fraction of inspired oxygen; SOC, standard of care.
*Modified Lee et al 2014. (Lee, et al. Blood. 2014; 124: 188-95.)

Patients were carefully monitored for signs and symptoms of neurologic events. Patients who experienced grade ≥2 neurologic events had brain imaging, a lumbar puncture (with opening pressure assessment), regular neurologic exams, and were monitored with continuous cardiac telemetry and pulse oximetry. Transfer to intensive care was considered for potentially severe or life-threatening neurologic events. Non-sedating, anti-seizure medicines (eg, levetiracetam) for prophylaxis against seizures were considered for grade ≥2 neurologic events in the absence of contraindications. Tapering for levetiracetam was only done when the neurologic event was grade ≤1. Endotracheal intubation may have been required for airway protection in severe cases. In some cases, multiple anti-epileptic medications may have been needed to control seizures. Medications with sedative properties were avoided unless required.

Leukoencephalopathy cases were managed based on clinical symptoms and follow-up magnetic resonance imaging was recommended for monitoring. Table 21 outlines the recommended management of neurologic events associated with treatment with axicabtagene ciloleuce.

TABLE 21
recommended management of neurologic events associated with treatment with axicabtagene ciloleucel.

| | | | | |
|---|---|---|---|---|
| Neurologic Event Grade | Supportive Care | Concurrent CRS | No Concurrent CRS | Follow-up |

Grade 1

| | | | | |
|---|---|---|---|---|
| Examples include: | Supportive care per | N/A | N/A | Not improving |
| Somnolence-mild | institutional SOC | | | Continue supportive care |
| drowsiness or sleepiness | Closely monitor neurologic | | | |
| Confusion-mild | status | | | |
| disorientation | Consider prophylactic non- | | | |
| Encephalopathy-mild | sedating anti-seizure | | | |
| limiting of ADLs | medication | | | |
| Dysphasia-not impairing | | | | |
| ability to communicate | | | | |

Grade 2

| | | | | |
|---|---|---|---|---|
| Examples include: | Continuous cardiac | Tocilizumab | Tocilizumab not indicated | Improving |
| Somnolence-moderate, | telemetry and pulse | 8 mg/kg IV over | Dexamethasone at 10 mg | Manage as above |
| limiting instrumental | oximetry as indicated | 1 hour (not to exceed | IV every | Continue dexamethasone |
| ADLs | Closely monitor neurologic | 800 mg) | 6 hours | use until the event is Grade |
| Confusion-moderate | status with serial neuro | Repeat tocilizumab every 8 | | 1 or less, then taper over 3 |
| disorientation | exams to include | hours as needed if not | | days |
| Encephalopathy-limiting | fundoscopy and Glasgow | responsive to IV fluids or | | Not improving |
| instrumental ADLs | Coma Score. Consider | increasing supplemental | | Manage as Grade 3 |
| Dysphasia-moderate | neurology consult. | oxygen; maximum of 3 doses | | (below) |
| impairing ability to | Perform brain imaging (eg, | in a 24-hour period. | | |
| communicate | MRI), EEG, and lumbar | Maximum total of 4 doses if | | |
| spontaneously | puncture (with opening | no clinical improvement in | | |
| Seizure(s) | pressure) if no | the signs and symptoms of | | |
| | contraindications | CRS | | |
| | Consider prophylactic | If no improvement within | | |
| | nonsedating, anti seizure | 24 hours after starting | | |
| | medication | tocilizumab, give | | |
| | | dexamethasone 10 mg IV | | |
| | | every | | |
| | | 6 hours*, if not already | | |
| | | taking other | | |
| | | corticosteroids. | | |
| | | Continue dexamethasone use | | |
| | | until the event is Grade 1 or | | |
| | | less, then taper over 3 days | | |

Grade 3

| | | | | |
|---|---|---|---|---|
| Examples include: | Management in monitored | Administer tocilizumab per | Dexamethasone at 10 mg | Improving |
| Somnolence-obtundation | care or intensive care unit | Grade 2 | IV every 6 hours. | Manage as above |
| or stupor | | In addition, administer | Continue dexamethasone | Continue dexamethasone |
| Confusion-severe | | dexamethasone 10 mg IV | use until the event is | use until the event is Grade |
| disorientation | | with the first dose of | Grade 1 or less, then taper | 1 or less, then taper over 3 |
| Encephalopathy-limiting | | tocilizumab and repeat dose | over 3 days | days |
| self-care ADLs | | every 6 hours. Continue | | Not improving |
| Dysphasia-severe | | dexamethasone use until the | | Manage as Grade 4 |
| receptive or expressive | | event is Grade 1 or less, then | | (below) |
| characteristics, | | taper over 3 days. | | |
| impairing ability to | | | | |
| read, write, or | | | | |
| communicate intelligibly | | | | |

Grade 4

| | | | | |
|---|---|---|---|---|
| Life-threatening | Per Grade 3 | Administer tocilizumab per | High-dose corticosteroids: | Improving |
| consequences | Mechanical | Grade 2 | methylprednisolone 1000 | Manage as Grade 3 (above) |
| Urgent intervention | ventilation may | In addition, administer | mg/day IV × 3 days; if it | Continue |
| indicated | be required | methylprednisolone 1000 mg | improves, then manage as | methylprednisolone use |
| Requirement for | | IV per day with first dose of | above. | until the event is Grade 1 |
| mechanical ventilation | | tocilizumab and continue | | or less, then taper over 3 |
| Consider cerebral | | methylprednisolone 1000 mg | | days |
| edema (refer to table | | intravenously per day for 2 | | Not improving |
| below for management | | more days; if improves, then | | Consider alternate |
| of suspected cerebral | | manage as above | | immunosuppressants |
| edema) | | | | Contact Medical Monitor |

ADL, activities of daily life; CRS, cytokine release syndrome; CTCAE, Common Terminology Criteria for Adverse Events; EEG, electroencephalogram; MRI, magnetic resonance imaging; NA, not application; SOC, standard of care.
*Or equivalent methylprednisolone dose (1 mg/kg).
^{†}Equivalent dose of dexamethasone is 188 mg/day.

Cerebral edema was considered in patients with progressive neurologic symptoms at any grade of neurologic event. Diagnostics included serial neurologic exams. Guidelines for management of suspected cerebral edema are included Table 22.

TABLE 22
recommended management of suspected cerebral edema.

| Supportive Therapy | Tocilizumab | Corticosteroids | Follow-up |
|---|---|---|---|
| As above for neurologic | Tocilizumab as | High-dose | Improving: |
| events Grade 4, to | above in Grade 4 | corticosteroids: | Very slow |
| include: | neurologic event | methylprednisolone | corticosteroid taper |
| Intensive care unit | management | 1000 mg/day × | recommended |
| supportive therapy | (tocilizumab should | 3 days | Serial neurologic |
| Neuro-Intensivist | be given only if | | exams as indicated |
| consult | concurrent CRS) | | Consider early |
| If cerebral edema | | | neuro-rehabilitation |
| documented or strongly | | | Not improving: |
| suspected, recommend | | | Repeat neuro- |
| neurosurgical consult | | | imaging as indicated |
| Optimal head position | | | Consider alternate |
| with elevation of head of | | | immunosuppressants |
| bed and straight neck | | | Consult medical |
| positioning | | | monitor |
| Administration of | | | |
| diuretics and | | | |
| osmotherapy per | | | |
| institutional practice | | | |
| guidelines | | | |
| Early tracheal | | | |
| intubation with | | | |
| controlled mechanical | | | |
| mild hyperventilation | | | |
| and good oxygenation | | | |
| Maintain cerebral | | | |
| perfusion pressure with | | | |
| mild hypervolemia | | | |
| Avoid hypertension with | | | |
| use of anti-hypertensives | | | |
| (labetalol, nicardipine) | | | |
| Avoid potent | | | |
| vasodilators | | | |
| Pharmacological | | | |
| cerebral metabolic | | | |
| suppression | | | |
| (barbiturates, sedation, | | | |
| analgesia, and | | | |
| neuromuscular | | | |
| paralysis, as indicated) | | | |
| Maintain rigorous | | | |
| glycemic control | | | |

CRS, cytokine release syndrome.
Note:
Information is based on a review of treatment for cerebral edema by Rabinstein, 2006. (Rabinstein. Neurologist. 2006; 12: 59-73.)

Cytopenias, including prolonged cytopenias, were managed with a thorough evaluation for a source of infection and administration of prophylactic broad-spectrum antibiotics per institutional practice guidelines. Granulocyte colony-stimulating factor (G-CSF) was given according to published guidelines. Fevers were treated with supportive measures and antipyretics. Euvolemia was maintained with addition of isotonic intravenous fluids (eg, crystalloids) as clinically indicated and per institutional practice guidelines. Prolonged cytopenias beyond 30 days following axicabtagene ciloleucel administration may have required clinical investigation, including bone marrow biopsy. Patients received platelets and packed red blood cells as needed for anemia and thrombocytopenia.

Patients were monitored for signs and symptoms of infection, and treatment with antibiotics for suspected or confirmed infections was recommended. Patients received prophylaxis for infection with pneumocystis pneumonia, herpes virus, and fungal infections according to National Comprehensive Cancer Network guidelines or standard institutional practice guidelines. Fevers were treated with acetaminophen and comfort measures, and corticosteroids were avoided. Patients who were neutropenic and febrile received broad-spectrum antibiotics and maintenance intravenous fluids were started on most patients with high fevers. G-CSF was given according to published guidelines (eg, Infectious Disease Society of America). Patients with B-cell aplasia leading to hypogammaglobulinemia received intravenous immunoglobulin per institutional practice guidelines. Screening for hepatitis B virus, hepatitis C virus, and HIV were performed in accordance with clinical guidelines before collection of cells for manufacturing.

All patients experienced ≥1 any-grade AE. Grade ≥3 AEs occurred in 91% (155/170) and 83% (140/168) of patients who received axicabtagene ciloleucel and SOC therapies, respectively. The most commonly reported grade ≥3 AEs was neutropenia (69% axicabtagene ciloleucel; 41% SOC; Table 23). Serious AEs of any grade occurred in 50% and 46% of patients in the axicabtagene ciloleucel and SOC cohorts, respectively (Table 24); any-grade infections occurred in 41% and 30% of patients with grade ≥3 infections occurring in 14% and 11%.

TABLE 23
Most Common Adverse Events, Cytokine Release
Syndrome, and Neurologic Events.

| | Axicabtagene ciloleucel | | | | SOC | | | |
|---|---|---|---|---|---|---|---|---|
| | N = 170 | | | | N = 168 | | | |
| n (%)* | Any Grade | | Grade ≥3 | | Any Grade | | Grade ≥3 | |
| Any adverse event | 170 | (100) | 155 | (91) | 168 | (100) | 140 | (83) |
| Pyrexia | 158 | (93) | 15 | (9) | 43 | (26) | 1 | (1) |
| Neutropenia | 121 | (71) | 118 | (69) | 70 | (42) | 69 | (41) |
| Hypotension | 75 | (44) | 19 | (11) | 25 | (15) | 5 | (3) |
| Fatigue | 71 | (42) | 11 | (6) | 87 | (52) | 4 | (2) |
| Anemia | 71 | (42) | 51 | (30) | 91 | (54) | 65 | (39) |
| Diarrhea | 71 | (42) | 4 | (2) | 66 | (39) | 7 | (4) |
| Headache | 70 | (41) | 5 | (3) | 43 | (26) | 2 | (1) |
| Nausea | 69 | (41) | 3 | (2) | 116 | (69) | 9 | (5) |
| Sinus tachycardia | 58 | (34) | 3 | (2) | 17 | (10) | 1 | (1) |
| Leukopenia | 55 | (32) | 50 | (29) | 43 | (26) | 37 | (22) |
| Thrombocytopenia | 50 | (29) | 25 | (15) | 101 | (60) | 95 | (57) |
| Chills | 47 | (28) | 1 | (1) | 14 | (8) | 0 | (0) |
| Hypokalemia | 44 | (26) | 10 | (6) | 49 | (29) | 11 | (7) |
| Hypophosphatemia | 45 | (26) | 31 | (18) | 29 | (17) | 21 | (13) |
| Cough | 42 | (25) | 1 | (1) | 18 | (11) | 0 | (0) |
| Decreased appetite | 42 | (25) | 7 | (4) | 42 | (25) | 6 | (4) |
| Hypoxia | 37 | (22) | 16 | (9) | 13 | (8) | 7 | (4) |
| Dizziness | 36 | (21) | 2 | (1) | 21 | (13) | 1 | (1) |
| Constipation | 34 | (20) | 0 | (0) | 58 | (35) | 0 | (0) |
| Vomiting | 33 | (19) | 0 | (0) | 55 | (33) | 1 | (1) |
| Febrile neutropenia | 4 | (2) | 4 | (2) | 46 | (27) | 46 | (27) |
| CRS | 157 | (92) | 11 | (6) | — | | — | |
| Pyrexia | 155 | (99) | 14 | (9) | — | | — | |
| Hypotension | 68 | (43) | 18 | (11) | — | | — | |
| Sinus tachycardia | 49 | (31) | 3 | (2) | — | | — | |
| Chills | 38 | (24) | 0 | (0) | — | | — | |
| Hypoxia | 31 | (20) | 13 | (8) | — | | — | |
| Headache | 32 | (20) | 2 | (1) | — | | — | |
| Neurologic events | 102 | (60) | 36 | (21) | 33 | (20)^{†} | 1 | (1) |
| Tremor | 44 | (26) | 2 | (1) | 1 | (1) | 0 | (0) |
| Confusional state | 40 | (24) | 9 | (5) | 4 | (2) | 0 | (0) |
| Aphasia | 36 | (21) | 12 | (7) | 0 | (0) | 0 | (0) |
| Encephalopathy | 29 | (17) | 20 | (12) | 2 | (1) | 0 | (0) |
| Paresthesia | 8 | (5) | 1 | (1) | 14 | (8) | 0 | (0) |
| Delirium | 3 | (2) | 3 | (2) | 5 | (3) | 1 | (1) |

CRS, cytokine release syndrome; SOC, standard of care.
*Included are any adverse events of any grade occurring in ≥20% of patients in either the axicabtagene ciloleucel or SOC cohort, and CRS and neurologic events of any grade occurring in ≥15% of patients in the axicabtagene ciloleucel cohort or ≥3% in the SOC cohort. CRS was graded according to Lee et al. (Lee, et al. Blood. 2014; 124: 188-95.) Neurologic events were identified per prespecified search list of Medical Dictionary for Regulatory Activities preferred terms, based on known neurotoxicities associated with anti-CD 19 immunotherapy and were specifically identified using methods based on the blinatumomab registrational study. (Topp, et al. Lancet Oncol. 2015; 16: 57-66.) The severity of all adverse events, including neurologic events and symptoms of CRS, was graded with the use of the National Cancer Institute Common Terminology Criteria for Adverse Events, version 4.03.
^{†}Other preferred terms reported in the SOC cohort (in ≤2 patients) included somnolence, agitation, hypoesthesia, lethargy, depressed level of consciousness, cognitive disorder, memory impairment, bradyphrenia, taste disorder, hallucination, nystagmus, head discomfort, and neuralgia.

TABLE 24
Serious Adverse Events Occurring in at Least 3 Patients in the Overall Population.

| | Axicabtagene ciloleucel | | | | SOC | | | |
|---|---|---|---|---|---|---|---|---|
| | N = 170 | | | | N = 168 | | | |
| n (%) | Any Grade | | Grade ≥3 | | Any Grade | | Grade ≥3 | |
| Any serious adverse event | 85 | (50) | 72 | (42) | 77 | (46) | 67 | (40) |
| Pyrexia | 27 | (16) | 1 | (1) | 8 | (5) | 0 | (0) |
| Encephalopathy | 17 | (10) | 15 | (9) | 1 | (1) | 0 | (0) |
| Hypotension | 15 | (9) | 7 | (4) | 3 | (2) | 3 | (2) |
| Pneumonia | 8 | (5) | 6 | (4) | 4 | (2) | 3 | (2) |
| Aphasia | 9 | (5) | 8 | (5) | 0 | (0) | 0 | (0) |
| B-cell lymphoma | 7 | (4) | 7 | (4) | 5 | (3) | 5 | (3) |
| Confusional state | 6 | (4) | 4 | (2) | 0 | (0) | 0 | (0) |
| Neutropenia | 6 | (4) | 5 | (3) | 4 | (2) | 4 | (2) |
| Somnolence | 5 | (3) | 3 | (2) | 0 | (0) | 0 | (0) |
| Tremor | 5 | (3) | 1 | (1) | 0 | (0) | 0 | (0) |
| Acute kidney injury | 3 | (2) | 2 | (1) | 8 | (5) | 4 | (2) |
| Atrial fibrillation | 4 | (2) | 3 | (2) | 2 | (1) | 0 | (0) |
| Febrile neutropenia | 4 | (2) | 4 | (2) | 22 | (13) | 22 | (13) |
| Abdominal pain | 3 | (2) | 2 | (1) | 2 | (1) | 1 | (1) |
| Hypoxia | 3 | (2) | 1 | (1) | 2 | (1) | 2 | (1) |
| Dyspnea | 3 | (2) | 3 | (2) | 1 | (1) | 1 | (1) |
| Headache | 4 | (2) | 3 | (2) | 0 | (0) | 0 | (0) |
| Fatigue | 3 | (2) | 2 | (1) | 0 | (0) | 0 | (0) |
| COVID-19 | 3 | (2) | 3 | (2) | 0 | (0) | 0 | (0) |
| Muscular weakness | 3 | (2) | 2 | (1) | 0 | (0) | 0 | (0) |
| Anemia | 1 | (1) | 1 | (1) | 3 | (2) | 3 | (2) |
| Decreased appetite | 1 | (1) | 1 | (1) | 3 | (2) | 3 | (2) |
| Hyponatremia | 2 | (1) | 2 | (1) | 1 | (1) | 1 | (1) |
| Malaise | 2 | (1) | 0 | (0) | 1 | (1) | 0 | (0) |
| Sinus tachycardia | 2 | (1) | 1 | (1) | 2 | (1) | 1 | (1) |
| Syncope | 1 | (1) | 1 | (1) | 3 | (2) | 3 | (2) |
| Back pain | 1 | (1) | 0 | (0) | 2 | (1) | 2 | (1) |
| Sepsis | 2 | (1) | 2 | (1) | 4 | (2) | 4 | (2) |
| Nausea | 1 | (1) | 0 | (0) | 2 | (1) | 2 | (1) |
| Dehydration | 0 | (0) | 0 | (0) | 3 | (2) | 3 | (2) |
| Thrombocytopenia | 0 | (0) | 0 | (0) | 6 | (4) | 6 | (4) |

Axicabtagene ciloleucel, axicabtagene ciloleucel; SOC, standard of care.

Frequency of cytopenias is summarized in Table 22. Prolonged grade ≥3 cytopenia present on or after day 30 from initiation of therapy occurred in 49 (29%) and 101 (60%) patients in the axicabtagene ciloleucel and SOC cohorts, respectively (Table 25). There were no cases of replication-competent retrovirus or axicabtagene ciloleucel treatment-related secondary malignancies reported.

TABLE 25
Summary of Cytopenias Present on or After Day 30 After Treatment Initiation*

| | Axicabtagene ciloleucel | | | | SOC | | | |
|---|---|---|---|---|---|---|---|---|
| | N = 170 | | | | N = 168 | | | |
| n (%) | Any Grade | | Grade ≥3 | | Any Grade | | Grade ≥3 | |
| Any prolonged cytopenia | 70 | (41) | 49 | (29) | 117 | (70) | 101 | (60) |
| Prolonged thrombocytopenia^{†} | 32 | (19) | 11 | (6) | 85 | (51) | 78 | (46) |
| Platelet count decreased | 17 | (10) | 5 | (3) | 53 | (32) | 47 | (28) |
| Thrombocytopenia | 16 | (9) | 6 | (4) | 35 | (21) | 33 | (20) |
| Prolonged neutropenia^{‡} | 56 | (33) | 44 | (26) | 61 | (36) | 60 | (36) |
| Neutrophil count decreased | 26 | (15) | 20 | (12) | 28 | (17) | 28 | (17) |
| Neutropenia | 29 | (17) | 22 | (13) | 21 | (13) | 20 | (12) |
| Febrile neutropenia | 4 | (2) | 4 | (2) | 36 | (21) | 36 | (21) |
| Prolonged anemia^{§} | 23 | (14) | 5 | (3) | 84 | (50) | 57 | (34) |
| Anemia | 22 | (13) | 5 | (3) | 83 | (49) | 57 | (34) |
| Anemia macrocytic | 1 | (1) | 0 | (0) | 0 | (0) | 0 | (0) |
| Hematocrit decreased | 1 | (1) | 0 | (0) | 0 | (0) | 0 | (0) |
| Hemoglobin decreased | 0 | (0) | 0 | (0) | 1 | (1) | 0 | (0) |

*Day 0 is defined as the day the patient received axicabtagene ciloleucel infusion or the first dose of salvage chemoimmunotherapy.
^{†}Thrombocytopenia was identified with SMQ hematopoietic thrombocytopenia (narrow).
^{‡}Neutropenia was identified using the MedDRA preferred terms of neutropenia, neutrophil count decreased, and febrile neutropenia.
^{§}Anemia was identified using the SMQ hematopoietic erythropenia (broad).
Multiple instances of the same adverse event in 1 patient are counted once at the worst grade for each patient. Adverse events were coded using MedDRA version 23.1 and graded per Common Terminology Criteria for Adverse Events version 4.03.
Axicabtagene ciloleucel, axicabtagene ciloleucel; MedDRA, Medical Dictionary for Regulatory Activities; SMQ, Standardized MedDRA Queries; SOC, standard of care.

Sixty-four (38%) and 78 (46%) patients died in the axicabtagene ciloleucel and SOC cohorts, respectively. Of those, 47 (28%) and 64 (38%) patients died from progressive disease. Grade 5 AEs occurred in 7 (4%) patients in the axicabtagene ciloleucel cohort (of which only 1 was axicabtagene ciloleucel-related: hepatitis B reactivation), and 2 (1%) patients in the SOC cohort (both of which were SOC-related: cardiac arrest and acute respiratory distress syndrome; Table 26).

TABLE 26
Deaths in Axicabtagene ciloleucel and SOC Cohorts.

| | Axicabtagene | |
|---|---|---|
| | ciloleucel | SOC |
| | n = 64 | n = 78 |
| Reason for death, n | | |
| Progressive disease | 47 | 64 |
| Grade 5 adverse event | 7 | 2 |
| COVID-19 | 2 | 0 |
| Lung adenocarcinoma | 1 | 0 |
| Myocardial infarction | 1 | 0 |
| Progressive multifocal | 1 | 0 |
| leukoencephalopathy | | |
| Sepsis | 1 | 0 |
| Hepatitis B reactivation | 1* | 0 |
| Cardiac arrest | 0 | 1^{†} |
| Acute respiratory distress syndrome | 0 | 1^{†} |
| Other reason for death | | |
| COVID-19 | 10 | 12 |
| Stroke | 2 | 2 |
| Ischemic colitis | 1 | 0 |
| Progression from prior subdural | 1 | 0 |
| hematoma | 1 | 0 |
| Respiratory failure | 1 | 0 |
| Euthanasia due to progressive disease | 1 | 0 |
| Pulmonary infection | 1 | 0 |
| Unexplained/unknown | 1 | 3 |
| Septic shock | 1 | 1 |
| Cardiopulmonary arrest | 0 | 1 |
| Cryptogenic organizing pneumonia | 0 | 1 |
| Sepsis | 0 | 2 |
| Urosepsis | 0 | 1 |
| Hyperinflammation | 0 | 1 |

*Axicabtagene ciloleucel-related grade 5 adverse event;
^{†}HDT-related grade 5 adverse event. Grade 5 adverse events are those that occurred during the protocol-specified adverse event reporting period.
HDT, high-dose therapy; SOC, standard of care.

CRS occurred in 92% (157/170) of axicabtagene ciloleucel patients (Table 22). Grade ≥3 CRS occurred in 6% (11/170) of patients. No grade 5 CRS events occurred. Tocilizumab, corticosteroids, and vasopressors were administered to 65%, 24%, and 6% of patients, respectively, for CRS management. Median cumulative tocilizumab use, regardless of indication, was 1396 mg (range, 430-7200); most patients received ≤4 doses of tocilizumab (102/170; 60%). The median time to onset of CRS was 3 days post-infusion (range, 1-10) and the median duration of CRS was 7 days (range, 2-43). All events in the setting of CRS resolved.

Neurologic events occurred in 60% (102/170) and 20% (33/168) of patients in the axicabtagene ciloleucel and SOC cohorts, respectively; grade ≥3 neurologic events occurred in 21% (36/170) and 1% (1/168) of patients, respectively. No grade 5 neurologic events occurred. In the axicabtagene ciloleucel cohort, corticosteroids were used in 32% of patients for management of neurologic events. The median time to onset of neurologic events was 5 days (range, 1-133) and 10 days (range, 1-146) in the axicabtagene ciloleucel and SOC cohorts, respectively. The median duration of neurologic events was 14 (range, 1-817) and 26 days (range, 1-588) in the axicabtagene ciloleucel and SOC cohorts, respectively. At data cutoff, 2 patients had ongoing neurologic events (1 axicabtagene ciloleucel patient with grade 2 paresthesia and grade 1 memory impairment; 1 SOC patient with grade 1 paresthesia).

The median time to peak CAR T-cell levels post-axicabtagene ciloleucel infusion was 8 days (range, 2-233; Table 27). The median peak CAR T-cell level was 25.84 cells/μL (range, 0.04-1173), with CAR T cells remaining detectable in 12/30 (40%) evaluable patients by 24 months. CAR T-cell peak and area under the curve within the first 28 days after treatment correlated with objective response (not shown), consistent with Locke, et al. Mol Ther. 2017; 25:285-295. No occurrence of anti-axicabtagene ciloleucel antibodies were detected.

TABLE 27
CAR T-Cell Levels.

| | Axicabtagene ciloleucel | |
|---|---|---|
| CAR T-cell levels (cells/μL) | N = 170 | |
| Baseline, median (Q1, Q3) | 0 | (0, 0) |
| Treatment day 1, median (Q1, Q3) | 4.06 × 10⁻³ | (4.12 × 10⁻⁴, 0.01) |
| Treatment day 3, median (Q1, Q3) | 0.01 | (0.00, 0.08) |
| Treatment day 7, median (Q1, Q3) | 21.37 | (5.16, 57.04) |
| 2 weeks post-treatment, median (Q1, Q3) | 6.28 | (2.31, 24.10) |
| 4 weeks post-treatment, median (Q1, Q3) | 1.57 | (0.72, 5.40) |
| 3 months post-treatment, median (Q1, Q3) | 0.35 | (0.05, 1.02) |
| 6 months post-treatment, median (Q1, Q3) | 0.17 | (0.00, 0.47) |
| 9 months post-treatment, median (Q1, Q3) | 0.14 | (0.00, 0.49) |
| 12 months post-treatment, median (Q1, Q3) | 0.08 | (0.00, 0.37) |
| 18 months post-treatment, median (Q1, Q3) | 0.03 | (0.00, 0.27) |
| 24 months post-treatment, median (Q1, Q3) | 0.00 | (0.00, 0.14) |
| Peak, median (range) | 25.84 | (0.04-1173) |
| AUC₀₋₂₈, cells/μL × days, median (range) | 236.23 | (0.00-1.65 × 10⁴) |
| Time to peak, days, median (range) | 8* | (2-233) |

Axicabtagene ciloleucel, axicabtagene ciloleucel; AUC₀₋₂₈; area under the curve from days 0 to 28; CAR, chimeric antigen receptor.
*Day 8 equals 7 days after the day of axicabtagene ciloleucel infusion (axicabtagene ciloleucel infusion day is day 1 for the purpose of calculating time to peak).

Summary statistics were provided for anti-CD19 CAR T cells measured in blood. The presence, expansion, and persistence of CAR T cells were measured in peripheral blood mononuclear cells as previously reported. (Locke, et al. Mol Ther. 2017; 25:285-295.) Briefly, blood-derived and cryopreserved peripheral blood mononuclear cells were analyzed by quantitative PCR (qPCR) to assess the levels of anti-CD19 CAR-T cell levels over time. qPCR values were converted into cells/uL of blood. Post-infusion peak, time to peak, area under the curve (AUC) from day 0 to day 28 (AUC₀₋₂₈), and the persistence of anti-CD19 CART cells up to 24 months in patients with evaluable samples are presented herein.

Potential immunogenicity was initially identified by the development of antibodies that tested positive for reactivity against the murine monoclonal antibody FMC63 (parent antibody for the single-chain variable region fragment [scFv] used for production of the anti-CD19 CAR in axicabtagene ciloleucel), as measured by a traditional sandwich-based enzyme-linked immunosorbent assay (ELISA). Positive samples underwent further testing with a confirmatory flow cytometry cell-based assay to determine whether the signal observed in the initial screening assay (ELISA) was due to the antibody binding to a properly folded scFv expressed on the surface of an anti-CD19 CART cell.

Although OS outcomes in the current study are immature, interim analysis trended toward favoring axicabtagene ciloleucel. Patients who progressed in the SOC cohort could receive CAR T-cell therapy off protocol, which may have blunted the survival difference as traditional intent-to-treat analysis can underestimate the treatment effect on OS following treatment switching. (Danner and Sarkar. PharmaSUG. 2018; EP-04) After adjusting for the survival benefit from subsequent cellular immunotherapy among SOC patients using the randomization-based RPSFT model, (Danner and Sarkar. PharmaSUG. 2018; EP-04) axicabtagene ciloleucel demonstrated a statistically significant improvement in OS versus SOC.

The safety profile of axicabtagene ciloleucel in this study was manageable and consistent with previous studies in refractory LBCL. (Neelapu, et al. N Engl J Med. 2017; 377:2531-2544; Locke, et al. Blood. 2017; 130:2826-2826.) Grade ≥3 AEs were numerically similar between patients in the axicabtagene ciloleucel and SOC cohorts (91% and 83%, respectively), with the exception of CRS and neurologic events, as expected. Grade ≥3 CRS and neurologic events were generally consistent with those reported in third-line, (Neelapu, et al. N Engl J Med. 2017; 377:2531-2544.) though notably there were no grade 5 CRS or neurologic events in this study.

Importantly, nearly three times the number of axicabtagene ciloleucel patients received definitive therapy compared to SOC patients. While nearly all patients randomized to axicabtagene ciloleucel were infused with axicabtagene ciloleucel, (Neelapu, et al. N Engl J Med. 2017; 377:2531-2544.) only a minority of patients in the SOC cohort received protocol-defined HDT-ASCT (36%), consistent with historical studies. (Gisselbrecht, et al. J Clin Oncol. 2010; 28:4184-90; van Imhoff, et al. J Clin Oncol. 2017; 35:544-551; Crump, et al. J Clin Oncol. 2014; 32:3490-6.) Given that it is not known a priori which patients will respond to salvage therapy, and since the majority of patients never reach HDT-ASCT definitive therapy, outcomes with the current SOC therapy are sub-optimal.

In this study, bridging therapy was limited to corticosteroids, such as dexamethasone at a dose of 20-40 mg or equivalent, either per os or IV daily for 1-4 days, at the investigator's discretion for patients with high disease burden at screening, administered after leukapheresis, and completed ≥5 days before axicabtagene ciloleucel. Choice of corticosteroid and dosing was adjusted for age/comorbidities or per clinical judgement. Although this potentially limited enrollment of patients requiring emergent therapy, 74% of patients were primary refractory. Prohibiting the use of chemotherapy bridging, which could alone result in a response rate of 40-50%, (Gisselbrecht, et al. J Clin Oncol. 2010; 28:4184-90; van Imhoff, et al. J Clin Oncol. 2017; 35:544-551; Crump, et al. J Clin Oncol. 2014; 32:3490-6.) ensured that results in the axicabtagene ciloleucel cohort were not confounded. In some cases, however, bridging chemotherapy must be started emergently. If a patient has received and responded to salvage chemoimmunotherapy, the improvement in outcomes with axicabtagene ciloleucel over SOC in this study may not apply. This is suggested by the fact that the DOR, while numerically different, was not statistically significant. Once a response with salvage chemotherapy is achieved, a patient proceeding to HDT-ASCT could be expected to have similar benefit as a patient that proceeded directly to axicabtagene ciloleucel without salvage. However, as chemosensitivity is unknown prior to treatment initiation, use of second-line axicabtagene ciloleucel may avoid additional chemotherapy in patients who would ultimately not receive transplant, shorten the time to definitive therapy, and avoid the potential impact on CAR T-cell fitness with greater prior lines of therapy. (Neelapu, et al. ASH Annual Meeting. 2020.).

While the majority of patients with LBCL relapse <12 months after induction in the post-rituximab era, (Vannata, et al. Br J Haematol. 2019; 187:478-487; Hamadani, et al. Biology of Blood and Marrow Transplantation. 2014; 20:1729-1736.) Patients with LBCL relapses occurring >12 months after induction were not enrolled. However, the 2-year EFS with axicabtagene ciloleucel of 40.5% compares favorably with that of patients who received SOC in CORAL following prior rituximab and with relapsed disease >12 months from diagnosis, (Gisselbrecht, et al. J Clin Oncol. 2010; 28:4184-90.) which is generally associated with a greater probability of second-line response. Hence, patients who relapse >12 months of first-line therapy may also benefit from axicabtagene ciloleucel as a therapeutic option regardless of the timing of relapse after first-line therapy.

### Example 6

This study is related to previous Examples as the results were obtained from the same CLINICAL TRIAL-1 registrational Phase 1/2 study of axicabtagene ciloleucel, in patients with refractory LBCL. In CLINICAL TRIAL-1 Cohorts 1+2 (C1+2; N=101), rates of Grade (Gr) ≥3 cytokine release syndrome (CRS) and neurologic events (NEs) were 13% and 28%, respectively, at the 6-month primary analysis; the ORR was 82% (54% CR; Neelapu et al. NEJM. 2017). CLINICAL TRIAL-1 safety management cohort 6 (C6) assessed whether prophylactic and earlier corticosteroids and/or tocilizumab could reduce incidence and severity of CRS and NEs. With a median follow-up of 8.9 months (N=40) for C6, there were no Gr ≥3 CRS, a low rate of Gr ≥3 NEs (13%), and high response rates (Oluwole et al. BJH. 2021). Here, the results of a 1-yr updated analysis of C6 supported by propensity score matching (PSM) analysis to compare outcomes for patients in C6 vs C1+2 are presented. Eligible patients could receive optional bridging therapy after leukapheresis. Patients received conditioning chemotherapy for 3 days prior to a single axicabtagene ciloleucel infusion. Patients received once-daily oral dexamethasone 10 mg on Days 0 (before axicabtagene ciloleucel), 1, and 2, and earlier corticosteroids and/or tocilizumab for AE management. The primary endpoints were incidence and severity of CRS and NEs. Other endpoints included efficacy outcomes and biomarker analyses. To accurately compare results for patients in C6 and C1+2, an exploratory PSM analysis was performed after balancing for key baseline disease characteristics (tumor burden, IPI score, no. of prior lines of chemotherapy, disease stage, and LDH level).

As of Dec. 16, 2020, the median follow-up time was 14.9 months. Median cumulative cortisone-equivalent corticosteroid dose was 1252 mg including prophylaxis (N=40) and 2504 mg excluding prophylaxis (n=25; 15 patients did not receive corticosteroids for AE management). Gr ≥3 AEs were reported in all 40 treated patients, and the most common were neutropenia (45%), neutrophil count decreased (33%), and white blood cell count decreased (23%). No Gr ≥3 CRS occurred. Gr ≥3 NEs were reported in 15% of patients. Median time to CRS and NE onset was 5 and 6 days, respectively, after axicabtagene ciloleucel infusion. Infections of any grade occurred in 50% of patients (20% Gr ≥3). Since the 6-month analysis, no new cases of CRS were observed. Four new axicabtagene ciloleucel-related NEs occurred in 2 patients (patient 1: Gr 2 mental status changes and seizure-like phenomena; patient 2: Gr 1 dementia [occurred on Day 93 but was reported late] and Gr 5 toxic encephalopathy). Two new infections of Gr 2 pneumonia and Gr 1 bronchitis were observed; the latter was axicabtagene ciloleucel-related. One death due to progressive disease occurred. The investigator-assessed ORR was 95% (80% CR). Median DOR, PFS, and OS were not reached. Kaplan-Meier estimates of the 12-mo DOR, PFS, and OS rates were 60%, 63%, and 82%, respectively. At data cutoff, 53% of patients were in ongoing response. Median peak CAR T-cell levels were comparably high in patients with ongoing response and relapse (64 cells/μL [n=21] and 66 cells/μL [n=15], respectively) at 12 months and considerably lower in nonresponders (18 cells/μL [n=2]).

In all, 32 patients each were identified in C6 and matched C1+2 during PSM analysis. Lower incidence and longer median time to onset of Gr ≥3 CRS was observed in C6 (0% and not applicable, respectively) vs C1+2 (13% and 6d). Incidence and median time to onset of Gr ≥3 NEs were 19% and 12 days, respectively, in C6 vs 22% and 7 days in C1+2. The ORR was 94% in both C6 and matched C1+2 (75% and 78% CR rates, respectively); 47% and 59% of patients were in ongoing response, respectively. Median peak CAR T-cell levels were 65 and 43 cells/μL, respectively, in C6 and C1+2. Serum levels of inflammatory biomarkers associated with CAR T-cell treatment-related AEs (IFN-γ, IL-2, GM-CSF, and ferritin) were lower in C6 vs C1+2. Median cumulative corticosteroid dose including prophylaxis was 1252 mg in C6 (n=32) and 7418 mg in C1+2 (n=6).

With ≥1-y follow-up, prophylactic and earlier corticosteroid and/or tocilizumab intervention continued to demonstrate a manageable safety profile, no new safety signals, and high, durable response rates, which was corroborated by PSM analysis. Although fewer patients in C1+2 received corticosteroids after matching, the median cumulative corticosteroid dose was 4-fold lower in C6 vs C1+2.

### Example 7

As described in previous Examples, axicabtagene ciloleucel, an autologous anti-CD19 CAR T-cell therapy, approved for the treatment of patients with relapsed/refractory LBCL with ≥2 prior systemic therapies. In the 2-year analysis of CLINICAL TRIAL-1 (NCT02348216), the multicenter, single-arm phase 1/2 study evaluating axicabtagene ciloleucel in patients with refractory LBCL, the ORR was 83%, including a CR rate of 58%, and 39% of patients had ongoing response with a median follow-up of 27.1 months (Locke et al. Lancet Oncol. 2019). Event-Free Survival (EFS) is emerging as a robust surrogate endpoint for OS in hematologic malignancies. A recent systematic analysis demonstrated a linear correlation between EFS and OS in patients with diffuse LBCL after immunochemotherapy (Zhu et al. Leukemia. 2020). Here, updated survival findings from CLINICAL TRIAL-1 after 4-years of follow-up, including an evaluation of the association of OS with EFS are provided. Eligible patients had refractory LBCL (diffuse LBCL, primary mediastinal B cell lymphoma, transformed follicular lymphoma). After leukapheresis at enrollment, patients received low-dose conditioning chemotherapy (fludarabine and cyclophosphamide) followed by a target dose of 2×10⁶ anti-CD19 CAR T cells/kg (Neelapu et al. N Engl J Med. 2017). The primary endpoint was ORR, with the first response assessment occurring 4 weeks following infusion. Additional endpoints included safety and translational evaluations. An exploratory analysis of OS by EFS at 12 and 24 months was performed. EFS was defined as the time from axicabtagene ciloleucel infusion until disease progression, initiation of new lymphoma therapy (excluding stem cell transplant), or death from any cause. Comparisons of OS by EFS outcomes were analyzed via Kaplan-Meier estimates.

Since the 2-year analysis (Locke et al. Lancet Oncol. 2019), there have been no new safety signals reported, including no new serious adverse events, no axicabtagene ciloleucel-related secondary malignancy, and no confirmed cases of replication-competent retrovirus. Twenty-six patients received subsequent anti-cancer therapy; median time to next therapy was 8.7 months (range, 0.3-53.8). Two patients in axicabtagene ciloleucel-induced remission received allogeneic stem cell transplant. Overall, 66 patients have died (59%), primarily due to progressive disease (47%; n=52), followed by other reasons (7%; n=8), adverse events (5%; n=5), and secondary malignancy unrelated to axicabtagene ciloleucel (1%; n=1).

### Example 8

CLINICAL TRIAL-5 is a Phase 2, multicenter, single-arm study evaluating axicabtagene ciloleucel in patients with R/R iNHL (including FL and marginal zone lymphoma [MZL]). In the primary analysis of CLINICAL TRIAL-5 (N=104), the ORR was 92% (76% CR rate), and median peak CAR T-cell levels were numerically greater in patients with FL who were in ongoing response at 12 months than in those who relapsed (Jacobson et al. ASH 2020. Abstract 700). Here, updated clinical and pharmacologic outcomes from CLINICAL TRIAL-5 are presented. Eligible adults with FL or MZL and R/R disease after ≥2 lines of therapy (including an anti-CD20 mAb plus an alkylating agent) underwent leukapheresis and conditioning chemotherapy followed by a single axicabtagene ciloleucel infusion at 2×10⁶ CART cells/kg. The primary endpoint was centrally assessed ORR per Lugano classification (Cheson, et al. J Clin Oncol. 2014). The updated efficacy analysis occurred when ≥80 consecutively treated patients with FL had ≥2 years of follow-up post-infusion and included patients with MZL who had ≥4 weeks of follow-up post-infusion.

As of Mar. 31, 2021, 149 patients with iNHL (124 FL; 25 MZL) were treated with axicabtagene ciloleucel. Of those, 110 patients (86 FL; 24 MZL) were eligible for efficacy analyses, with a median follow-up of 29.7 months (range, 7.4-44.3). The ORR was consistent with the primary analysis (Jacobson et al. ASH 2020. Abstract 700), with a 94% ORR in patients with FL (79% CR rate) and an 83% ORR in those with MZL (63% CR rate). At data cutoff, 57% of efficacy eligible patients with FL and 50% with MZL had ongoing responses; among those who achieved a CR, 68% with FL and 73% with MZL had ongoing responses. The median DOR was 38.6 months in patients with FL and not reached in those with MZL. Among patients with FL, those who progressed <2 years after initial chemoimmunotherapy (POD24; n=62) had a median DOR of 38.6 months, while median DOR was not reached for those without POD24 (n=37). Median progression-free survival was 39.6 months in FL and 17.3 months in MZL; median time to next treatment was 39.6 months in FL and not reached in MZL. Median OS was not reached in either disease type, with an estimated OS at 24 months of 81% in FL and 70% in MZL, respectively. Common Grade ≥3 AEs in all treated patients with iNHL were consistent with prior reporting: neutropenia (33%), decreased neutrophil count (28%), and anemia (25%). Grade ≥3 cytopenias present ≥30 days post-infusion were reported in 34% of patients with iNHL (33% FL; 36% MZL). Consistent with previous reports, Grade ≥3 cytokine release syndrome (CRS) and neurologic events (NEs) occurred in 7% of patients with iNHL (6% FL; 8% MZL) and 19% of patients (15% FL; 36% MZL), respectively. Most CRS cases (120/121) and NEs (82/87) of any grade resolved by data cutoff. Among patients with FL who had evaluable samples, 76% (65/86) had detectable CAR gene-marked cells at low levels by 12 months post-infusion; 53% (23/43) had detectable cells 24 months post-infusion. Among evaluable patients with MZL, 67% (8/12) had detectable CAR gene-marked cells 12 months post-infusion; 60% (3/5) had detectable cells 24 months post-infusion. B cells were detectable in 59% of evaluable patients with FL (49/83) and 71% of those with MZL (5/7) by 12 months post-infusion.

With nearly 30 months of median follow-up in CLINICAL TRIAL-5, axicabtagene ciloleucel demonstrated substantial and continued long-term benefit in patients with iNHL. In FL, high response rates translated to durability, with a median DOR of 38.6 months and 57% responses ongoing at data cutoff. In MZL, efficacy outcomes appeared to improve with longer follow-up, with the median DOR and OS not yet reached.

### Example 9

The standard of care (SOC) treatment (Tx) in the curative setting for patients with relapsed/refractory (R/R) large B-cell lymphoma (LBCL) after 1st-line (1L) chemoimmunotherapy (CIT) is high-dose therapy with autologous stem cell rescue (HDT-ASCT) if responsive to second line (2L) CIT; however, as many patients do not respond to or cannot tolerate 2L CIT, or are not intended for HDT-ASCT, outcomes remain poor. Axicabtagene ciloleucel has been approved for R/R LBCL after ≥2 prior systemic therapies. Since CAR T-cell therapy may benefit patients in earlier lines of therapy, a global, randomized, Phase 3 trial of axicabtagene ciloleucel vs SOC in patients with 2L R/R LBCL was conducted, and the results of the primary analysis (PA) are reported here. Eligible patients were ≥18 y with LBCL, ECOG PS 0-1, R/R disease ≤12 mo of adequate 1L CIT (including anti-CD20 monoclonal antibody and an anthracycline), and intended to proceed to HDT-ASCT. Patients were randomized 1:1 to axicabtagene ciloleucel or SOC, stratified by 1L Tx response and 2L age-adjusted IPI (sAAIPI). In the axicabtagene ciloleucel arm, patients received a single infusion of 2×10⁶ CAR T cells/kg after conditioning (3 days; cyclophosphamide 500 mg/m²/day and fludarabine 30 mg/m²/day). Optional bridging Tx was limited to corticosteroids (CIT was not allowed). In the SOC arm, patients received 2-3 cycles of an investigator-selected, protocol defined, platinum-based CIT regimen; patients with partial response or CR proceeded to HDT-ASCT. Disease assessments by PET-CT per Lugano Classification occurred at timepoints specified from randomization. Although there was no planned trial crossover between arms, patients not responding to SOC could receive CAR T-cell therapy off protocol. Axicabtagene ciloleucel was hypothesized to result in a 50% improvement in event-free survival (EFS: time to earliest date of disease progression, death from any cause, or new lymphoma Tx) vs SOC. The PA was event-driven, and the primary endpoint was EFS by blinded central review. Key secondary endpoints, tested hierarchically, were objective response rate (ORR) and overall survival (OS; interim analysis); safety was also a secondary endpoint.

As of Mar. 18, 2021, 359 patients were enrolled globally. The median age of patients was 59 years (range, 21-81; 30%≥65 y). Overall, 74% of patients had primary refractory disease and 46% had high sAAIPI (2-3). Of 180 patients randomized to axicabtagene ciloleucel, 170 (94%) were infused. Among 179 patients randomized to SOC, 168 (94%) initiated 2L CIT, 90 (50%) responded, and 64 (36%) reached HDT-ASCT. At 24.9 months median follow-up, median EFS was significantly longer with axicabtagene ciloleucel vs SOC (8.3 mo [95% CI: 4.5-15.8] vs 2 mo [95% CI: 1.6-2.8], respectively; HR: 0.398; P<0.0001), and Kaplan-Meier estimates of the 24-mo EFS rates were significantly higher with axicabtagene ciloleucel (41% vs 16%). Among randomized patients, ORR and CR rates were higher with axicabtagene ciloleucel vs SOC (ORR: 83% vs 50%, odds ratio: 5.31 [95% CI: 3.1-8.9; P<0.0001]; CR: 65% vs 32%). Median OS, evaluated here as a preplanned interim analysis, favored axicabtagene ciloleucel vs SOC, though it did not meet statistical significance (not reached vs 35.1 months, respectively; HR: 0.730; P=0.027). For SOC patients, 100 (56%) received commercially available or investigational CAR T-cell therapy off protocol as subsequent Tx. Grade ≥3 treatment-emergent adverse events occurred in 155 (91%) and 140 (83%) patients, and Tx-related deaths occurred in 1 and 2 patients in the axicabtagene ciloleucel and SOC arms, respectively. In patients treated with axicabtagene ciloleucel, Grade ≥3 cytokine release syndrome (CRS) occurred in 11 (6%) patients (median time to onset 3 days; median duration 7 days) and Grade ≥3 neurologic events (NEs) occurred in 36 (21%) patients (median time to onset 7 days; median duration 8.5 days). No Grade 5 CRS or NEs occurred. Median peak CAR T-cell levels were 25.8 cells/μL; median time to peak was 8 days after infusion.

### Example 10

High-risk LBCL is associated with poor prognosis after first-line anti-CD20 mAb-containing regimens, highlighting the need for novel treatments. Axicabtagene ciloleucel is approved for treatment of relapsed/refractory (R/R) LBCL after ≥2 lines of systemic therapy. Here the primary analysis of a Phase 2, multicenter, single-arm study of axicabtagene ciloleucel as part of first-line therapy in patients with high-risk R/R LBCL after ≥2 lines of systemic therapy is reported. Eligible adults had high-risk LBCL, defined by histology (double- or triple-hit status [MYC and BCL2 and/or BCL6 translocations] per investigator) or an IPI score ≥3, plus a positive interim PET per Lugano Classification (Deauville score [DS] 4/5) after 2 cycles of an anti-CD20 mAb and anthracycline-containing regimen. Patients were leukapheresed and received conditioning chemotherapy (cyclophosphamide and fludarabine) followed by a single axicabtagene ciloleucel infusion at 2×10⁶ CAR T cells/kg. Non-chemotherapy bridging could be administered before conditioning per investigator discretion. The primary endpoint was investigator-assessed complete response (CR) rate per Lugano. Secondary endpoints included objective response rate (ORR; CR+partial response), duration of response (DOR), event-free survival (EFS), progression-free survival (PFS), overall survival (OS), incidence of adverse events (AEs), and levels of CAR T cells in blood and cytokines in serum. The primary analysis occurred after all treated patients had ≥6 months of follow-up.

As of May 17, 2021, 42 patients were enrolled and 40 were treated with axicabtagene ciloleucel. Median age was 61 years (range, 23-86); 68% of patients were male, 63% had ECOG 1, 95% had stage III/IV disease, 48%/53% had DS 4/5; 25% had double- or triple-hit status per central assessment, and 78% had IPI score ≥3. A total of 37 patients had centrally confirmed double- or triple-hit histology or an IPI score ≥3 and were evaluable for response, with 15.9 months of median follow-up (range, 6.0-26.7). The CR rate was 78% (n=29; 95% CI, 62-90); 89% had an objective response, and median time to initial response was 1 month. Among all 40 treated patients, 90% had an objective response (80% CR rate). At data cutoff, 73% of response-evaluable patients had ongoing responses. Medians for DOR, EFS, and PFS were not reached; 12-month estimates were 81%, 73%, and 75%, respectively. The estimated OS at 12 months was 91%. All 40 treated patients had AEs of any grade; 85% of patients had Grade ≥3 AEs, most commonly cytopenias (68%). Grade ≥3 cytokine release syndrome (CRS) and neurologic events (NEs) occurred in 3 patients (8%) and 9 patients (23%), respectively. Median times to onset of CRS and NEs were 4 days (range, 1-10) and 9 days (range, 2-44), respectively, with median durations of 6 days and 7 days. All CRS and most NEs (28/29) of any grade resolved by data cutoff (1 ongoing Grade 1 tremor); 39/40 CRS events resolved by 14 days post-infusion and 19/29 NEs resolved by 21 days post-infusion. Tocilizumab was administered to 63% and 3% of patients for management of CRS or NEs, respectively; corticosteroids were administered to 35% and 33% of patients for CRS and NE management. One Grade 5 event of COVID-19 occurred (Day 350). Median peak CAR T-cell level in all treated patients was 36 cells/μL (range, 7-560) and median expansion by AUC₀₋₂₈ was 495 cells/μL x days (range, 74-4288). CAR T-cell levels peaked at a median of 8 days post-infusion (range, 8-37). Higher frequency of CCR7+CD45RA+ T cells in axicabtagene ciloleucel product, previously associated with greater expansion of CAR T cells (Locke et al. Blood Adv. 2020), was observed, compared with the CLINICAL TRIAL-1 study in R/R LBCL (Neelapu et al, New Engl J Med. 2017).

In the primary analysis axicabtagene ciloleucel showed a high rate of rapid and complete responses in patients with high-risk LBCL, a population with high unmet need. With 15.9 months of median follow-up, responses were durable as medians for DOR, EFS, and PFS were not yet reached and over 70% of patients remained in response at data cutoff. No new safety signals were reported with axicabtagene ciloleucel in an earlier line.

### Example 11

This example relates to and expands upon Example 10. Between Feb. 6, 2019 and Oct. 22, 2020, a total of 42 patients were enrolled and underwent leukapheresis (Table 28). Axicabtagene ciloleucel was manufactured for all 42 patients and administered to 40. One patient did not receive treatment at their request, and one patient was withdrawn from the study prior to treatment due to the discovery of a second primary malignancy. The median time from leukapheresis to delivery of axicabtagene ciloleucel product to the treatment facility was 18 days (range, 14-32; Table 29). The date of data cutoff for the primary analysis was May 17, 2021. The median follow-up time among patients included in the primary efficacy analysis (N=37) was 15.9 months (range, 6.0-26.7), and the median follow-up time among all patients treated with axicabtagene ciloleucel (N=40) was 17.4 months (range, 6.0-26.7).

TABLE 28
Patient Enrollment by Country and Study Site (N = 42)

| | Number of Patients | |
|---|---|---|
| Site | n (%) | |
| United States | 33 | (79) |
| City of Hope National Medical Center | 1 | (2) |
| Moffitt Cancer Center | 16 | (38) |
| The University of Texas MD Anderson | 11 | (26) |
| Cancer Center | | |
| Vanderbilt - Ingram Cancer Center | 1 | (2) |
| Banner MD Anderson Cancer Center | 4 | (10) |
| Australia | 7 | (17) |
| Peter MacCallum Cancer Centre | 7 | (17) |
| France | 2 | (5) |
| Hopital Saint-Louis (AP-HP) - Service | 2 | (5) |
| Hematologie Seniors | | |

TABLE 29
Axicabtagene ciloleucel Product Characteristics
in All Treated Patients (N = 40)

| | All Patients | |
|---|---|---|
| Parameter, Median (Range) | (N = 40) | |
| Total no. of T cells infused × 10⁶, n | 304 | (165-603) |
| Total no. of CAR T cells infused × 10⁶, n | 165 | (95-200) |
| Total no. of CCR7+CD45RA+ T cells* | 105 | (33-254) |
| infused × 10⁶, n | | |
| CCR7+CD45RA+ T cells*, % | 35 | (7-80) |
| Doubling time, days | 1.6 | (1.3-3.4) |
| Time from leukapheresis to delivery to | 18 | (14-32) |
| study site, days | | |

*Data are reported based on the total number of T cells infused and not the CAR+ T-cell population.
Axicabtagene ciloleucel, axicabtagene ciloleucel; CAR, chimeric antigen receptor; CCR7, C-C chemokine receptor type 7.

Among the 40 patients treated with axicabtagene ciloleucel, the median age was 61 years (range, 23-86; Table 30). Patients included 23 (58%) with diffuse LBCL (DLBCL), 12 (30%) with double- or triple-hit lymphomas, 2 (5%) with high-grade B-cell lymphoma-not otherwise specified, and 3 (8%) with their disease classified as other (Table 30). Most of the patients (95%) had stage III or IV disease and 78% had an IPI score ≥3 (Table 30). All patients received 2 cycles of 1 prior systemic therapy, most commonly R-CHOP (48%) or DA-EPOCH-R (45%). The median time from the last dose of prior therapy to leukapheresis was 1 month. All patients were considered high risk either by double- or triple-hit status and/or if they had an IPI score ≥3 anytime between initial diagnosis and enrollment, and all patients were PET2+ per local review with a Deauville PET score of 4 (48%) or 5 (53%). Seven patients received non-chemotherapy bridging therapy after leukapheresis and before conditioning chemotherapy. Five patients received central nervous system (CNS) prophylaxis.

TABLE 30
Baseline patient characteristics for all treated patients (N = 40)

| | Patients | |
|---|---|---|
| Baseline Characteristic | (N = 40) | |
| Age, median (range), years | 61 | (23-86) |
| ≥65 years, n (%) | 15 | (38) |
| Male sex, n (%) | 27 | (68) |

Histological disease type per investigator, n (%)

| | | |
|---|---|---|
| DLBCL not otherwise specified | 23 | (58) |
| HGBL-NOS | 2 | (5) |
| Double- or triple-hit lymphomas | 12 | (30) |
| Other^{a} | 3 | (8) |
| ECOG performance status score of 1^{b}, n (%) | 25 | (63) |

Disease stage, n (%)

| | | |
|---|---|---|
| I or II | 2 | (5) |
| III or IV | 38 | (95) |

IPI total score^{c}, n (%)

| | | |
|---|---|---|
| 1 or 2 | 9 | (23) |
| 3 or 4 | 31 | (78) |

Deauville five-point Scale, n (%)

| | | |
|---|---|---|
| 4 | 19 | (48) |
| 5 | 21 | (53) |

Bone marrow assessment at enrollment^{d}, n (%)

| | | |
|---|---|---|
| Lymphoma present | 10 | (25) |

Double/triple hit status by FISH per
central lab and IPI total score, n (%)

| | | |
|---|---|---|
| Double-/Triple-hit and IPI ≥3 | 4 | (10) |
| Double-/Triple-hit only | 6 | (15) |
| IPI ≥3 only | 20 | (50) |
| Neither Double-/Triple-hit nor IPI ≥3 | 2 | (5) |
| Double-/Triple-hit not done and IPI ≥3 | 7 | (18) |
| Double-/Triple-hit not done and non-IPI ≥3 | 1 | (3) |
| Double expression per central lab, n (%) | 13 | (33) |
| c-Myc expression per central lab, n (%) | 21 | (53) |

Alterations by FISH, per investigator, n (%)

| | | |
|---|---|---|
| MYC | 20 | (50) |
| BCL-2 | 16 | (40) |
| BCL-6 | 11 | (28) |

Prior systemic therapy regimen (2 cycles)^{e}, n (%)

| | | |
|---|---|---|
| R-CHOP | 19 | (48) |
| DA-EPOCH-R | 18 | (45) |
| Neither R-CHOP nor DA-EPOCH-R | 6 | (15) |

Best response to 2 cycles of prior systemic therapy, n (%)

| | | |
|---|---|---|
| PR | 21 | (53) |
| SD | 2 | (5) |
| PD | 16 | (40) |
| NE | 1 | (3) |
| Prior radiotherapy, n (%) | 2 | (5) |
| Received bridging therapy, n (%) | 7 | (17.5) |

^{a}Other disease types included non-GCB subtype, germinal center DLBCL, and high grade B cell lymphoma.
^{b}Four patients had ECOG ≥2 at the time of diagnosis, which was changed to ECOG ≤1 before enrollment.
^{c}IPI measured at initial diagnosis or anytime between initial diagnosis and enrollment.
^{d}Bone marrow assessment at baseline is the last assessment based on biopsy or PET/CT on or before first dose of conditioning chemotherapy.
^{e}Three patients received both R-CHOP and DA-EPOCH-R. Of the 6 patients who did not receive R-CHOP or DA-EPOCH-R, 2 received EPOCH-R, 1 received EPOCH, 1 received EPOCH-R and intrathecal chemotherapy, 1 received R-mini-CHOP, and 1 received CODOX-M.
CODOX-M, cyclophosphamide, vincristine, doxorubicin, high-dose methotrexate; DA-EPOCH-R, dose-adjusted etoposide, prednisone, vincristine, cyclophosphamide, doxorubicin, and rituximab; DLBCL, diffuse large B-cell lymphoma; ECOG, Eastern Cooperative Oncology Group; EPOCH, etoposide, prednisone, vincristine, cyclophosphamide, and doxorubicin; EPOCH-R, etoposide, prednisone, vincristine, cyclophosphamide, doxorubicin, and rituximab; FISH, fluorescent in situ hybridization; GCB, germinal center B-cell; HGBL-NOS, high grade B-cell lymphoma-not otherwise specified; IPI, International Prognostic Index; NE, not evaluable; PD, progressive disease; PR, partial response; R-CHOP, rituximab, cyclophosphamide, doxorubicin, vincristine, and prednisone; R-mini-CHOP, rituximab and reduced dose cyclophosphamide, doxorubicin, vincristine, and prednisone; SD, stable disease.

Per protocol, the primary efficacy analysis included patients with centrally confirmed disease type (double- or triple-hit lymphomas) or IPI score ≥3 who received ≥1×106 CAR T cells/kg. Among the 37 patients included in the primary efficacy analysis, the complete response rate was 78% (95% CI, 62-90). The median time to first complete response was 30 days (range, 27-207). The objective response rate was 89% (95% CI, 75-97), and the median time to first objective response was 29 days (range, 27-207). As of the data cutoff date, 25 patients (86% of complete responders; 68% of patients in the primary efficacy analysis) had an ongoing complete response and 27 patients (82% of objective responders; 73% of patients in the primary efficacy analysis) had an ongoing objective response.

Complete response rates and objective response rates among key subgroups generally aligned with the overall patient population. All 4 patients with double-hit or triple-hit lymphoma and an IPI score ≥3 achieved a complete response; and all 13 patients aged ≥65 years achieved an objective response. The complete response rate for the 6 patients with double-hit or triple-hit lymphoma and an IPI score ≤2 was lower than that of the overall population (50% vs 78%), though sample size was small.

With a median follow-up of 15.9 months at the time of data cutoff, the medians for duration of response, progression-free survival, and event-free survival had not yet been reached. The estimated rates for duration of response, progression-free survival, and event-free survival at 12 months were 81%, 75%, and 73% respectively. The 12-month estimated overall survival rate was 91%. Of the 37 patients included in the primary efficacy analysis, 32 (86%) were still alive at the time of data cutoff. Efficacy outcomes were similar among all patients treated with axicabtagene ciloleucel (N=40; Table 31).

TABLE 31
Key Efficacy Results for Both Patients Included
in the Primary Analysis and All Treated Patients

| | Included in Primary | |
|---|---|---|
| | Efficacy Analysis | All Treated |
| Efficacy analysis | (N = 37) | (N = 40) |
| Complete response, n (%) | 29 (78) | 32 (80) |
| Objective response, n (%) | 33 (89) | 36 (90) |
| Ongoing complete response | 25 (68) | 27 (68) |
| at data cutoff, n (%) | | |
| Ongoing objective response | 27 (73) | 29 (73) |
| at data cutoff, n (%) | | |
| Alive at data cutoff, n (%) | 32 (86) | 34 (85) |

Duration of response rate % by Kaplan-Meier estimate at:

| | | |
|---|---|---|
| 6 months | 89.7 | 90.7 |
| 9 months | 85.3 | 86.8 |
| 12 months | 80.8 | 78.9 |

Overall survival rate % by Kaplan-Meier estimate at:

| | | |
|---|---|---|
| 6 months | 97.3 | 97.5 |
| 9 months | 97.3 | 97.5 |
| 12 months | 90.6 | 87.9 |

Five patients experienced disease progression after an initial response to axicabtagene ciloleucel at the time of data cutoff: one patient was retreated with axicabtagene ciloleucel and achieved a partial response; two patients received subsequent therapies and did not respond; one patient was screened for axicabtagene ciloleucel retreatment and awaits treatment; and one patient is still alive as of the data cutoff date with subsequent therapies unknown. No patients experienced CNS relapse. One patient achieved a partial response as best response to axicabtagene ciloleucel and then proceeded to subsequent therapy which included autologous stem cell transplantation, after which the patient achieved a complete response. Three patients achieved a best response of stable disease to axicabtagene ciloleucel. At the time of data cutoff, one patient had not received subsequent therapy but was still alive, and two patients had received subsequent therapy but died of progressive disease. The one patient who had progressive disease as their best response to axicabtagene ciloleucel went on to receive subsequent therapies but died of progressive disease.

All 40 treated patients experienced at least one adverse event of any grade, with grade ≥3 adverse events experienced by 34 patients (85%). The most common treatment-emergent adverse events of any grade were pyrexia (100%), headache (70%), and decreased neutrophil count (55%). The most common treatment-emergent adverse events of grade ≥3 were decreased neutrophil count (53%), leukopenia (43%), and anemia (30%; Table 32).

TABLE 32
Adverse events occurring in ≥15% of all treated
patients (N = 40) by worst grade

| Adverse Event^{a}, n (%) | Grade 1 | | Grade 2 | | Grade ≥3^{c} | | Total | |
|---|---|---|---|---|---|---|---|---|
| Any adverse event^{b} | 1 | (3) | 5 | (13) | 34 | (85) | 40 | (100) |
| Pyrexia | 8 | (20) | 28 | (70) | 4 | (10) | 40 | (100) |
| Headache | 19 | (48) | 9 | (23) | 0 | (0) | 28 | (70) |
| Neutrophil count | 0 | (0) | 1 | (3) | 21 | (53) | 22 | (55) |
| decreased | | | | | | | | |
| Nausea | 9 | (23) | 11 | (28) | 1 | (3) | 21 | (53) |
| Diarrhoea | 14 | (35) | 6 | (15) | 0 | (0) | 20 | (50) |
| Fatigue | 8 | (20) | 12 | (30) | 0 | (0) | 20 | (50) |
| White blood cell count | 0 | (0) | 1 | (3) | 17 | (43) | 18 | (45) |
| decreased | | | | | | | | |
| Hypotension | 8 | (20) | 5 | (13) | 1 | (3) | 14 | (35) |
| Anaemia | 0 | (0) | 1 | (3) | 12 | (30) | 13 | (33) |
| Chills | 10 | (25) | 1 | (3) | 0 | (0) | 11 | (28) |
| Confusional state | 7 | (18) | 2 | (5) | 2 | (5) | 11 | (28) |
| Hypokalaemia | 8 | (20) | 2 | (5) | 1 | (3) | 11 | (28) |
| Hypoxia | 3 | (8) | 3 | (8) | 5 | (13) | 11 | (28) |
| Encephalopathy | 2 | (5) | 2 | (5) | 6 | (15) | 10 | (25) |
| Sinus tachycardia | 9 | (23) | 1 | (3) | 0 | (0) | 10 | (25) |
| Tremor | 8 | (20) | 2 | (5) | 0 | (0) | 10 | (25) |
| Constipation | 6 | (15) | 2 | (5) | 0 | (0) | 8 | (20) |
| Decreased appetite | 3 | (8) | 5 | (13) | 0 | (0) | 8 | (20) |
| Platelet count decreased | 1 | (3) | 1 | (3) | 6 | (15) | 8 | (20) |
| Vomiting | 3 | (8) | 5 | (13) | 0 | (0) | 8 | (20) |
| Alanine aminotransferase | 1 | (3) | 3 | (8) | 3 | (8) | 7 | (18) |
| increased | | | | | | | | |
| Hypophosphataemia | 0 | (0) | 5 | (13) | 2 | (5) | 7 | (18) |
| Muscular weakness | 4 | (10) | 2 | (5) | 1 | (3) | 7 | (18) |
| Insomnia | 5 | (13) | 1 | (3) | 0 | (0) | 6 | (15) |
| Neutropenia | 0 | (0) | 1 | (3) | 5 | (13) | 6 | (15) |

^{a}Adverse events include those with onset on or after the axicabtagene ciloleucel infusion date and coded using MedDRA Version 23.1 and graded per CTCAE 5.0.
^{b}The first row, showing any adverse event, displays the worst grade event experienced by each of the 40 treated patients.
^{c}One grade 5 event occurred and was reported as COVID-19.

Cytokine release syndrome (CRS) of any grade occurred in all 40 patients (Table 3). Most cases of CRS were grade 1 or 2 (93%), with 3 (8%) being grade ≥3 and no patient died from CRS. The most common CRS symptoms of any grade were pyrexia (100%), hypotension (30%), chills (25%), and hypoxia (23%). The median time to onset for CRS after infusion with axicabtagene ciloleucel was 4 days (range, 1-10; Table 33). All 40 patients (100%) had their CRS resolve by data cutoff, with a median event duration of 6 days. CRS was managed with tocilizumab in 25 patients (63%), steroids in 14 patients (35%), and vasopressors in 1 patient (3%).

TABLE 33
Adverse events of interest occurring in ≥15% of all
treated patients (N = 40) by worst grade

| Adverse Event^{a}, n (%) | Grade 1 | | Grade 2 | | Grade ≥3 | | Total | |
|---|---|---|---|---|---|---|---|---|
| Subjects with any TE | 27 | (68) | 10 | (25) | 3 | (8) | 40 | (100) |
| CRS^{a} | | | | | | | | |
| Pyrexia | 8 | (20) | 28 | (70) | 4 | (10) | 40 | (100) |
| Hypotension | 7 | (18) | 5 | (13) | 0 | (0) | 12 | (30) |
| Chills | 9 | (23) | 1 | (3) | 0 | (0) | 10 | (25) |
| Hypoxia | 2 | (5) | 2 | (5) | 5 | (13) | 9 | (23) |
| Sinus tachycardia | 6 | (15) | 0 | (0) | 0 | (0) | 6 | (15) |
| Subjects with any TE | 14 | (35) | 6 | (15) | 9 | (23) | 29 | (73) |
| neurologic events | | | | | | | | |
| Confusional state | 7 | (18) | 2 | (5) | 2 | (5) | 11 | (28) |
| Encephalopathy | 2 | (5) | 2 | (5) | 6 | (15) | 10 | (25) |
| Tremor | 8 | (20) | 2 | (5) | 0 | (0) | 10 | (25) |

^{a}Adverse events include those with onset on or after the axicabtagene ciloleucel infusion date and coded using MedDRA Version 23.1. Neurologic events were identified using the modified blinatumomab registrational study. Cytokine release syndrome was graded according to Lee et al.³¹ The severity of all adverse events, including neurologic events and symptoms of cytokine release syndrome was graded per CTCAE 5.0.
CRS, cytokine release syndrome, TE, treatment emergent.

Neurologic events of any grade were experienced by 29 (73%) patients, with 9 (23%) cases being grade ≥3. No patient died from a neurologic event. The most common neurologic events of any grade were confusional state (28%), encephalopathy (25%) and tremor (25%). Grade 4 serious adverse events of encephalopathy were experienced by 2 patients (5%); both events fully resolved by data cutoff. The median time to onset for neurologic events was 9 days (range, 2-44) and the median event duration was 7 days. As of the data cutoff, neurologic events had resolved in 28 patients, with 1 patient experiencing an ongoing neurologic event of grade 1 tremor. Neurologic events were managed with steroids in 13 patients (33%) and tocilizumab in 1 patient (3%). Additionally, no patient required mechanical ventilation for the management of neurologic events and no patient died of neurological toxicity.

Serious adverse events of any grade were experienced by 18 patients (45%; Table 34). A total of 13 patients (33%) experienced infection of any grade (Table 35); 3 of these events were COVID-19 infection, including one each grade 2 and grade 5 COVID-19 infections (patients did not report receiving a vaccination against COVID-19) and one grade 3 COVID-19 pneumonia (patient was fully vaccinated against COVID-19). The remaining 10 adverse events of infection were grade 3 (n=4), grade 2 (n=3), or grade 1 (n=3) and included a grade 1 event of cytomegalovirus infection. A total of 4 patients (10%) had adverse events of hypogammaglobulinemia; all 4 events were grade 2. Grade ≥3 cytopenias were present in 68% of patients (n=27). Grade ≥3 cytopenias present on or after day 30 were experienced by 8 patients (20%). All cytopenias of any grade resolved by the data cutoff, with a median duration of 0.5 months. No cases of tumor lysis syndrome, replication-competent retrovirus, or secondary malignancies related to axicabtagene ciloleucel were reported.

TABLE 34
Serious Adverse Events Occurring in
at Least 2 Treated Patients (N = 40)

| MedDRA | | | | | | |
|---|---|---|---|---|---|---|
| Preferred | | | | | | |
| Term, | Any | Worst | Worst | Worst | Worst | Worst |
| n (%) | Grade | Grade 1 | Grade 2 | Grade 3 | Grade 4 | Grade 5 |
| Patients | 18 (45) | 3 (8) | 1 (3) | 10 (25) | 3 (8) | 1 (3) |
| with any | | | | | | |
| serious | | | | | | |
| TEAEs | | | | | | |
| Encepha- | 5 (13) | 0 (0) | 0 (0) | 3 (8) | 2 (5) | 0 (0) |
| lopathy | | | | | | |
| Confu- | 4 (10) | 1 (3) | 1 (3) | 2 (5) | 0 (0) | 0 (0) |
| sional | | | | | | |
| state | | | | | | |
| Pyrexia | 3 (8) | 3 (8) | 0 (0) | 0 (0) | 0 (0) | 0 (0) |
| Back pain | 2 (5) | 0 (0) | 1 (3) | 1 (3) | 0 (0) | 0 (0) |
| Non- | 2 (5) | 0 (0) | 1 (3) | 1 (3) | 0 (0) | 0 (0) |
| cardiac | | | | | | |
| chest | | | | | | |
| pain | | | | | | |

TEAE include all AEs with onset on or after axicabtagene ciloleucel infusion date. AEs with onset during retreatment period are excluded. Multiple incidences of the same AE in one patient are counted once at the worst grade for tht patient.
Preferred terms are sorted in descending order of frequency count in any grade.
AEs are coded using MedDRA Version 23.1 and graded per CTCAE 5.0.AE, adverse event; CTCAE, Common Terminology Criteria for Adverse Event; MedDRA, Medical Dictionary for Regulatory Activities; TEAE, treatment-emergent adverse event.

TABLE 35
Infections Occurring Among All Treated Patients (N = 40)

| Preferred | Any | Worst | Worst | Worst | Worst | Worst |
|---|---|---|---|---|---|---|
| Term, n (%) | Grade | Grade 1 | Grade 2 | Grade 3 | Grade 4 | Grade 5 |
| Infections | 13 (33) | 3 (8) | 4 (10) | 5 (13) | 0 (0) | 1 (3) |
| Urinary | 3 (8) | 2 (5) | 0 (0) | 1 (3) | 0 (0) | 0 (0) |
| tract | | | | | | |
| infection | | | | | | |
| COVID-19 | 2 (5) | 0 (0) | 1 (3) | 0 (0) | 0 (0) | 1 (3) |
| Bronchitis | 1 (3) | 0 (0) | 1 (3) | 0 (0) | 0 (0) | 0 (0) |
| COVID-19 | 1 (3) | 0 (0) | 0 (0) | 1 (3) | 0 (0) | 0 (0) |
| pneumonia | | | | | | |
| Cytomega- | 1 (3) | 0 (0) | 0 (0) | 1 (3) | 0 (0) | 0 (0) |
| lovirus | | | | | | |
| infection | | | | | | |
| Reactivation | | | | | | |
| Lower | 1 (3) | 1 (3) | 0 (0) | 0 (0) | 0 (0) | 0 (0) |
| respiratory | | | | | | |
| tract | | | | | | |
| infection | | | | | | |
| Periorbital | 1 (3) | 0 (0) | 0 (0) | 1 (3) | 0 (0) | 0 (0) |
| infection | | | | | | |
| Sinusitis | 1 (3) | 0 (0) | 1 (3) | 0 (0) | 0 (0) | 0 (0) |
| Skin | 1 (3) | 0 (0) | 0 (0) | 1 (3) | 0 (0) | 0 (0) |
| infection | | | | | | |
| Urethritis | 1 (3) | 0 (0) | 1 (3) | 0 (0) | 0 (0) | 0 (0) |
| Wound | 1 (3) | 0 (0) | 1 (3) | 0 (0) | 0 (0) | 0 (0) |
| infection | | | | | | |
| Wound | 1 (3) | 0 (0) | 1 (3) | 0 (0) | 0 (0) | 0 (0) |
| infection | | | | | | |
| staphylo- | | | | | | |
| coccal | | | | | | |

A total of 6 patients (15%) among those treated with axicabtagene ciloleucel died, four of whom died from progressive disease after proceeding to subsequent therapies (10%). The other 2 deaths were due to COVID-19 (day 350 postinfusion) and septic shock (day 287 postinfusion). Only the death from COVID-19 was reported as an adverse event. The septic shock was reported after the patient had proceeded to subsequent therapy.

CAR T-cell expansion was observed in peripheral blood in all 40 patients. Median peak CAR T cell levels was 36.27 cells/μL, and median area under the curve in a plot of CAR T cells in blood against scheduled visit from Day 0 to Day 28 (AUC₀₋₂₈) was 495.38 cells/μL x days. Median time to peak anti-CD19 CAR T-cell levels in blood was 8 days (range, 8-37; Table S6). Pharmacokinetic profiles were similar across patients of different diagnostic categories, including patients with double- or triple-hit lymphoma and IPI score ≥3 (Table 36). At 6 months after infusion, 13 of 21 patients (62%) with evaluable samples maintained low, but detectable levels CAR gene-marked cells in blood. Three patients had samples evaluable at the approximate time of their relapse, 2 of whom had detectable CAR gene-marked cells in the blood. Two additional patients who relapsed did not have evaluable samples at the time of relapse; however, they had detectable CAR gene-marked cells in blood at the last time point assessed prior to relapse (days 85 and 145).

TABLE 36
Number of Anti-CD19 CAR T Cells in Blood Over Time by Double-/Triple-hit Status Per Central Lab

| | Double-/Triple-hit | Double-/Triple-hit | Double-/Triple-hit | Non-double-/Triple-hit | |
|---|---|---|---|---|---|
| Parameter, | lymphomas | with IPI score ≥3 | with IPI score <3 | with IPI score ≥3 | Overall^{a} |
| Median (Range) | (N = 10) | (N = 4) | (N = 6) | (N = 20) | (N = 40) |
| AUC₀₋₂₈ (cells/μL*days) | 516.58 | 508.45 | 516.58 | 400.69 | 495.38 |
| | (151.42-1374.34) | (355.17-1374.34) | (151.42-1168.76) | (249.01-1133.99) | (74.46-4287.97) |
| Peak (cells/μL) | 44.24 | 36.80 | 50.26 | 35.81 | 36.27 |
| | (10.40-139.19) | (19.90-130.65) | (10.40-139.19) | (12.60-560.33) | (6.79-560.33) |
| Time to Peak (Days) | 8 | 12 | 8 | 8 | 8 |
| | (8-15) | (8-15) | (8-14) | (8-37) | (8-37) |

All data have units of cells/μL except AUC₀₋₂₈ is measured in cells/μL*days and time to peak is measured in days.
AUC₀₋₂₈ is defined as the AUC in a plot of number of CAR T cells in blood against scheduled visit from Day 0 to Day 28. Peak is defined as the maximum number of CAR T cells in blood measured after infusion. Time to peak is defined as the number of days from axicabtagene ciloleucel infusion to the date when the number of CAR T cells in blood first reached the maximum post-baseline level.
^{a}All patients in the analysis set including 2 patients in Non-double-/Triple-hit with IPI score <3 and 8 patients in Double-/Triple-hit Status Not Done.
AUC, area under the curve; CAR, chimeric antigen receptor; IPI, International Prognostic Index.

The median peak levels of CART cells and AUC₀₋₂₈ among patients who relapsed or did not respond trended higher but were not significantly different from those who had an ongoing response as of the data cutoff date. CAR T-cell persistence declined similarly among patients who had an ongoing response compared with those who had relapsed disease or did not respond to axicabtagene ciloleucel. Additionally, no trend was found between peak or AUC₀₋₂₈ and response.

Patients with a tumor burden per sum of product diameters below the median baseline tumor burden value (2778 mm2) had a lower median peak level of CAR T cells, a lower AUC₀₋₂₈, and a lower average time to peak compared with patients who had a baseline tumor burden above 2778 mm² (though differences were not statistically significant). Patients who experienced grade ≥3 CRS (n=3) had peak levels of CAR T cells in blood and AUC₀₋₂₈ that had a median ratio of 4.0× and 2.2× that of patients who experienced grade 2, grade 1, or no CRS. Patients who experienced grade ≥3 neurologic events had peak levels of CART cells in blood and AUC0-28 that had a median ration of 2.1× and 2.3× that of patients who experienced grade 2, grade 1, or no neurologic event, although the difference between the 2 groups was not significantly different.

Median time to peak of most serum cytokines was within 8 days. Several serum analytes were elevated in patients experiencing grade ≥3 CRS or neurologic events, compared with those who had grade 2, grade 1, or no CRS or neurologic events. Among the serum analytes that were at least twice as high at peak among patients who experienced grade ≥3 neurologic events compared with those who did not, interleukin (IL)-5, MIP-1α, IFN-γ, granulocyte-macrophage colony-stimulating factor (GM-CSF), ferritin, TNF-α, IL-10, IL-8, and PDL1 were all determined to be significantly higher (P<0.05). Serum cytokine peak values that were at least four times as high among patients who experienced grade ≥3 CRS compared with those who did not were analyzed but not assessed for significance due to the small patient population size who experienced grade ≥3 CRS (n=3). The most highly elevated serum cytokines among those experiencing grade ≥3 CRS were IL-6, IL-8, and GM-CSF.

### Example 12

A Phase 3 randomized clinical trial in 2L R/R LBCL demonstrated axicabtagene ciloleucel superiority over standard of care (SOC) salvage chemotherapy and high-dose chemotherapy with autologous transplant in event-free survival (EFS; hazard ratio [HR], 0.398; P<0.0001; Locke et al. N Eng J Med. 2021). Disclosed herein are the exploratory endpoint of tumor characteristics, including preTx tumor burden (TB), tissue hypoxia-related lactate dehydrogenase (LDH) level, and tumor microenvironment (TME).

Methods:

TB was calculated as the sum of product diameters (SPD) of ≤6 reference lesions. Serum LDH was assessed. PreTx tumor samples in both treatment arms were used for molecular assessments. Tumor RNA expression was analyzed by the NanoString IO 360™ panel and prespecified immune contexture indexes related to T-cell involvement (Immunosign 15 [IS15] and 21 [IS21]). Tumor RNA expression data from a previous clinical study were used for comparison to pts with 3L R/R LBCL. H-score of CD19 protein expression was assessed by immunohistochemistry. Associations between tumor-related molecular signatures and clinical outcomes were assessed. Descriptive statistics were performed (P<0.05 indicates significance).

Results:

EFS in axicabtagene ciloleucel pts was not associated with preTx TB (HR, 1.01 [95% CI, 0.88-1.16]; P=0.89) or LDH (HR, 0.98 [95% CI, 0.74-1.29]; P=0.86), but was worse in SOC pts with higher preTx TB (HR, 1.17 [95% CI, 1.03-1.32]; P=0.01) or higher LDH (HR, 1.29 [95% CI, 1.02-1.63], P=0.03). PreTx TB was lower in SOC pts with ongoing response versus nonresponders or those who relapsed (P=0.16), but not in axicabtagene ciloleucel pts (P=1). Non-GCB cell-of-origin subtypes is a poor prognostic factor for EFS in SOC but not in axicabtagene ciloleucel. EFS was significantly worse in SOC pts with non-GCB versus GCB (HR, 1.82 [95% CI, 1.12-2.96]; P=0.02). 10360 analysis showed that gene expression of B-cell lineage antigens (CD19, CD20, and BCMA) and markers highly expressed by tumor cells (CD45RA, IRF8, and BTLA) positively associated with objective and durable responses to axicabtagene ciloleucel. Although axicabtagene ciloleucel remained superior to SOC regardless of CD19 expression level, the probability of an ongoing response increased with a higher CD19 H-score. PreTx TME IS15 and IS21 scores were generally higher in 2L versus 3L.

Conclusions:

In pts with R/R LBCL, axicabtagene ciloleucel was superior to SOC across major prognostic groups, like higher TB and LDH. Axicabtagene ciloleucel showed greatest potential for durable response in tumors with prominent B-cell features but was superior to SOC regardless of these features. Earlier intervention with axicabtagene ciloleucel is further supported by a TME with higher immune infiltration in the 2L versus 3L setting, suggesting that a deeper response to 2L axicabtagene ciloleucel in pts with high TB may be attributed to a more favorable immune contexture.

### Example 13

Background:

Elderly pts with R/R LBCL are at risk of inferior outcomes, increased toxicity, and inability to tolerate second-line (2L) SOC treatment (Tx). Further 2L SOC Tx is often associated with poor health-related quality of life. In a clinical study, we assessed outcomes, including PROs, of 2L axicabtagene ciloleucel vs SOC in elderly pts with R/R LBCL.

Methods:

Pts aged ≥65 y were assessed in a planned subgroup analysis. Pts with ECOG PS 0-1 and R/R LBCL ≤12 mo after 1L chemoimmunotherapy (CIT) were randomized 1:1 to axicabtagene ciloleucel or SOC (2-3 cycles of platinum-based CIT; pts with partial or complete response (CR) proceeded to HDT-ASCT). PRO instruments, including the EORTC QLQ-C30 (Global Health [GH] and Physical Functioning [PF]) and the EQ-5D-5L VAS, were administered at timepoints including baseline (BL; prior to Tx), Day (D) 50, D100, D150, and Month (M) 9, then every 3 mo up to 24 mo or time of event-free survival event (EFS), whichever occurred first. The QoL analysis set included all pts who had a BL PRO and >1 completed measure at D50, D100, or D150. A clinically meaningful change was defined as 10 points for each EORTC QLQ-C30 score, 7 points for EQ-5D-5L VAS score.

Results:

51 and 58 elderly pts were randomized to the axicabtagene ciloleucel and SOC arms, respectively, with median ages (range) of 70 y (65-80) and 69 y (65-81). At BL, more axicabtagene ciloleucel vs SOC pts had high-risk features, including 2L age-adjusted IPI 2-3 (53% vs 31%) and elevated LDH (61% vs 41%). EFS was superior with axicabtagene ciloleucel vs SOC (HR, 0.276, P<0.0001), with higher CR rates (75% vs 33%). Grade ≥3 Tx-emergent adverse events (AEs) occurred in 94% and 82% of axicabtagene ciloleucel and SOC pts, respectively, and Grade 5 Tx-related AEs occurred in 0 and 1 pt. In the QoL analysis set comprising 46 axicabtagene ciloleucel and 42 SOC pts, there were statistically significant and clinically meaningful differences in mean change of scores from BL at D100 favoring axicabtagene ciloleucel for EORTC QLQ-C30 GH (P<0.0001) and PF (P=0.0019) and EQ-5D-5L VAS (P<0.0001). For all 3 domains, scores also favored (P<0.05) axicabtagene ciloleucel over SOC at D150. The mean estimated scores numerically returned to or exceeded BL scores earlier in the axicabtagene ciloleucel arm (by D150) but never equaled or exceed BL scores by M15 in the SOC arm.

Conclusions:

Axicabtagene ciloleucel demonstrated superiority over 2L SOC in pts ≥65 y with significantly improved EFS and a manageable safety profile. Compared with SOC, axicabtagene ciloleucel also showed meaningful improvement in QoL over SOC, measured by multiple validated PRO instruments, with suggested faster recovery to pre-Tx QoL. The superior clinical outcomes and pt experience with axicabtagene ciloleucel over SOC should help inform Tx choices in 2L R/R LBCL for pts ≥65 y.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

We claim:

1. A method for treating a malignancy in a patient comprising:
assessing a level of myeloid inflammation in a tumor of the patient comprising measuring a gene expression level of at least one gene selected from the group consisting of ARG2, TREM2, IL8, IL13, CBG, CCL20, IFNL2, OSM, IL11RA, CCL11, MCAM, PTGDR2, and CCL16;
determining whether the patient should be administered an effective dose of engineered lymphocytes, or an effective dose of engineered lymphocytes and a combination therapy at least in part from the measuring the gene expression level of at least one gene; and
administering the effective dose of engineered lymphocytes, or the effective dose of engineered lymphocytes and the combination therapy based on the determining step,
wherein the patient is administered the effective dose of engineered lymphocytes if the gene expression level of the at least one gene is below a predetermined level, and wherein the patient is administered the effective dose of engineered lymphocytes and the combination therapy if the gene expression level of the at least one gene is above the predetermined level.

2. The method of claim 1, wherein the combination therapy comprises at least one of an agent that enhances T-cell proliferation, and an agent that reduces a myeloid population in the tumor.

3. The method of claim 2, wherein the at least one agent comprises an anti-CD47 antagonist, a STING agonist, an ARG1/2 inhibitor, a CD73xTGFβ mAb, a CD40 agonist, a FLT3 agonist, a CSF/CSF1R inhibitor, an IDO1 inhibitor, a TLR agonist, a PD-1 inhibitor, an immunomodulatory imide drug, a CD20xCD3 bispecific antibody, an agent that targets an epigenetic landscape within the tumor or a T-cell costimulatory agonist, or combinations thereof.

4. The method of claim 1, further comprising:
determining a tumor burden in the patient; and
administering the effective dose of engineered lymphocytes, or the effective dose of engineered lymphocytes and the combination therapy based on the determining the tumor burden in the patient,
wherein the patient is administered the effective dose of engineered lymphocytes if the tumor burden is below a reference tumor burden value, and wherein the patient is administered the effective dose of engineered lymphocytes and the combination therapy if the tumor burden is above the reference tumor burden value.

5. The method of claim 4, wherein the reference tumor burden value comprises a baseline tumor burden (SPD) of greater than 2500 mm² or a tumor metabolic volume above a median for a representative tumor population.

6. The method of claim 4, wherein the combination therapy comprises at least one of an agent that enhances T-cell proliferation, and an agent that reduces a myeloid population in the tumor.

7. The method of claim 1, further comprising
quantifying a tumor myeloid cell density in the tumor; and
administering the effective dose of engineered lymphocytes, or the effective dose of engineered lymphocytes and the combination therapy based on the quantifying a tumor myeloid cell density in the tumor,
wherein the patient is administered the effective dose of engineered lymphocytes if the tumor myeloid cell density in the tumor is below a predetermined myeloid cell density level, and wherein the patient is administered the effective dose of engineered lymphocytes and the combination therapy if the tumor myeloid cell density in the tumor is above the predetermined myeloid cell density level.

8. The method of claim 7, wherein the tumor myeloid cell density is quantified comprising measuring levels of CD14+ cells, CD68+ cells, CD68+CD163+ cells, CD68+CD206+ cells, CD11b+CD15+CD14− LOX-1+ cells, or CD11b+CD15− CD14+S100A9+CD68− cells.

9. The method of claim 1, wherein the predetermined level is a median expression level of the at least one gene in a representative tumor population.

10. The method of claim 1, wherein the engineered lymphocytes are chimeric antigen receptor T-cells.

11. The method of claim 1, wherein the effective dose of engineered lymphocytes or the effective dose of engineered lymphocytes and a combination therapy are administered as a first line therapy or as a second line therapy.

12. The method of claim 1, wherein the malignancy is a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder, monoclonal gammapathy of undetermined significance (MGUS), plasmacytomas, systemic amyloid light chain amyloidosis, POEMS syndrome, head and neck cancers, cervical cancers, ovarian cancers, non-small cell lung carcinomas, hepatocellular carcinomas, prostate cancers, breast cancers, or a combination thereof.

13. A method of predicting a clinical efficacy of an immunotherapy in a patient in need thereof comprising:
assessing a level of myeloid inflammation in a tumor of the patient comprising measuring a gene expression level of at least one gene selected from the group consisting of ARG2, TREM2, IL8, IL13, CBG, CCL20, IFNL2, OSM, IL11RA, CCL11, MCAM, PTGDR2, and CCL16; and
determining a likelihood of clinical efficacy of the immunotherapy in the patient at least in part from the gene expression level,
wherein the likelihood of clinical efficacy is inversely related to the gene expression level.

14. The method of claim 13, further comprising measuring a ratio of activated T-cells to suppressive myeloid cells in the tumor, wherein the likelihood of clinical efficacy is related to the ratio of activated T cells to suppressive myeloid cells in the tumor such that a higher ratio of an activated T cells index to a suppressive myeloid cells index in the tumor is indicative of an increased likelihood of clinical efficacy.

15. The method of claim 14, wherein the activated T-cell index is determined comprising measuring a gene expression level of one or more of CD3D, CD8A, CTLA4, and TIGIT in the tumor.

16. The method of claim 13, further comprising determining a tumor burden of the patient, wherein the likelihood of clinical efficacy is related to the tumor burden of the patient such that a tumor burden above a reference tumor burden value is indicative of a reduced likelihood of clinical efficacy and a tumor burden below a reference tumor burden value is indicative of an increased likelihood of clinical efficacy, and wherein the reference tumor burden is 2500 mm².

17. The method of claim 13, wherein the clinical efficacy is assessed comprising evaluating a complete response rate, an objective response rate, an ongoing response rate, a median durability of response, a median progression-free survival, a median overall survival, or any combination thereof.

18. A method of predicting a suppressive tumor microenvironment (TME) in a patient comprising:
assessing a level of myeloid inflammation in a tumor of the patient comprising measuring a gene expression level of at least one gene selected from the group consisting of ARG2, TREM2, IL8, IL13, CBG, CCL20, IFNL2, OSM, IL11RA, CCL11, MCAM, PTGDR2, and CCL16; and
determining a level of the tumor suppressive microenvironment at least in part from the gene expression level,
wherein the level of the tumor suppressive microenvironment is related to the gene expression level such that a higher gene expression level is indicative of a higher suppressive tumor microenvironment.

19. The method of claim 18, further comprising quantifying a tumor myeloid cell density in the tumor, wherein the level of the tumor suppressive microenvironment is related to the tumor myeloid cell density, such that a higher tumor myeloid cell density is indicative of a higher suppressive tumor microenvironment.

20. The method of claim 18, further comprising measuring a ratio of activated T-cells to suppressive myeloid cells in the tumor, wherein the level of the tumor suppressive microenvironment is related to the ratio of activated T-cells to suppressive myeloid cells in the tumor, such that a lower ratio of an activated T-cells index to a suppressive myeloid cells index in the tumor is indicative of a higher suppressive tumor microenvironment.
